(19) United States
(12) Patent Application Publication
(10) Pub. No.: US 2024/0324449 A1
(43) Pub. Date: Sep. 26, 2024

(54) LIGHT EMITTING ELEMENT AND AMINE COMPOUND FOR THE SAME

(71) Applicant: Samsung Display Co., Ltd., Yongin-si (KR)

(72) Inventor: Akinori YAMATANI, Yokohama (JP)

(21) Appl. No.: 18/593,746

(22) Filed: Mar. 1, 2024

(30) Foreign Application Priority Data
Mar. 2, 2023 (KR) 10-2023-0027782

(51) Int. Cl. H10K 85/60; C07C 211/61; C07D 307/91; C09K 11/06; H10K 50/18

(52) U.S. Cl. CPC H10K 85/633; C07C 211/61; C07D 307/91; C09K 11/06; H10K 85/636; C07C 2603/18; C07C 2603/97; C09K 2211/1003; C09K 2211/1018; H10K 50/181; H10K 85/624; H10K 85/626; H10K 85/6574

(57) ABSTRACT

A light emitting element and an amine compound for the light emitting element are provided. The light emitting element includes a first electrode, a second electrode provided on the first electrode, and at least one functional layer provided between the first electrode and the second electrode and including an amine compound represented by a specific formula structure, and may thus have improved element service life (i.e., lifespan) and efficiency.

### CROSS-REFERENCE TO RELATED APPLICATION

This application claims priority to and the benefit of Korean Patent Application No. 10-2023-0027782, filed on Mar. 2, 2023, the entire content of which is hereby incorporated by reference.

### BACKGROUND

### 1. Field

The present disclosure herein relates to a light emitting element and an amine compound for the light emitting element, and for example, a light emitting element including a novel amine compound in a functional layer.

### 2. Description of the Related Art

As image display devices, organic electroluminescence display devices and/or the like have been actively developed lately. The organic electroluminescence display devices and/or the like are display devices including so-called “self-luminescent light emitting elements” in which holes and electrons injected from a first electrode and a second electrode recombine in an emission layer. Subsequently, a luminescent material in the emission layer emits light to accomplish display (e.g., of an image).

Implementation of the light emitting elements to image display devices, requires (desires or there is a demand for) relatively high efficiency and/or long lifespan. Therefore, the need or desire for the development of materials for light emitting elements which are capable of stably attaining such positive characteristics is continuously desired or required.

### SUMMARY

One or more aspects of embodiments of the present disclosure are directed toward a light emitting element exhibiting relatively high efficiency and long service life (i.e., lifespan). One or more aspects of embodiments of the present disclosure are directed toward an amine compound included in the light emitting element.

One or more embodiments of the present disclosure provide a light emitting element including a first electrode, a second electrode provided on the first electrode, and an amine compound of one or more embodiments in at least one functional layer provided between the first electrode and the second electrode. The amine compound of one or more embodiments may be represented by Formula 1.

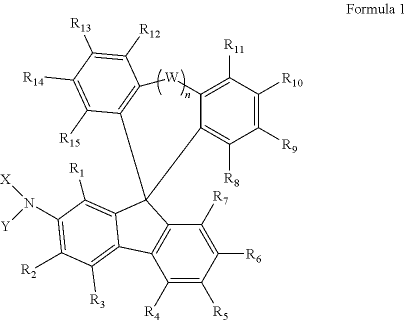

In Formula 1, R₁ to R₁₅ may each independently be a hydrogen atom, a deuterium atom, a halogen atom, a substituted or unsubstituted amine group, a substituted or unsubstituted silyl group, a substituted or unsubstituted aryl group having 6 to 30 ring-forming carbon atoms, or a substituted or unsubstituted heteroaryl group having 2 to 30 ring-forming carbon atoms. In Formula 1, at least one selected from among R₃ to R₅, and R₇ to R₁₅ may be a substituted or unsubstituted aryl group having 6 to 30 ring-forming carbon atoms, or a substituted or unsubstituted heteroaryl group having 2 to 30 ring-forming carbon atoms. In Formula 1, W may be O, S, NRₐ₁, CRₐ₂Rₐ₃, or a direct linkage, n may be 0 or 1, Rₐ₁ may be a substituted or unsubstituted aryl group having 6 to 30 ring-forming carbon atoms, or a substituted or unsubstituted heteroaryl group having 2 to 30 ring-forming carbon atoms, Rₐ₂ and Rₐ₃ may each independently be a substituted or unsubstituted aryl group having 6 to 30 ring-forming carbon atoms, or a substituted or unsubstituted heteroaryl group having 2 to 30 ring-forming carbon atoms, or Rₐ₂ and Rₐ₃ may be bonded to form a ring. In Formula 1, X and Y may each independently be represented by Formula 2 or Formula 3, at least one of X or Y may be represented by Formula 2 and the other may be represented by Formula 3.

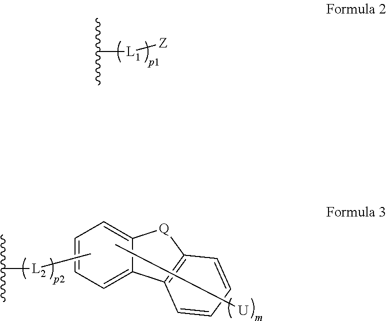

In Formulas 2 and 3, L₁ and L₂ may each independently be a substituted or unsubstituted arylene group having 6 to 50 ring-forming carbon atoms, or a substituted or unsubstituted heteroarylene group having 2 to 50 ring-forming carbon atoms, p1 may be an integer of 1 to 3, and p2 may be an integer of 0 to 3. In Formula 2, Z may be a substituted or unsubstituted naphthyl group, or a substituted or unsubstituted phenanthrenyl group. In Formula 3, U may be a hydrogen atom, a deuterium atom, a halogen atom, a substituted or unsubstituted amine group, a substituted or unsubstituted silyl group, a substituted or unsubstituted aryl group having 6 to 30 ring-forming carbon atoms, or a substituted or unsubstituted heteroaryl group having 2 to 30 ring-forming carbon atoms, and m may be an integer of 0 to 7. In Formula 3, Q may be O, S, NRₐ₄, or CRₐ₅Rₐ₆, Rₐ₄ may be a substituted or unsubstituted aryl group having 6 to 30 ring-forming carbon atoms, or a substituted or unsubstituted heteroaryl group having 2 to 30 ring-forming carbon atoms, Rₐ₅ and Rₐ₆ may each independently be a substituted or unsubstituted aryl group having 6 to 30 ring-forming carbon atoms, or a substituted or unsubstituted heteroaryl group having 2 to 30 ring-forming carbon atoms, or Rₐ₅ and Rₐ₆ may be bonded to form a ring. However, a case in which Formula 3 is a substituted or unsubstituted 2-dibenzothiophene group is excluded.

In one or more embodiments, the at least one functional layer may include an emission layer, a hole transport region provided between the first electrode and the emission layer, and an electron transport region provided between the emission layer and the second electrode, and the hole transport region may include the amine compound represented by Formula 1.

In one or more embodiments, the hole transport region may include at least one of a hole injection layer, a hole transport layer, or an electron blocking layer, and the at least one of the hole injection layer, the hole transport layer, or the electron blocking layer may include the amine compound represented by Formula 1.

In one or more embodiments, the hole transport region may include a hole injection layer or a hole transport layer provided on the first electrode, and an electron blocking layer provided on the hole injection layer or the hole transport layer, and the electron blocking layer may include the amine compound.

In one or more embodiments, Formula 1 may be represented by Formula 1-1 or Formula 1-2.

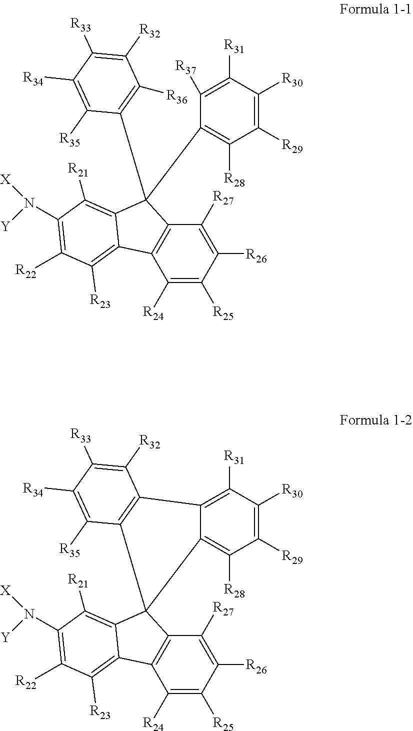

In Formulas 1-1 and 1-2, R₂₁ to R₃₅ may each independently be a hydrogen atom, a deuterium atom, or a substituted or unsubstituted aryl group having 6 to 30 ring-forming carbon atoms, at least one selected from among R₂₃ to R₂₅, and R₂₇ to R₃₅ is a substituted or unsubstituted aryl group having 6 to 30 ring-forming carbon atoms, R₃₆ and R₃₇ may each independently be a hydrogen atom or a deuterium atom, and X and Y may each independently be the same as defined in Formula 1.

In one or more embodiments, Formula 1 may be represented by Formula 4-1 or Formula 4-2.

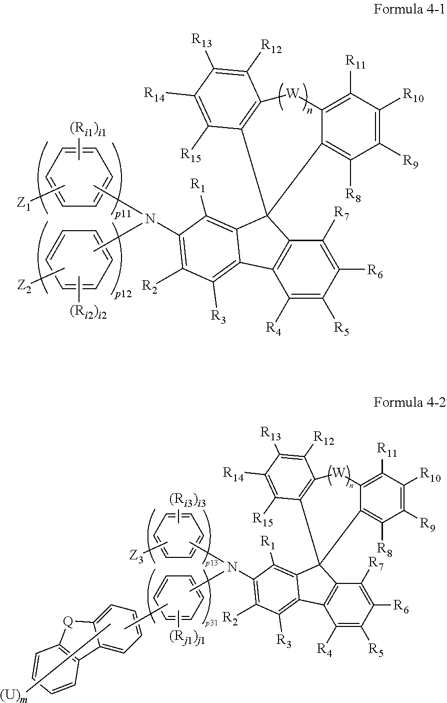

In Formulas 4-1 and 4-2, Z₁ to Z₃ may each independently be a substituted or unsubstituted naphthyl group or a substituted or unsubstituted phenanthrenyl group, Rᵢ₁ to Rᵢ₃, and Rⱼ₁ may each independently be a hydrogen atom, a deuterium atom, a substituted or unsubstituted silyl group, a substituted or unsubstituted amine group, a substituted or unsubstituted aryl group having 6 to 15 ring-forming carbon atoms, or a substituted or unsubstituted heteroaryl group having 2 to 15 ring-forming carbon atoms, i1 to i3, and j1 may each independently be an integer of 0 to 4, p11 to p13 may each independently be an integer of 1 to 3, p21 is an integer of 0 to 3, and R₁ to R₁₅, W, Q, U, n, and m may each independently be the same as defined in Formula 1.

In one or more embodiments, Formula 4-1 may be represented by Formula 4-1-1 or Formula 4-1-2.

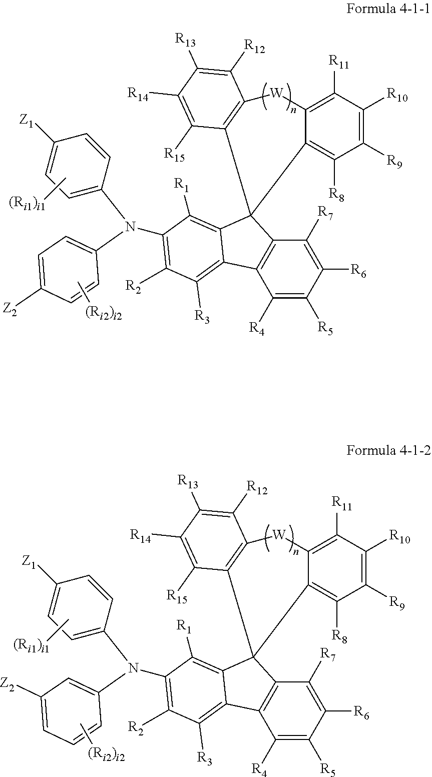

In Formulas 4-1-1 and 4-1-2, R₁ to R₁₅, Rᵢ₁, Rᵢ₂, W, Z₁, Z₂, i1, i2, and n may each independently be as defined in Formulas 1 and 4-1.

In one or more embodiments, Formula 4-2 may be represented by Formula 4-2-1 or Formula 4-2-2.

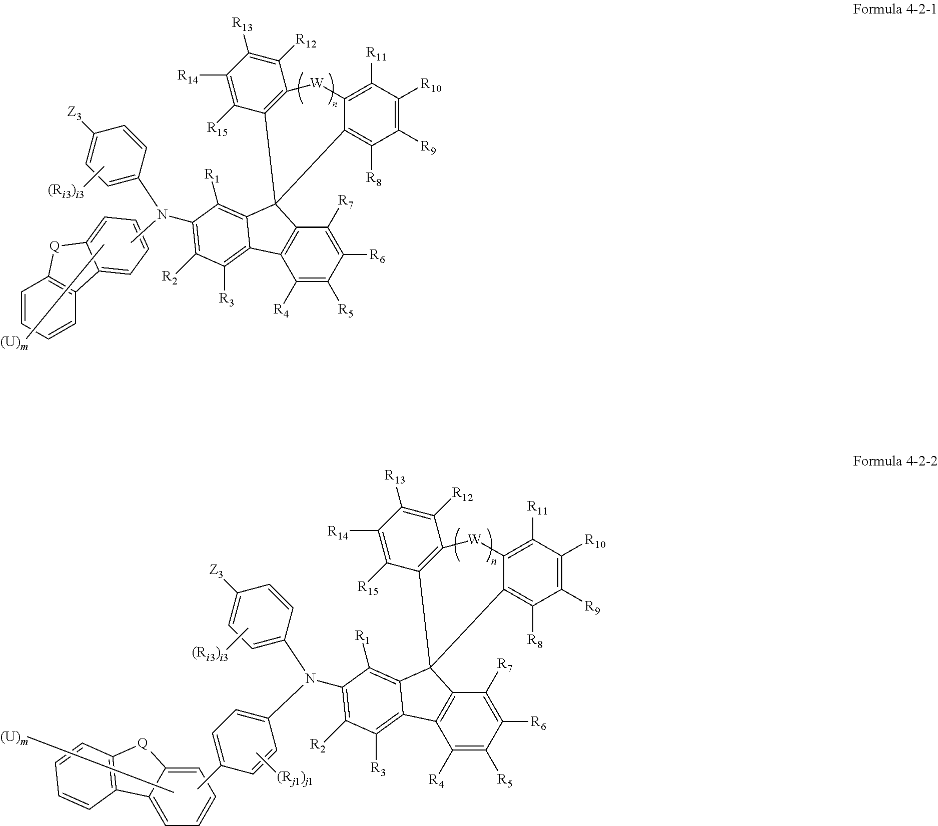

In Formulas 4-2-1 and 4-2-2, R₁ to R₁₅, Rᵢ₃, Rⱼ₁, W, Q, U, Z₃, i3, j1, n, and m may each independently be as defined in Formulas 1 and 4-2.

In one or more embodiments, Formula 1 may be represented by Formula 5.

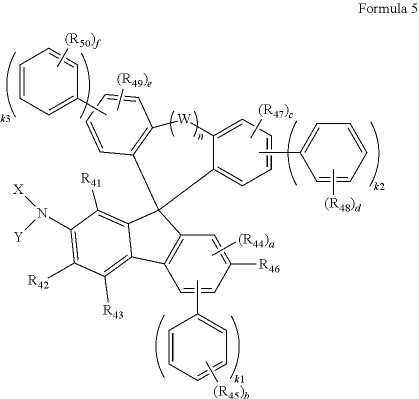

In Formula 5, R₄₁ to R₄₄, R₄₆, R₄₇, and R₄₉ may each independently be a hydrogen atom, a deuterium atom, a halogen atom, a substituted or unsubstituted amine group, a substituted or unsubstituted silyl group, a substituted or unsubstituted aryl group having 6 to 30 ring-forming carbon atoms, or a substituted or unsubstituted heteroaryl group having 2 to 30 ring-forming carbon atoms, R₄₅, R₄₈, and R₅₀ may each independently be a hydrogen atom or a deuterium atom, a is an integer of 0 to 3, c and e may each independently be an integer of 0 to 4, b, d, and f may each independently be an integer of 0 to 6, k1 to k3 may each independently be 0 or 1 and at least one of k1 to k3 is 1, and X, Y, W, and n may each independently be the same as defined in Formula 1.

In one or more embodiments, the amine compound represented by Formula 1 may be a monoamine compound.

### BRIEF DESCRIPTION OF THE FIGURES

The accompanying drawings are included to provide a further understanding of the present disclosure, and are incorporated in and constitute a part of this specification. The drawings illustrate example embodiments of the present disclosure and, together with the description, serve to explain principles of the present disclosure. In the drawings:

FIG. 1 is a plan view showing a display device according to one or more embodiments;

FIG. 2 is a cross-sectional view of a display device according to one or more embodiments of the present disclosure;

FIG. 3 is a cross-sectional view schematically showing a light emitting element according to one or more embodiments of the present disclosure;

FIG. 4 is a cross-sectional view schematically showing a light emitting element according to one or more embodiments of the present disclosure;

FIG. 5 is a cross-sectional view schematically showing a light emitting element according to one or more embodiments of the present disclosure;

FIG. 6 is a cross-sectional view schematically showing a light emitting element according to one or more embodiments of the present disclosure;

FIG. 7 is a cross-sectional view schematically showing a light emitting element according to one or more embodiments of the present disclosure;

FIG. 8 is a cross-sectional view of a display device according to one or more embodiments of the present disclosure;

FIG. 9 is a cross-sectional view of a display device according to one or more embodiments of the present disclosure;

FIG. 10 is a cross-sectional view showing a display device according to one or more embodiments of the present disclosure;

FIG. 11 is a cross-sectional view showing a display device according to one or more embodiments of the present disclosure; and

FIG. 12 is a view showing a vehicle in which a display device according to one or more embodiments of the present disclosure is provided.

### DETAILED DESCRIPTION

The present disclosure may be modified in one or more suitable manners and have many forms, and thus specific embodiments will be exemplified in the drawings and described in more detail in the detailed description of the present disclosure. It should be understood, however, that it is not intended to limit the present disclosure to the particular forms disclosed, but rather, is intended to cover all modifications, equivalents, and alternatives falling within the spirit and scope of the present disclosure.

When explaining each of drawings, like reference numbers are utilized for referring to like elements. In the accompanying drawings, the dimensions of each structure are exaggeratingly illustrated for clarity of the present disclosure. It will be understood that, although the terms “first,” “second,” and/or the like, may be utilized herein to describe one or more suitable components, these components should not be limited by these terms. These terms are only utilized to distinguish one component from another. For example, a first component could be termed a second component, and, similarly, a second component could be termed a first component, without departing from the scope of example embodiments of the present disclosure. As utilized herein, the singular forms, “a,” “an,” and “the” are intended to include the plural forms as well, unless the context clearly indicates otherwise.

In the present application, it will be understood that the terms “comprise,” “comprises,” “comprising,” “include,” “includes,” “including,” “have,” “has,” “having,” and/or the like specify the presence of features, numbers, steps, operations, component, parts, or combinations thereof disclosed in the specification, but do not exclude the possibility of presence or addition of one or more other features, numbers, steps, operations, component, parts, or combinations thereof.

In the present application, when a layer, a film, a region, or a plate is referred to as being “on,” “connected to,” “coupled to,” or “in an upper portion of” another layer, film, region, or plate, it may be not only “directly on” the layer, film, region, or plate, but intervening layers, films, regions, or plates may also be present. On the contrary to this, when a layer, a film, a region, or a plate is referred to as being “in a lower portion of” another layer, film, region, or plate, it can be not only directly under the layer, film, region, or plate, but intervening layers, films, regions, or plates may also be present. In some embodiments, it will be understood that when a part is referred to as being “on” another part, it can be provided above the other part, or provided under the other part as well. When an element is referred to as being “directly on,” “directly connected to,” or “directly coupled to” another element, there are no intervening elements present.

As used herein, the terms “use,” “using,” and “used” may be considered synonymous with the terms “utilize,” “utilizing,” and “utilized,” respectively.

As used herein, expressions such as “at least one of,” “one of,” and “selected from,” when preceding a list of elements, modify the entire list of elements and do not modify the individual elements of the list. For example, the expressions “at least one of a to c,” “at least one of a, b or c,” and “at least one of a, b and/or c” may indicate only a, only b, only c, both (e.g., simultaneously) a and b, both (e.g., simultaneously) a and c, both (e.g., simultaneously) b and c, all of a, b, and c, or variations thereof.

As used herein, the term “and/or” includes any and all combinations of one or more of the associated listed items.

The term “may” will be understood to refer to “one or more embodiments of the present disclosure,” some of which include the described element and some of which exclude that element and/or include an alternate element. Similarly, alternative language such as “or” refers to “one or more embodiments of the present disclosure,” each including a corresponding listed item.

Spatially relative terms, such as “beneath,” “below,” “lower,” “above,” “upper,” “bottom,” “top,” and the like, may be used herein for ease of description to describe one element or feature's relationship to another element(s) or feature(s) as illustrated in the drawings. It will be understood that the spatially relative terms are intended to encompass different orientations of the device in use or operation in addition to the orientation depicted in the drawings. For example, if the device in the drawings is turned over, elements described as “below” or “beneath” other elements or features would then be oriented “above” or “over” the other elements or features. Thus, the term “below” may encompass both an orientation of above and below. The device may be otherwise oriented (rotated 90 degrees or at other orientations), and the spatially relative descriptors used herein should be interpreted accordingly.

Unless otherwise defined, all terms (including technical and scientific terms) used herein have the same meaning as commonly understood by those of ordinary skill in the art to which the present disclosure pertains. It is also to be understood that terms defined in commonly used dictionaries should be interpreted as having meanings consistent with meanings in the context of the related art, unless expressly defined herein, and should not be interpreted in an ideal or overly formal sense.

In this context, “consisting essentially of” means that any additional components will not materially affect the chemical, physical, optical, or electrical properties of the semiconductor film.

Further, in this specification, the phrase “on a plane,” or “plan view,” means viewing a target portion from the top, and the phrase “on a cross-section” means viewing a cross-section formed by vertically cutting a target portion from the side.

### Definitions

In the specification, the term “substituted or unsubstituted” may refer to substituted or unsubstituted with at least one substituent selected from the group consisting of a deuterium atom, a halogen atom, a cyano group, a nitro group, an amino group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, a hydrocarbon ring group, an aryl group, and a heterocyclic group. In some embodiments, each of the substituents exemplified herein may be substituted or unsubstituted. For example, a biphenyl group may be interpreted as an aryl group or a phenyl group substituted with a phenyl group.

In the specification, the phrase “bonded to an adjacent group to form a ring” may refer to that a group is bonded to an adjacent group to form a substituted or unsubstituted hydrocarbon ring, or a substituted or unsubstituted heterocycle. The hydrocarbon ring includes an aliphatic hydrocarbon ring and an aromatic hydrocarbon ring. The heterocycle includes an aliphatic heterocycle and an aromatic heterocycle.

The hydrocarbon ring and the heterocycle may be monocyclic or polycyclic. In some embodiments, the rings formed by being bonded to each other may be connected to another ring to form a spiro structure.

In the specification, the term “adjacent group” may refer to a substituent substituted for an atom which is directly linked to an atom substituted with a corresponding substituent, another substituent substituted for an atom which is substituted with a corresponding substituent, or a substituent sterically positioned at the nearest position to a corresponding substituent. For example, two methyl groups in 1,2-dimethylbenzene may be interpreted as “adjacent groups” to each other and two ethyl groups in 1,1-diethylcyclopentane may be interpreted as “adjacent groups” to each other. In some embodiments, two methyl groups in 4,5-dimethylphenanthrene may be interpreted as “adjacent groups” to each other.

In the specification, examples of the halogen atom may include a fluorine atom, a chlorine atom, a bromine atom, or an iodine atom.

In the specification, the alkyl group may be linear or branched. The number of carbons in the alkyl group is 1 to 50, 1 to 30, 1 to 20, 1 to 10, or 1 to 6. Examples of the alkyl group may include a methyl group, an ethyl group, an n-propyl group, an isopropyl group, an n-butyl group, an s-butyl group, a t-butyl group, an i-butyl group, a 2-ethylbutyl group, a 3,3-dimethylbutyl group, an n-pentyl group, an i-pentyl group, a neopentyl group, a t-pentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-heneicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, and/or the like, but the embodiment of the present disclosure is not limited thereto.

In the specification, a cycloalkyl group may refer to a cyclic alkyl group. The number of carbons in the cycloalkyl group is 3 to 50, 3 to 30, 3 to 20, or 3 to 10. Examples of the cycloalkyl group may include a cyclopropyl group, a cyclobutyl group, a cyclopentyl group, a cyclohexyl group, a 4-methylcyclohexyl group, a 4-t-butylcyclohexyl group, a cycloheptyl group, a cyclooctyl group, a cyclononyl group, a cyclodecyl group, a norbornyl group, a 1-adamantyl group, a 2-adamantyl group, an isobornyl group, a bicycloheptyl group, and/or the like, but the embodiment of the present disclosure is not limited thereto.

In the specification, an alkenyl group refers to a hydrocarbon group including at least one carbon double bond in the middle or terminal of an alkyl group having 2 or more carbon atoms. The alkenyl group may be linear or branched. The number of carbon atoms in the alkenyl group is not specifically limited, but is 2 to 30, 2 to 20, or 2 to 10. Examples of the alkenyl group include a vinyl group, a 1-butenyl group, a 1-pentenyl group, a 1,3-butadienyl group, a styrenyl group, a styryl vinyl group, and/or the like, but the embodiment of the present disclosure is not limited thereto.

In the specification, an alkynyl group refers to a hydrocarbon group including at least one carbon triple bond in the middle or terminal of an alkyl group having 2 or more carbon atoms. The alkynyl group may be linear or branched. Although the number of carbon atoms is not specifically limited, it is 2 to 30, 2 to 20, or 2 to 10. Specific examples of the alkynyl group may include an ethynyl group, a propynyl group, and/or the like, but are not limited thereto.

In the specification, the hydrocarbon ring group refers to any functional group or substituent derived from an aliphatic hydrocarbon ring. The hydrocarbon ring group may be a saturated hydrocarbon ring group having 5 to 20 ring-forming carbon atoms.

In the specification, an aryl group refers to any functional group or substituent derived from an aromatic hydrocarbon ring. The aryl group may be a monocyclic aryl group or a polycyclic aryl group. The number of ring-forming carbon atoms in the aryl group may be 6 to 30, 6 to 20, or 6 to 15. Examples of the aryl group may include a phenyl group, a naphthyl group, a fluorenyl group, an anthracenyl group, a phenanthryl group, a biphenyl group, a terphenyl group, a quaterphenyl group, a quinquephenyl group, a sexiphenyl group, a triphenylenyl group, a pyrenyl group, a benzofluoranthenyl group, a chrysenyl group, and/or the like, but the embodiment of the present disclosure is not limited thereto.

In the specification, the fluorenyl group may be substituted, and two substituents may be bonded to each other to form a spiro structure. Examples of the substituted fluorenyl group are as follows. However, the embodiment of the present disclosure is not limited thereto.

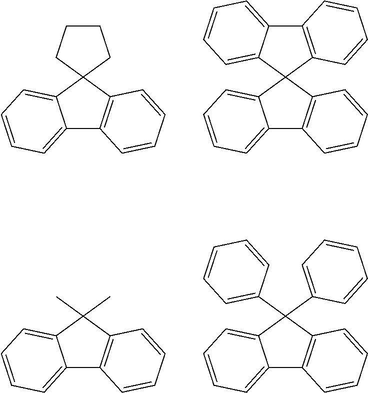

The heterocyclic group herein refers to any functional group or substituent derived from a ring containing at least one of B, O, N, P, Si, or Se as a heteroatom. The heterocyclic group includes an aliphatic heterocyclic group and an aromatic heterocyclic group. The aromatic heterocyclic group may be a heteroaryl group. The aliphatic heterocycle and the aromatic heterocycle may be monocyclic or polycyclic.

In the specification, the heterocyclic group may contain at least one of B, O, N, P, Si or S as a heteroatom. When the heterocyclic group contains two or more heteroatoms, the two or more heteroatoms may be the same as or different from each other. The heterocyclic group may be a monocyclic heterocyclic group or a polycyclic heterocyclic group, and includes a heteroaryl group. The number of ring-forming carbon atoms in the heterocyclic group may be 2 to 30, 2 to 20, or 2 to 10.

In the specification, the aliphatic heterocyclic group may include at least one of B, O, N, P, Si, or S as a heteroatom. The number of ring-forming carbon atoms in the aliphatic heterocyclic group may be 2 to 30, 2 to 20, or 2 to 10. Examples of the aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, and/or the like, but the embodiment of the present disclosure is not limited thereto.

In the specification, the heteroaryl group may contain at least one of B, O, N, P, Si, or S as a heteroatom. When the heteroaryl group contains two or more heteroatoms, the two or more heteroatoms may be the same as or different from each other. The heteroaryl group may be a monocyclic heterocyclic group or a polycyclic heterocyclic group. The number of ring-forming carbon atoms in the heteroaryl group may be 2 to 30, 2 to 20, or 2 to 10. Examples of the heteroaryl group may include a thiophene group, a furan group, a pyrrole group, an imidazole group, a pyridine group, a bipyridine group, a pyrimidine group, a triazine group, a triazole group, an acridyl group, a pyridazine group, a pyrazinyl group, a quinoline group, a quinazoline group, a quinoxaline group, a phenoxazine group, a phthalazine group, a pyrido pyrimidine group, a pyrido pyrazine group, a pyrazino pyrazine group, an isoquinoline group, an indole group, a carbazole group, an N-arylcarbazole group, an N-heteroarylcarbazole group, an N-alkylcarbazole group, a benzoxazole group, a benzimidazole group, a benzothiazole group, a benzocarbazole group, a benzothiophene group, a dibenzothiophene group, a thienothiophene group, a benzofuran group, a phenanthroline group, a thiazole group, an isoxazole group, an oxazole group, an oxadiazole group, a thiadiazole group, a phenothiazine group, a dibenzosilole group, a dibenzofuran group, and/or the like, but the embodiment of the present disclosure is not limited thereto.

In the specification, the description of the aryl group may be applied to an arylene group except that the arylene group is a divalent group. The description of the heteroaryl group may be applied to a heteroarylene group except that the heteroarylene group is a divalent group.

In the specification, the silyl group includes an alkylsilyl group and an arylsilyl group. Examples of the silyl group may include a trimethylsilyl group, a triethylsilyl group, a t-butyldimethylsilyl group, a propyldimethylsilyl group, a triphenylsilyl group, a diphenylsilyl group, a phenylsilyl group, and/or the like, but the embodiment of the present disclosure is not limited thereto.

In the specification, the number of ring-forming carbon atoms in the carbonyl group is not specifically limited, but may be 1 to 40, 1 to 30, or 1 to 20, 1 to 20, or 1 to 10. For example, the carbonyl group may have the following structures, but the embodiment of the present disclosure is not limited thereto.

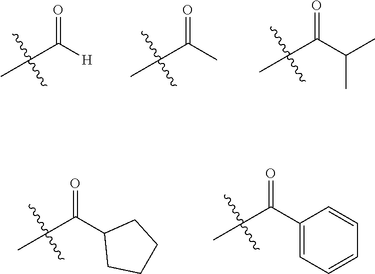

In the specification, the number of carbon atoms in the sulfinyl group and the sulfonyl group is not particularly limited, but may be 1 to 30. The sulfinyl group may include an alkyl sulfinyl group and an aryl sulfinyl group. The sulfonyl group may include an alkyl sulfonyl group and an aryl sulfonyl group.

In the specification, the thio group may include an alkylthio group and an arylthio group. The thio group may refer to that a sulfur atom is bonded to the alkyl group or the aryl group as defined above. Examples of the thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, but the embodiment of the present disclosure is not limited thereto.

In the specification, an oxy group may refer to that an oxygen atom is bonded to the alkyl group or the aryl group as defined above. The oxy group may include an alkoxy group and an aryl oxy group. The alkoxy group may be a linear chain, a branched chain or a ring chain. The number of carbon atoms in the alkoxy group is not specifically limited, but may be, for example, 1 to 20 or 1 to 10. Examples of the oxy group may include methoxy, ethoxy, n-propoxy, isopropoxy, butoxy, pentyloxy, hexyloxy, octyloxy, nonyloxy, decyloxy, benzyloxy, and/or the like, but the embodiment of the present disclosure is not limited thereto.

The boron group herein may refer to that a boron atom is bonded to the alkyl group or the aryl group as defined above. The boron group includes an alkyl boron group and an aryl boron group. Examples of the boron group may include a dimethylboron group, a diphenylboron group, a phenylboron group, and/or the like, but the embodiment of the present disclosure is not limited thereto.

In the specification, the number of carbon atoms in an amine group is not specifically limited, but may be 1 to 30, 1 to 20, or 1 to 10. The amine group may include an alkyl amine group and an aryl amine group. Examples of the amine group may include a methylamine group, a dimethylamine group, a phenylamine group, a diphenylamine group, a naphthylamine group, a 9-methyl-anthracenylamine group, and/or the like, but the embodiment of the present disclosure is not limited thereto.

In the specification, the alkyl group among an alkylthio group, an alkylsulfoxy group, an alkylaryl group, an alkylamino group, an alkyl boron group, an alkyl silyl group, and an alkyl amine group is the same as the examples of the alkyl group described herein.

In the specification, the aryl group among an aryloxy group, an arylthio group, an arylsulfoxy group, an arylamino group, an arylboron group, an arylsilyl group, an arylamine group is the same as the examples of the aryl group described herein.

In the specification, a direct linkage may refer to a single bond.

In some embodiments, in the specification,

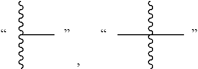

and “----*” refer to a position to be connected.

Hereinafter, embodiments of the present disclosure will be described with reference to the accompanying drawings.

### Display Apparatus

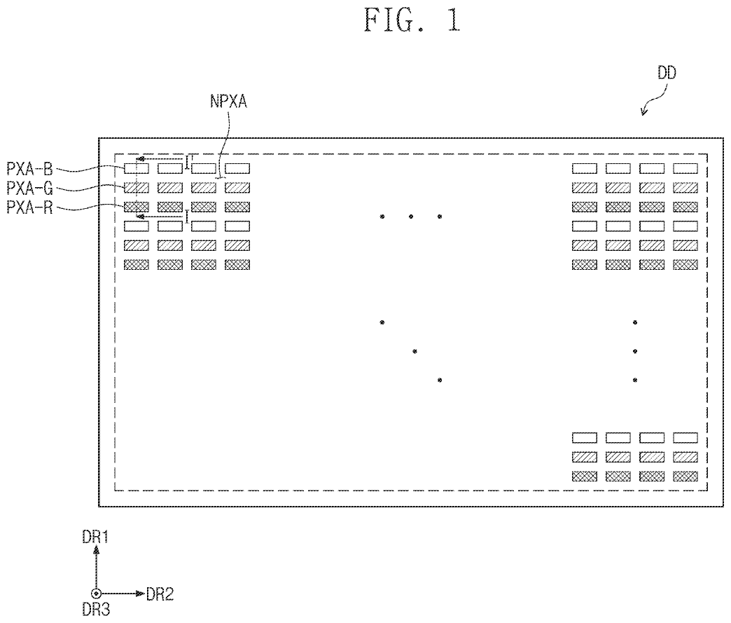

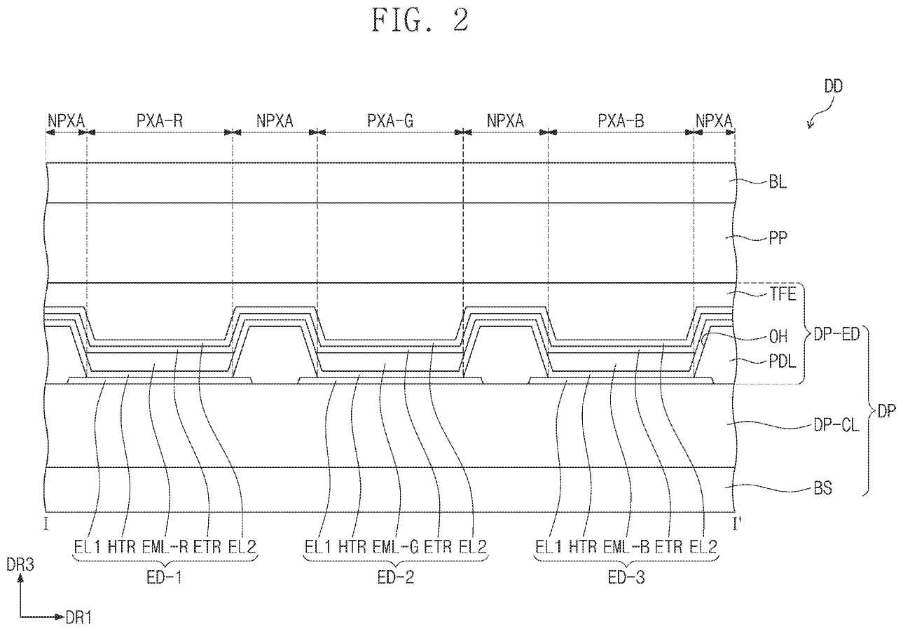

FIG. 1 is a plan view illustrating one or more embodiments of a display apparatus DD. FIG. 2 is a cross-sectional view of the display apparatus DD of the embodiment. FIG. 2 is a cross-sectional view illustrating a part taken along line I-I′ of FIG. 1.

The display apparatus DD may include a display panel DP and an optical layer PP provided on the display panel DP. The display panel DP includes light emitting elements ED-1, ED-2, and ED-3. The display apparatus DD may include a plurality of light emitting elements ED-1, ED-2, and ED-3. The optical layer PP may be provided on the display panel DP to control reflected light in the display panel DP due to external light. The optical layer PP may include, for example, a polarization layer or a color filter layer. In some embodiments, unlike the configuration illustrated in the drawing, the optical layer PP may not be provided from the display apparatus DD of one or more embodiments.

A base substrate BL may be provided on the optical layer PP. The base substrate BL may be a member which provides a base surface on which the optical layer PP provided. The base substrate BL may be a glass substrate, a metal substrate, a plastic substrate, and/or the like. However, the embodiment of the present disclosure is not limited thereto, and the base substrate BL may be an inorganic layer, an organic layer, or a composite material layer. In some embodiments, unlike the configuration illustrated, in one or more embodiments, the base substrate BL may not be provided.

The display apparatus DD according to one or more embodiments may further include a filling layer. The filling layer may be provided between a display device layer DP-ED and the base substrate BL. The filling layer may be an organic material layer. The filling layer may include at least one of an acrylic-based resin, a silicone-based resin, and/or an epoxy-based resin (i.e., at least one of an acrylic-based resin, a silicone-based resin, or an epoxy-based resin).

The display panel DP may include a base layer BS, a circuit layer DP-CL provided on the base layer BS, and the display device layer DP-ED. The display device layer DP-ED may include a pixel defining film PDL, the light emitting elements ED-1, ED-2, and ED-3 provided between portions of the pixel defining film PDL, and an encapsulation layer TFE provided on the light emitting elements ED-1, ED-2, and ED-3.

The base layer BS may be a member which provides a base surface on which the display device layer DP-ED is provided. The base layer BS may be a glass substrate, a metal substrate, a plastic substrate, and/or the like. However, the embodiment is not limited thereto, and the base layer BS may be an inorganic layer, an organic layer, or a composite material layer.

In one or more embodiments, the circuit layer DP-CL is provided on the base layer BS, and the circuit layer DP-CL may include a plurality of transistors. Each of the transistors may include a control electrode, an input electrode, and an output electrode. For example, the circuit layer DP-CL may include a switching transistor and a driving transistor for driving the light emitting elements ED-1, ED-2, and ED-3 of the display device layer DP-ED.

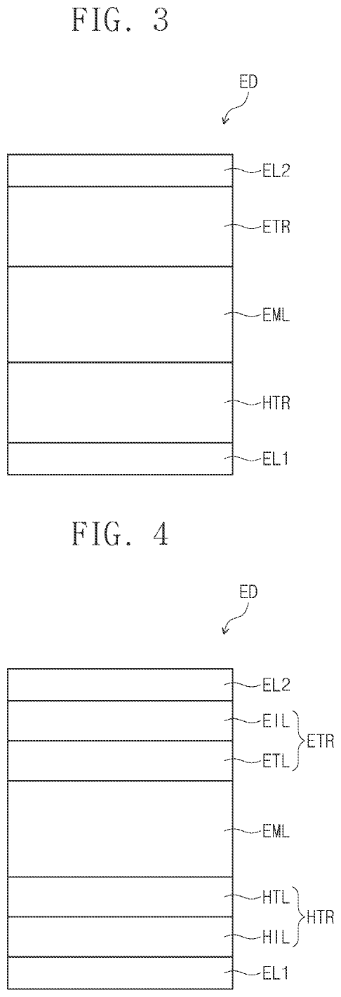

Each of the light emitting elements ED-1, ED-2, and ED-3 may have a structure of each light emitting element ED of embodiments according to FIGS. 3 to 6, as described in more detail elsewhere herein. Each of the light emitting elements ED-1, ED-2, and ED-3 may include a first electrode EL1, a hole transport region HTR, a layer selected from among emission layers EML-R, EML-G, and EML-B, an electron transport region ETR, and a second electrode EL2.

FIG. 2 illustrates one or more embodiments in which the emission layers EML-R, EML-G, and EML-B of the light emitting elements ED-1, ED-2, and ED-3 are provided in openings OH defined in the pixel defining film PDL, and the hole transport region HTR, the electron transport region ETR, and the second electrode EL2 are provided as a common layer in the entire light emitting elements ED-1, ED-2, and ED-3. However, the embodiment of the present disclosure is not limited thereto, and unlike the configuration illustrated in FIG. 2, the hole transport region HTR and the electron transport region ETR in one or more embodiments may be provided by being patterned inside the openings OH defined in the pixel defining film PDL. For example, the hole transport region HTR, the emission layers EML-R, EML-G, and EML-B, and the electron transport region ETR of the light emitting elements ED-1, ED-2, and ED-3 in one or more embodiments may be provided by being patterned in an inkjet printing method.

The encapsulation layer TFE may cover the light emitting elements ED-1, ED-2 and ED-3. The encapsulation layer TFE may seal the display device layer DP-ED. The encapsulation layer TFE may be a thin film encapsulation layer. The encapsulation layer TFE may be formed by laminating one layer or a plurality of layers.

The encapsulation layer TFE includes at least one insulation layer. The encapsulation layer TFE according to one or more embodiments may include at least one inorganic film (hereinafter, an encapsulation-inorganic film). The encapsulation layer TFE according to one or more embodiments may also include at least one organic film (hereinafter, an encapsulation-organic film) and at least one encapsulation-inorganic film.

The encapsulation-inorganic film protects the display device layer DP-ED from moisture/oxygen, and the encapsulation-organic film protects the display device layer DP-ED from foreign substances such as dust particles. The encapsulation-inorganic film may include silicon nitride, silicon oxynitride, silicon oxide, titanium oxide, aluminum oxide, and/or the like, but the embodiment of the present disclosure is not particularly limited thereto. The encapsulation-organic film may include an acrylic-based compound, an epoxy-based compound, and/or the like. The encapsulation-organic film may include a photopolymerizable organic material, but the embodiment of the present disclosure is not particularly limited thereto.

The encapsulation layer TFE may be provided on the second electrode EL2 and may be provided filling the opening OH.

Referring to FIGS. 1 and 2, the display apparatus DD may include one or more non-light emitting region(s) NPXA and light emitting regions PXA-R, PXA-G, and PXA-B. The light emitting regions PXA-R, PXA-G, and PXA-B may be regions in which light generated by the respective light emitting elements ED-1, ED-2, and ED-3 is emitted. The light emitting regions PXA-R, PXA-G, and PXA-B may be spaced apart from each other on a plane.

Each of the light emitting regions PXA-R, PXA-G, and PXA-B may be a region divided by the pixel defining film PDL. The non-light emitting areas NPXA may be areas between the adjacent light emitting areas PXA-R, PXA-G, and PXA-B, which correspond to the pixel defining film PDL. In some embodiments, in the specification, the light emitting regions PXA-R, PXA-G, and PXA-B may respectively correspond to pixels. The pixel defining film PDL may divide the light emitting elements ED-1, ED-2, and ED-3. The emission layers EML-R, EML-G, and EML-B of the light emitting elements ED-1, ED-2, and ED-3 may be provided in openings OH defined in the pixel defining film PDL and separated from each other.

The light emitting regions PXA-R, PXA-G, and PXA-B may be divided into a plurality of groups according to the color of light generated from the light emitting elements ED-1, ED-2, and ED-3. In the display apparatus DD of one or more embodiments illustrated in FIGS. 1 and 2, three light emitting regions PXA-R, PXA-G, and PXA-B, which emit red light, green light, and blue light, respectively, are illustrated as an example. For example, the display apparatus DD of one or more embodiments may include the red light emitting region PXA-R, the green light emitting region PXA-G, and the blue light emitting region PXA-B that are separated from each other.

In the display apparatus DD according to one or more embodiments, the plurality of light emitting elements ED-1, ED-2 and ED-3 may be to emit (e.g., configured to emit) light beams having wavelengths different from each other. For example, in one or more embodiments, the display apparatus DD may include a first light emitting element ED-1 that emits red light, a second light emitting element ED-2 that emits green light, and a third light emitting elementED-3 that emits blue light. For example, the red light emitting region PXA-R, the green light emitting region PXA-G, and the blue light emitting region PXA-B of the display apparatus DD may correspond to the first light emitting element ED-1, the second light emitting element ED-2, and the third light emitting element ED-3, respectively.

However, the embodiment of the present disclosure is not limited thereto, and the first to third light emitting elements ED-1, ED-2, and ED-3 may be to emit (e.g., configured to emit) light beams in substantially the same wavelength range or at least one light emitting element may be to emit (e.g., configured to emit) a light beam in a wavelength range different from the others. For example, the first to third light emitting elements ED-1, ED-2, and ED-3 may all emit blue light.

The light emitting regions PXA-R, PXA-G, and PXA-B in the display apparatus DD according to one or more embodiments may be arranged in a stripe form. Referring to FIG. 1, the plurality of red light emitting regions PXA-R may be arranged with each other along a second directional axis DR2, the plurality of green light emitting regions PXA-G may be arranged with each other along a second directional axis DR2, and the plurality of blue light emitting regions PXA-B may be arranged with each other along the second directional axis DR2. In some embodiments, the red light emitting region PXA-R, the green light emitting region PXA-G, and the blue light emitting region PXA-B may be alternately arranged with each other in this order along a first directional axis DR1.

FIGS. 1 and 2 illustrate that all the light emitting regions PXA-R, PXA-G, and PXA-B have similar area, but the embodiment of the present disclosure is not limited thereto. Thus, the light emitting regions PXA-R, PXA-G, and PXA-B may have different areas from each other according to the wavelength range of the emitted light. In this case, the areas of the light emitting regions PXA-R, PXA-G, and PXA-B may refer to areas when viewed on a plane defined by the first directional axis DR1 and the second directional axis DR2.

In some embodiments, an arrangement form of the light emitting regions PXA-R, PXA-G, and PXA-B is not limited to the configuration illustrated in FIG. 1, and the order in which the red light emitting region PXA-R, the green light emitting region PXA-G, and the blue light emitting region PXA-B are arranged may be provided in one or more suitable combinations according to the characteristics of display quality required in the display apparatus DD. For example, the arrangement form of the light emitting regions PXA-R, PXA-G, and PXA-B may be a pentile (PENTILE®) arrangement form or a diamond (Diamond Pixel®) arrangement form, (PENTILE® and Diamond Pixel® are registered trademarks owned by Samsung Display Co., Ltd.).

In some embodiments, the areas of the light emitting regions PXA-R, PXA-G, and PXA-B may be different from each other. For example, in one or more embodiments, the area of the green light emitting region PXA-G may be smaller than that of the blue light emitting region PXA-B, but the embodiment of the present disclosure is not limited thereto.

Hereinafter, FIG. 3 to FIG. 7 are cross-sectional views schematically showing light emitting elements according to embodiments. The light emitting element ED according to one or more embodiments may include a first electrode EL1, a second electrode EL2 oppositely provided to the first electrode EL1, and at least one functional layer provided between the first electrode EL1 and the second electrode EL2. The light emitting element ED of one or more embodiments may include an amine compound of one or more embodiments, as described elsewhere herein, in the at least one functional layer.

The light emitting element ED may include a hole transport region HTR, an emission layer EML, an electron transport region ETR, and/or the like, stacked in order, as the at least one functional layer. Referring to FIG. 3, the light emitting element ED of one or more embodiments may include a first electrode EL1, a hole transport region HTR, an emission layer EML, an electron transport region ETR, and a second electrode EL2, stacked in order.

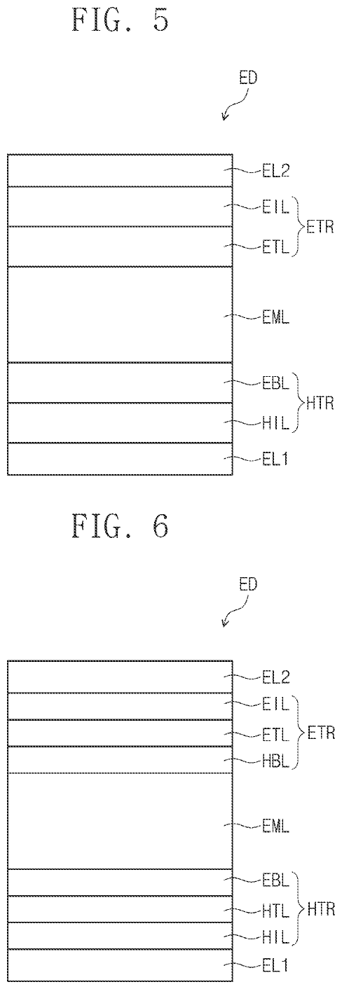

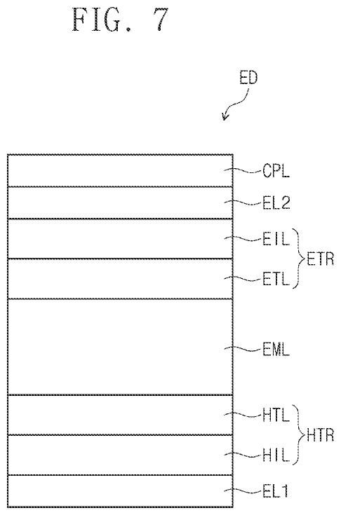

Compared with FIG. 3, FIG. 4 illustrates a cross-sectional view of a light emitting element ED of one or more embodiments, in which a hole transport region HTR includes a hole injection layer HIL and a hole transport layer HTL, and an electron transport region ETR includes an electron injection layer EIL and an electron transport layer ETL. Compared with FIG. 3, FIG. 5 illustrates a cross-sectional view of a light emitting element ED of one or more embodiments, in which a hole transport region HTR includes a hole injection layer HIL and an electron blocking layer EBL, and an electron transport region ETR includes an electron injection layer EIL and an electron transport layer ETL. In some embodiments, compared with FIG. 3, FIG. 6 illustrates a cross-sectional view of a light emitting element ED of one or more embodiments, in which a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. Compared with FIG. 4, FIG. 7 illustrates a cross-sectional view of a light emitting element ED of one or more embodiments including a capping layer CPL provided on a second electrode EL2.

The light emitting element ED of one or more embodiments may include an amine compound of one or more embodiments, as described elsewhere herein, in the hole transport region HTR. The light emitting element ED of one or more embodiments may include the amine compound of one or more embodiments in at least one of the hole injection layer HIL, the hole transport layer HTL, or the electron blocking layer EBL of the hole transport region HTR. For example, in the light emitting element ED of one or more embodiments, the hole transport layer HTL or the electron blocking layer EBL may include the amine compound of one or more embodiments.

In the light emitting element ED according to one or more embodiments, the first electrode EL1 has conductivity (e.g., is a conductor). The first electrode EL1 may be formed of a metal material, a metal alloy, or a conductive compound. The first electrode EL1 may be an anode or a cathode. However, the embodiment of the present disclosure is not limited thereto. In some embodiments, the first electrode EL1 may be a pixel electrode. The first electrode EL1 may be a transmissive electrode, a transflective electrode, or a reflective electrode. The first electrode EL1 may include at least one selected from among Ag, Mg, Cu, Al, Pt, Pd, Au, Ni, Nd, Ir, Cr, Li, Ca, LiF, Mo, Ti, W, In, Sn, and Zn, a compound of two or more selected from among these, a mixture of two or more selected from among these, and/or an oxide thereof.

When the first electrode EL1 is the transmissive electrode, the first electrode EL1 may include a transparent metal oxide such as indium tin oxide (ITO), indium zinc oxide (IZO), zinc oxide (ZnO), or indium tin zinc oxide (ITZO). When the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, a compound or mixture thereof (e.g., a mixture of Ag and Mg). In some embodiments, the first electrode EL1 may have a multilayer structure including a reflective film or a transflective film formed of the herein-described materials, and a transparent conductive film formed of ITO, IZO, ZnO, ITZO, and/or the like. For example, the first electrode EL1 may have a three-layer structure of ITO/Ag/ITO, but the embodiment of the present disclosure is not limited thereto. In some embodiments, the embodiment of the present disclosure is not limited thereto, and the first electrode EL1 may include the herein-described metal materials, combinations of at least two metal materials of the herein-described metal materials, oxides of the herein-described metal materials, and/or the like. The thickness of the first electrode EL1 may be from about 700 Å to about 10,000 Å. For example, the thickness of the first electrode EL1 may be from about 1,000 Å to about 3,000 Å.

The hole transport region HTR is provided on the first electrode EL1. The hole transport region HTR may have a single layer formed utilizing a single material, a single layer formed utilizing multiple different materials, or a multilayer structure including multiple layers formed utilizing multiple different materials.

The hole transport region HTR may include at least one of a hole injection layer HIL, a hole transport layer HTL, or an electron blocking layer EBL. In some embodiments, the hole transport region HTR may include multiple hole transport layers stacked.

In some embodiments, otherwise, the hole transport region HTR may have the structure of a single layer of a hole injection layer HIL or a hole transport layer HTL, and may have a structure of a single layer formed utilizing a hole injection material and a hole transport material. In one or more embodiments, the hole transport region HTR may have a structure of a single layer formed utilizing multiple different materials, or a structure stacked from the first electrode EL1 of hole injection layer HIL/hole transport layer HTL, hole injection layer HIL/hole transport layer HTL/buffer layer, hole injection layer HIL/buffer layer, or hole transport layer HTL/buffer layer, without limitation.

The thickness of the hole transport region HTR may be, for example, about 50 Å to about 15,000 Å. The hole transport region HTR may be formed utilizing one or more suitable methods such as a vacuum deposition method, a spin coating method, a cast method, a Langmuir-Blodgett (LB) method, an inkjet printing method, a laser printing method, and/or a laser induced thermal imaging (LITI) method.

The light emitting element ED of one or more embodiments may include an amine compound of one or more embodiments in the hole transport region HTR. In the light emitting element ED of one or more embodiments, the hole transport region HTR may include at least one of the hole injection layer HIL, the hole transport layer HTL, or the electron blocking layer EBL, and may include the amine compound of one or more embodiments in at least one of the hole injection layer HIL, the hole transport layer HTL, or the electron blocking layer EBL. For example, the electron blocking layer EBL may include the amine compound of one or more embodiments.

### Amine Compound

The amine compound of one or more embodiments includes a structure in which one first substituent and one second substituent are linked to the nitrogen atom of amine, and any one of the second substituent or a third substituent is further linked to the nitrogen atom of amine. For example, the amine compound of one or more embodiments may include a structure in which the first to third substituents are each linked the nitrogen atom of amine. In some embodiments, the amine compound of one or more embodiments may include a structure in which one first substituent and two second substituents are linked to the nitrogen atom of amine. In the amine compound of one or more embodiments, the first to third substituents may each be directly or indirectly linked to the nitrogen atom of amine.

The first substituent may include a fluorene moiety substituted with at least one substituent selected from a substituted or unsubstituted aryl group or a substituted or unsubstituted heteroaryl group. In some embodiments, the first substituent may be directly linked to the nitrogen atom of amine. For example, when the first substituent is a fluorenyl group, the fluorenyl group may be directly linked to the nitrogen atom of amine at the 2-position of fluorene. In one or more embodiments, the fluorene moiety included in the first substituent may include a skeleton of 9,9-diphenyl-9H-fluorene, a skeleton of 9,9′-spirobifluorene, a skeleton of 10H-spiro[acridine]-9,9′-fluorene] (10H-spiro[acridine-9,9′-fluorene]), a skeleton of spiro[fluorene-9,9′-xanthene](spiro[fluorene-9,9′-xanthene]), or a skeleton of spiro[fluorene-9,9′-thioxanthene].

The second substituent is linked to the nitrogen atom of amine through a linker, and may be a substituted or unsubstituted naphthyl group or a substituted or unsubstituted phenanthrenyl group. The linker between the second substituent and the nitrogen atom of amine may be a substituted or unsubstituted arylene group having 6 to 50 ring-forming carbon atoms, or a substituted or unsubstituted heteroarylene group having 2 to 50 ring-forming carbon atoms. The third substituent may be a substituted or unsubstituted dibenzofuran group, a substituted or unsubstituted dibenzothiophene group, a substituted carbazole group, or a substituted fluorenyl group. The third substituent may be directly linked to the nitrogen atom of amine or indirectly linked through a linker. The linker between the third substituent and the nitrogen atom of amine may be a substituted or unsubstituted arylene group having 6 to 50 ring-forming carbon atoms, or a substituted or unsubstituted heteroarylene group having 2 to 50 ring-forming carbon atoms.

The amine compound of one or more embodiments may be a monoamine compound including a singular amine group. The amine compound of one or more embodiments may be a monoamine compound having only one amine group present in a molecular structure without forming a ring. However, the embodiment of the present disclosure is not limited thereto, and the amine compound of one or more embodiments may include two or more amine groups in the molecular structure.

In one or more embodiments, the amine compound may be represented by Formula 1. In Formula 1, the fluorene moiety to which R₁ to R₁₅ are linked may correspond to the first substituent described herein. In some embodiments, in Formula 1, at least one of X or Y may correspond to the second substituent described herein, and the other may correspond to the third substituent described herein.

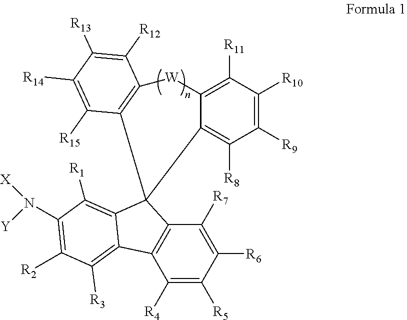

In Formula 1, R₁ to R₁₅ may each independently be a hydrogen atom, a deuterium atom, a halogen atom, a substituted or unsubstituted amine group, a substituted or unsubstituted silyl group, a substituted or unsubstituted aryl group having 6 to 30 ring-forming carbon atoms, or a substituted or unsubstituted heteroaryl group having 2 to 30 ring-forming carbon atoms. For example, R₁ to R₁₅ may each independently be a hydrogen atom, a deuterium atom, an unsubstituted phenyl group, or a phenyl group substituted with a deuterium atom. In one or more embodiments, at least one of R₃ to R₅, and R₇ to R₁₅ may be a substituted or unsubstituted aryl group having 6 to 30 ring-forming carbon atoms, or a substituted or unsubstituted heteroaryl group having 2 to 30 ring-forming carbon atoms. For example, in Formula 1, at least one selected from among R₃ to R₅, and R₇ to R₁₅ may be a substituted or unsubstituted phenyl group. In Formula 1, when at least one of R₃ to R₅, and R₇ to R₁₅ is a substituted phenyl group, the phenyl group may be substituted with a deuterium atom.

In Formula 1, n may be 0 or 1. W may be O, S, NRₐ₁, CRₐ₂Rₐ₃, or a direct linkage. For example, when n is 0, a benzene ring in which R₈ to R₁₁ are substituted and a benzene ring in which R₁₂ to R₁₅ are substituted may not be linked through W. For example, when n is 0, the amine compound of one or more embodiments represented by Formula 1 may include a skeleton of 9,9-diphenyl-9H-fluorene. In some embodiments, when n is 1, a benzene ring in which R₈ to R₁₁ are substituted and a benzene ring in which R₁₂ to R₁₅ are substituted may be linked through O, S, NRₐ₁, CRₐ₂Rₐ₃, or a direct linkage, and may form a spiro structure with a fluorenyl group in which R₁ to R₇ are substituted.

In one or more embodiments, Rai may be a substituted or unsubstituted aryl group having 6 to 30 ring-forming carbon atoms, or a substituted or unsubstituted heteroaryl group having 2 to 30 ring-forming carbon atoms. Rₐ₂ and Rₐ₃ may each independently be a substituted or unsubstituted aryl group having 6 to 30 ring-forming carbon atoms, or a substituted or unsubstituted heteroaryl group having 2 to 30 ring-forming carbon atoms. In some embodiments, Rₐ₂ and Rₐ₃ may be bonded to form a ring.

In Formula 1, X and Y may each independently be represented by Formula 2 or Formula 3. In one or more embodiments, at least one of X or Y may be represented by Formula 2, and the other of X or Y, which is not represented by Formula 2 may be represented by Formula 3. For example, both (e.g., simultaneously) X and Y may be represented by Formula 2. In some embodiments, any one of X and Y may be represented by Formula 2, and the other may be represented by Formula 3. When both (e.g., simultaneously) X and Y are represented by Formula 2, X and Y may be the same or different. In one or more embodiments, Formula 2 may correspond to the second substituent described herein, and Formula 3 may correspond to the third substituent described herein.

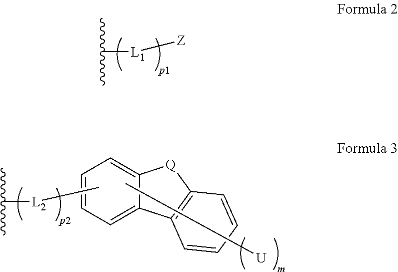

In Formulas 2 and 3, L₁ and L₂ may each independently be a substituted or unsubstituted arylene group having 6 to 50 ring-forming carbon atoms, or a substituted or unsubstituted heteroarylene group having 2 to 50 ring-forming carbon atoms. For example, L₁ and L₂ may each independently be a substituted or unsubstituted phenylene group. When L₁ and L₂ are each substituted with another substituent, the substituent may be a deuterium atom, but the embodiment of the present disclosure is not limited thereto. For example, the substituents substituted on each of L₁ and L₂ may be a substituted or unsubstituted amine group, a substituted or unsubstituted silyl group, a substituted or unsubstituted aryl group having 6 to 15 ring-forming carbon atoms, or a substituted or unsubstituted heteroaryl group having 2 to 15 ring-forming carbon atoms. In Formulas 2 and 3,

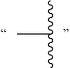

is a site linked to the nitrogen atom of amine.

In Formula 2, p1 may be an integer of 1 to 3. For example, p1 may be 1. When p1 is an integer of 2 or greater, a plurality of L₁'s may all be the same or different.

In Formula 2, Z may be a substituted or unsubstituted naphthyl group, or a substituted or unsubstituted phenanthrenyl group. When Z is a substituted naphthyl group or a substituted phenanthrenyl group, Z may be a deuterium atom, a substituted or unsubstituted amine group, a substituted or unsubstituted silyl group, a substituted or unsubstituted aryl group having 6 to 15 ring-forming carbon atoms, or a substituted or unsubstituted heteroaryl group having 2 to 15 ring-forming carbon atoms. For example, Z may be an unsubstituted naphthyl group, a naphthyl group substituted with a deuterium atom, a naphthyl group substituted with an unsubstituted phenyl group, an unsubstituted phenanthrenyl group, or a phenanthrenyl group substituted with a deuterium atom, but the embodiment of the present disclosure is not limited thereto.

In Formula 3, p2 may be an integer of 0 to 3. For example, p2 may be 0 or 1. When p2 is 0, the amine compound of one or more embodiments may include a third substituent directly bonded to the nitrogen atom of amine. In some embodiments, when p2 is 1, 2, or 3, the amine compound of one or more embodiments may include a third substituent linked to the nitrogen atom of amine through L₂. When p2 is an integer of 2 or greater, a plurality of L₂'s may all be the same or different.

In Formula 3, U may be a hydrogen atom, a deuterium atom, a halogen atom, a substituted or unsubstituted amine group, a substituted or unsubstituted silyl group, a substituted or unsubstituted aryl group having 6 to 30 ring-forming carbon atoms, or a substituted or unsubstituted heteroaryl group having 2 to 30 ring-forming carbon atoms. For example, U may be a hydrogen atom, a deuterium atom, or a substituted or unsubstituted phenyl group. When U is substituted with another substituent, U may be substituted with a deuterium atom, but the embodiment of the present disclosure is not limited thereto.

In Formula 3, m may be an integer of 0 to 7. When m is 0 in Formula 3, Formula 3 may not be substituted with U. For example, when m is 0 in Formula 3, the amine compound of one or more embodiments may include a third substituent which is not substituted with U. In Formula 3, a case in which m is 7 and all 7 U's are hydrogen atoms may be the same as the case in which m is 0. When m is an integer of 2 or greater, U provided in plurality may all be the same, or at least one of the plurality of U's may be different from the others.

In Formula 3, Q may be O, S, NRₐ₄, or CRₐ₅Rₐ₆. For example, the amine compound of one or more embodiments represented by Formula 1 may include a dibenzofuran moiety, a dibenzothiophene moiety, a carbazole moiety, or a fluorene moiety as a third substituent.

In one or more embodiments, a case in which Formula 3 is a substituted or unsubstituted 2-dibenzothiophene group may be excluded. For example, when the amine compound of one or more embodiments includes a dibenzothiophene moiety as a third substituent, a case in which the dibenzothiophene moiety is directly bonded to the nitrogen atom of amine at carbon 2 of dibenzothiophene may be excluded.

In Formula 3, Rₐ₄ may be a substituted or unsubstituted aryl group having 6 to 30 ring-forming carbon atoms, or a substituted or unsubstituted heteroaryl group having 2 to 30 ring-forming carbon atoms. For example, Rₐ₄ may be a phenyl group substituted with a deuterium atom or an unsubstituted phenyl group.

In Formula 3, Rₐ₅ and Rₐ₆ may each independently be a substituted or unsubstituted aryl group having 6 to 30 ring-forming carbon atoms, or a substituted or unsubstituted heteroaryl group having 2 to 30 ring-forming carbon atoms. In some embodiments, Rₐ₅ and Rₐ₆ may be bonded to form a ring. For example, Rₐ₅ and Rₐ₆ may each independently be a phenyl group substituted with a deuterium atom or an unsubstituted phenyl group. In some embodiments, Rₐ₅ and Rₐ₆ may be bonded to form a substituted or unsubstituted hydrocarbon ring. The hydrocarbon ring formed by the bonding of Rₐ₅ and Rₐ₆ may be linked to a ring in which U is substituted to form a spiro structure.

The amine compound of one or more embodiments may include a deuterium atom as a substituent. For example, in the amine compound represented by Formula 1, at least one of R₁ to R₁₅, W, X, and Y may include a deuterium atom or a substituent including a deuterium atom. However, this is presented as an example, and the embodiment of the present disclosure is not limited thereto.

In one or more embodiments, the amine compound represented by Formula 1 may be represented by Formula 1-1 or Formula 1-2. Formula 1-1 and Formula 1-2 each specify (W)ₙ in Formula 1. Formula 1-1 shows a case in which n is 0 in Formula 1, and Formula 1-2 shows a case in which n is 1 and W is a direct linkage in Formula 1 as an example. In Formulas 1-1 and 1-2, the same descriptions as in Formula 1 may be applied to X and Y.

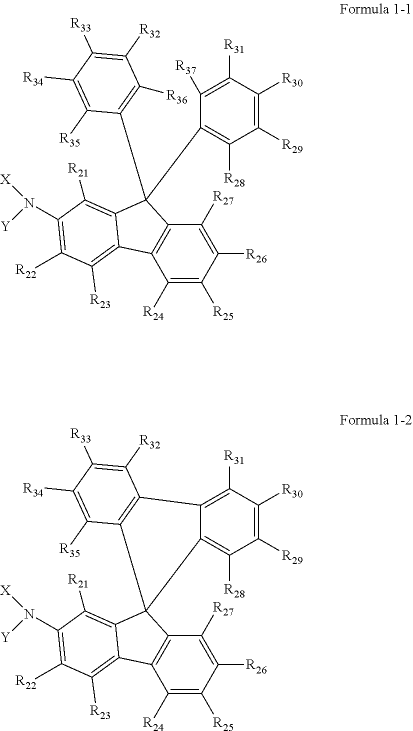

In Formulas 1-1 and 1-2, R₂₁ to R₃₅ may each independently be a hydrogen atom, a deuterium atom, or a substituted or unsubstituted aryl group having 6 to 30 ring-forming carbon atoms. For example, R₂₁ to R₃₅ may each independently be a hydrogen atom, a deuterium atom, an unsubstituted phenyl group, or a phenyl group substituted with a deuterium atom. In one or more embodiments, at least one selected from among R₂₃ to R₂₅, and R₂₇ to R₃₅ may be a substituted or unsubstituted aryl group having 6 to 30 ring-forming carbon atoms. For example, at least one of R₂₃ to R₂₅, and R₂₇ to R₃₅ may be a phenyl group substituted with a deuterium atom or an unsubstituted phenyl group, but the embodiment of the present disclosure is not limited thereto. For example, R₃₆ and R₃₇ may each independently be a hydrogen atom or a deuterium atom.

In one or more embodiments, the amine compound represented by Formula 1 may be represented by Formula 4-1 or Formula 4-2. Formulas 4-1 and 4-2 each show a structure in which Formula 2 and/or Formula 3 are linked to the nitrogen atom of amine of Formula 1 as an example. In Formulas 4-1 and 4-2, the same descriptions as in Formula 1 may be applied to R₁ to R₁₅, W, Q, U, n, and m.

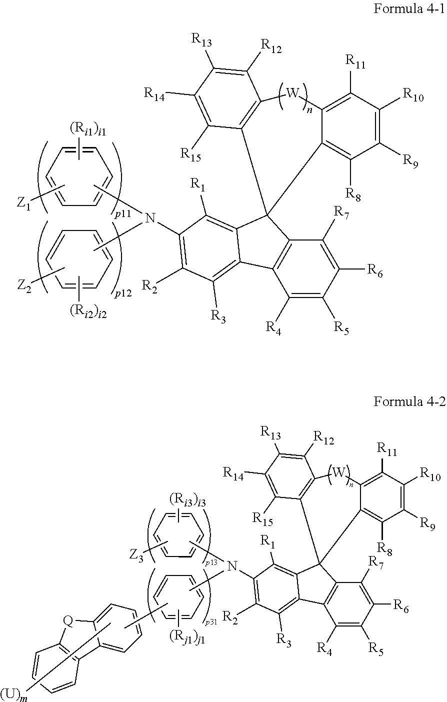

In Formulas 4-1 and 4-2, Z₁ to Z₃ may each independently be a substituted or unsubstituted naphthyl group or a substituted or unsubstituted phenanthrenyl group. For example, Z₁ to Z₃ may each independently be an unsubstituted naphthyl group, a naphthyl group substituted with a deuterium atom, a naphthyl group substituted with an unsubstituted phenyl group, an unsubstituted phenanthrenyl group, or a phenanthrenyl group substituted with a deuterium atom.

In Formulas 4-1 and 4-2, Rᵢ₁ to Rᵢ₃, and Rⱼ₁ may each independently be a hydrogen atom, a deuterium atom, a substituted or unsubstituted silyl group, a substituted or unsubstituted amine group, a substituted or unsubstituted aryl group having 6 to 15 ring-forming carbon atoms, or a substituted or unsubstituted heteroaryl group having 2 to 15 ring-forming carbon atoms. For example, Rᵢ₁ to Rᵢ₃, and Rⱼ₁ may each independently be a hydrogen atom or a deuterium atom.

In Formulas 4-1 and 4-2, i1 to i3, and j1 may each independently be an integer of 0 to 4. A case in which i1 to i3 and j1 are each 0 may be the same as a case in which i1 to i3 and j1 are 4, and Rᵢ₁ to Rᵢ₃, and Rⱼ₁ are hydrogen atoms. In one or more embodiments, when Rᵢ₁ to Rᵢ₃, and Rⱼ₁ are each an integer of 2 or greater, Rᵢ₁ to Rᵢ₃, and Rⱼ₁ provided in plurality may each be the same or at least one may be different from the others. In some embodiments, when i1 to i3 and j1 are each 0, the amine compound of one or more embodiments represented by Formulas 4-1 and 4-2 may not be substituted with Rᵢ₁ to Rᵢ₃, and Rⱼ1.

In Formulas 4-1 and 4-2, p11 to p13 may each independently be an integer of 1 to 3. For example, p11 to p13 may each be 1. p21 may be an integer of 0 to 3. For example, p21 may be 0 or 1.

In one or more embodiments, the amine compound represented by Formula 4-1 may be represented by Formula 4-1-1 or Formula 4-1-2. Formulas 4-1-1 and 4-1-2 each represent a bonding relationship of a second substituent linked to the nitrogen atom of amine through a linker, and show, as an example, bonding positions of Z₁ and Z₂ linked to the nitrogen atom of amine through a linker in Formula 4-1.

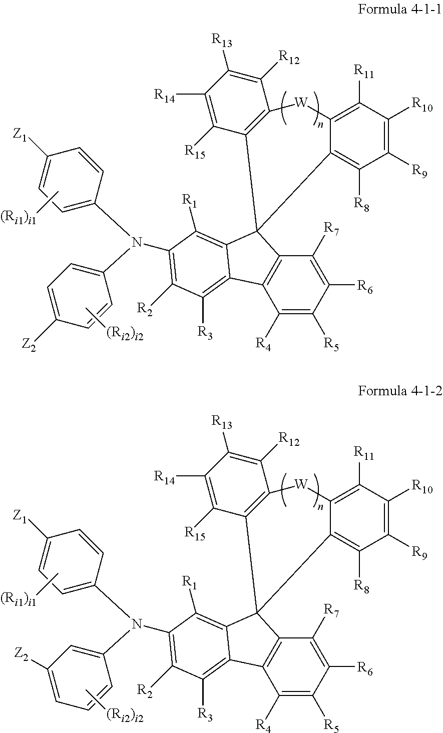

Referring to Formulas 4-1-1 and 4-1-2, Z₁ and Z₂ may each be linked to be positioned para or meta to the nitrogen atom of amine through a linker of a phenylene group.

In Formulas 4-1-1 and 4-1-2, the descriptions as in Formulas 1 and 4-1 may be applied to R₁ to R₁₅, Rᵢ₁, Rᵢ₂, W, Z₁, Z₂, i1, i2, and n.

In one or more embodiments, the amine compound represented by Formula 4-2 may be represented by Formula 4-2-1 or Formula 4-2-2. Formulas 4-2-1 and 4-2-2 shows, as an example, a connection relationship between the second substituent and the third substituent in Formula 4-2.

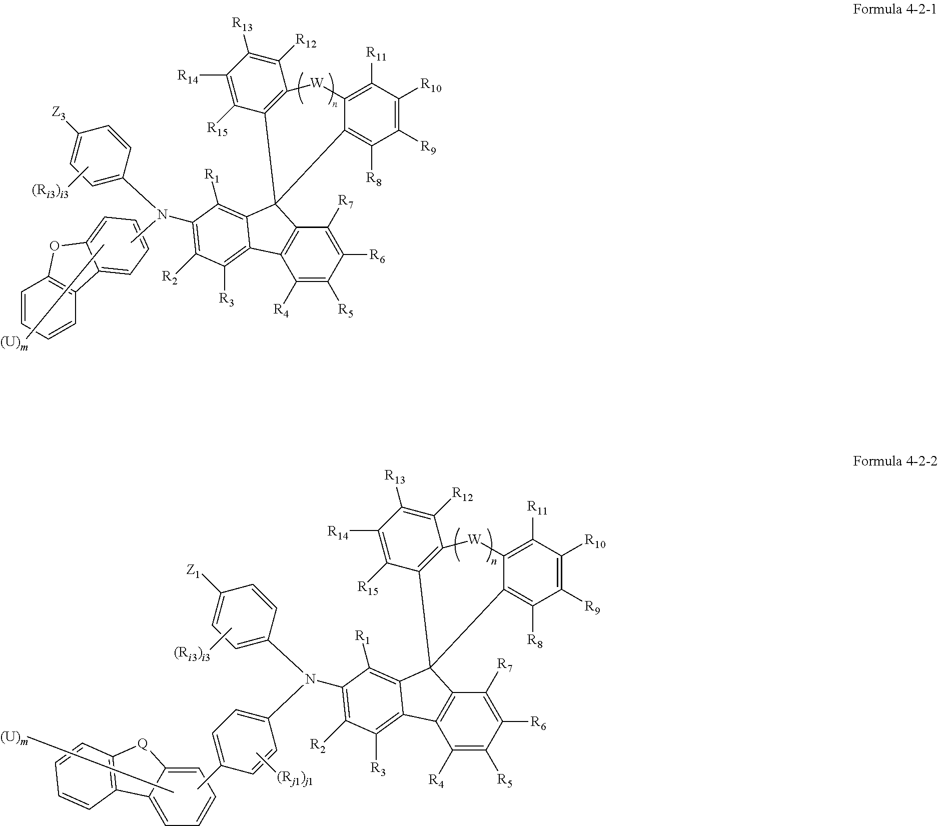

Referring to Formulas 4-2-1 and 4-2-2, Z₃ may be linked to be positioned para to the nitrogen atom of amine through a linker of a phenylene group unsubstituted or substituted with Rᵢ₃. In some embodiments,

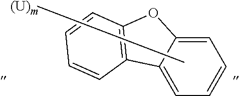

may be directly bonded to the nitrogen atom of amine, or may be linked to be positioned para to the nitrogen atom of amine through a linker of a phenylene group unsubstituted or substituted with Rⱼ₁.

In Formulas 4-2-1 and 4-2-2, the descriptions as in Formulas 1 and 4-1 may be applied to R₁ to R₁₅, Rᵢ₃, Rⱼ₁, W, Q, U, Z₃, i3, j1, n, and m.

In one or more embodiments, the amine compound represented by Formula 1 may be represented by Formula 5. Formula 5 shows, as an example, a structure in which a substituted or unsubstituted phenyl group is linked to at least one of R₃ to R₅, and R₇ to R₁₅ in Formula 1. In Formula 5, the descriptions as in Formula 1 may be applied to X, Y, W, and n.

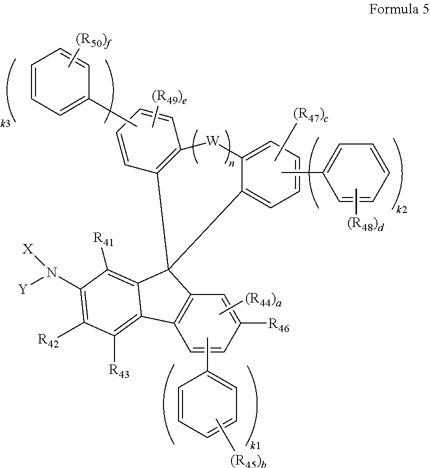

Referring to Formula 5, the amine compound of one or more embodiments may include at least one substituted or unsubstituted phenyl group in a fluorene moiety. In one or more embodiments, k1 to k3 may each independently be 0 or 1, and at least one of k1 to k3 may be 1. For example, any one or two of k1 to k3 may be 1 and the others may be 0. For example, the amine compound of one or more embodiments may include one or two substituted or unsubstituted phenyl groups directly bonded to the fluorene moiety.

In Formula 5, R₄₁ to R₄₄, R₄₆, R₄₇, and R₄₉ may each independently be a hydrogen atom, a deuterium atom, a halogen atom, a substituted or unsubstituted amine group, a substituted or unsubstituted silyl group, a substituted or unsubstituted aryl group having 6 to 30 ring-forming carbon atoms, or a substituted or unsubstituted heteroaryl group having 2 to 30 ring-forming carbon atoms. For example, R₄₁ to R₄₄, R₄₆, R₄₇, and R₄₉ may each be a hydrogen atom or a deuterium atom.

In Formula 5, a may be an integer of 0 to 3, and c and e may each independently be an integer of 0 to 4. In Formula 5, when a, c, and e in Formula 5 are each 0, the amine compound represented by Formula 5 may not be substituted with each (any) of R₄₄, R₄₇, and R₄₉. For example, when a, c, and e in Formula 5 are each 0, the amine compound of one or more embodiments may include a first substituent unsubstituted with each (any) of R₄₄, R₄₇, and R₄₉. In Formula 5, a case in which a is 3 and all 3 R₄₄'s are hydrogen atoms may be the same as the case in which a is 0. In some embodiments, a case in which c and e are each 4 and all four R₄₇'s and R₄₉'s are hydrogen atoms may be the same as the case in which c and e are 0. When a, c, and e are each an integer of 2 or greater, R₄₄, R₄₇, and R₄₉ provided in plurality may each be the same, or at least one of R₄₄, R₄₇, or R₄₉ provided in plurality may each be different from the others.

In Formula 5, R₄₅, R₄₈, and R₅₀ may each independently be a hydrogen atom or a deuterium atom. b, d, and f may each independently be an integer of 0 to 5. In Formula 5, when b, d, and f are each 0, the amine compound represented by Formula 5 may not be substituted with each of R₄₅, R₄₈, and R₅₀. For example, a case in which b, d, and f are each 5, and all 6 R₄₅'s, R₄'s, and R₅₀'s are each a hydrogen atom may be the same as the case in which b, d, and f are each 0. In some embodiments, a case in which b, d, and f are each 6 and all 5 R₄₅'s, R₄'s, and R₅₀'s are hydrogen atoms may be the same as the case in which b, d, and f are 0. When b, d, and f are each an integer of 2 or greater, R₄₅, R₄₈, and R₅₀ provided in plurality may each be the same, or at least one of R₄₅, R₄₈, or R₅₀ provided in plurality may each be different from the others.

In one or more embodiments, the amine compound represented by Formula 5 may be represented by any one selected from among Formulas 6-1, 6-2, and 7-1 to 7-3. Formula 6-1, Formula 6-2, and Formula 7-1 to Formula 7-3 may each have a structure in which X and Y in Formula 5 are specified. For example, the amine compound represented by Formula 5 may include two second substituents and may be represented by Formula 6-1 or Formula 6-2. In some embodiments, the amine compound represented by Formula 5 may include one second substituent and one third substituent, and may be represented by any one among Formulas 7-1 to 7-3.

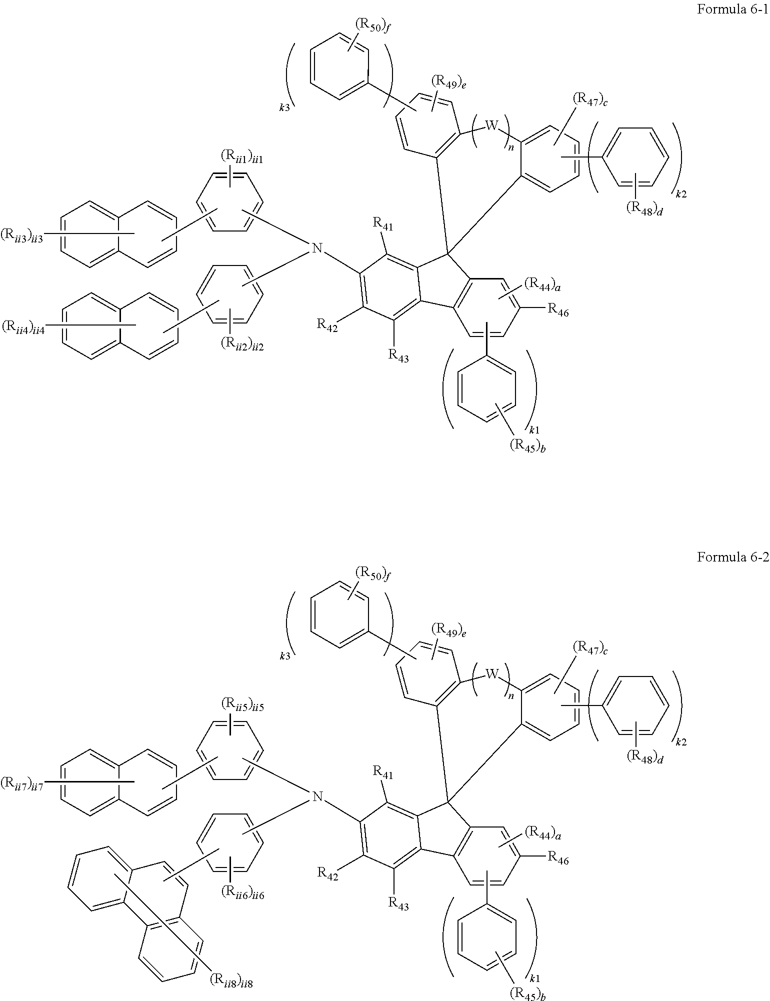

In Formulas 6-1 and 6-2, the descriptions as in Formulas 1 and 5 may be applied to R₄₁ to R₅₀, W, a to f, k1 to k3, and n.

In Formulas 6-1 and 6-2, Rᵢᵢ₁ to Rᵢᵢ₈ may each independently be a hydrogen atom, a deuterium atom, a substituted or unsubstituted silyl group, a substituted or unsubstituted amine group, a substituted or unsubstituted aryl group having 6 to 15 ring-forming carbon atoms, or a substituted or unsubstituted heteroaryl group having 2 to 15 ring-forming carbon atoms. For example, Rᵢᵢ₁, Rᵢᵢ₂, Rᵢᵢ₅, and Rᵢᵢ₆ may each independently be a hydrogen atom or a deuterium atom. In some embodiments, Rᵢᵢ₁, Rᵢᵢ₂, Rᵢᵢ₆, and Rᵢᵢ₆ may each independently be a hydrogen atom, a deuterium atom, an unsubstituted phenyl group, or a phenyl group substituted with a deuterium atom.

In Formulas 6-1 and 6-2, ii1, ii2, ii5, and ii6 may each independently be an integer of 0 to 4. A case in which ii1, ii2, ii5, and ii6 are each 0 may be the same as a case in which ii1, ii2, ii5, and ii6 are 4, and Rᵢᵢ₁, Rᵢᵢ₂, Riis, and Rᵢᵢ₆ are hydrogen atoms. In one or more embodiments, when ii1, ii2, ii5, and ii6 are each an integer of 2 or greater, Rᵢᵢ₁, Rᵢᵢ₂, Rᵢᵢ₆, and Rᵢᵢ₆ provided in plurality may each be the same or at least one may be different from the others. In some embodiments, when ii1, ii2, ii5, and ii6 are each 0, the amine compound of one or more embodiments represented by Formulas 6-1 and 6-2 may not be substituted with Rᵢᵢ₁, Rᵢᵢ₂, Rᵢᵢ₆, and Rᵢᵢ₆.

In Formulas 6-1 and 6-2, ii3, ii4, and ii7 may each independently be an integer of 0 to 7, and ii8 may be an integer of 0 to 9. A case in which ii3, ii4, and ii7 are each 0 may be the same as a case in which ii3, ii4, and ii7 are 7, and all 7 Rᵢᵢ₃'s, Rᵢᵢ₄'s, and Rᵢᵢ₇'s are each a hydrogen atom. In some embodiments, a case in which ii8 is 0 may be the same as a case in which ii8 is 9 and all 9 Rᵢᵢ₈'s are hydrogen atoms. In one or more embodiments, when ii3, ii4, ii7, and ii8 are each an integer of 2 or greater, Rᵢᵢ₃, Rᵢᵢ₄, Rᵢᵢ₇, and Riis provided in plurality may each be the same or at least one may be different from the others. In some embodiments, when ii3, ii4, ii7, and ii8 are each 0, the amine compound of one or more embodiments represented by Formula 6-1 or Formula 6-2 may not be substituted with Rᵢᵢ₃, Rᵢᵢ₄, Rᵢᵢ₇, and Rᵢᵢ₈.

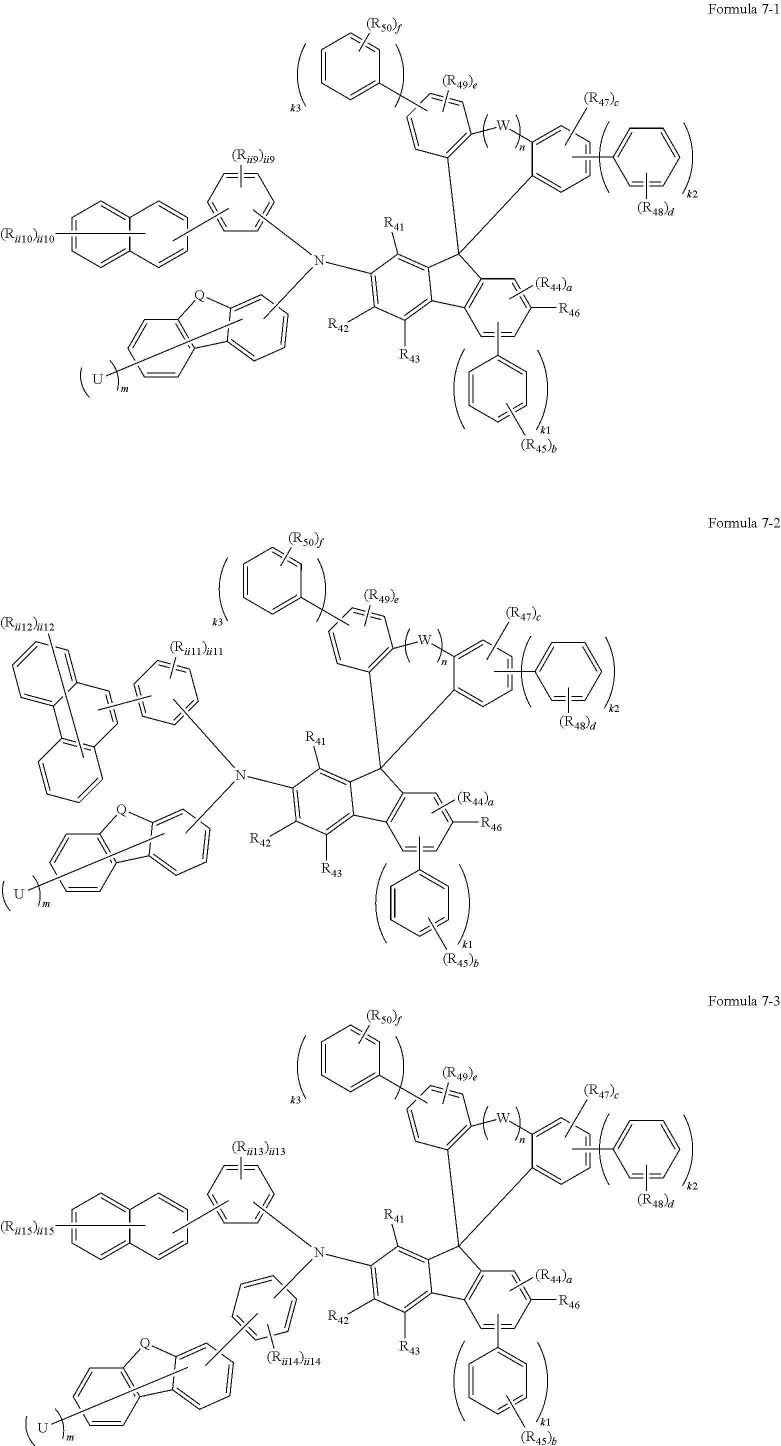

In Formulas 7-1 to 7-3, the descriptions as in Formulas 1 and 5 may be applied to R₄₁ to R₅₀, Q, U, W, a to f, k1 to k3, m, and n.

In Formulas 7-1 to 7-3, Rᵢᵢ₉ to Rᵢᵢ₁₅ may each independently be a hydrogen atom, a deuterium atom, a substituted or unsubstituted silyl group, a substituted or unsubstituted amine group, a substituted or unsubstituted aryl group having 6 to 15 ring-forming carbon atoms, or a substituted or unsubstituted heteroaryl group having 2 to 15 ring-forming carbon atoms. For example, Rᵢᵢ₉, Rᵢᵢ₁₁, Rᵢᵢ₁₃, and Rᵢᵢ₁₄ may each independently be a hydrogen atom or a deuterium atom. In some embodiments, Rᵢᵢ₁₀, Rᵢᵢ₁₂, and Rᵢᵢ₁₅ may each independently be a hydrogen atom, a deuterium atom, an unsubstituted phenyl group, or a phenyl group substituted with a deuterium atom.

In Formulas 7-1 to 7-3, ii9, ii11, ii13, and ii14 may each independently be an integer of 0 to 4. A case in which ii9, ii11, ii13, and ii14 are each 0 may be the same as a case in which ii9, ii11, ii13, and ii14 are 4, and Rᵢᵢ₉, Rᵢᵢ₁₁, Rᵢᵢ₁₃, and Rᵢᵢ₁₄ are hydrogen atoms. In one or more embodiments, when ii9, ii11, ii13, and ii14 are each an integer of 2 or greater, Rᵢᵢ₉, Rᵢᵢ₁₁, Rᵢᵢ₁₃, and Rᵢᵢ₁₄ provided in plurality may each be the same or at least one may be different from the others. In some embodiments, when ii9, ii11, ii13, and ii14 are each 0, the amine compound of one or more embodiments represented by Formulas 7-1 to 7-3 may not be substituted with Rᵢᵢ₉, Rᵢᵢ₁₁, Rᵢᵢ₁₃, and Rᵢᵢ₁₄.

In Formulas 7-1 to 7-3, ii10 and ii15 may each independently be an integer of 0 to 7, and ii12 may be an integer of 0 to 9. A case in which ii10 and ii15 are each 0 may be the same as a case in which ii10 and ii15 are 7, and all 7 Rᵢᵢ₁₀'s, and Rᵢᵢ₁₅'s are each a hydrogen atom. In some embodiments, a case in which ii12 is 0 may be the same as a case in which ii12 is 9 and all 9 Rᵢᵢ₁₂'s are hydrogen atoms. In one or more embodiments, when ii10, ii12, and ii15 are each an integer of 2 or greater, Rᵢᵢ₁₀, Rᵢᵢ₁₂, and Rᵢᵢ₁₅ provided in plurality may each be the same or at least one may be different from the others. In some embodiments, when ii10, ii12, and ii15 are each 0, the amine compound of one or more embodiments represented by Formulas 7-1 to 7-3 may not be substituted with Rᵢᵢ₁₀, Rᵢᵢ₁₂, and Rᵢᵢ₁₅.

The amine compound of one or more embodiments represented by Formula 1 may be represented by any one selected from among the amine compounds of Compound Group 1. The hole transport region HTR of the light emitting element ED of one or more embodiments may include at least one of the amine compounds disclosed in Compound Group 1. For example, the electron blocking layer EBL of the light emitting element ED may include at least one of the amine compounds disclosed in Compound Group 1. In Compound Group 1, “D” is a deuterium atom.

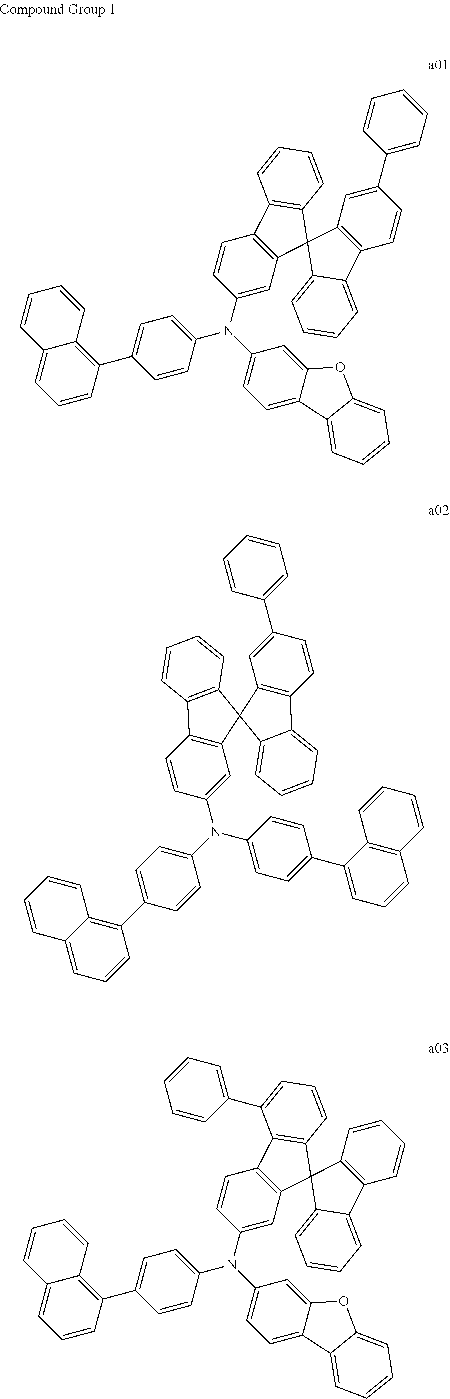 [SMILES: c1ccc(-c2ccc3c(c2)C2(c4ccccc4-3)c3ccccc3-c3ccc(N(c4ccc(-c5cccc6ccccc56)cc4)c4ccc5c(c4)oc4ccccc45)cc32)cc1 ; c1ccc(-c2ccc3c(c2)C2(c4ccccc4-3)c3ccccc3-c3ccc(N(c4ccc(-c5cccc6ccccc56)cc4)c4ccc(-c5cccc6ccccc56)cc4)cc32)cc1 ; c1ccc(-c2cccc3c2-c2ccc(N(c4ccc(-c5cccc6ccccc56)cc4)c4ccc5c(c4)oc4ccccc45)cc2C32c3ccccc3-c3ccccc32)cc1]

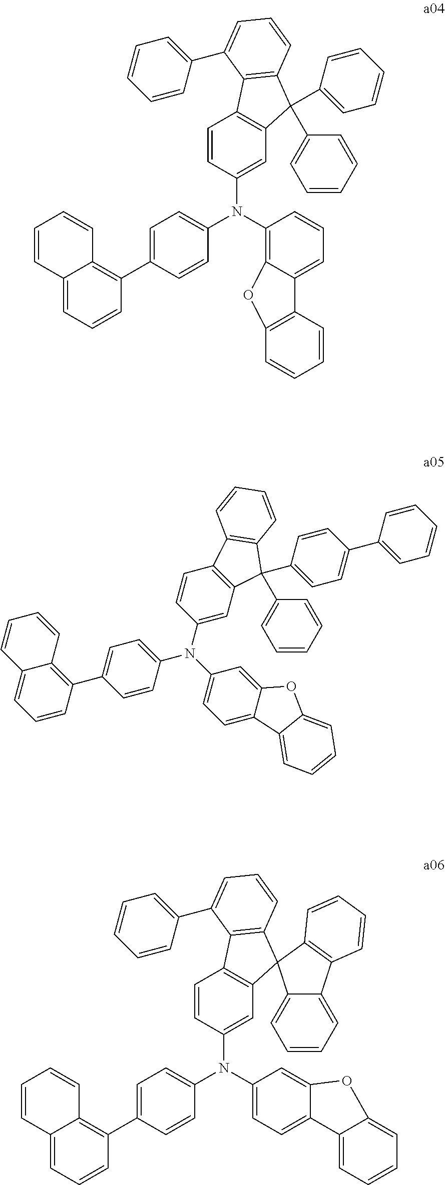 [SMILES: c1ccc(-c2cccc3c2-c2ccc(N(c4ccc(-c5cccc6ccccc56)cc4)c4cccc5c4oc4ccccc45)cc2C3(c2ccccc2)c2ccccc2)cc1 ; c1ccc(-c2ccc(C3(c4ccccc4)c4ccccc4-c4ccc(N(c5ccc(-c6cccc7ccccc67)cc5)c5ccc6c(c5)oc5ccccc56)cc43)cc2)cc1 ; c1ccc(-c2cccc3c2-c2ccc(N(c4ccc(-c5cccc6ccccc56)cc4)c4ccc5c(c4)oc4ccccc45)cc2C32c3ccccc3-c3ccccc32)cc1]

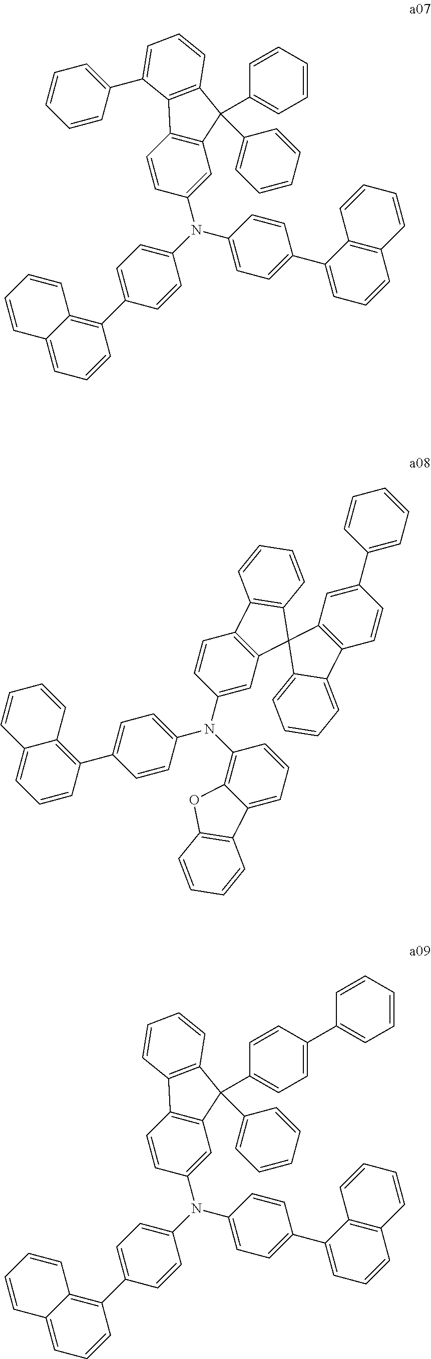 [SMILES: c1ccc(-c2cccc3c2-c2ccc(N(c4ccc(-c5cccc6ccccc56)cc4)c4ccc(-c5cccc6ccccc56)cc4)cc2C3(c2ccccc2)c2ccccc2)cc1 ; c1ccc(-c2ccc3c(c2)C2(c4ccccc4-3)c3ccccc3-c3ccc(N(c4ccc(-c5cccc6ccccc56)cc4)c4cccc5c4oc4ccccc45)cc32)cc1 ; c1ccc(-c2ccc(C3(c4ccccc4)c4ccccc4-c4ccc(N(c5ccc(-c6cccc7ccccc67)cc5)c5ccc(-c6cccc7ccccc67)cc5)cc43)cc2)cc1]

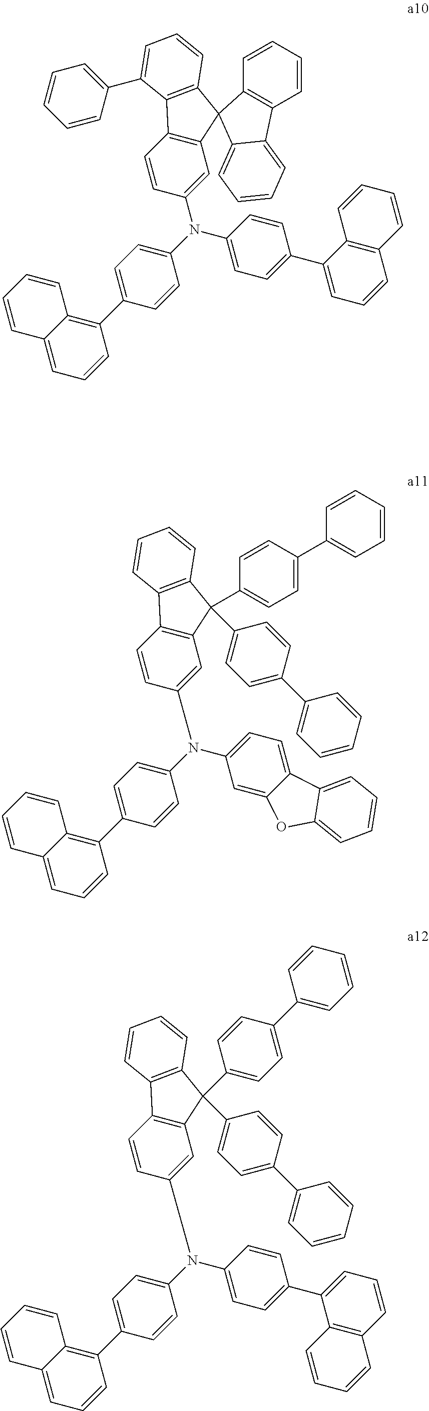 [SMILES: c1ccc(-c2cccc3c2-c2ccc(N(c4ccc(-c5cccc6ccccc56)cc4)c4ccc(-c5cccc6ccccc56)cc4)cc2C32c3ccccc3-c3ccccc32)cc1 ; c1ccc(-c2ccc(C3(c4ccc(-c5ccccc5)cc4)c4ccccc4-c4ccc(N(c5ccc(-c6cccc7ccccc67)cc5)c5ccc6c(c5)oc5ccccc56)cc43)cc2)cc1 ; c1ccc(-c2ccc(C3(c4ccc(-c5ccccc5)cc4)c4ccccc4-c4ccc(N(c5ccc(-c6cccc7ccccc67)cc5)c5ccc(-c6cccc7ccccc67)cc5)cc43)cc2)cc1]

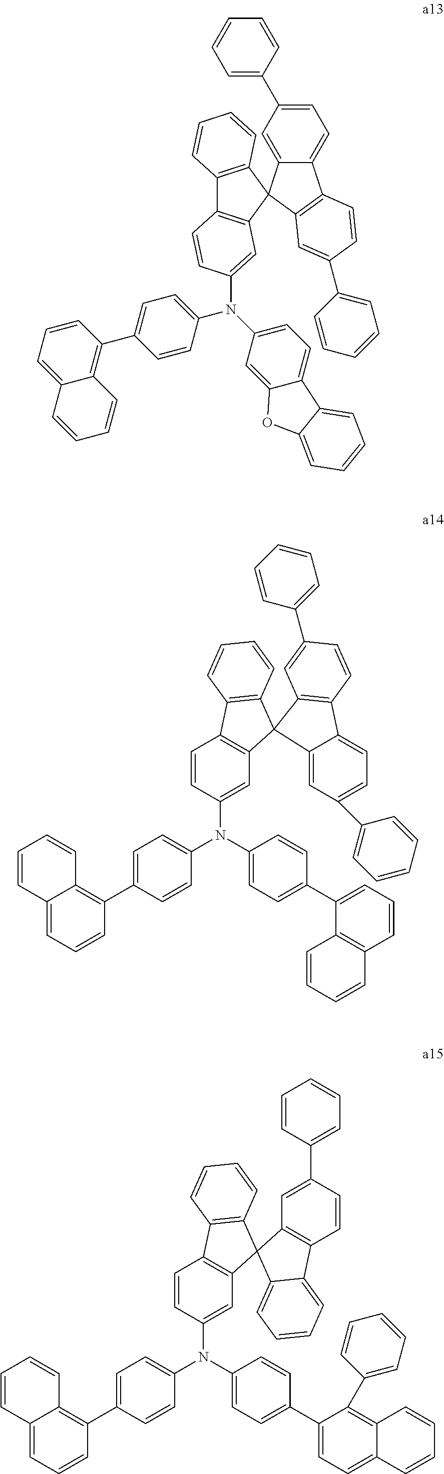 [SMILES: c1ccc(-c2ccc3c(c2)C2(c4ccccc4-c4ccc(N(c5ccc(-c6cccc7ccccc67)cc5)c5ccc6c(c5)oc5ccccc56)cc42)c2cc(-c4ccccc4)ccc2-3)cc1 ; c1ccc(-c2ccc3c(c2)C2(c4ccccc4-c4ccc(N(c5ccc(-c6cccc7ccccc67)cc5)c5ccc(-c6cccc7ccccc67)cc5)cc42)c2cc(-c4ccccc4)ccc2-3)cc1 ; c1ccc(-c2ccc3c(c2)C2(c4ccccc4-3)c3ccccc3-c3ccc(N(c4ccc(-c5ccc6ccccc6c5-c5ccccc5)cc4)c4ccc(-c5cccc6ccccc56)cc4)cc32)cc1]

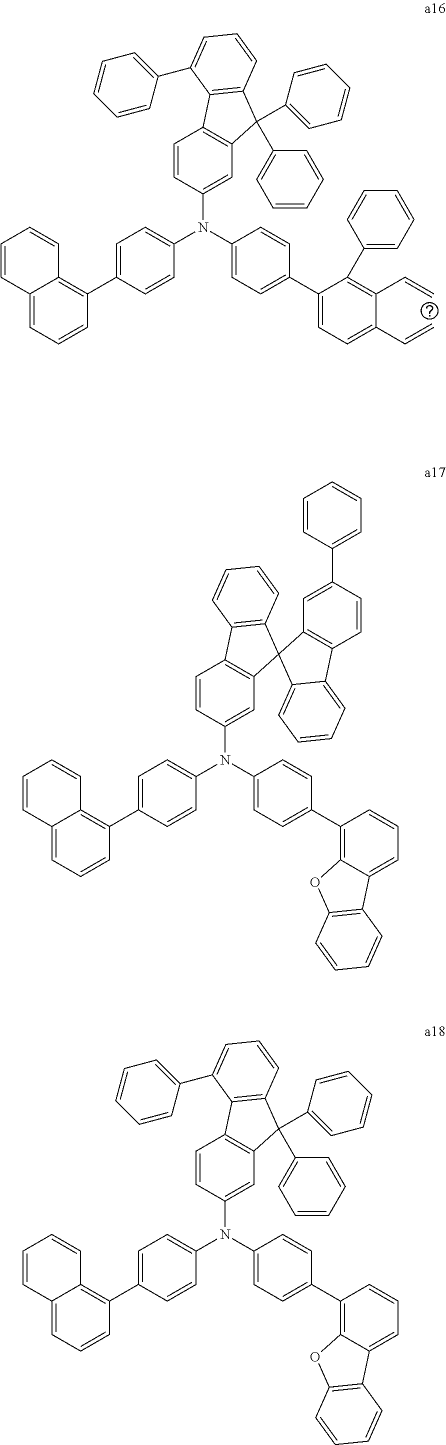 [SMILES: C=Cc1ccc(-c2ccc(N(c3ccc(-c4cccc5ccccc45)cc3)c3ccc4c(c3)C(c3ccccc3)(c3ccccc3)c3cccc(-c5ccccc5)c3-4)cc2)c(-c2ccccc2)c1C=C ; c1ccc(-c2ccc3c(c2)C2(c4ccccc4-3)c3ccccc3-c3ccc(N(c4ccc(-c5cccc6ccccc56)cc4)c4ccc(-c5cccc6c5oc5ccccc56)cc4)cc32)cc1 ; c1ccc(-c2cccc3c2-c2ccc(N(c4ccc(-c5cccc6ccccc56)cc4)c4ccc(-c5cccc6c5oc5ccccc56)cc4)cc2C3(c2ccccc2)c2ccccc2)cc1]

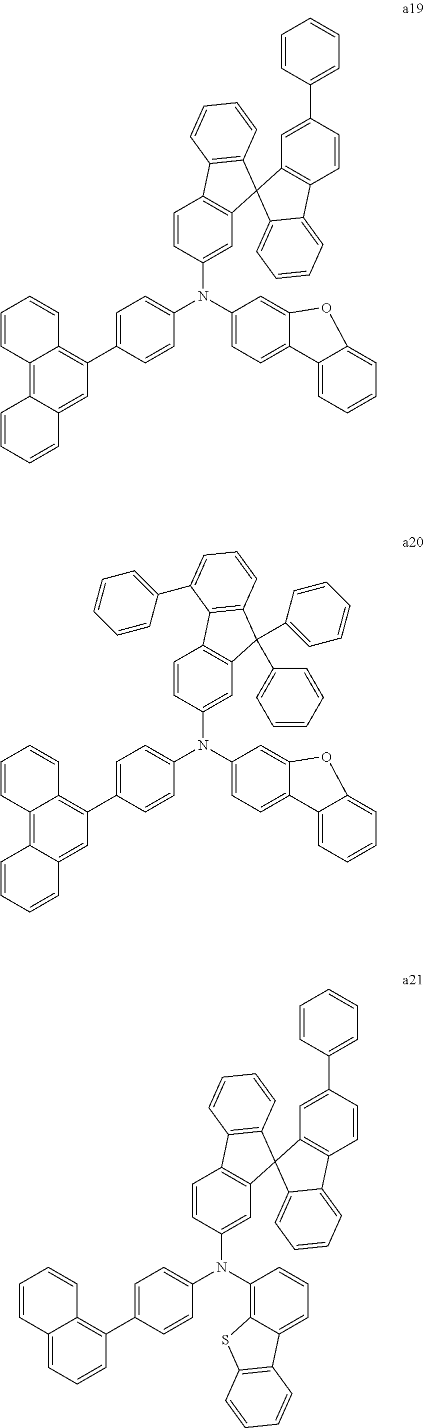 [SMILES: c1ccc(-c2ccc3c(c2)C2(c4ccccc4-3)c3ccccc3-c3ccc(N(c4ccc(-c5cc6ccccc6c6ccccc56)cc4)c4ccc5c(c4)oc4ccccc45)cc32)cc1 ; c1ccc(-c2cccc3c2-c2ccc(N(c4ccc(-c5cc6ccccc6c6ccccc56)cc4)c4ccc5c(c4)oc4ccccc45)cc2C3(c2ccccc2)c2ccccc2)cc1 ; c1ccc(-c2ccc3c(c2)C2(c4ccccc4-3)c3ccccc3-c3ccc(N(c4ccc(-c5cccc6ccccc56)cc4)c4cccc5c4sc4ccccc45)cc32)cc1]

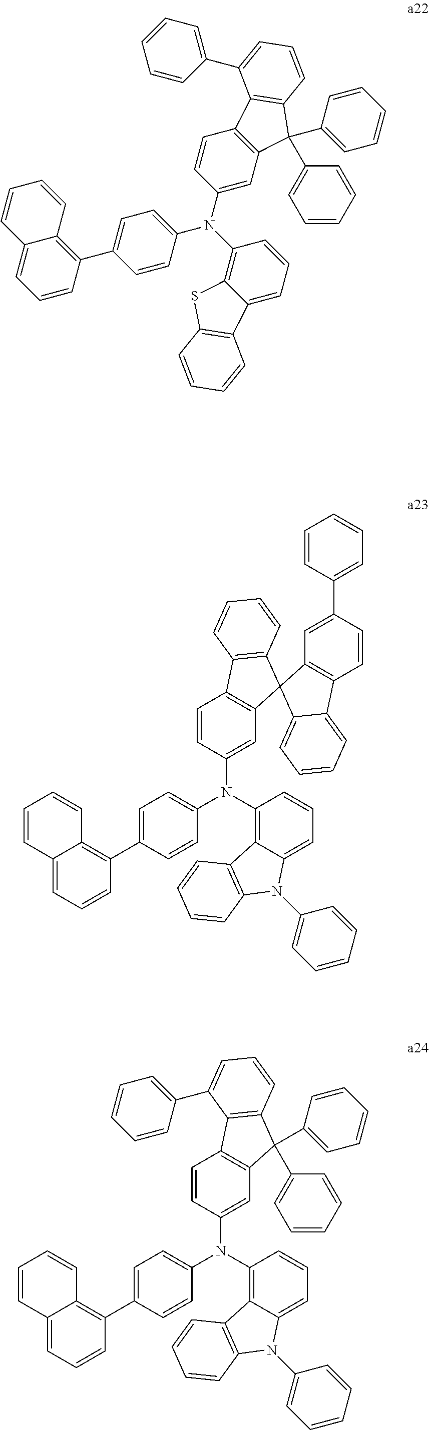 [SMILES: c1ccc(-c2cccc3c2-c2ccc(N(c4ccc(-c5cccc6ccccc56)cc4)c4cccc5c4sc4ccccc45)cc2C3(c2ccccc2)c2ccccc2)cc1 ; c1ccc(-c2ccc3c(c2)C2(c4ccccc4-3)c3ccccc3-c3ccc(N(c4ccc(-c5cccc6ccccc56)cc4)c4cccc5c4c4ccccc4n5-c4ccccc4)cc32)cc1 ; c1ccc(-c2cccc3c2-c2ccc(N(c4ccc(-c5cccc6ccccc56)cc4)c4cccc5c4c4ccccc4n5-c4ccccc4)cc2C3(c2ccccc2)c2ccccc2)cc1]

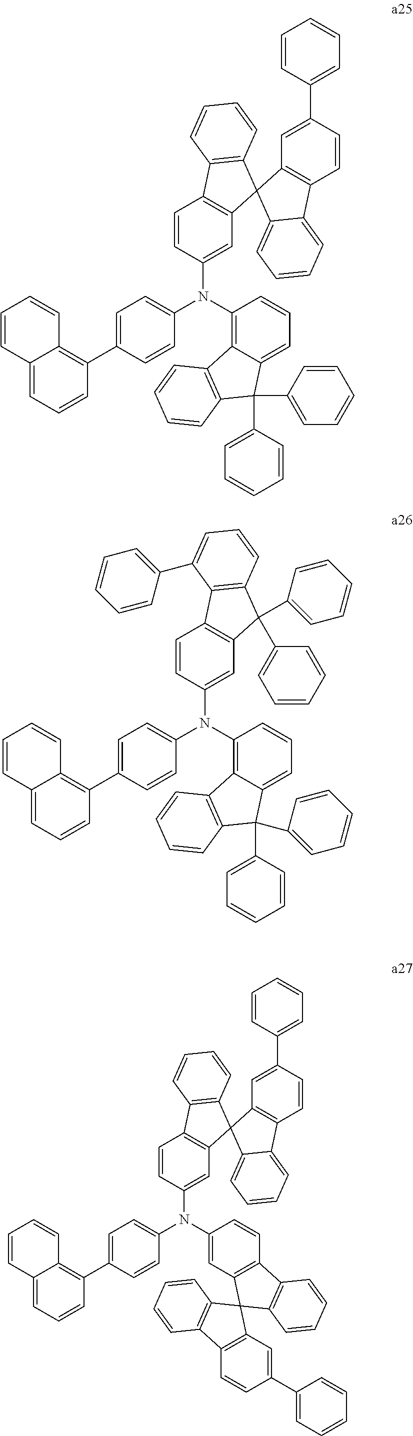

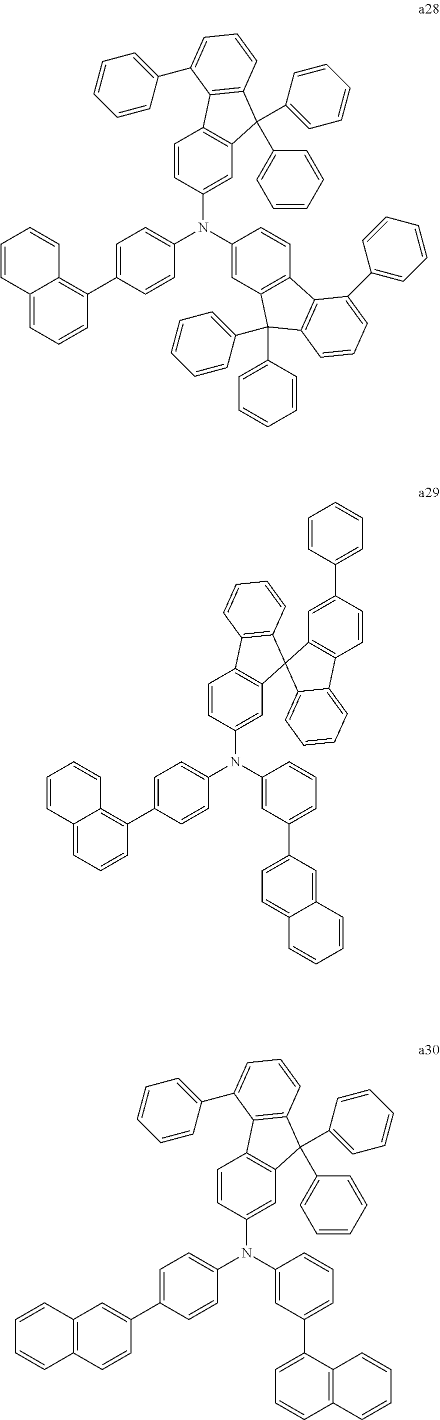 [SMILES: c1ccc(-c2cccc3c2-c2ccc(N(c4ccc(-c5cccc6ccccc56)cc4)c4ccc5c(c4)C(c4ccccc4)(c4ccccc4)c4cccc(-c6ccccc6)c4-5)cc2C3(c2ccccc2)c2ccccc2)cc1 ; c1ccc(-c2ccc3c(c2)C2(c4ccccc4-3)c3ccccc3-c3ccc(N(c4ccc(-c5cccc6ccccc56)cc4)c4cccc(-c5ccc6ccccc6c5)c4)cc32)cc1 ; c1ccc(-c2cccc3c2-c2ccc(N(c4ccc(-c5ccc6ccccc6c5)cc4)c4cccc(-c5cccc6ccccc56)c4)cc2C3(c2ccccc2)c2ccccc2)cc1]

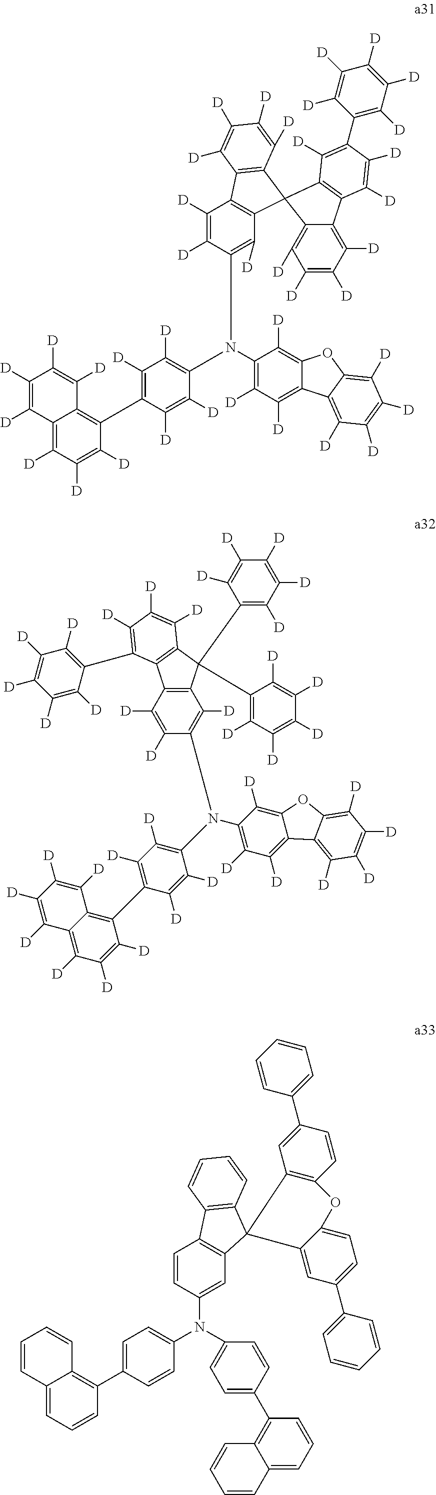

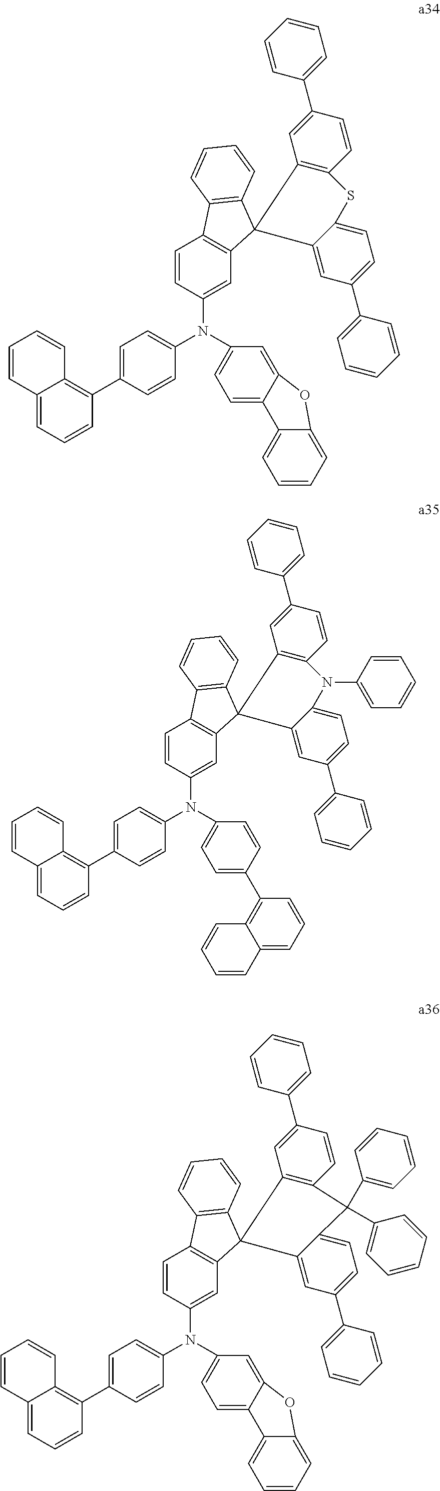

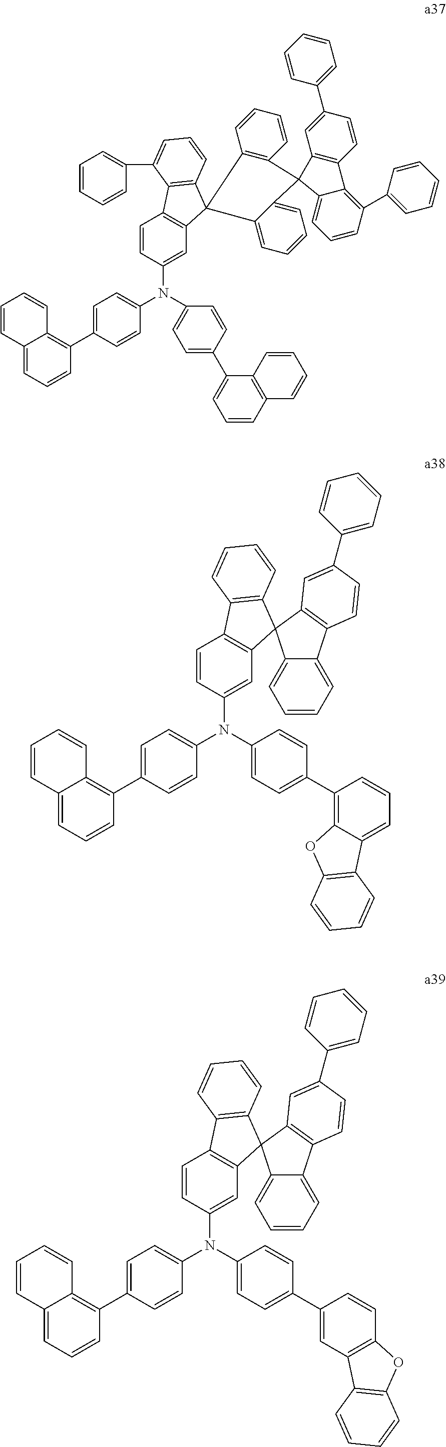 [SMILES: c1ccc(-c2ccc3c(c2)C2(c4ccccc4C4(c5cc(N(c6ccc(-c7cccc8ccccc78)cc6)c6ccc(-c7cccc8ccccc78)cc6)ccc5-c5c(-c6ccccc6)cccc54)c4ccccc42)c2cccc(-c4ccccc4)c2-3)cc1 ; c1ccc(-c2ccc3c(c2)C2(c4ccccc4-3)c3ccccc3-c3ccc(N(c4ccc(-c5cccc6ccccc56)cc4)c4ccc(-c5cccc6c5oc5ccccc56)cc4)cc32)cc1 ; c1ccc(-c2ccc3c(c2)C2(c4ccccc4-3)c3ccccc3-c3ccc(N(c4ccc(-c5ccc6oc7ccccc7c6c5)cc4)c4ccc(-c5cccc6ccccc56)cc4)cc32)cc1]

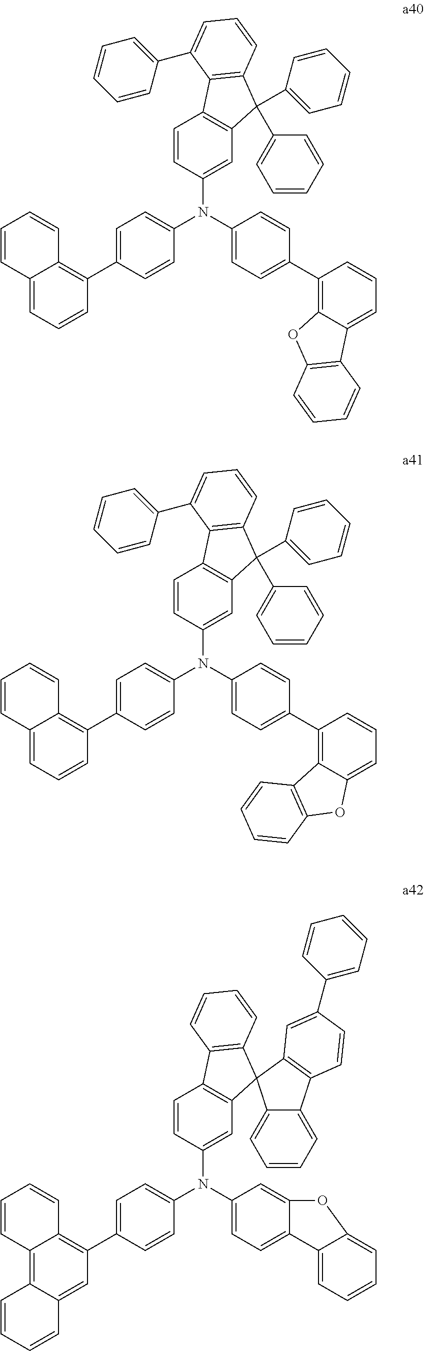 [SMILES: c1ccc(-c2cccc3c2-c2ccc(N(c4ccc(-c5cccc6ccccc56)cc4)c4ccc(-c5cccc6c5oc5ccccc56)cc4)cc2C3(c2ccccc2)c2ccccc2)cc1 ; c1ccc(-c2cccc3c2-c2ccc(N(c4ccc(-c5cccc6ccccc56)cc4)c4ccc(-c5cccc6oc7ccccc7c56)cc4)cc2C3(c2ccccc2)c2ccccc2)cc1 ; c1ccc(-c2ccc3c(c2)C2(c4ccccc4-3)c3ccccc3-c3ccc(N(c4ccc(-c5cc6ccccc6c6ccccc56)cc4)c4ccc5c(c4)oc4ccccc45)cc32)cc1]

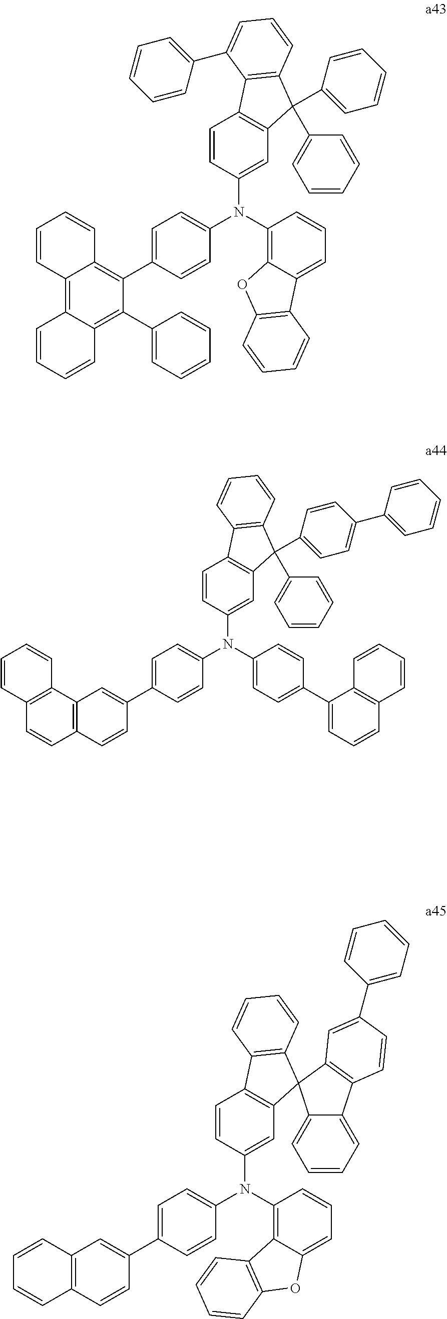 [SMILES: c1ccc(-c2cccc3c2-c2ccc(N(c4ccc(-c5c(-c6ccccc6)c6ccccc6c6ccccc56)cc4)c4cccc5c4oc4ccccc45)cc2C3(c2ccccc2)c2ccccc2)cc1 ; c1ccc(-c2ccc(C3(c4ccccc4)c4ccccc4-c4ccc(N(c5ccc(-c6ccc7ccc8ccccc8c7c6)cc5)c5ccc(-c6cccc7ccccc67)cc5)cc43)cc2)cc1 ; c1ccc(-c2ccc3c(c2)C2(c4ccccc4-3)c3ccccc3-c3ccc(N(c4ccc(-c5ccc6ccccc6c5)cc4)c4cccc5oc6ccccc6c45)cc32)cc1]

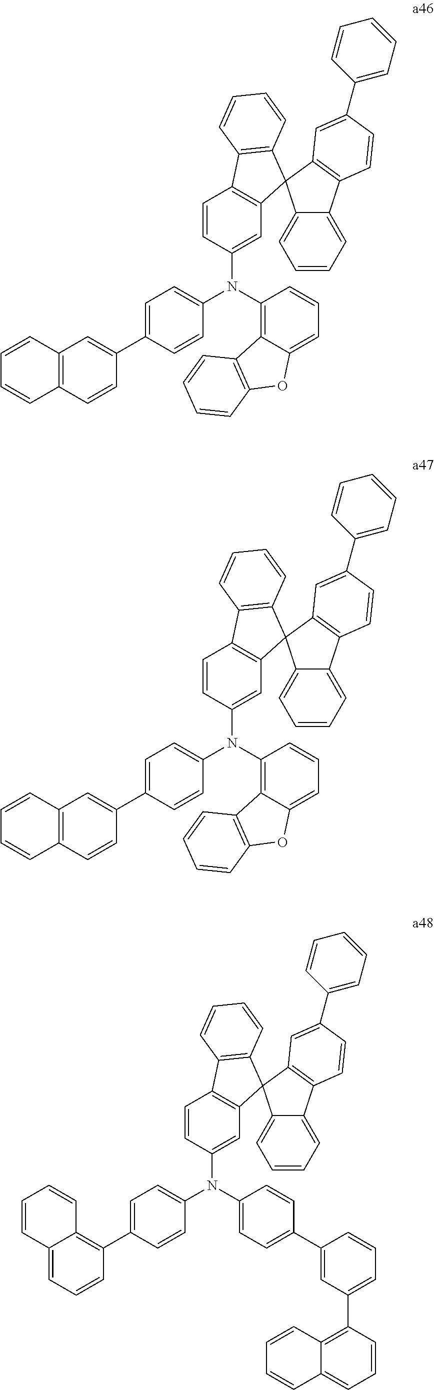 [SMILES: c1ccc(-c2ccc3c(c2)C2(c4ccccc4-3)c3ccccc3-c3ccc(N(c4ccc(-c5ccc6ccccc6c5)cc4)c4cccc5oc6ccccc6c45)cc32)cc1 ; c1ccc(-c2ccc3c(c2)C2(c4ccccc4-3)c3ccccc3-c3ccc(N(c4ccc(-c5ccc6ccccc6c5)cc4)c4cccc5oc6ccccc6c45)cc32)cc1 ; c1ccc(-c2ccc3c(c2)C2(c4ccccc4-3)c3ccccc3-c3ccc(N(c4ccc(-c5cccc(-c6cccc7ccccc67)c5)cc4)c4ccc(-c5cccc6ccccc56)cc4)cc32)cc1]

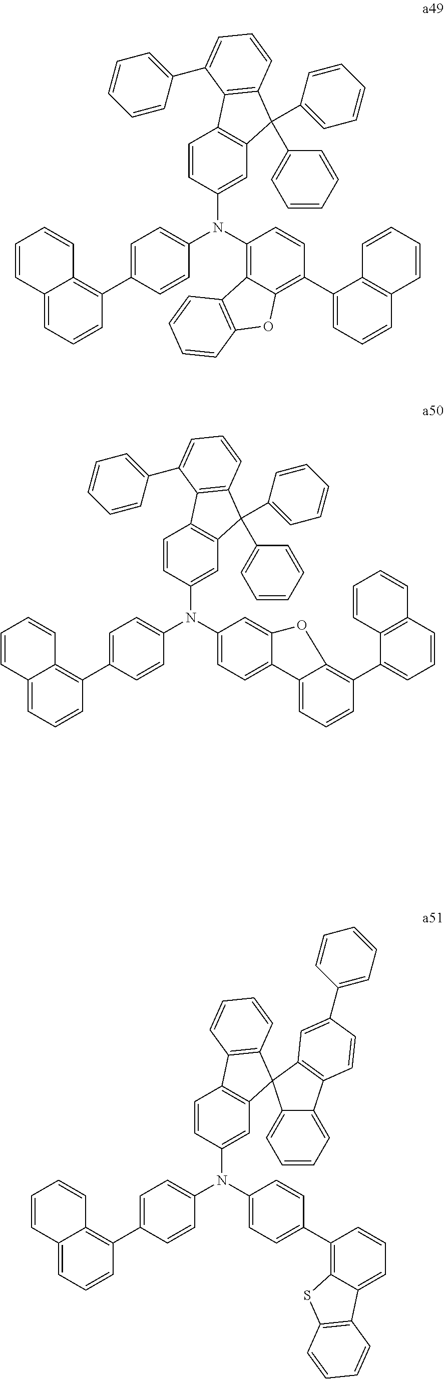 [SMILES: c1ccc(-c2cccc3c2-c2ccc(N(c4ccc(-c5cccc6ccccc56)cc4)c4ccc(-c5cccc6ccccc56)c5oc6ccccc6c45)cc2C3(c2ccccc2)c2ccccc2)cc1 ; c1ccc(-c2cccc3c2-c2ccc(N(c4ccc(-c5cccc6ccccc56)cc4)c4ccc5c(c4)oc4c(-c6cccc7ccccc67)cccc45)cc2C3(c2ccccc2)c2ccccc2)cc1 ; c1ccc(-c2ccc3c(c2)C2(c4ccccc4-3)c3ccccc3-c3ccc(N(c4ccc(-c5cccc6ccccc56)cc4)c4ccc(-c5cccc6c5sc5ccccc56)cc4)cc32)cc1]

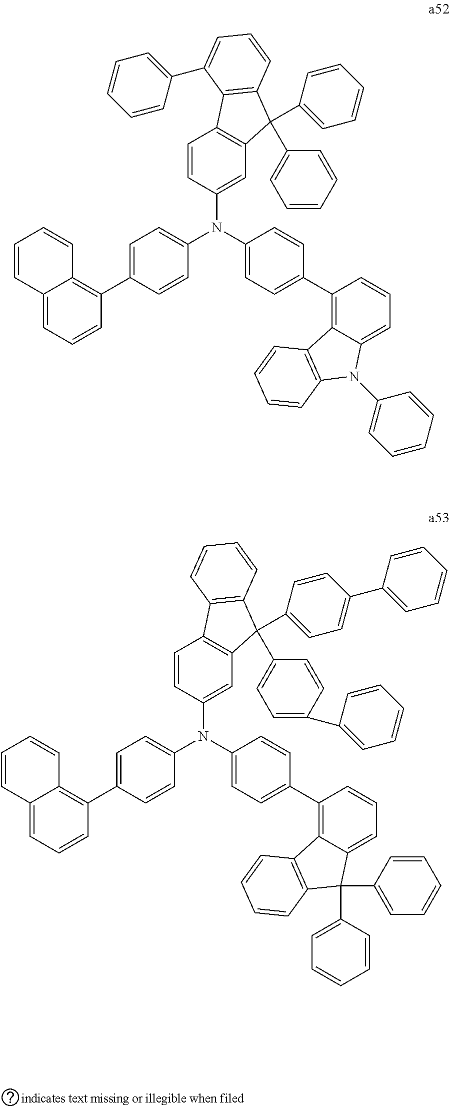 [SMILES: c1ccc(-c2cccc3c2-c2ccc(N(c4ccc(-c5cccc6ccccc56)cc4)c4ccc(-c5cccc6c5c5ccccc5n6-c5ccccc5)cc4)cc2C3(c2ccccc2)c2ccccc2)cc1 ; c1ccc(-c2ccc(C3(c4ccc(-c5ccccc5)cc4)c4ccccc4-c4ccc(N(c5ccc(-c6cccc7c6-c6ccccc6C7(c6ccccc6)c6ccccc6)cc5)c5ccc(-c6cccc7ccccc67)cc5)cc43)cc2)cc1]

The light emitting element ED of one or more embodiments may include at least one selected from among the amine compounds disclosed in Compound Group 1 in the hole transport region HTR. The amine compound according to one or more embodiments includes a first substituent and a second substituent directly or indirectly linked to the nitrogen atom of amine, and optionally further includes a third substituent, and may thus allow a light emitting element ED to obtain relatively high efficiency and long lifespan.

For example, the amine compound of one or more embodiments essentially includes a first substituent directly linked to the nitrogen atom of amine and a second substituent linked to the nitrogen atom of amine through a linker. The amine compound of one or more embodiments may include a fluorene moiety directly linked to the nitrogen atom of amine at carbon 2 as the first substituent. In one or more embodiments of the present disclosure, at least one substituent of a substituted or unsubstituted aryl group or a substituted or unsubstituted heteroaryl group may be bonded to the fluorene moiety to sterically suppress or reduce intermolecular proximity between fluorenes, thereby improving stability.

The amine compound of one or more embodiments includes a naphthyl group unsubstituted or substituted with the second substituent or a phenanthrenyl group unsubstituted or substituted with the second substituent. The amine compound of one or more embodiments includes the second substituent linked to the nitrogen atom of amine through a linker, and may thus exhibit excellent or suitable electrical stability. In some embodiments, the amine compound of one or more embodiments includes the first substituent and the second substituent, and optionally further includes the second substituent and the third substituent, and may thus have excellent or suitable hole transport properties and electron resistance, and may not be easily pyrolyzed. Accordingly, when the amine compound of one or more embodiments is applied to a light emitting element, element lifespan and light emitting efficiency may be improved.

In the light emitting element ED of one or more embodiments, the hole transport region HTR may further include a compound represented by Formula H-1.

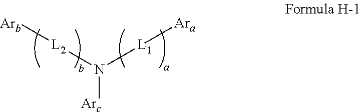

In Formula H-1 herein, L₁ and L₂ may each independently be a direct linkage, a substituted or unsubstituted arylene group having 6 to 30 ring-forming carbon atoms, or a substituted or unsubstituted heteroarylene group having 2 to 30 ring-forming carbon atoms. a and b may each independently be an integer of 0 to 10. In some embodiments, when a or b is an integer of 2 or greater, a plurality of L₁'s and L₂'s may each independently be a substituted or unsubstituted arylene group having 6 to 30 ring-forming carbon atoms, or a substituted or unsubstituted heteroarylene group having 2 to 30 ring-forming carbon atoms.

In Formula H-1, Arₐ and Ar_{b} may each independently be a substituted or unsubstituted aryl group having 6 to 30 ring-forming carbon atoms, or a substituted or unsubstituted heteroaryl group having 2 to 30 ring-forming carbon atoms. In some embodiments, in Formula H-1, Ar_{c} may be a substituted or unsubstituted aryl group having 6 to 30 ring-forming carbon atoms, or a substituted or unsubstituted heteroaryl group having 2 to 30 ring-forming carbon atoms.

In Formula H-1, Arₐ and Ar_{b} may each independently be a substituted or unsubstituted aryl group of 6 to 30 ring-forming carbon atoms, or a substituted or unsubstituted heteroaryl group of 2 to 30 ring-forming carbon atoms. In some embodiments, in Formula H-1, Ar_{c} may be a substituted or unsubstituted aryl group of 6 to 30 ring-forming carbon atoms.

The compound represented by Formula H-1 may be a monoamine compound. In some embodiments, the compound represented by Formula H-1 may be a diamine compound in which at least one selected from among Arₐ to Ar_{c} includes an amine group as a substituent. In some embodiments, the compound represented by Formula H-1 may be a carbazole-based compound in which at least one selected from among Arₐ and Ar_{b} includes a substituted or unsubstituted carbazole group, or a fluorene-based compound in which at least one among Arₐ and Ar_{b} includes a substituted or unsubstituted fluorene group.

The compound represented by Formula H-1 may be represented by any one selected from among the compounds in Compound Group H. However, the compounds listed in Compound Group H are only illustrations, and the compound represented by Formula H-1 is not limited to the compounds represented in Compound Group H.

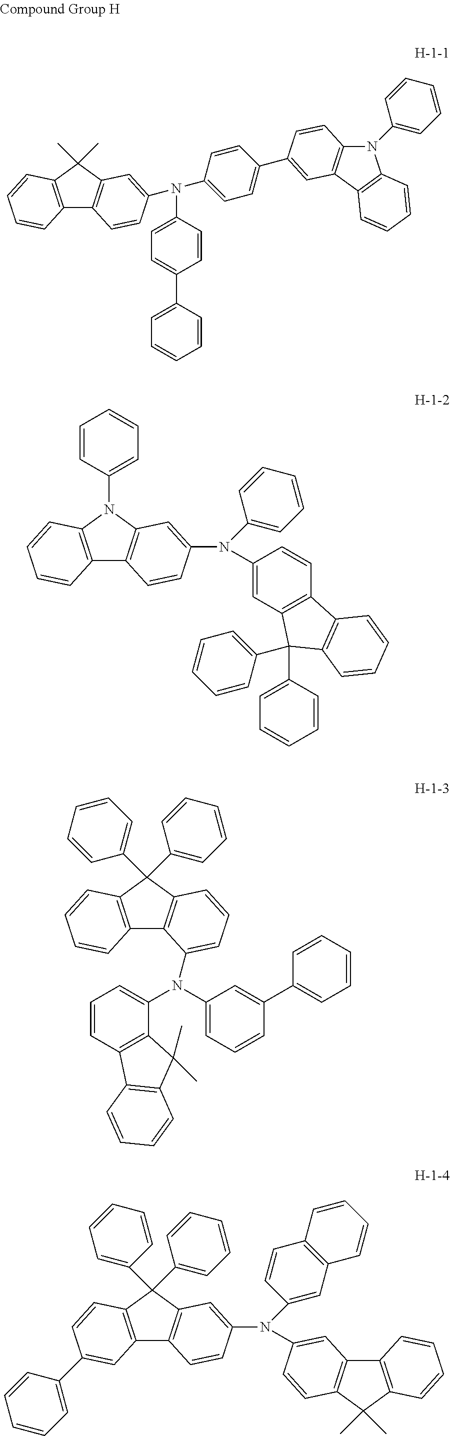 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21 ; CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21]

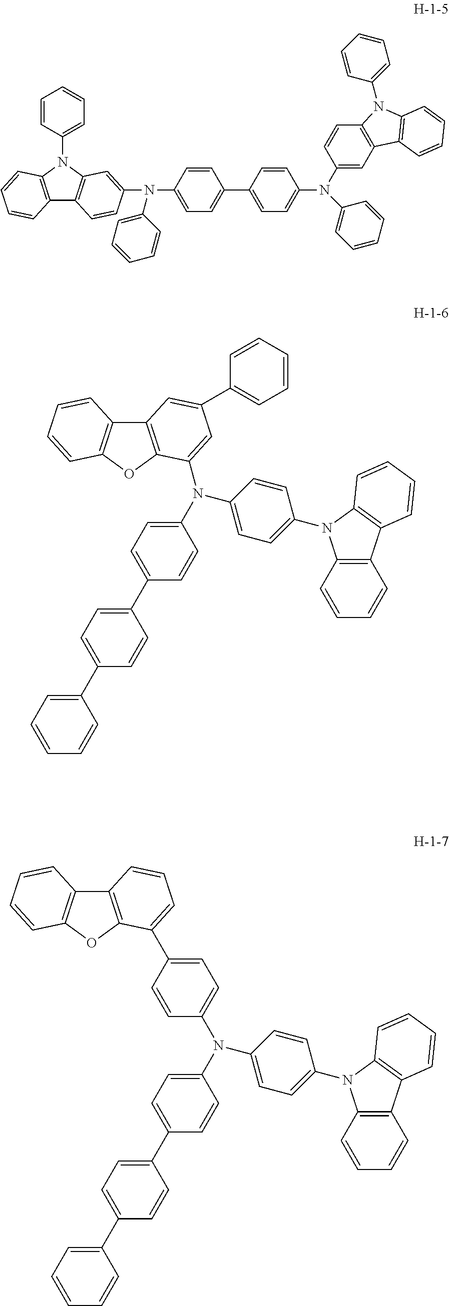 [SMILES: c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; c1ccc(-c2ccc(-c3ccc(N(c4ccc(-c5cccc6c5oc5ccccc56)cc4)c4ccc(-n5c6ccccc6c6ccccc65)cc4)cc3)cc2)cc1]

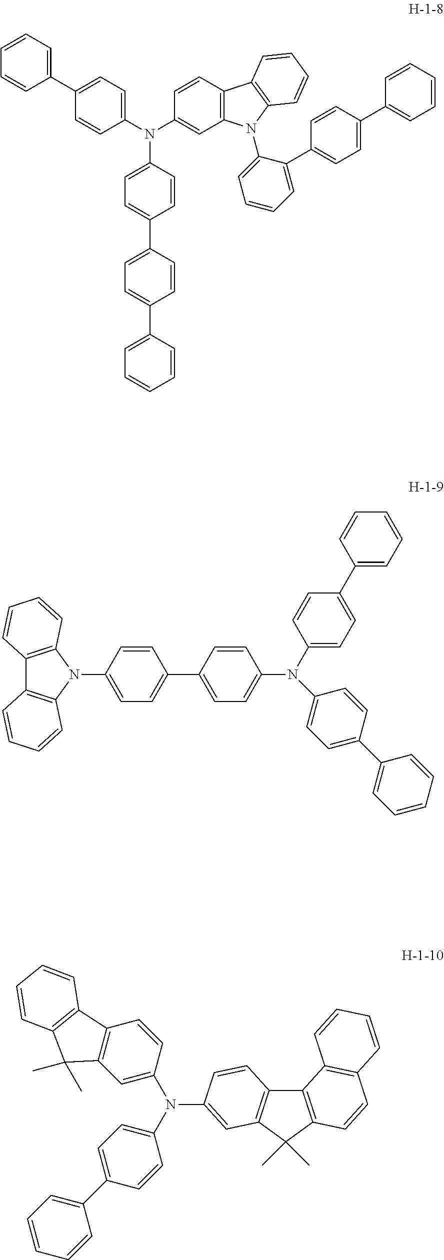 [SMILES: c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2ccccc2-c2ccc(N(c3ccc(-c4ccccc4)cc3)c3ccc4c(c3)C(C)(C)c3ccc5ccccc5c3-4)cc21]

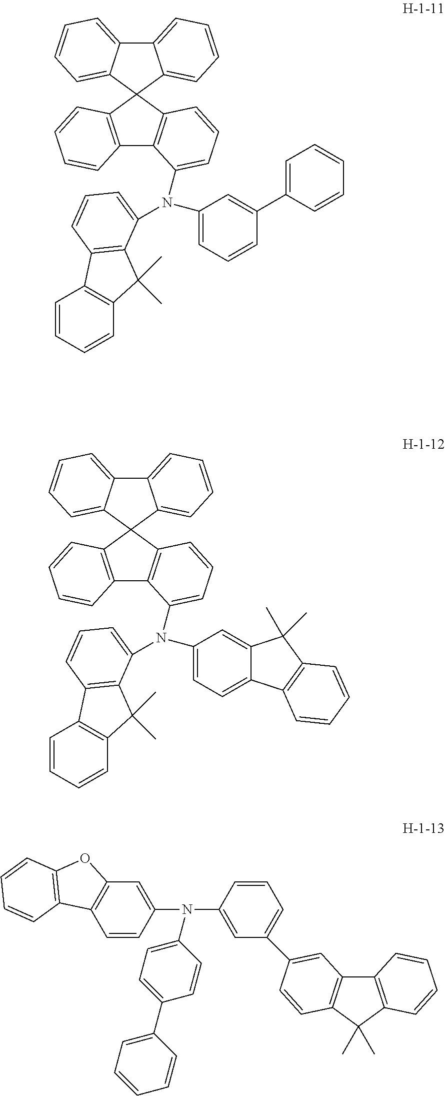 [SMILES: CC1(C)c2ccccc2-c2cccc(N(c3cccc(-c4ccccc4)c3)c3cccc4c3-c3ccccc3C43c4ccccc4-c4ccccc43)c21 ; CC1(C)c2ccccc2-c2ccc(N(c3cccc4c3-c3ccccc3C43c4ccccc4-c4ccccc43)c3cccc4c3C(C)(C)c3ccccc3-4)cc21 ; CC1(C)c2ccccc2-c2cc(-c3cccc(N(c4ccc(-c5ccccc5)cc4)c4ccc5c(c4)oc4ccccc45)c3)ccc21]

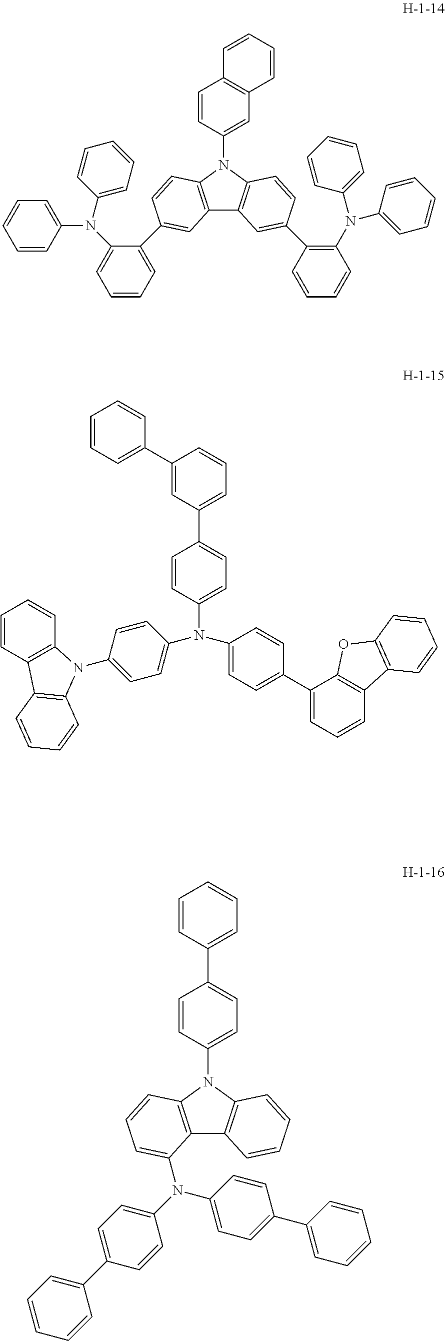 [SMILES: c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1 ; c1ccc(-c2ccc(N(c3ccc(-c4ccccc4)cc3)c3cccc4c3c3ccccc3n4-c3ccc(-c4ccccc4)cc3)cc2)cc1]

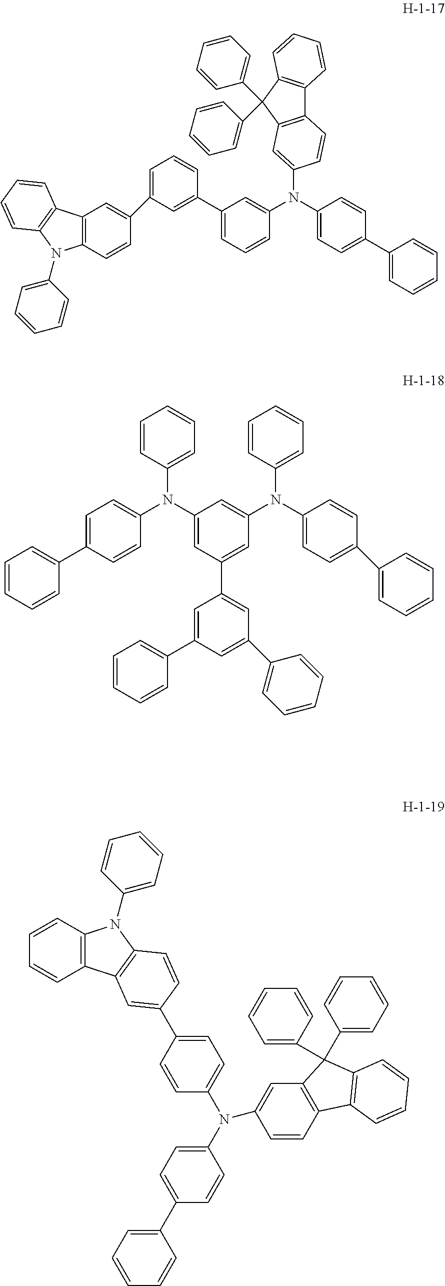 [SMILES: c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

In some embodiments, the hole transport region HTR may further include a suitable hole transport material.

For example, the hole transport region HTR may include at least one selected from among a phthalocyanine compound such as copper phthalocyanine, N¹,N^{1′}-([1,1′-biphenyl]-4,4′-diyl)bis(N¹-phenyl-N⁴,N⁴-di-m-tolylbenzene-1,4-diamine) (DNTPD), 4,4′,4″-[tris(3-methyl phenyl) phenylamino] triphenylamine (m-MTDATA), 4,4′,4″-tris(N,N-diphenylamino)triphenylamine (TDATA), 4,4′,4″-tris[N-(1-naphthyl)-N-phenylamino]-triphenylamine (1-TNATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(naphthalen-1-yl)-N,N′-diphenyl-benzidine (NPB or NPD), triphenylamine-containing polyetherketone (TPAPEK), 4-isopropyl-4′-methyldiphenyliodonium [tetrakis(pentafluorophenyl)borate], and dipyrazino[2,3-f:2′,3′-h] quinoxaline-2,3,6,7,10,11-hexacarbonitrile (HAT-CN).

The hole transport region HTR may include at least one selected from among carbazole derivatives such as N-phenyl carbazole and polyvinyl carbazole, fluorene-based derivatives, triphenylamine-based derivatives such as 4,4′,4″-tris(N-carbazolyl)triphenylamine (TCTA), N,N′-bis(3-methylphenyl)-N,N′-diphenyl-[1,1′-biphenyl]-4,4′-diamine (TPD), N,N′-di(naphthalene-1-yl)-N,N′-diphenyl-benzidine (NPB), 4,4′-cyclohexylidene bis[N,N-bis(4-methylphenyl)benzeneamine] (TAPC), 4,4′-bis[N,N′-(3-tolyl)amino]-3,3′-dimethylbiphenyl (HMTPD), 1,3-bis(N-carbazolyl)benzene (Mcp), and/or the like.

In some embodiments, the hole transport region HTR may include 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (Mdcp), and/or the like.

The hole transport region HTR may include the compounds of the hole transport region HTR in at least one selected from among a hole injection layer HIL, hole transport layer HTL, and electron blocking layer EBL.

The thickness of the hole transport region HTR may be from about 100 Å to about 10,000 Å, for example, from about 100 Å to about 5,000 Å. When the hole transport region HTR includes a hole injection layer HIL, the thickness of the hole injection layer HIL may be, for example, from about 30 Å to about 1,000 Å. When the hole transport region HTR includes a hole transport layer HTL, the thickness of the hole transport layer HTL may be from about 30 Å to about 1,000 Å. For example, when the hole transport region HTR includes an electron blocking layer EBL, the thickness of the electron blocking layer EBL may be from about 10 Å to about 1,000 Å. When the thicknesses of the hole transport region HTR, the hole injection layer HIL, the hole transport layer HTL and the electron blocking layer EBL satisfy the herein-described ranges, satisfactory hole transport properties may be achieved without substantial increase of a driving voltage.

The hole transport region HTR may further include a charge generating material to increase conductivity in addition to the herein-described materials. The charge generating material may be dispersed uniformly or non-uniformly in the hole transport region HTR. The charge generating material may be, for example, a p-dopant. The p-dopant may include at least one selected from among metal halide compounds, quinone derivatives, metal oxides, and cyano group-containing compounds, without limitation. For example, the p-dopant may include metal halide compounds such as CuI and RbI, quinone derivatives such as tetracyanoquinodimethane (TCNQ) and 2,3,5,6-tetrafluoro-7,7′,8,8′-tetracyanoquinodimethane (F4-TCNQ), metal oxides such as tungsten oxide and molybdenum oxide, cyano group-containing compounds such as dipyrazino[2,3-f: 2′,3′-h] quinoxaline-2,3,6,7,10,11-hexacarbonitrile (HAT-CN) and 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), and/or the like, without limitation.

As described herein, the hole transport region HTR may further include a buffer layer in addition to the hole injection layer HIL, the hole transport layer HTL and the electron blocking layer EBL. The buffer layer may compensate resonance distance according to the wavelength of light emitted from an emission layer EML and may increase emission efficiency. The materials included in the buffer layer may utilize materials which may be included in the hole transport region HTR.

The emission layer EML is provided on the hole transport region HTR. The emission layer EML may have a thickness of, for example, about 100 Å to about 1,000 Å or about 100 Å to about 300 Å. The emission layer EML may have a single layer formed utilizing a single material, a single layer formed utilizing multiple different materials, or a multilayer structure having multiple layers formed utilizing multiple different materials.

In the light emitting element ED of one or more embodiments, the emission layer EML may be to emit blue light. The light emitting element ED of one or more embodiments includes the amine compound of one or more embodiments described herein in the hole transport region HTR, and may thus exhibit relatively high light emitting efficiency and long lifespan in a blue light emitting region. However, the embodiment of the present disclosure is not limited thereto.

In the light emitting element ED according to one or more embodiments, the emission layer EML may include an anthracene derivative, a pyrene derivative, a fluoranthene derivative, a chrysene derivative, a dihydrobenzanthracene derivative, or a triphenylene derivative. To be specific, the emission layer EML may include an anthracene derivative or a pyrene derivative.

In the light emitting element ED of the embodiment shown in FIGS. 3 to 7, the emission layer EML may include a host and a dopant, and the emission layer EML may include a compound represented by Formula E-1. The compound represented by Formula E-1 may be utilized as a fluorescent host material.

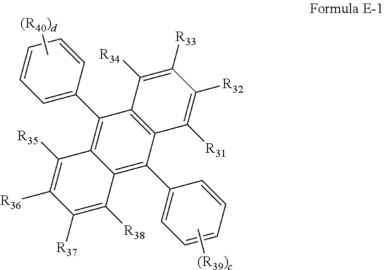

In Formula E-1, R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group of 1 to 10 carbon atoms, a substituted or unsubstituted alkenyl group of 2 to 10 carbon atoms, a substituted or unsubstituted aryl group of 6 to 30 ring-forming carbon atoms, or a substituted or unsubstituted heteroaryl group of 2 to 30 ring-forming carbon atoms, and/or may be combined with an adjacent group to form a ring. In some embodiments, R₃₁ to R₄₀ may be combined with an adjacent group to form a saturated hydrocarbon ring, an unsaturated hydrocarbon ring, a saturated heterocycle, or an unsaturated heterocycle.

In Formula E-1, “c” and “d” may each independently be an integer of 0 to 5.

Formula E-1 may be represented by any one among Compound E1 to Compound E19.

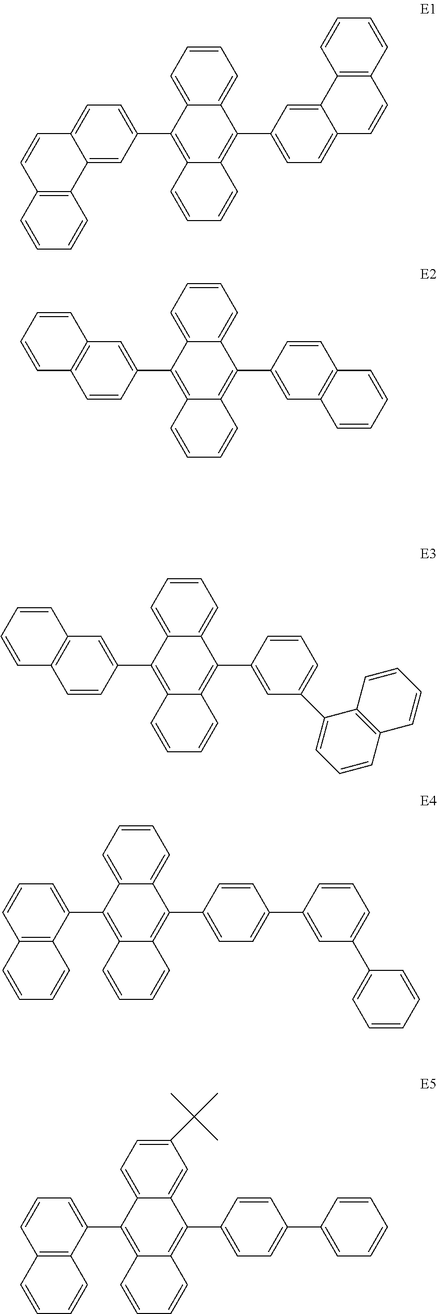

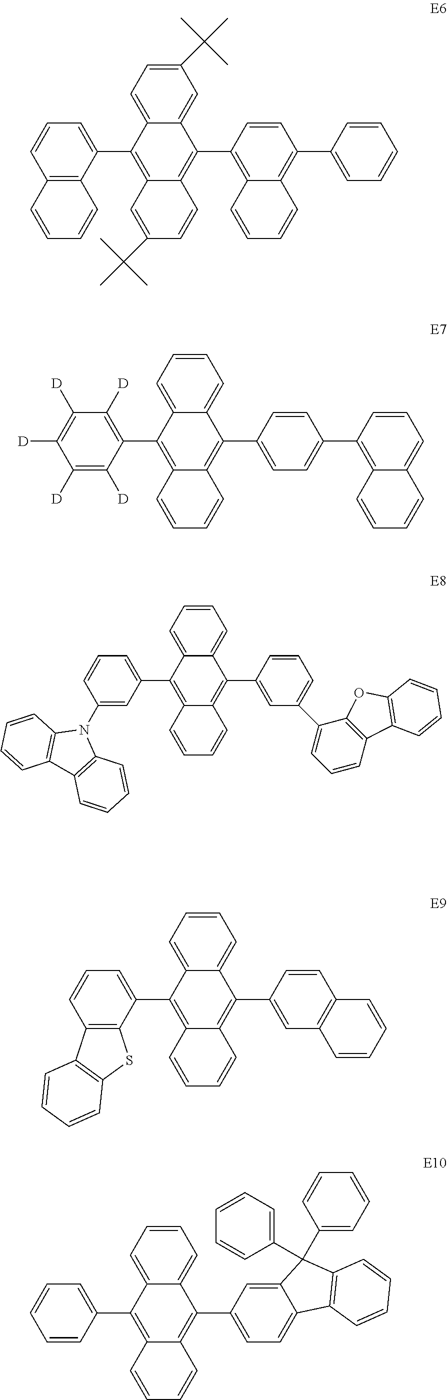

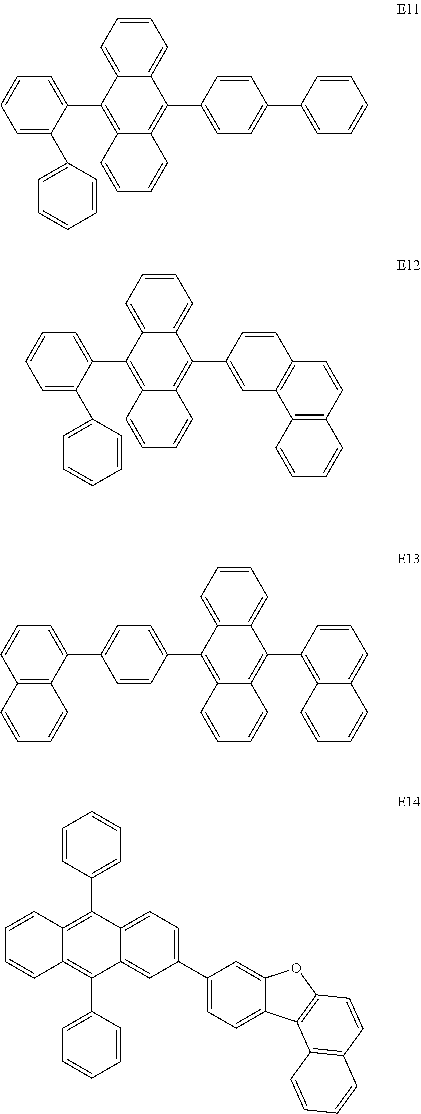

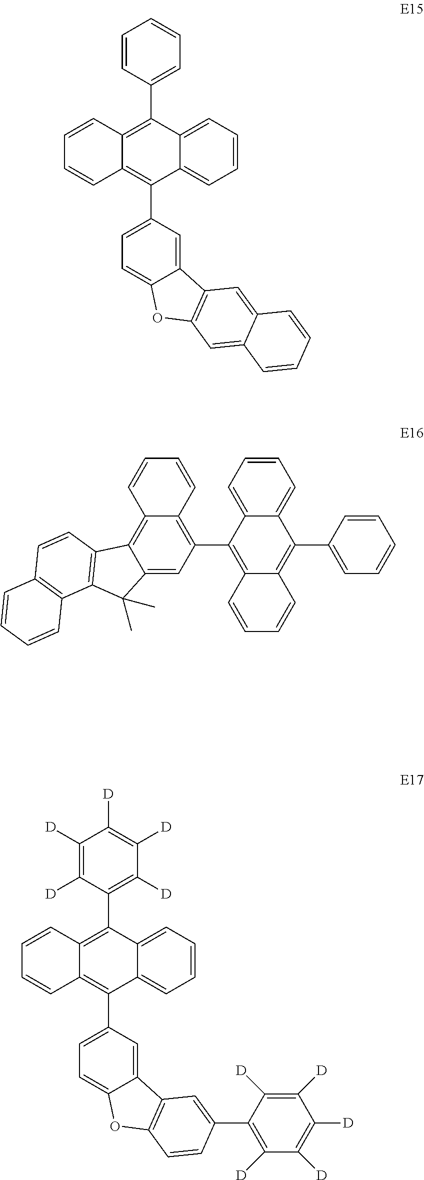

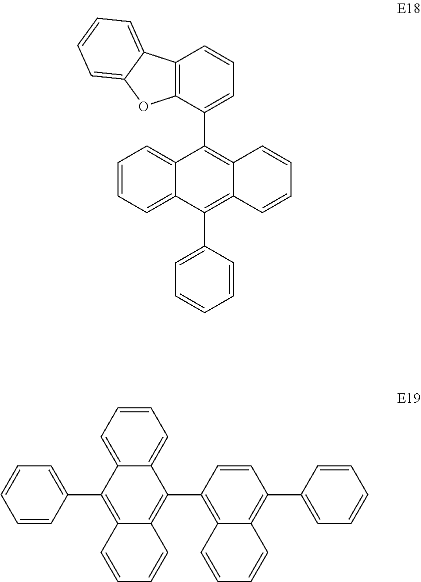

In one or more embodiments, the emission layer EML may include a compound represented by Formula E-2a or Formula E-2b. The compound represented by Formula E-2a or Formula E-2b may be utilized as a phosphorescent host material.

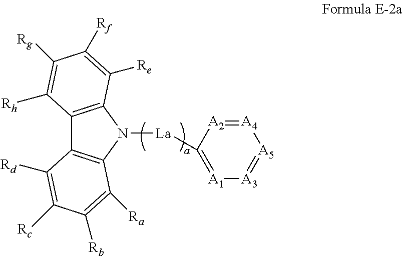

In Formula E-2a, a may be an integer of 0 to 10, and La may be a direct linkage, a substituted or unsubstituted arylene group having 6 to 30 ring-forming carbon atoms, or a substituted or unsubstituted heteroarylene group having 2 to 30 ring-forming carbon atoms. In some embodiments, when a is an integer of 2 or greater, a plurality of La's may each independently be a substituted or unsubstituted arylene group having 6 to 30 ring-forming carbon atoms, or a substituted or unsubstituted heteroarylene group having 2 to 30 ring-forming carbon atoms.

In some embodiments, in Formula E-2a, A1 to A5 may each independently be N or CRi. Ra to Ri may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, and/or may be bonded to an adjacent group to form a ring. Ra to Ri may be bonded to an adjacent group to form a hydrocarbon ring or a heterocycle containing N, O, S, and/or the like, as a ring-forming atom.

In some embodiments, in Formula E-2a, two or three selected from among A1 to A5 may be N, and the rest may be CRi.

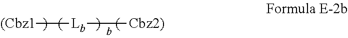

In Formula E-2b, Cbz1 and Cbz2 may each independently be an unsubstituted carbazole group, or a carbazole group substituted with an aryl group having 6 to 30 ring-forming carbon atoms. L_{b} is a direct linkage, a substituted or unsubstituted arylene group having 6 to 30 ring-forming carbon atoms, or a substituted or unsubstituted heteroarylene group having 2 to 30 ring-forming carbon atoms. In some embodiments, b is an integer of 0 to 10, and when b is an integer of 2 or more, a plurality of L_{b}'s may each independently be a substituted or unsubstituted arylene group having 6 to 30 ring-forming carbon atoms, or a substituted or unsubstituted heteroarylene group having 2 to 30 ring-forming carbon atoms.

The compound represented by Formula E-2a or Formula E-2b may be represented by any one among the compounds of Compound Group E-2. However, the compounds listed in Compound Group E-2 are example, and the compound represented by Formula E-2a or Formula E-2b is not limited to those represented in Compound Group E-2.

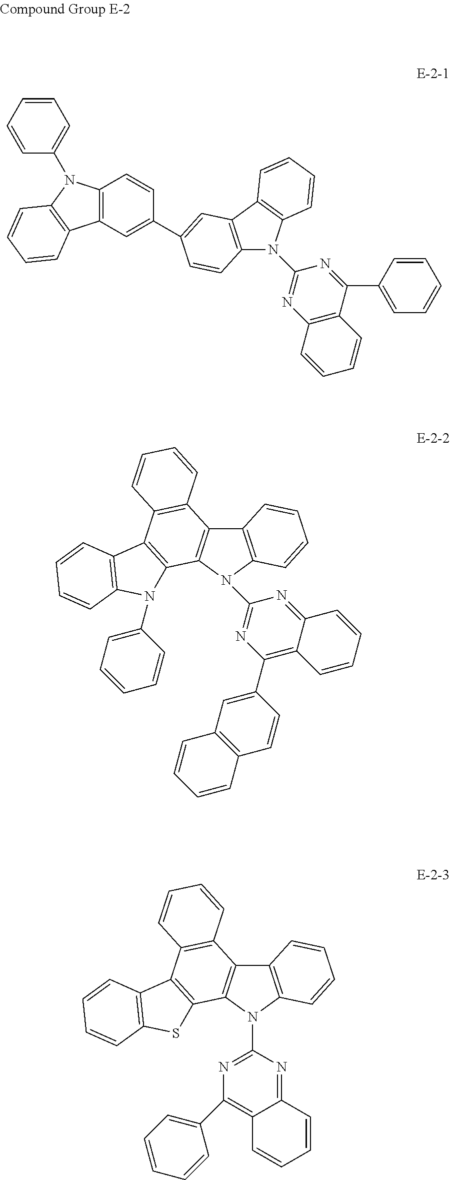 [SMILES: c1ccc(-c2nc(-n3c4ccccc4c4cc(-c5ccc6c(c5)c5ccccc5n6-c5ccccc5)ccc43)nc3ccccc23)cc1 ; c1ccc(-n2c3ccccc3c3c4ccccc4c4c5ccccc5n(-c5nc(-c6ccc7ccccc7c6)c6ccccc6n5)c4c32)cc1 ; c1ccc(-c2nc(-n3c4ccccc4c4c5ccccc5c5c6ccccc6sc5c43)nc3ccccc23)cc1]

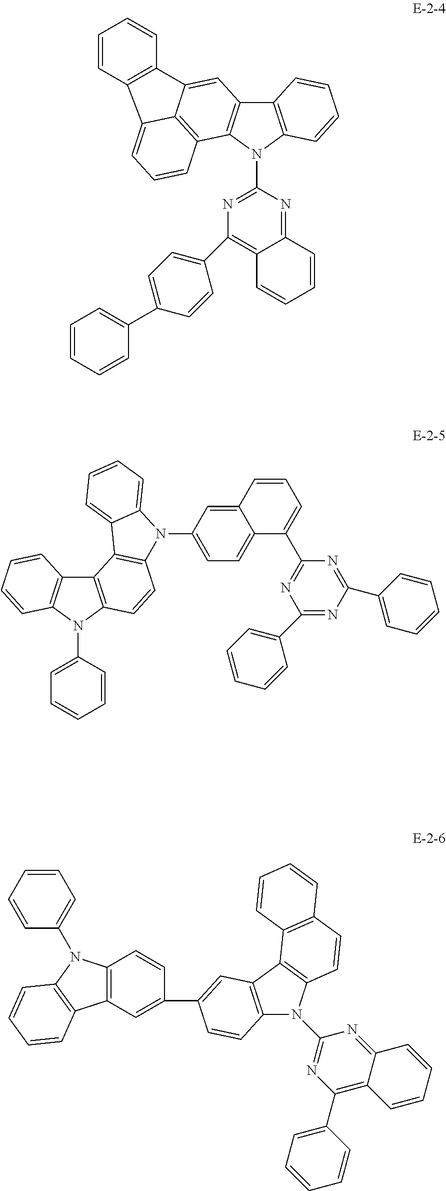 [SMILES: c1ccc(-c2ccc(-c3nc(-n4c5ccccc5c5cc6c7c(cccc7c54)-c4ccccc4-6)nc4ccccc34)cc2)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1]

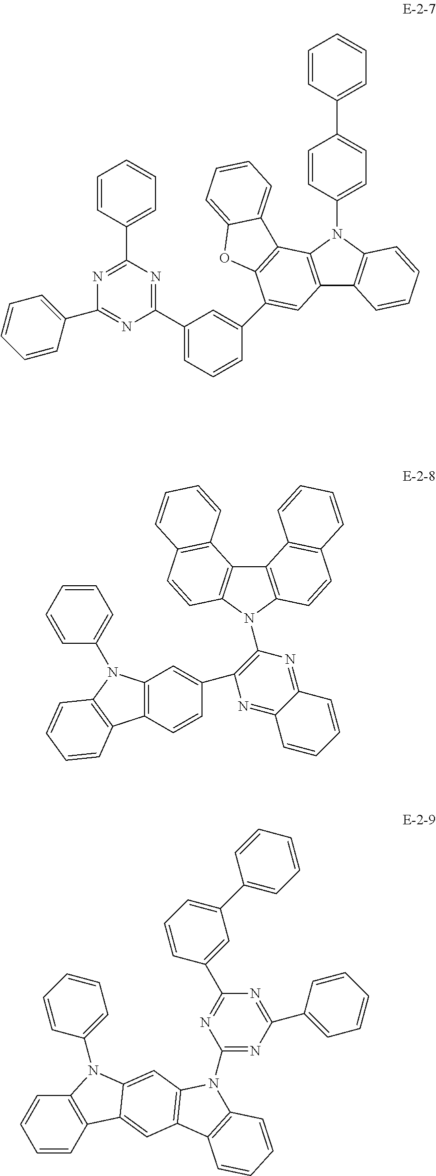 [SMILES: c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-n2c3ccccc3c3ccc(-c4nc5ccccc5nc4-n4c5ccc6ccccc6c5c5c6ccccc6ccc54)cc32)cc1 ; c1ccc(-c2cccc(-c3nc(-c4ccccc4)nc(-n4c5ccccc5c5cc6c7ccccc7n(-c7ccccc7)c6cc54)n3)c2)cc1]

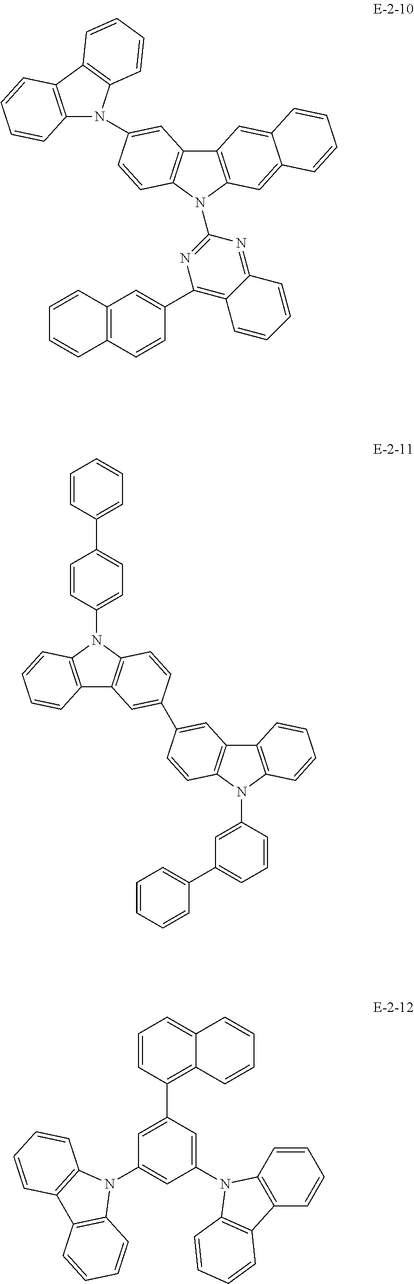 [SMILES: c1ccc2cc(-c3nc(-n4c5ccc(-n6c7ccccc7c7ccccc76)cc5c5cc6ccccc6cc54)nc4ccccc34)ccc2c1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc2c(-c3cc(-n4c5ccccc5c5ccccc54)cc(-n4c5ccccc5c5ccccc54)c3)cccc2c1]

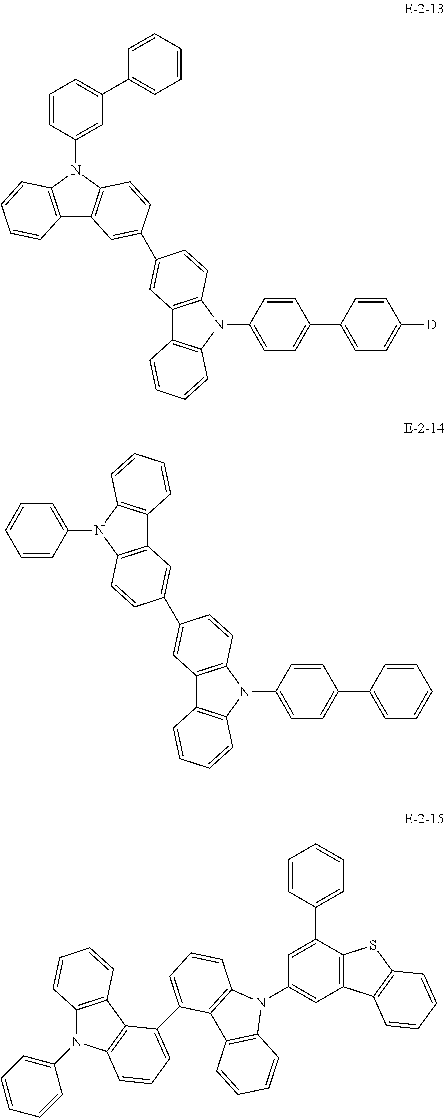 [SMILES: [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

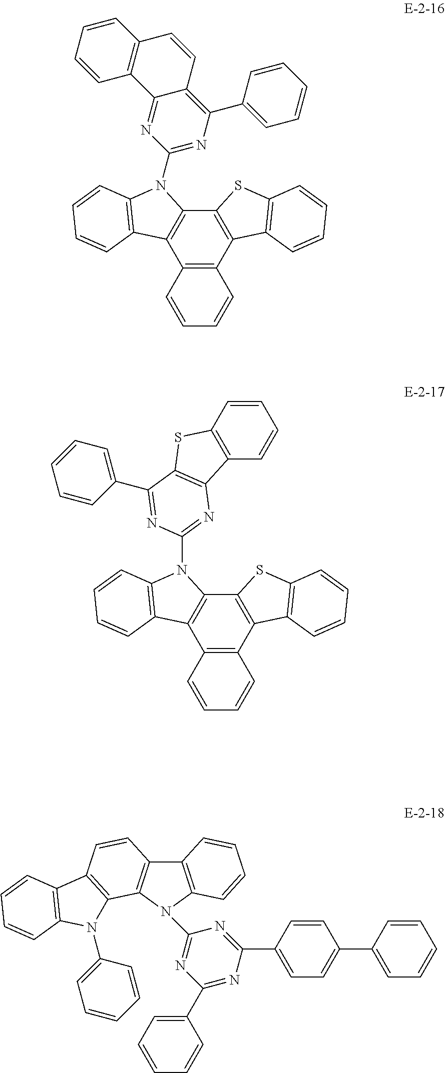 [SMILES: c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1 ; c1ccc(-c2ccc(-c3nc(-c4ccccc4)nc(-n4c5ccccc5c5ccc6c7ccccc7n(-c7ccccc7)c6c54)n3)cc2)cc1]

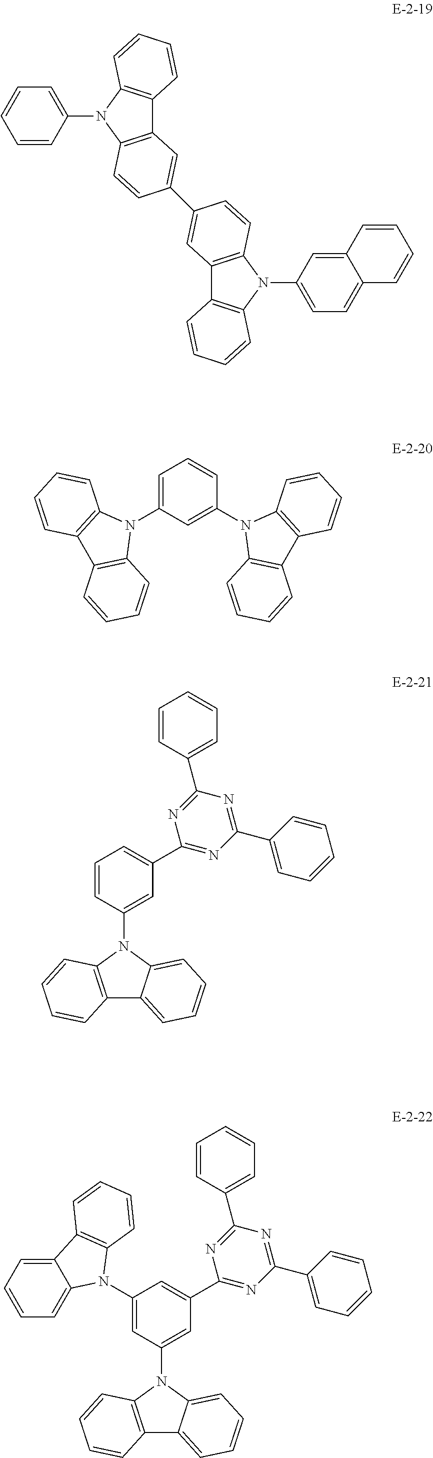 [SMILES: c1ccc(-n2c3ccccc3c3cc(-c4ccc5c(c4)c4ccccc4n5-c4ccc5ccccc5c4)ccc32)cc1 ; c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1]

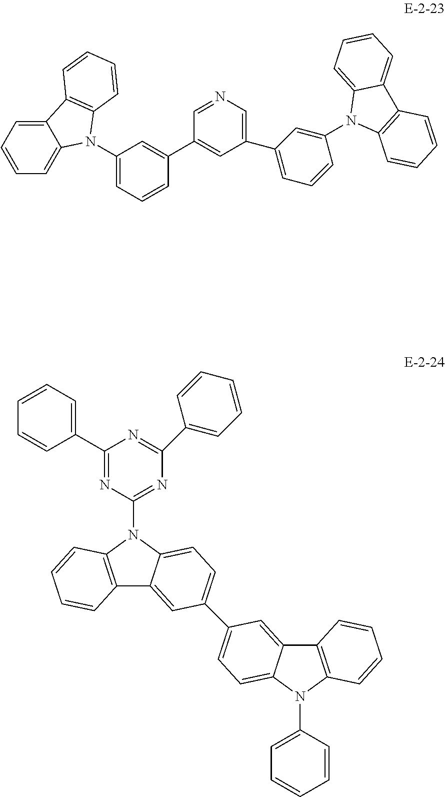 [SMILES: c1cc(-c2cncc(-c3cccc(-n4c5ccccc5c5ccccc54)c3)c2)cc(-n2c3ccccc3c3ccccc32)c1 ; c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include a general material suitable in the art as a host material. For example, the emission layer EML may include, as a host material, at least one selected from among bis(4-(9H-carbazol-9-yl)phenyl)diphenylsilane (BCPDS), (4-(1-(4-(diphenylamino)phenyl)cyclohexyl)phenyl)diphenyl-phosphine oxide (POPCPA), bis[2-(diphenylphosphino)phenyl]ether oxide (DPEPO), 4,4′-bis(N-carbazolyl)-1,1′-biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), and/or 1,3,5-tris(1-phenyl-1H-benzo[d]imidazol-2-yl)benzene (TPBi). However, the embodiment of the present disclosure is not limited thereto, for example, tris(8-hydroxyquinolino)aluminum (Alq₃), 9,10-di(naphthalen-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetrasiloxane (DPSiO₄), and/or the like may be utilized as a host material.

The emission layer EML may include a compound represented by Formula M-a or Formula M-b. The compound represented by Formula M-a or Formula M-b may be utilized as a phosphorescence dopant material. In some embodiments, in one or more embodiments, the compound represented by Formula M-a or Formula M-b may be utilized as an auxiliary dopant material.

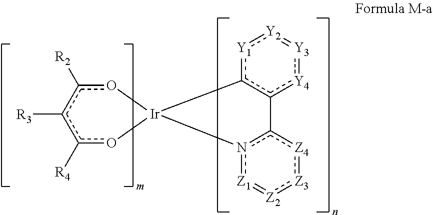

In Formula M-a, Y₁ to Y₄ and Z₁ to Z₄ may each independently be CR₁ or N, R₁ to R₄ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, and/or may be bonded to an adjacent group to form a ring. In Formula M-a, m is 0 or 1, and n is 2 or 3. In Formula M-a, when m is 0, n is 3, and when m is 1, n is 2.

The compound represented by Formula M-a may be utilized as a phosphorescent dopant.

The compound represented by Formula M-a may be represented by any one among Compound M-a1 to Compound M-a25. However, Compounds M-a1 to M-a25 are examples, and the compound represented by Formula M-a is not limited to those represented by Compounds M-a1 to M-a25.

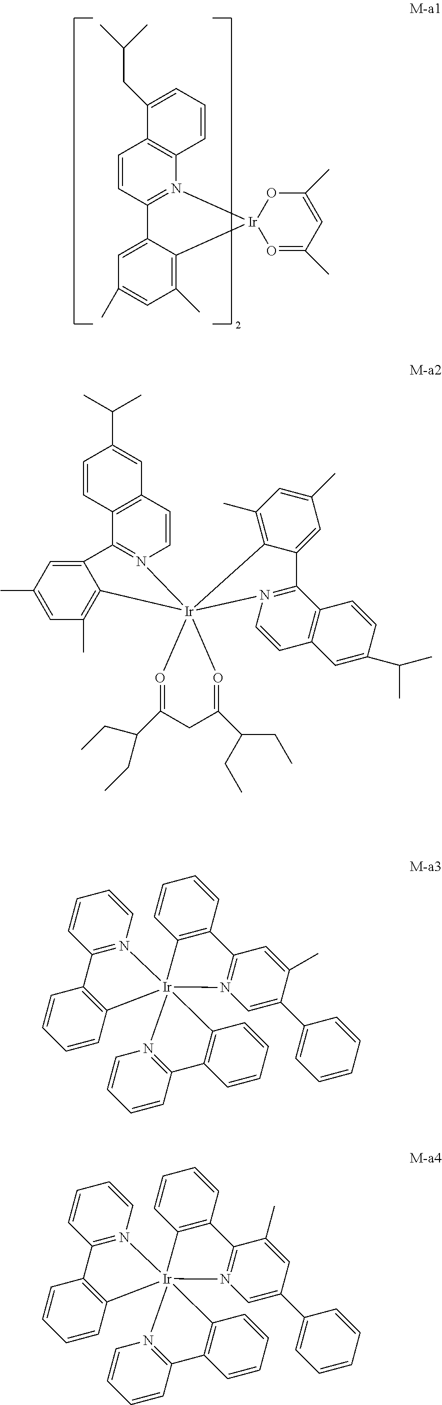

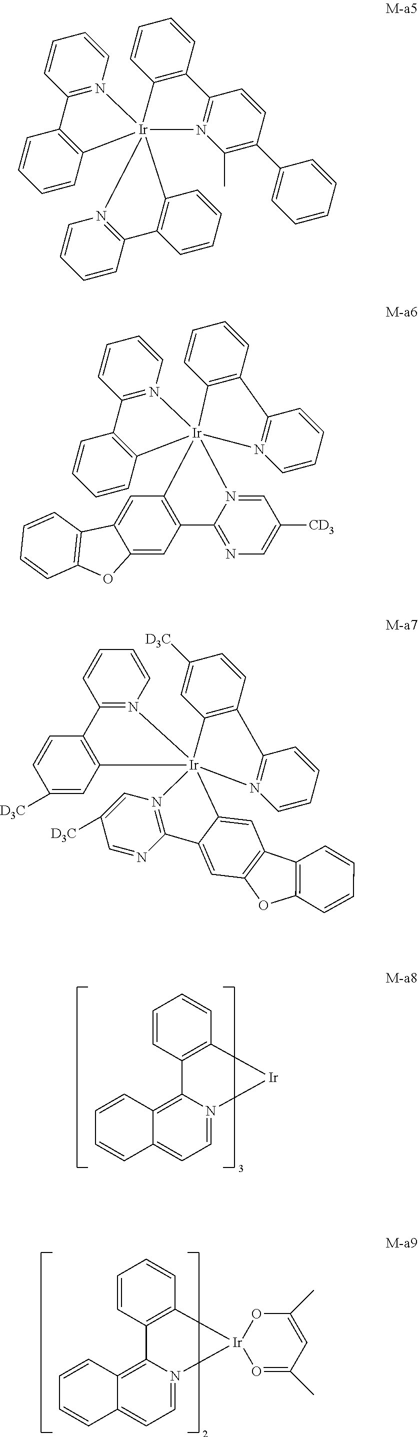

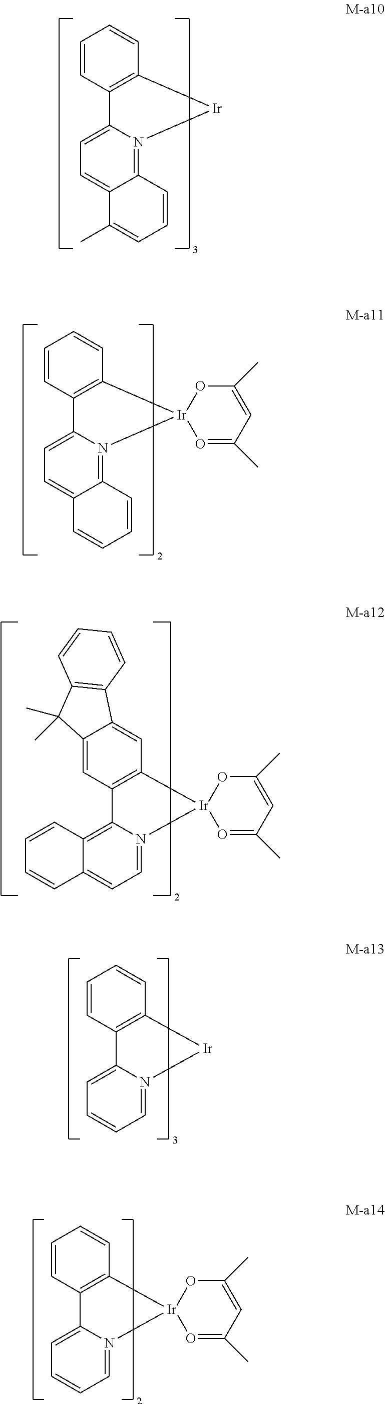

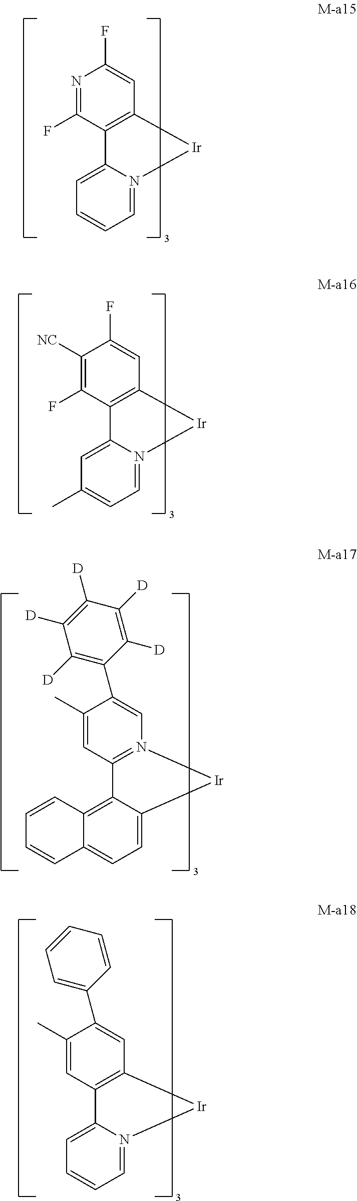

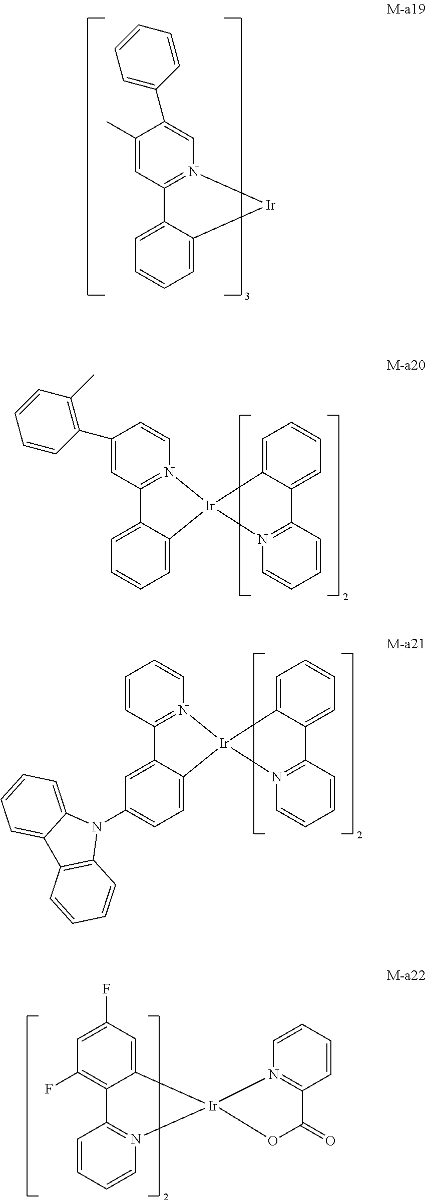

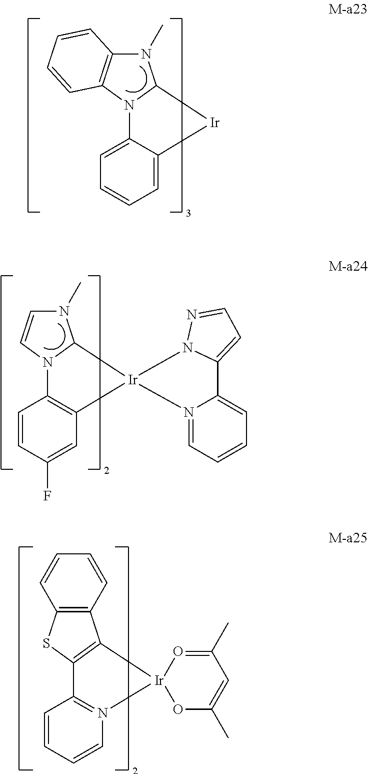

The compound M-a1 and the compound M-a2 may be utilized as a red dopant material, and the compound M-a3 to the compound M-a7 may be utilized as a green dopant material.

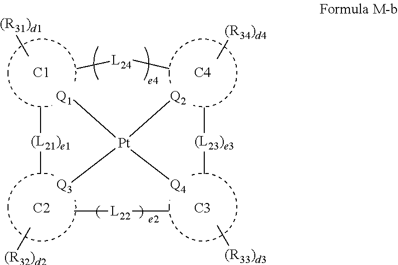

In Formula M-b, Q₁ to Q₄ may each independently be C or N, C1 to C4 may each independently be a substituted or unsubstituted hydrocarbon ring of 5 to 30 ring-forming carbon atoms, or a substituted or unsubstituted heterocycle of 2 to 30 ring-forming carbon atoms. L₂₁ to L₂₄ may each independently be a direct linkage,

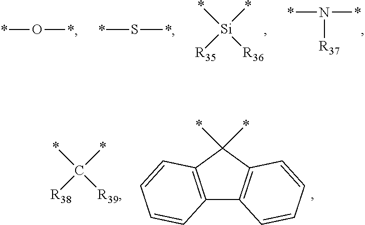

a substituted or unsubstituted divalent alkyl group of 1 to 20 carbon atoms, a substituted or unsubstituted arylene group of 6 to 30 ring-forming carbon atoms, or a substituted or unsubstituted heteroarylene group of 2 to 30 ring-forming carbon atoms, and e1 to e4 may each independently be 0 or 1. R₃₁ to R₃₉ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring, and d1 to d4 may each independently be an integer of 0 to 4.

The compound represented by Formula M-b may be utilized as a blue phosphorescence dopant or a green phosphorescence dopant. In some embodiments, the compound represented by Formula M-b may be an auxiliary dopant in one or more embodiments and may be further included in the emission layer EML.

The compound represented by Formula M-b may be represented by any one among Compound M-b-1 to Compound M-b-11. However, the compounds are illustrations, and the compound represented by Formula M-b is not limited to Compound M-b-1 to Compound M-b-11.

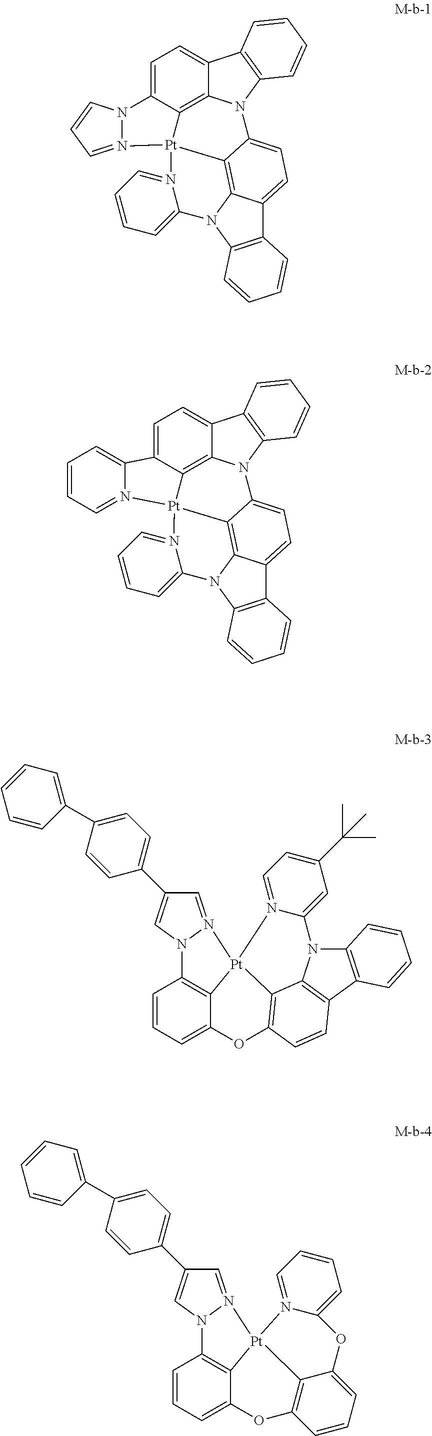

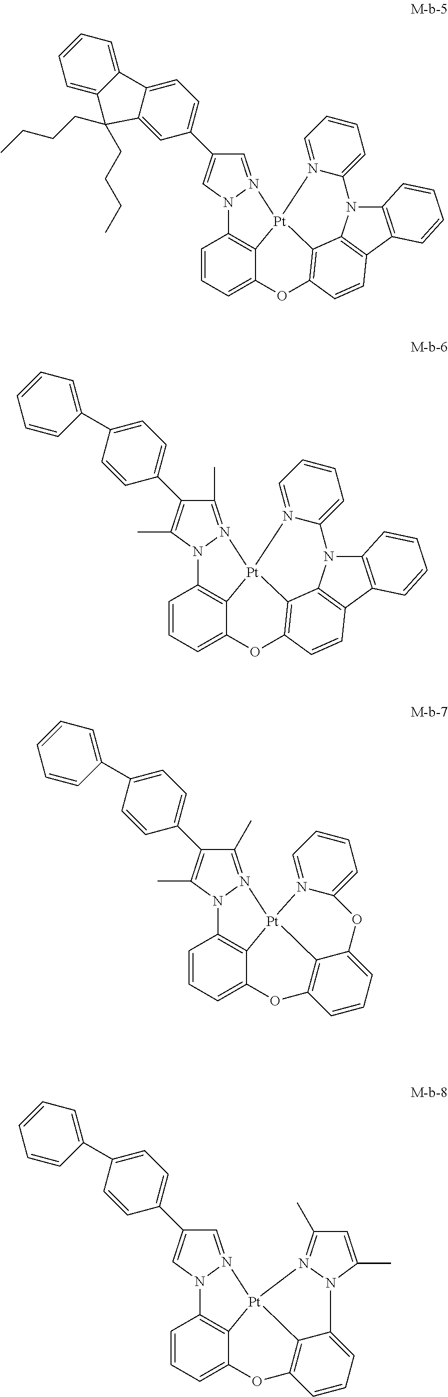

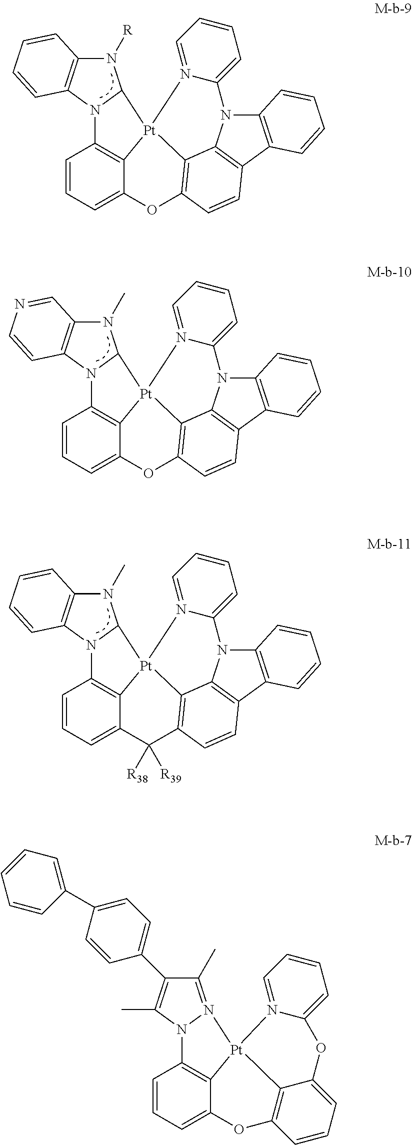

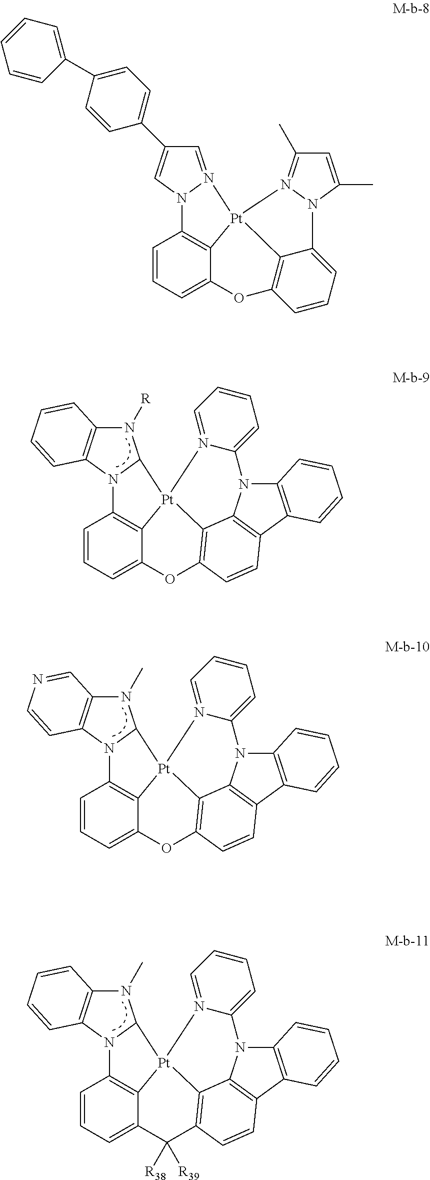

In the compounds, R, R38, and R39 may each independently be a hydrogen atom, a deuterium atom, a halogen atom, a cyano group, a substituted or unsubstituted amine group, a substituted or unsubstituted alkyl group having 1 to 20 carbons, a substituted or unsubstituted aryl group having 6 to 30 ring-forming carbon atoms, or a substituted or unsubstituted heteroaryl group having 2 to 30 ring-forming carbon atoms.

The emission layer EML may include a compound represented by any one among Formula F-a to Formula F-c. The compound represented by Formula F-a to Formula F-c may be utilized as a fluorescence dopant material.

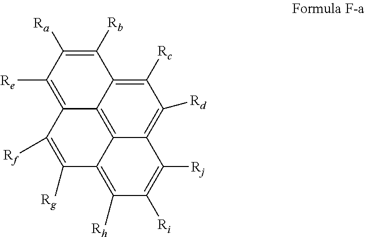

In Formula F-a, two selected from among Rₐ to Rⱼ may each independently be substituted with *—NAr₁Ar₂. The others, which are not substituted with *—NAr₁Ar₂, among Ra to Rⱼ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In *—NAr₁Ar₂, Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. For example, at least one of Ar₁ or Ar₂ may be a heteroaryl group containing O or S as a ring-forming atom.

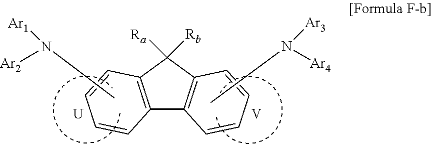

In Formula F-b, Rₐ and R_{b} may each independently be a hydrogen atom, a deuterium atom, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, and/or may be bonded to an adjacent group to form a ring. Ar₁ to Ar₄ may each independently be a substituted or unsubstituted aryl group having 6 to 30 ring-forming carbon atoms, or a substituted or unsubstituted heteroaryl group having 2 to 30 ring-forming carbon atoms.

In Formula F-b, U and V may each independently be a substituted or unsubstituted hydrocarbon ring having 5 to 30 ring-forming carbon atoms, or a substituted or unsubstituted heterocycle having 2 to 30 ring-forming carbon atoms. At least one among Ar₁ to Ar₄ may be a heteroaryl group containing O or S as a ring-forming atom.

In Formula F-b, the number of rings represented by U and V may each independently be 0 or 1. For example, in Formula F-b, it refers to that when the number of U or V is 1, one ring constitutes a fused ring at a portion indicated by U or V, and when the number of U or V is 0, a ring indicated by U or V does not exist. For example, when the number of U is 0 and the number of V is 1, or when the number of U is 1 and the number of V is 0, the fused ring having a fluorene core in Formula F-b may be a cyclic compound having four rings. In some embodiments, when each number of U and V is 0, the fused ring in Formula F-b may be a cyclic compound having three rings. In some embodiments, when each number of U and V is 1, the fused ring having a fluorene core in Formula F-b may be a cyclic compound having five rings.

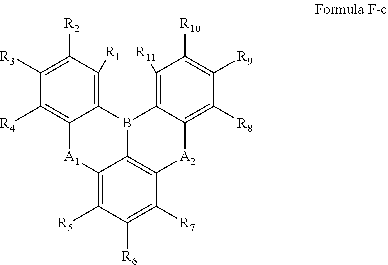

In Formula F-c, A₁ and A₂ may each independently be O, S, Se, or NRₘ, and Rₘ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. R₁ to R₁₁ may each independently be a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and/or are bonded to an adjacent group to form a ring.

In Formula F-c, A₁ and A₂ may each independently be bonded to substituents of an adjacent ring to form a fused ring. For example, when A₁ and A₂ may each independently be NRₘ, A₁ may be bonded to R₄ or R₅ to form a ring. In some embodiments, A₂ may be bonded to R₇ or R₈ to form a ring.

In one or more embodiments, the emission layer EML may further include, as a suitable dopant material, a styryl derivative (e.g., 1,4-bis[2-(3-N-ethylcarbazolyl)vinyl]benzene (BCzVB), 4-(di-p-tolylamino)-4′-[(di-p-tolylamino)styryl]stilbene (DPAVB), and N-(4-((E)-2-(6-((E)-4-(diphenylamino)styryl)naphthalen-2-yl)vinyl)phenyl)-N-phenylbenzenamine (N-BDAVBi), 4,4′-bis[2-(4-(N,N-diphenylamino)phenyl)vinyl]biphenyl (DPAVBi), perylene and a derivative thereof (e.g., 2,5,8,11-tetra-t-butylperylene (TBP)), pyrene and a derivative thereof (e.g., 1,1′-dipyrene, 1,4-dipyrenylbenzene, 1,4-bis(N,N-diphenylamino)pyrene), and/or the like.

The emission layer EML may further include a suitable phosphorescence dopant material. For example, a metal complex including iridium (Ir), platinum (Pt), osmium (Os), aurum (Au), titanium (Ti), zirconium (Zr), hafnium (Hf), europium (Eu), terbium (Tb), or thulium (Tm) may be utilized as a phosphorescent dopant. For example, iridium(Ill) bis(4,6-difluorophenylpyridinato-N,C2) (Flrpic), bis(2,4-difluorophenylpyridinato)-tetrakis(1-pyrazolyl)borate iridium(Ill) (Fir6), or platinum octaethyl porphyrin (PtOEP) may be utilized as a phosphorescent dopant. However, the embodiment of the present disclosure is not limited thereto.

In some embodiments, the emission layer EML may include a hole transport host and an electron transport host. In some embodiments, the emission layer EML may include an auxiliary dopant and a light emitting dopant. In some embodiments, the auxiliary dopant may include a phosphorescence dopant material or a thermally activated delayed fluorescence dopant. For example, in one or more embodiments, the emission layer EML may include a hole transport host, an electron transport host, an auxiliary dopant, and a light emitting dopant.

In some embodiments, in the emission layer EML, the hole transporting host and the electron transporting host may form an exciplex. In this case, the triplet energy of the exciplex formed by the hole transporting host and the electron transporting host may correspond to T1, which is a gap between LUMO energy level of the electron transporting host and HOMO energy level of the hole transporting host.

In one or more embodiments, the triplet energy level T1 of the exciplex formed by the hole transporting host and the electron transporting host may be about 2.4 electron volt (eV) to about 3.0 eV. In some embodiments, the triplet energy level T1 of the exciplex may have a value smaller than the energy gap of each host material. Accordingly, the exciplex may have a triplet energy level T1 of 3.0 eV or less, which is an energy gap between the hole transporting host and the electron transporting host.

In some embodiments, at least one emission layer EML may include a quantum dot material.

Herein, a quantum dot indicates a crystal of a semiconductor compound. The quantum dot may be to emit light of one or more suitable emission wavelengths depending on the size of the crystal. The quantum dot may be to emit light of one or more suitable emission wavelengths by regulating an element ratio in the quantum dot compound. The quantum dot (in the form of particles or dots) may have a diameter (e.g., average) of, for example, about 1 nanometer (nm) to about 10 nm.

In one or more embodiments, the quantum dot may be synthesized through a wet chemical process, a metal organic chemical vapor deposition process, a molecular beam epitaxy process, or a process similar thereto. For example, the wet chemical process is a method of mixing an organic solvent and a precursor material and then growing a quantum dot particle crystal. When the crystal grows, the organic solvent naturally serves as a dispersant coordinated to a surface of the quantum dot crystal and may control the growth of the crystal. Therefore, the wet chemical process is easier than vapor deposition methods such as metal organic chemical vapor deposition (MOCVD) or molecular beam epitaxy (MBE), and may control the growth of quantum dot particles through a low-cost process.

The core of the quantum dot may be selected from a Group II-VI compound, a Group III-VI compound, a Group I-III-VI compound, a Group III-V compound, a Group III-II-V compound, a Group IV-VI compound, a Group IV element, a Group IV compound, Group I-II-VI compound, Group II-IV-VI compound, Group I-II-IV-VI compound, Group II-IV-V compound, and/or a combination thereof.

The Group II-VI compound may be selected from the group consisting of a binary compound selected from the group consisting of CdSe, CdTe, CdS, ZnS, ZnSe, ZnTe, ZnO, HgS, HgSe, HgTe, MgSe, MgS, and a mixture thereof, a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and a mixture thereof, and a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, and a mixture thereof. In some embodiments, the Group II-VI semiconductor compound may further include a Group I metal and/or a Group IV element. The Group I-II-VI compound may be selected from CuSnS or CuZnS, and the Group II-IV-VI compound may be selected from ZnSnS and/or the like. The Group I-II-IV-VI compound may be selected from quaternary compounds selected from the group consisting of Cu₂ZnSnS₂, Cu₂ZnSnS₄, Cu₂ZnSnSe₄, Ag₂ZnSnS₂, and a mixture thereof.

The Group III-VI compound may include a binary compound such as GaS, Ga₂S₃, GaSe, Ga₂Se₃, GaTe, InS, InSe, In₂Se₃, InTe, and In₂S₃, a ternary compound such as InGaS₃ and InGaSe₃, or any combination thereof.

The Group I-III-VI compound may be selected from a ternary compound selected from the group consisting of AgInS, AgInS₂, CuInS, CuInS₂, AgGaS₂, CuGaS₂, CuGaO₂, AgGaO₂, AgAlO₂, AgInSe₂, AgGaS, AgGaSe₂, CuInSe₂, and CuGaSe₂, or a mixture thereof, or a quaternary compound such as AgInGaS₂, AgInGaSe₂, and CuInGaS₂.

The Group III-V compound may be selected from the group consisting of a binary compound selected from the group consisting of GaN, GaP, GaAs, GaSb, AlN, AIP, AIAs, AISb, InN, InP, InAs, InSb, and a mixture thereof, a ternary compound selected from the group consisting of GaNP, GaNAs, GaNSb, GaPAs, GaPSb, AlNP, AlNAs, AlNSb, AIPAs, AIPSb, InGaP, InAIP, InNP, InNAs, InNSb, InPAs, InPSb, and a mixture thereof, and a quaternary compound selected from the group consisting of GaAINP, GaAINAs, GaAINSb, GaAIPAs, GaAIPSb, GaInNP, GaInNAs, GalnNSb, GaInPAs, GalnPSb, InAINP, InAINAs, InAINSb, InAIPAs, InAIPSb, and a mixture thereof. In some embodiments, the Group III-V compound may further include a Group II metal. For example, InZnP, InGaZnP, InAIZnP, and/or the like may be included as a Group III-II-V compound.

The Group IV-VI compound may be selected from the group consisting of a binary compound selected from the group consisting of SnS, SnSe, SnTe, PbS, PbSe, PbTe, and a mixture thereof, a ternary compound selected from the group consisting of SnSeS, SnSeTe, SnSTe, PbSeS, PbSeTe, PbSTe, SnPbS, SnPbSe, SnPbTe, and a mixture thereof, and a quaternary compound selected from the group consisting of SnPbSSe, SnPbSeTe, SnPbSTe, and a mixture thereof. The Group IV element may be selected from the group consisting of Si, Ge, and a mixture thereof. The Group IV compound may be a binary compound selected from the group consisting of SiC, SiGe, and a mixture thereof.

The Group II-IV-V compound may be a ternary compound selected from the group consisting of ZnSnP, ZnSnP₂, ZnSnAs₂, ZnGeP₂, ZnGeAs₂, CdSnP₂, and CdGeP₂ and a mixture thereof.

Each element included in the multi-element compound such as the binary compound, ternary compound, and quaternary compound may be present in particles at a substantially uniform concentration or a non-substantially uniform concentration.

For example, Formula indicates the types (kinds) of elements included in a compound, and element ratios in the compound may be different. For example, AgInGaS₂ may indicate AgInₓGa₁₋ₓS₂ (x is a real number between 0 and 1).

In some embodiments, the quantum dot of one or more embodiments may include a core/shell structure in which one quantum dot surrounds another quantum dot. The core/shell structure may have a concentration gradient in which the concentration of an element present in the shell becomes lower towards the core.

In some embodiments, a quantum dot may have the core/shell structure including a core having nano-crystals, and a shell around (e.g., surrounding) the core, which are described above. The shell of the quantum dot may serve as a protection layer to prevent or reduce the chemical degeneration of the core so as to keep semiconductor properties, and/or a charging layer to impart electrophoresis properties to the quantum dot. The shell may be a single layer or multiple layers. Examples of the shell of the quantum dot may be a metal or non-metal oxide, a semiconductor compound, or a combination thereof.

For example, the metal or non-metal oxide may include a binary compound selected from among SiO₂, Al₂O₃, TiO₂, ZnO, MnO, Mn₂O₃, Mn₃O₄, CuO, FeO, Fe₂O₃, Fe₃O₄, CoO, Co₃O₄ and NiO, or a ternary compound selected from MgAl₂O₄, CoFe₂O₄, NiFe₂O₄ and CoMn₂O₄, but one or more embodiments of the present disclosure is not limited thereto.

Also, the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, and/or the like, but one or more embodiments of the present disclosure is not limited thereto.

The quantum dot may have a full width of half maximum (FWHM) of emission wavelength spectrum of about 45 nm or less, about 40 nm or less, or about 30 nm or less. Within this range, color purity or color reproducibility may be improved. In some embodiments, light emitted via such quantum dot is emitted in all directions, and light view angle properties may be improved (e.g., the size or width of the viewing angle may be enhanced or increased).

In some embodiments, the shape of the quantum dot may be generally utilized shapes in the art, without specific limitation. More particularly, the shape of spherical, pyramidal, multi-arm, or cubic nanoparticle, nanotube, nanowire, nanofiber, nanoplate particle, and/or the like may be utilized.

The quantum dot may control the color of emitted light according to the particle size thereof, and thus the quantum dot may have one or more suitable light emitting colors such as blue, red, green, and/or the like.

When the size of quantum dot particles or the ratio of elements in a quantum dot compound are regulated, an energy band gap may be controlled or selected. Accordingly, the quantum dot may be to emit light of one or more suitable wavelengths. Therefore, when quantum dots of different sizes are utilized or quantum dots whose element ratio is regulated in the quantum dot compound are utilized, a light emitting element ED emitting light of one or more suitable wavelengths may be obtained. For example, the size of the quantum dots or the ratio of elements in the quantum dot compound may be regulated to emit red, green, and/or blue light. In some embodiments, the quantum dots may be configured to emit white light by combining light of one or more suitable colors.

In each of the light emitting elements ED of embodiments illustrated in FIGS. 3 to 7, the electron transport region ETR is provided on the emission layer EML. The electron transport region ETR may include at least one selected from among the hole blocking layer HBL, the electron transport layer ETL, or the electron injection layer EIL, but the embodiment of the present disclosure is not limited thereto.

The electron transport region ETR may have a single layer formed of a single material, a single layer formed of a plurality of different materials, or a multilayer structure including a plurality of layers formed of a plurality of different materials.

For example, the electron transport region ETR may have a single layer structure of the electron injection layer EIL or the electron transport layer ETL, and may have a single layer structure formed of an electron injection material and an electron transport material. In some embodiments, the electron transport region ETR may have a single layer structure formed of a plurality of different materials, or may have a structure in which an electron transport layer ETL/electron injection layer EIL, a hole blocking layer HBL/electron transport layer ETL/electron injection layer EIL are stacked in order from the emission layer EML, but the embodiment of the present disclosure is not limited thereto. The electron transport region ETR may have a thickness, for example, from about 1,000 Å to about 1,500 Å.

The electron transport region ETR may be formed utilizing one or more suitable methods such as a vacuum deposition method, a spin coating method, a cast method, a Langmuir-Blodgett (LB) method, an inkjet printing method, a laser printing method, and a laser induced thermal imaging (LITI) method.

The electron transport region ETR may include a compound represented by Formula ET-1:

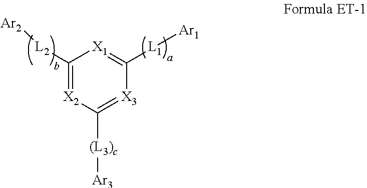

In Formula ET-1, at least one among X₁ to X₃ is N, and the rest are CRₐ. Rₐ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. Ar₁ to Ar₃ may each independently be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In Formula ET-1, a to c may each independently be an integer of 0 to 10. In Formula ET-1, L₁ to L₃ may each independently be a direct linkage, a substituted or unsubstituted arylene group having 6 to 30 ring-forming carbon atoms, or a substituted or unsubstituted heteroarylene group having 2 to 30 ring-forming carbon atoms. In some embodiments, when a to c may each independently be an integer of 2 or more, L₁ to L₃ may each independently be a substituted or unsubstituted arylene group having 6 to 30 ring-forming carbon atoms, or a substituted or unsubstituted heteroarylene group having 2 to 30 ring-forming carbon atoms.

The electron transport region ETR may include an anthracene-based compound. However, one or more embodiments of the present disclosure is not limited thereto, and the electron transport region ETR may include, for example, at least one selected from among tristris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazol-1-yl)phenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (BAIq), berylliumbis(benzoquinolin-10-olate (Bebq₂), 9,10-di(naphthalen-2-yl)anthracene (ADN), 1,3-bis[3,5-di(pyridin-3-yl)phenyl]benzene (BmPyPhB), and diphenyl(4-(triphenylsilyl)phenyl)phosphine oxide (TSPO1), and mixtures thereof, without limitation.

The electron transport region ETR may include at least one selected from among Compound ET1 to Compound ET36:

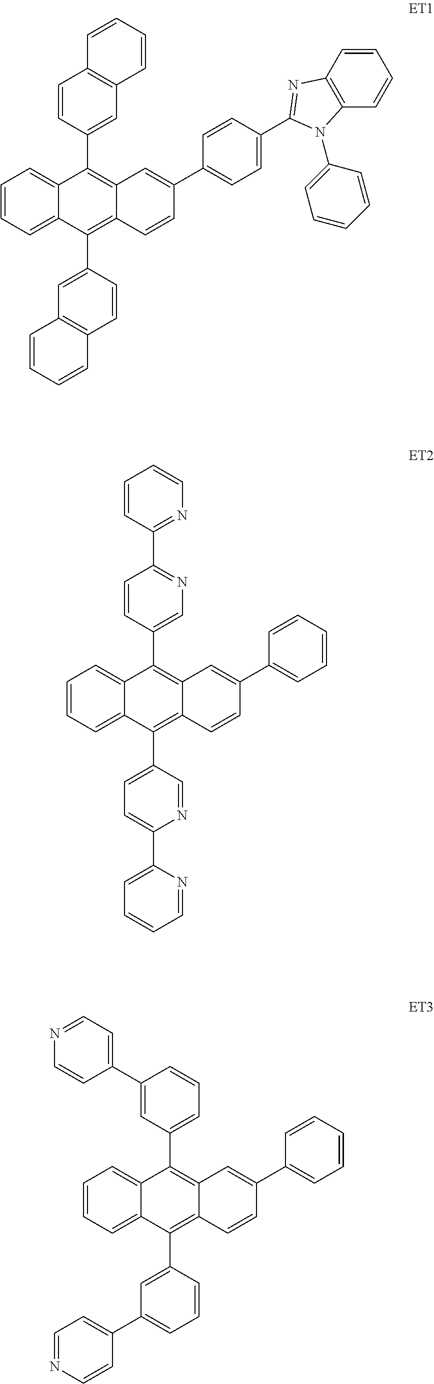 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

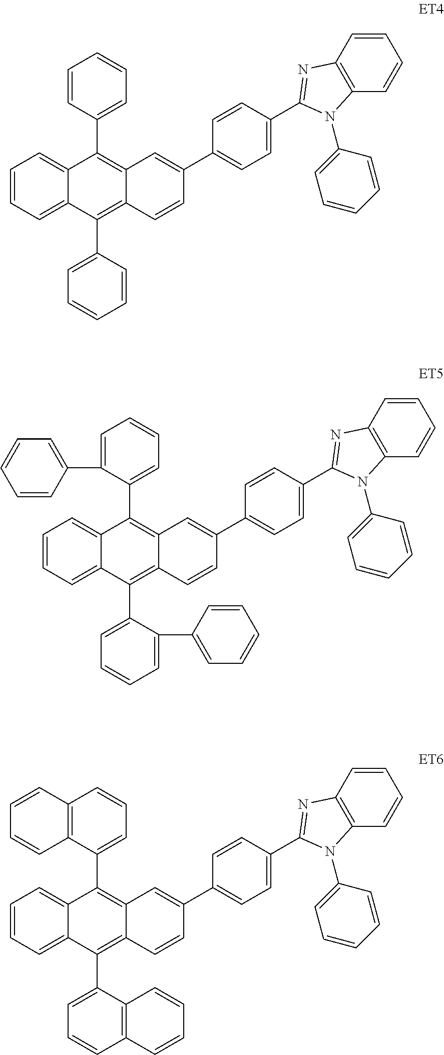 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

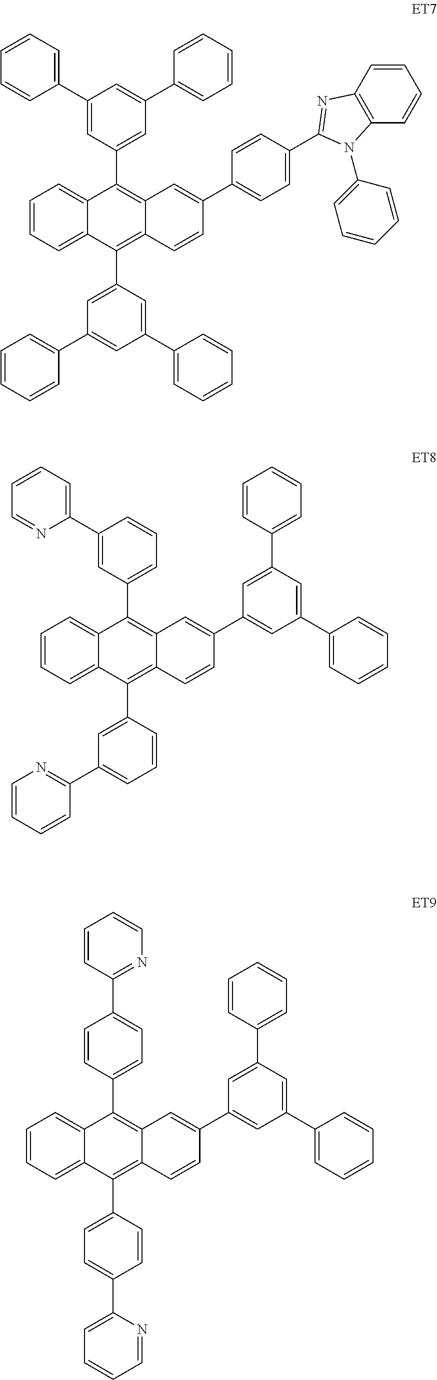 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

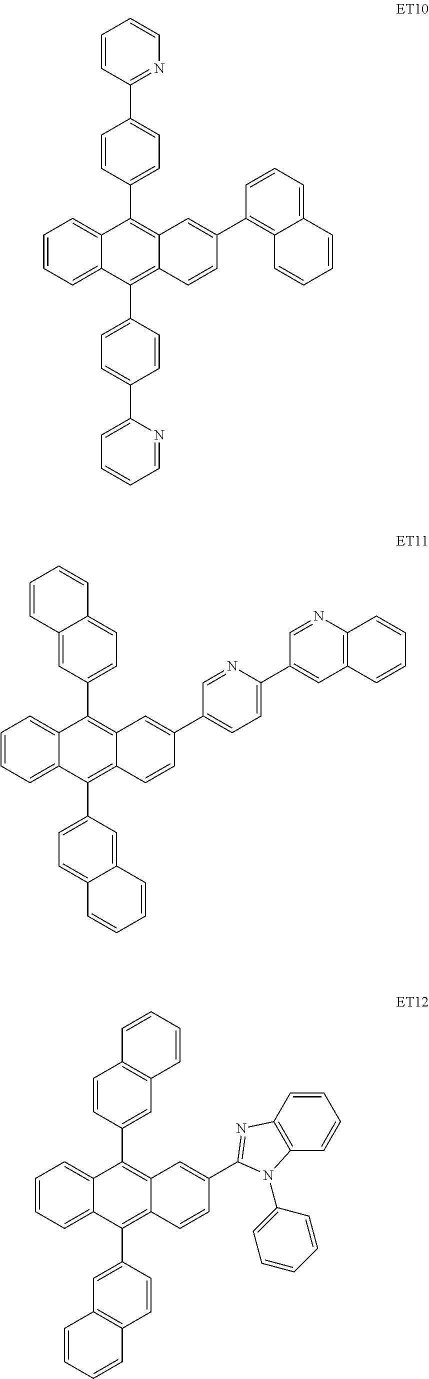 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

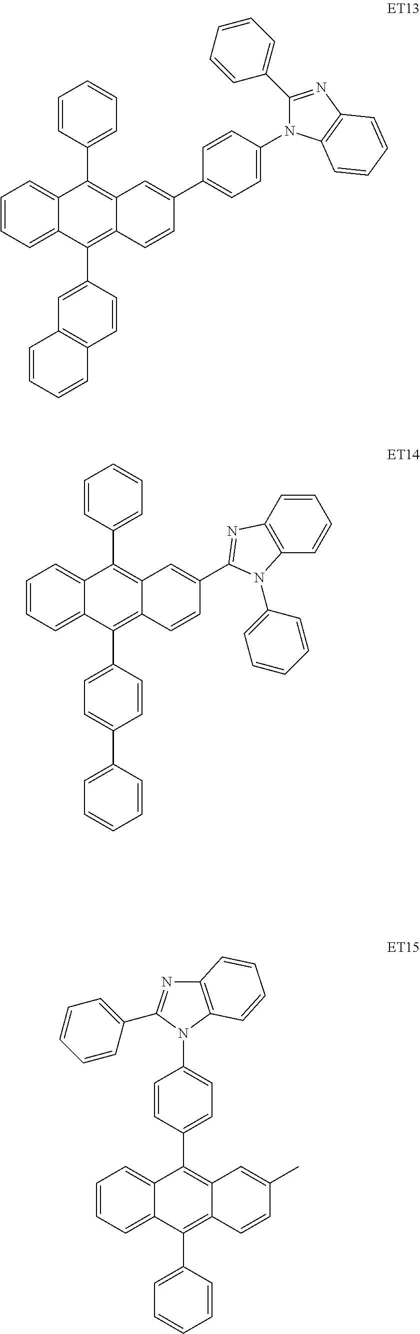 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

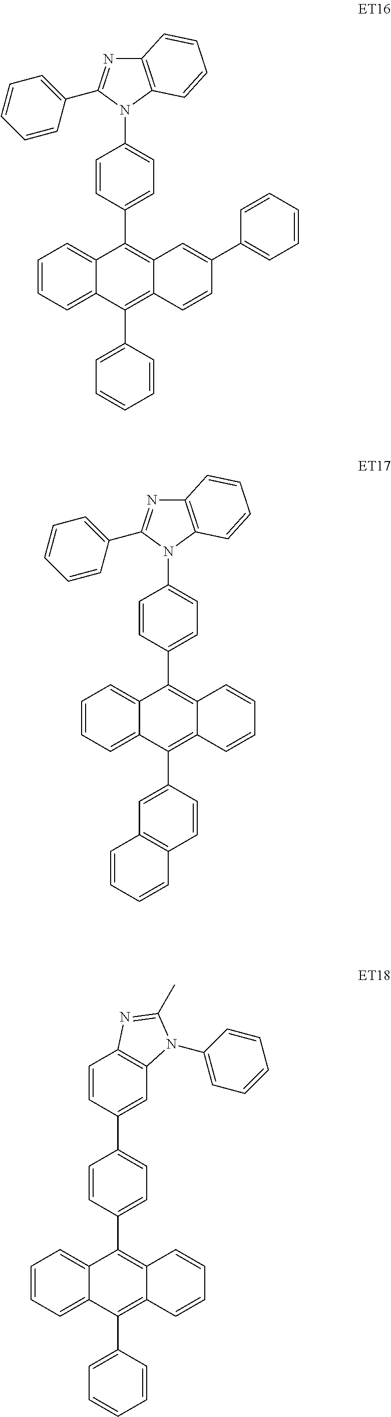 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

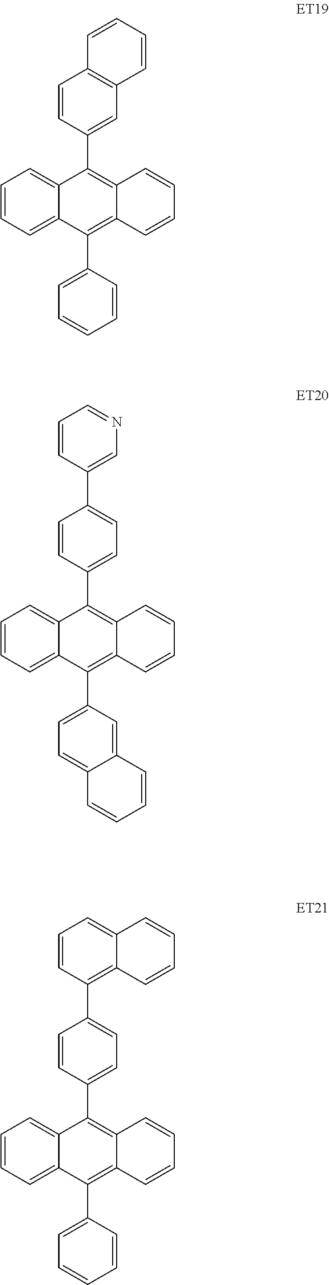

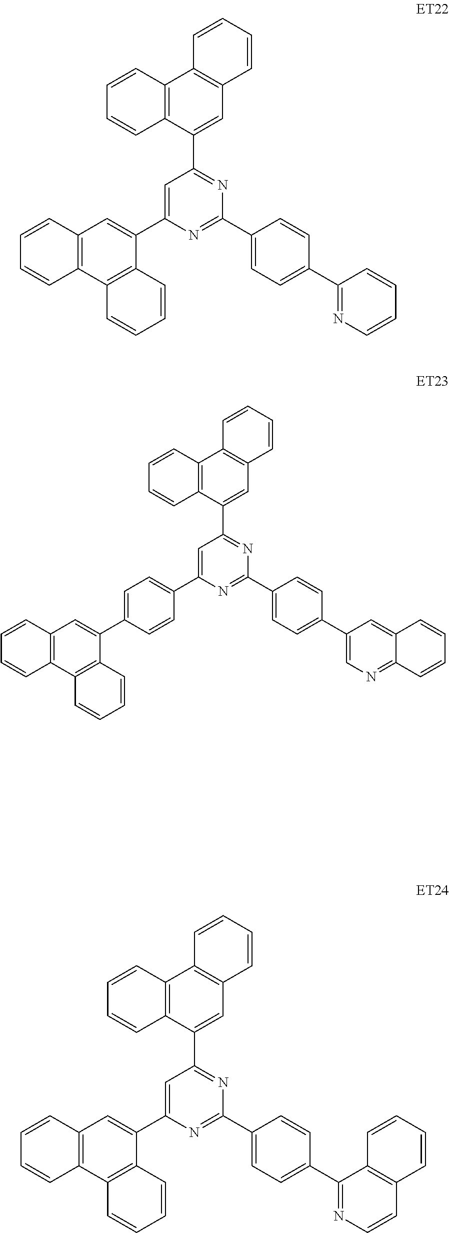 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

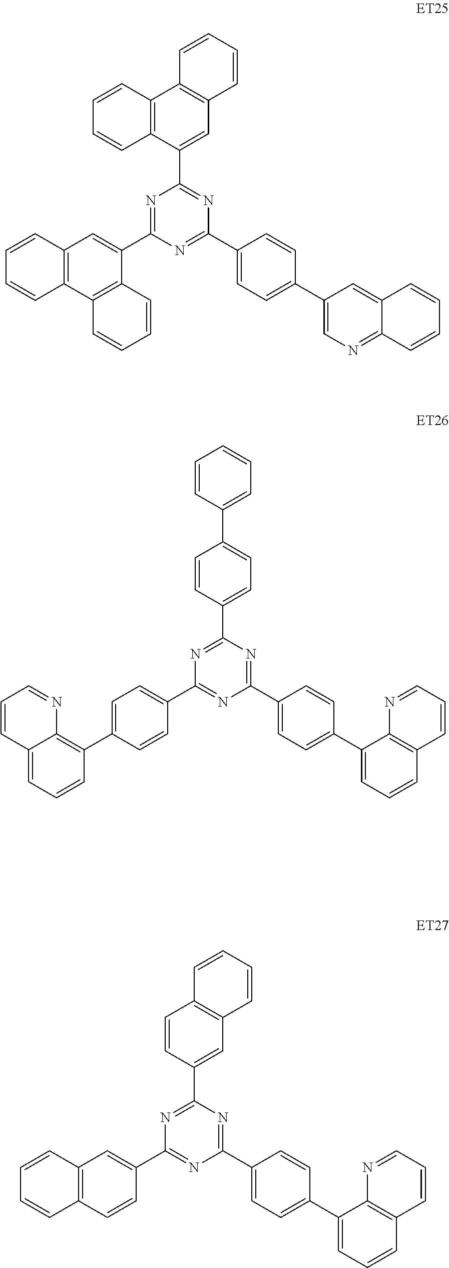 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

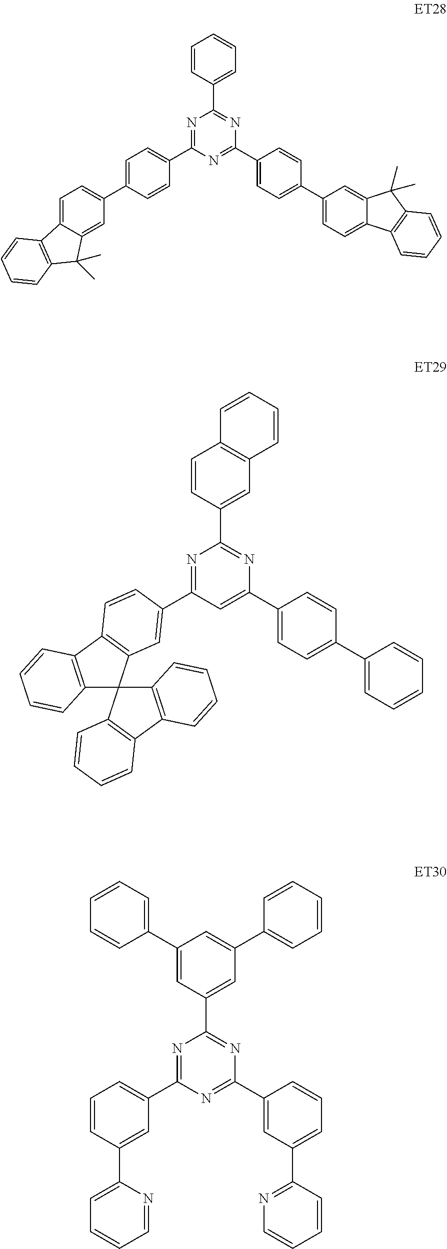 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

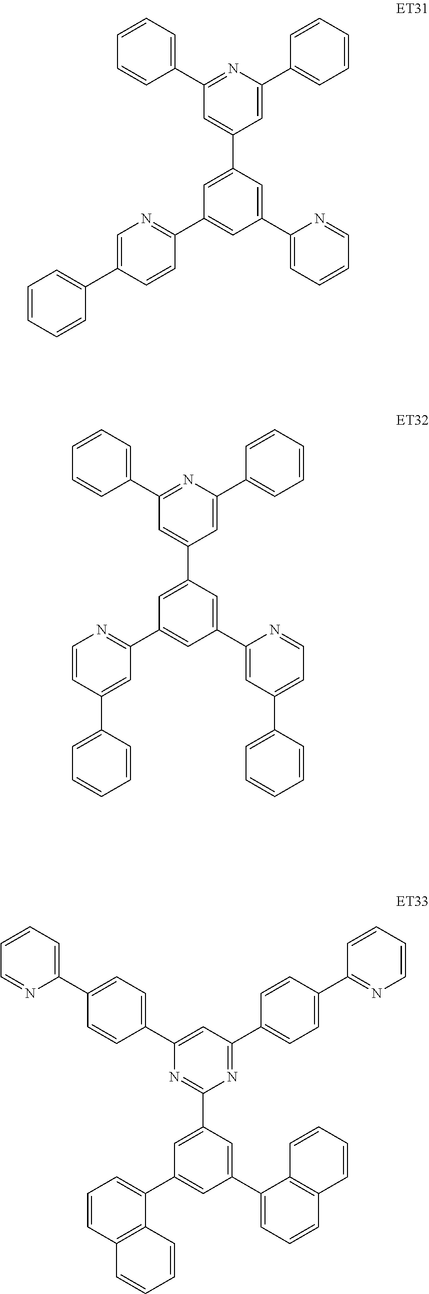 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

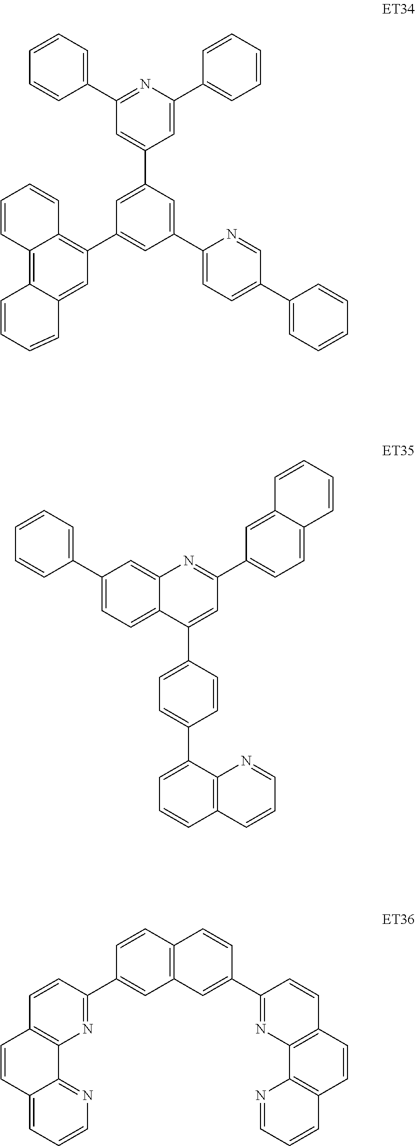 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

In some embodiments, the electron transport region ETR may include a metal halide such as LiF, NaCl, CsF, RbCl, RbI, CuI, and/or KI, a lanthanide metal such as Yb, and/or a co-deposited material of the metal halide and the lanthanide metal. For example, the electron transport region ETR may include KI:Yb, RbI:Yb, LiF:Yb, and/or the like, as a co-deposited material. In some embodiments, the electron transport region ETR may be formed utilizing a metal oxide such as Li₂O or BaO, or 8-hydroxyl-lithium quinolate (Liq), and/or the like, but the embodiment of the present disclosure is not limited thereto. The electron transport region ETR may also be formed of a mixture material of an electron transport material and an insulating organometallic salt. The organometallic salt may be a material having an energy band gap of about 4 eV or more. For example, the organometallic salt may include, for example, a metal acetate, a metal benzoate, a metal acetoacetate, a metal acetylacetonate, and/or a metal stearate.

The electron transport region ETR may further include at least one selected from among 2,9-dimethyl-4,7-diphenyl-1,10-phenanthroline (BCP), diphenyl(4-(triphenylsilyl)phenyl)phosphine oxide (TSPO1), or 4,7-diphenyl-1,10-phenanthroline (Bphen) in addition to the-described materials, but the embodiment of the present disclosure is not limited thereto.

The electron transport region ETR may include the-described compounds of the hole transport region in at least one selected from among the electron injection layer EIL, the electron transport layer ETL, or the hole blocking layer HBL.

When the electron transport region ETR includes the electron transport layer ETL, the electron transport layer ETL may have a thickness of about 100 Å to about 1,000 Å, for example, about 150 Å to about 500 Å. When the thickness of the electron transport layer ETL satisfies the aforementioned range, satisfactory electron transport characteristics may be obtained without a substantial increase in driving voltage. When the electron transport region ETR includes the electron injection layer EIL, the electron injection layer EIL may have a thickness of about 1 Å to about 100 Å, for example, about 3 Å to about 90 Å. When the thickness of the electron injection layer EIL satisfies the-described range, satisfactory electron injection characteristics may be obtained without a substantial increase in driving voltage.

The second electrode EL2 is provided on the electron transport region ETR. The second electrode EL2 may be a common electrode. The second electrode EL2 may be a cathode or an anode, but one or more embodiments of the present disclosure is not limited thereto. For example, when the first electrode EL1 is an anode, the second electrode EL2 may be a cathode, and when the first electrode EL1 is a cathode, the second electrode EL2 may be an anode. The second electrode EL2 may include at least one selected among Ag, Mg, Cu, Al, Pt, Pd, Au, Ni, Nd, Ir, Cr, Li, Ca, LiF, Mo, Ti, W, In, Sn, and Zn, compounds of two or more selected therefrom, mixtures of two or more selected therefrom, or oxides thereof.

The second electrode EL2 may be a transmissive electrode, a transflective electrode or a reflective electrode. When the second electrode EL2 is the transmissive electrode, the second electrode EL2 may include a transparent metal oxide, for example, ITO, IZO, ZnO, ITZO, and/or the like.

When the second electrode EL2 is the transflective electrode or the reflective electrode, the second electrode EL2 may include at least one selected from among Ag, Mg, Cu, Al, Pt, Pd, Au, Ni, Nd, Ir, Cr, Li, Ca, LiF/Ca, LiF/Al, Mo, Ti, Yb, and W, or a compound or mixture thereof (e.g., AgMg, AgYb, or MgYb). In some embodiments, the second electrode EL2 may have a multilayer structure including a reflective film or a transflective film formed of the-described materials, and a transparent conductive film formed of ITO, IZO, ZnO, ITZO, and/or the like. For example, the second electrode EL2 may include the-described metal materials, combinations of at least two metal materials of the-described metal materials, oxides of the-described metal materials, and/or the like.

In some embodiments, the second electrode EL2 may be connected with an auxiliary electrode. When the second electrode EL2 is connected with the auxiliary electrode, the resistance of the second electrode EL2 may be decreased.

In some embodiments, a capping layer CPL may further be provided on the second electrode EL2 of the light emitting element ED of one or more embodiments. The capping layer CPL may include a multilayer or a single layer.

In one or more embodiments, the capping layer CPL may be an organic layer or an inorganic layer. For example, when the capping layer CPL contains an inorganic material, the inorganic material may include an alkaline metal compound (e.g., LiF), an alkaline earth metal compound (e.g., MgF₂), SiON, SiNₓ, SiOy, and/or the like.

For example, when the capping layer CPL includes an organic material, the organic material may include at least one selected from among α-NPD, NPB, TPD, m-MTDATA, Alq₃, CuPc, N4,N4,N4′,N4′-tetra(biphenyl-4-yl)biphenyl-4,4′-diamine (TPD15), and 4,4′,4″-tris(carbazol-9-yl)triphenylamine (TCTA), and/or the like, and/or an epoxy resin, and/or acrylate such as methacrylate. However, the embodiment of the present disclosure is not limited thereto, and the capping layer CPL may include at least one selected from among Compounds P1 to P5:

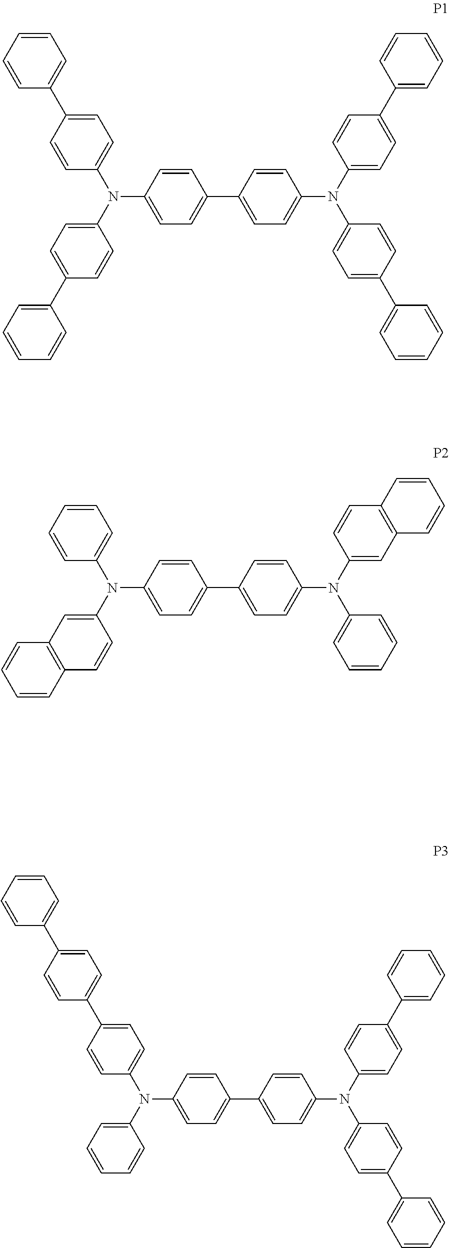 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

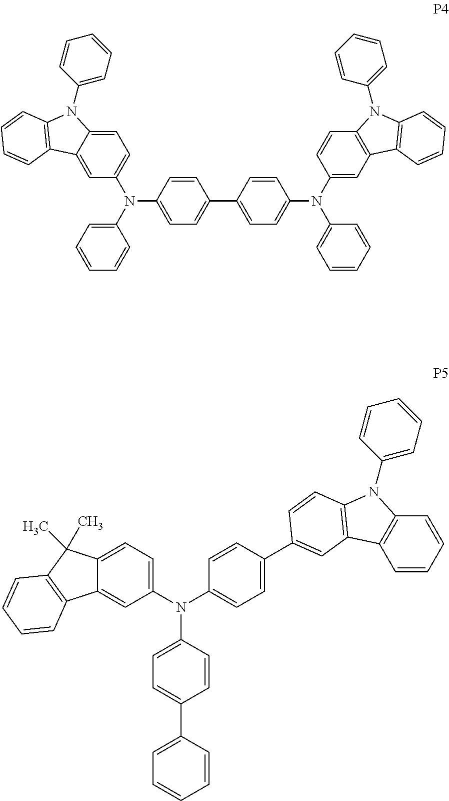 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2cc(N(c3ccc(-c4ccccc4)cc3)c3ccc(-c4ccc5c(c4)c4ccccc4n5-c4ccccc4)cc3)ccc21]

In some embodiments, the refractive index of the capping layer CPL may be about 1.6 or more. For example, the refractive index of the capping layer CPL may be about 1.6 or more with respect to light in a wavelength range of about 550 nm to about 660 nm.

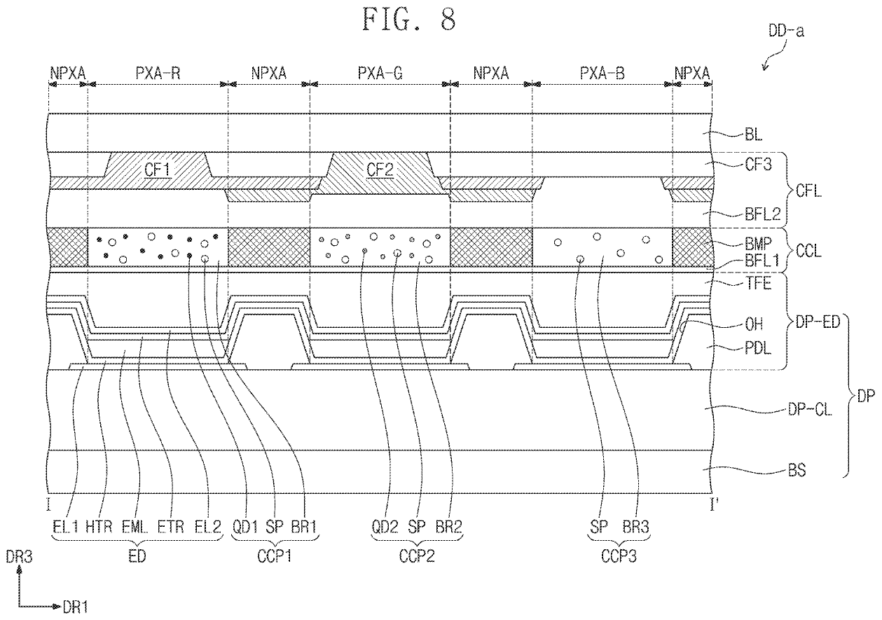

Each of FIGS. 8 to 11 is a cross-sectional view of a display apparatus according to one or more embodiments of the present disclosure. Hereinafter, in describing the display apparatuses of embodiments with reference to FIGS. 8 to 11, the duplicated features which have been described in FIGS. 1 to 7 are not described again, but their differences will be mainly described (e.g., in more detail).

Referring to FIG. 8, the display apparatus DD-a according to one or more embodiments may include a display panel DP including a display device layer DP-ED, a light control layer CCL provided on the display panel DP, and a color filter layer CFL. In one or more embodiments illustrated in FIG. 8, the display panel DP may include a base layer BS, a circuit layer DP-CL provided on the base layer BS, and the display device layer DP-ED, and the display device layer DP-ED may include a light emitting element ED.

The light emitting element ED may include a first electrode EL1, a hole transport region HTR provided on the first electrode EL1, an emission layer EML provided on the hole transport region HTR, an electron transport region ETR provided on the emission layer EML, and a second electrode EL2 provided on the electron transport region ETR. In some embodiments, the structures of the light emitting elements of FIGS. 3 to 7 as described herein may be equally applied to the structure of the light emitting element ED illustrated in FIG. 8. The hole transport region HTR of the light emitting element ED included in the display device DD-a according to one or more embodiments may include the amine compound of one or more embodiments.

Referring to FIG. 8, the emission layer EML may be provided in an opening OH defined in a pixel defining film PDL. For example, the emission layer EML which is divided by the pixel defining film PDL and provided corresponding to each light emitting regions PXA-R, PXA-G, and PXA-B may be to emit (e.g., configured to emit) light in substantially the same wavelength range. In the display apparatus DD-a of one or more embodiments, the emission layer EML may be to emit (e.g., configured to emit) blue light. In some embodiments, unlike the configuration illustrated, in one or more embodiments, the emission layer EML may be provided as a common layer in the entire light emitting regions PXA-R, PXA-G, and PXA-B.

The light control layer CCL may be provided on the display panel DP. The light control layer CCL may include a light conversion body. The light conversion body may be a quantum dot, a phosphor, and/or the like. The light conversion body may be to emit (e.g., configured to emit) provided light by converting the wavelength thereof. For example, the light control layer CCL may a layer containing the quantum dot or a layer containing the phosphor.

The light control layer CCL may include a plurality of light control parts CCP1, CCP2 and CCP3. The light control parts CCP1, CCP2, and CCP3 may be spaced (e.g., apart) from each other.

Referring to FIG. 8, divided patterns BMP may be provided between the light control parts CCP1, CCP2 and CCP3 which are spaced (e.g., apart) from each other, but the embodiment of the present disclosure is not limited thereto. FIG. 8 illustrates that the divided patterns BMP do not overlap the light control parts CCP1, CCP2 and CCP3, but at least a portion of the edges of the light control parts CCP1, CCP2 and CCP3 may overlap the divided patterns BMP.

The light control layer CCL may include a first light control part CCP1 containing a first quantum dot QD1 which converts first color light provided from the light emitting element ED into second color light, a second light control part CCP2 containing a second quantum dot QD2 which converts the first color light into third color light, and a third light control part CCP3 which transmits the first color light.

In one or more embodiments, the first light control part CCP1 may provide red light that is the second color light, and the second light control part CCP2 may provide green light that is the third color light. The third light control part CCP3 may provide blue light by transmitting the blue light that is the first color light provided from the light emitting element ED. For example, the first quantum dot QD1 may be a red quantum dot, and the second quantum dot QD2 may be a green quantum dot. The same as described herein may be applied with respect to the quantum dots QD1 and QD2.

In some embodiments, the light control layer CCL may further include a scatterer SP. The first light control part CCP1 may include the first quantum dot QD1 and the scatterer SP, the second light control part CCP2 may include the second quantum dot QD2 and the scatterer SP, and the third light control part CCP3 may not include (e.g., may exclude) any quantum dot but include the scatterer SP.

The scatterer SP may be inorganic particles. For example, the scatterer SP may include at least one selected from among TiO₂, ZnO, Al₂O₃, SiO₂, or hollow sphere silica. The scatterer SP may include any one selected from among TiO₂, ZnO, Al₂O₃, SiO₂, and hollow sphere silica, or may be a mixture of at least two materials selected from among TiO₂, ZnO, Al₂O₃, SiO₂, and hollow sphere silica.

The first light control part CCP1, the second light control part CCP2, and the third light control part CCP3 each may include base resins BR1, BR2, and BR3 in which the quantum dots QD1 and QD2 and the scatterer SP are dispersed. In one or more embodiments, the first light control part CCP1 may include the first quantum dot QD1 and the scatterer SP dispersed in a first base resin BR1, the second light control part CCP2 may include the second quantum dot QD2 and the scatterer SP dispersed in a second base resin BR2, and the third light control part CCP3 may include the scatterer SP dispersed in a third base resin BR3.

The base resins BR1, BR2, and BR3 are media in which the quantum dots QD1 and QD2 and the scatterer SP are dispersed, and may be formed of one or more suitable resin compositions, which may be generally referred to as a binder. For example, the base resins BR1, BR2, and BR3 may be acrylic-based resins, urethane-based resins, silicone-based resins, epoxy-based resins, and/or the like. The base resins BR1, BR2, and BR3 may be transparent resins. In one or more embodiments, the first base resin BR1, the second base resin BR2, and the third base resin BR3 may be the same as or different from each other.

The light control layer CCL may include a barrier layer BFL1. The barrier layer BFL1 may serve to prevent or reduce the penetration of moisture and/or oxygen (hereinafter, referred to as ‘moisture/oxygen’). The barrier layer BFL1 may block or reduce the light control parts CCP1, CCP2 and CCP3 from being exposed to moisture/oxygen. In some embodiments, the barrier layer BFL1 may cover the light control parts CCP1, CCP2, and CCP3. In some embodiments, the barrier layer BFL2 may be provided between the light control parts CCP1, CCP2, and CCP3 and the color filter layer CFL.

The barrier layers BFL1 and BFL2 may include at least one inorganic layer. For example, the barrier layers BFL1 and BFL2 may include an inorganic material. For example, the barrier layers BFL1 and BFL2 may include a silicon nitride, an aluminum nitride, a zirconium nitride, a titanium nitride, a hafnium nitride, a tantalum nitride, a silicon oxide, an aluminum oxide, a titanium oxide, a tin oxide, a cerium oxide, a silicon oxynitride, a metal thin film which secures a transmittance, and/or the like. In some embodiments, the barrier layers BFL1 and BFL2 may further include an organic film. The barrier layers BFL1 and BFL2 may be formed of a single layer or a plurality of layers.

In the display apparatus DD-a of one or more embodiments, the color filter layer CFL may be provided on the light control layer CCL. For example, the color filter layer CFL may be directly provided on the light control layer CCL. In this case, the barrier layer BFL2 may not be provided.

The color filter layer CFL may include color filters CF1, CF2, and CF3. The color filter layer CFL may include a first filter CF1 configured to transmit the second color light, a second filter CF2 configured to transmit the third color light, and a third filter CF3 configured to transmit the first color light. For example, the first filter CF1 may be a red filter, the second filter CF2 may be a green filter, and the third filter CF3 may be a blue filter. The filters CF1, CF2, and CF3 each may include a polymeric photosensitive resin and a pigment or dye. The first filter CF1 may include a red pigment or dye, the second filter CF2 may include a green pigment or dye, and the third filter CF3 may include a blue pigment or dye.

In some embodiments, the embodiment of the present disclosure is not limited thereto, and the third filter CF3 may not include (e.g., may exclude any of) a pigment or dye. The third filter CF3 may include a polymeric photosensitive resin and may not include (e.g., may exclude) a pigment or dye. The third filter CF3 may be transparent. The third filter CF3 may be formed of a transparent photosensitive resin.

Furthermore, in one or more embodiments, the first filter CF1 and the second filter CF2 may be yellow filters. The first filter CF1 and the second filter CF2 may be provided in one body without distinction. The first to third filters CF1, CF2 and CF3 may be provided corresponding to the red luminous area PXA-R, the green luminous area PXA-G and the blue luminous area PXA-B, respectively.

In some embodiments, the color filter layer CFL may further include alight blocking part. The color filter layer CFL may include a light blocking part provided at the boundaries to overlap with adjacent filters CF1, CF2 and CF3. The light blocking part may be a black matrix. The light blocking part may be formed by including an organic light blocking material or an inorganic light blocking material, including a black pigment or black dye. The light blocking part may divide the boundaries among adjacent filters CF1, CF2 and CF3. In some embodiments, the light blocking part may be formed as a blue filter.

A base substrate BL may be provided on the color filter layer CFL. The base substrate BL may be a member which provides a base surface in which the color filter layer CFL, the light control layer CCL, and/or the like are provided. The base substrate BL may be a glass substrate, a metal substrate, a plastic substrate, and/or the like. However, the embodiment of the present disclosure is not limited thereto, and the base substrate BL may be an inorganic layer, an organic layer, or a composite material layer. In some embodiments, unlike the configuration illustrated, in one or more embodiments, the base substrate BL may not be provided.

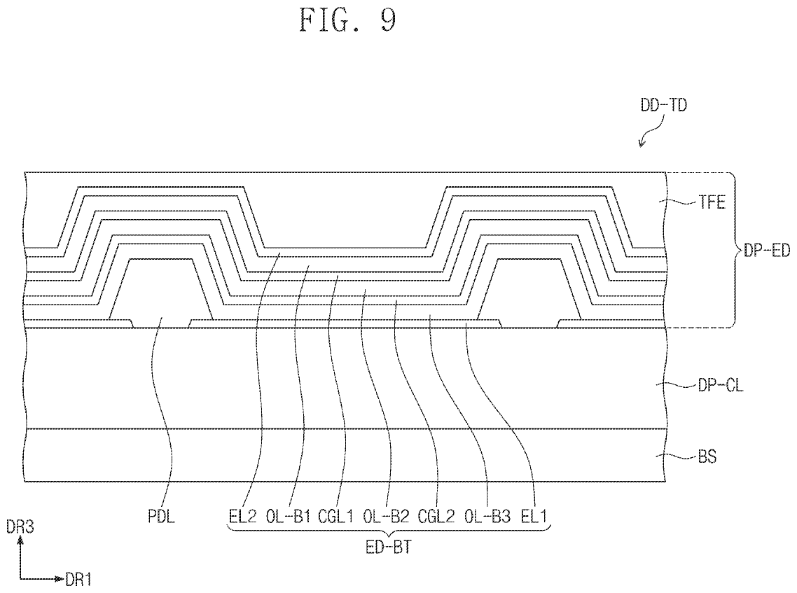

FIG. 9 is a cross-sectional view illustrating a portion of a display apparatus according to one or more embodiments. In the display apparatus DD-TD of one or more embodiments, the light emitting element ED-BT may include a plurality of light emitting structures OL-B1, OL-B2, and OL-B3. The light emitting element ED-BT may include a first electrode EL1 and a second electrode EL2 which face each other, and the plurality of light emitting structures OL-B1, OL-B2, and OL-B3 sequentially stacked in the thickness direction between the first electrode EL1 and the second electrode EL2. The light emitting structures OL-B1, OL-B2, and OL-B3 each may include an emission layer EML (FIG. 8) and a hole transport region HTR and an electron transport region ETR provided with the emission layer EML (FIG. 8) located therebetween.

For example, the light emitting element ED-BT included in the display apparatus DD-TD of one or more embodiments may be a light emitting element having a tandem structure and including a plurality of emission layers.

In one or more embodiments illustrated in FIG. 9, all light beams respectively emitted from the light emitting structures OL-B1, OL-B2, and OL-B3 may be blue light. However, the embodiment of the present disclosure is not limited thereto, and the light beams respectively emitted from the light emitting structures OL-B1, OL-B2, and OL-B3 may have wavelength ranges different from each other. For example, the light emitting element ED-BT including the plurality of light emitting structures OL-B1, OL-B2, and OL-B3 which emit light beams having wavelength ranges different from each other may be to emit (e.g., configured to emit) white light.

Charge generation layers CGL1 and CGL2 may be respectively provided between two of the neighboring light emitting structures OL-B1, OL-B2, and OL-B3. The charge generation layers CGL1 and CGL2 may include a p-type or kind charge generation layer (e.g., p-charge generation (or generating) layer) and/or an n-type or kind charge generation layer (e.g., n-charge generation (or generating) layer).

At least one of the light emitting structures OL-B1, OL-B2, and OL-B3 included in the display device DD-TD of one or more embodiments may include the amine compound of one or more embodiments described herein.

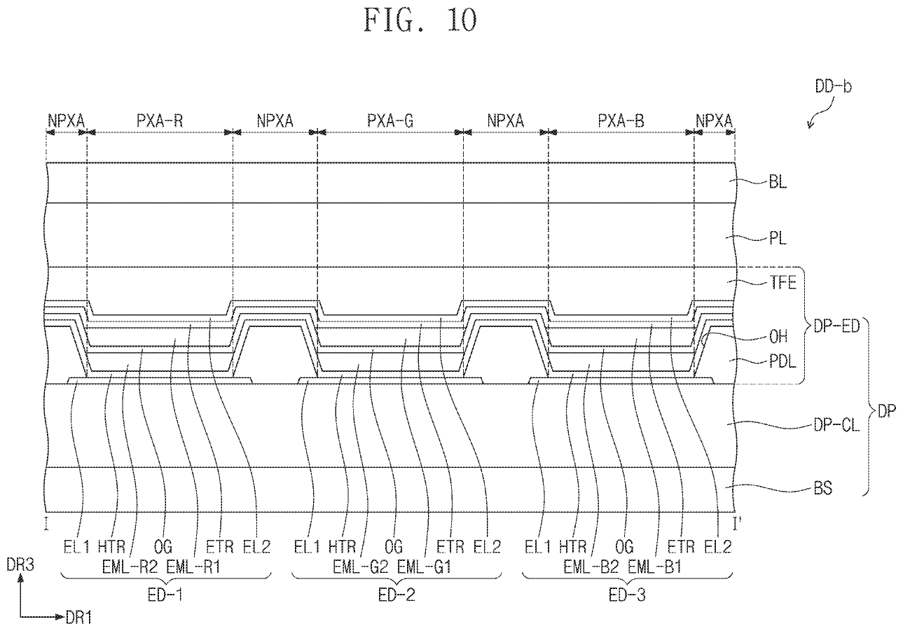

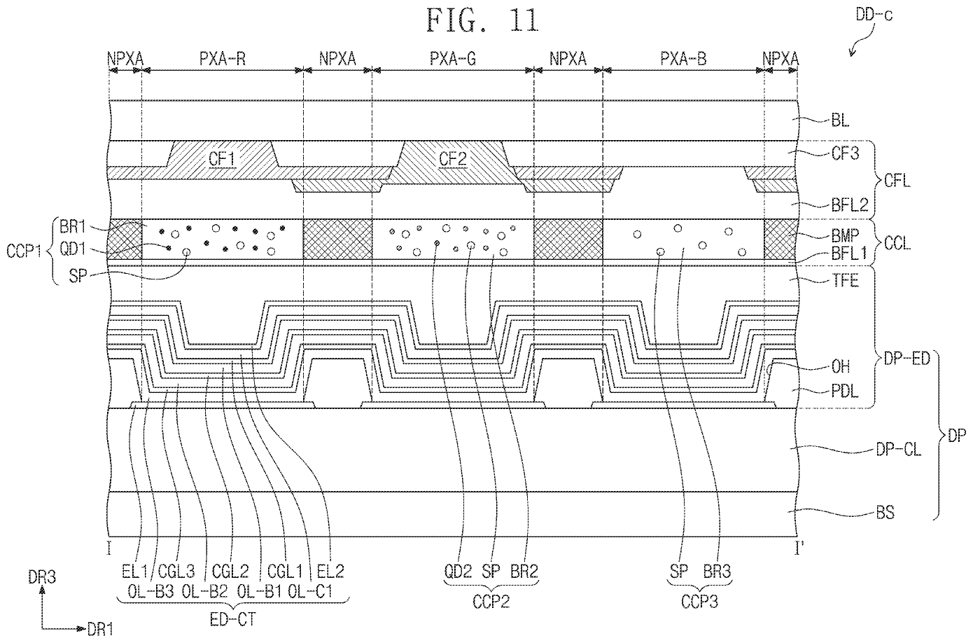

Referring to FIG. 10, the display apparatus DD-b according to one or more embodiments may include light emitting elements ED-1, ED-2, and ED-3 in which two emission layers are stacked. Compared with the display apparatus DD of one or more embodiments illustrated in FIG. 2, one or more embodiments illustrated in FIG. 10 has a difference in that the first to third light emitting elements ED-1, ED-2, and ED-3 each include two emission layers stacked in the thickness direction. In each of the first to third light emitting elements ED-1, ED-2, and ED-3, the two emission layers may be to emit (e.g., configured to emit) light in substantially the same wavelength region.

The first light emitting element ED-1 may include a first red emission layer EML-R1 and a second red emission layer EML-R2. The second light emitting element ED-2 may include a first green emission layer EML-G1 and a second green emission layer EML-G2. In some embodiments, the third light emitting element ED-3 may include a first blue emission layer EML-B1 and a second blue emission layer EML-B2. An emission auxiliary part OG may be provided between the first red emission layer EML-R1 and the second red emission layer EML-R2, between the first green emission layer EML-G1 and the second green emission layer EML-G2, and between the first blue emission layer EML-B1 and the second blue emission layer EML-B2.

The emission auxiliary part OG may include a single layer or a multilayer. The emission auxiliary part OG may include a charge generation layer. More specifically, the emission auxiliary part OG may include an electron transport region, a charge generation layer, and a hole transport region that are sequentially stacked. The emission auxiliary part OG may be provided as a common layer in the whole of the first to third light emitting elements ED-1, ED-2, and ED-3. However, the embodiment of the present disclosure is not limited thereto, and the emission auxiliary part OG may be provided by being patterned within the openings OH defined in the pixel defining film PDL.

The first red emission layer EML-R1, the first green emission layer EML-G1 and the first blue emission layer EML-B1 may be provided between the electron transport region ETR and the emission auxiliary part OG. The second red emission layer EML-R2, the second green emission layer EML-G2 and the second blue emission layer EML-B2 may be provided between the emission auxiliary part OG and the hole transport region HTR.

For example, the first light emitting element ED-1 may include a first electrode EL1, a hole transport region HTR, a second red emission layer EML-R2, an emission auxiliary part OG, a first red emission layer EML-R1, an electron transport region ETR, and a second electrode EL2, stacked in order. The second light emitting element ED-2 may include a first electrode EL1, a hole transport region HTR, a second green emission layer EML-G2, an emission auxiliary part OG, a first green emission layer EML-G1, an electron transport region ETR, and a second electrode EL2, stacked in order. The third light emitting element ED-3 may include a first electrode EL1, a hole transport region HTR, a second blue emission layer EML-B2, an emission auxiliary part OG, a first blue emission layer EML-B1, an electron transport region ETR, and a second electrode EL2, stacked in order.

In some embodiments, an optical auxiliary layer PL may be provided on the display device layer DP-ED. The optical auxiliary layer PL may include a polarizing layer. The optical auxiliary layer PL may be provided on the display panel DP and control reflected light in the display panel DP due to external light. Unlike the configuration illustrated, the optical auxiliary layer PL in the display apparatus according to one or more embodiments may not be provided.

Unlike FIGS. 9 and 10, FIG. 11 illustrates that a display apparatus DD-c includes four light emitting structures OL-B1, OL-B2, OL-B3, and OL-C1. A light emitting element ED-CT may include a first electrode EL1 and a second electrode EL2 which face each other, and first to fourth light emitting structures OL-B1, OL-B2, OL-B3, and OL-C1 that are sequentially stacked in the thickness direction between the first electrode EL1 and the second electrode EL2. Charge generation layers CGL1, CGL2, and CGL3 may be provided between the first to fourth light emitting structures OL-B1, OL-B2, OL-B3, and OL-C1. Among the four light emitting structures, the first to third light emitting structures OL-B1, OL-B2, and OL-B3 may be to emit (e.g., configured to emit) blue light, and the fourth light emitting structure OL-C1 may be to emit (e.g., configured to emit) green light. However, the embodiment of the present disclosure is not limited thereto, and the first to fourth light emitting structures OL-B1, OL-B2, OL-B3, and OL-C1 may be to emit (e.g., configured to emit) light beams in different wavelength regions.

The charge generation layers CGL1, CGL2, and CGL3 provided between adjacent light emitting structures OL-B1, OL-B2, OL-B3, and OL-C1 may include a p-type or kind charge generation layer (e.g., p-charge generation (or generating) layer) and/or an n-type or kind charge generation layer (e.g., n-charge generation (or generating) layer).

At least one of the light emitting structures OL-B1, OL-B2, OL-B3, and OL-C1 included in the display apparatus DD-c of one or more embodiments may include the amine compound of one or more embodiments described herein.

The light emitting element ED according to one or more embodiments of the present disclosure includes the amine compound of one or more embodiments described herein in at least one functional layer provided between the first electrode EL1 and the second electrode EL2, and may thus exhibit improved light emitting efficiency and improved lifespan. The light emitting element ED according to one or more embodiments may include the amine compound of one or more embodiments described herein in at least one of the hole transport region HTR, the emission layer EML, or the electron transport region ETR provided between the first electrode EL1 and the second electrode EL2, or in a capping layer CPL. For example, the amine compound according to one or more embodiments may be included in the hole transport region HTR of the light emitting element ED of one or more embodiments, and the light emitting element of one or more embodiments may exhibit relativley high efficiency and long lifespan.

The amine compound of one or more embodiments described herein includes a first core, and second and third substituents, and may thus increase stability of materials and improve hole transport properties. Accordingly, a light emitting element ED including the amine compound of one or more embodiments may have increased lifespan and efficiency. For example, the light emitting element ED of one or more embodiments includes the amine compound according to one or more embodiments in an electron blocking layer, may thus exhibit increased efficiency and lifespan.

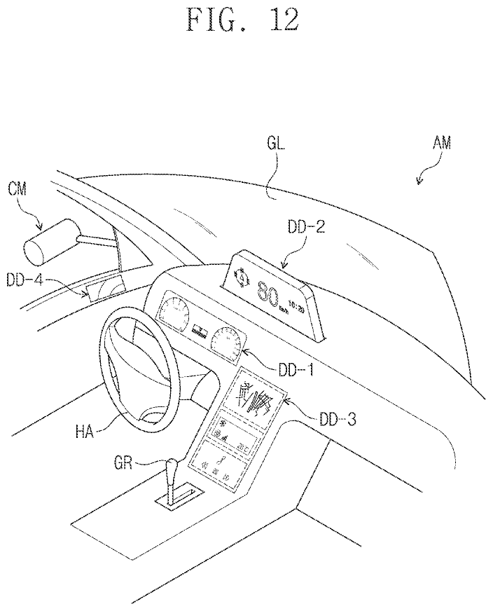

FIG. 12 is a view illustrating a vehicle AM in which first to fourth display apparatuses DD-1, DD-2, DD-3, and DD-4 are provided. At least one among the first to fourth display apparatuses DD-1, DD-2, DD-3, and DD-4 may include the same configuration as the display apparatuses DD, DD-TD, DD-a, DD-b, and DD-c as described with reference to FIGS. 1, and 2, and 8 to 11.

FIG. 12 illustrates a vehicle AM, but this is an example, and the first to fourth display apparatuses DD-1, DD-2, DD-3, and DD-4 may be provided in another transportation refers to such as bicycles, motorcycles, trains, ships, and airplanes. In some embodiments, at least one among the first to fourth display apparatuses DD-1, DD-2, DD-3, and DD-4 including the same configuration as the display apparatuses DD, DD-TD, DD-a, DD-b, and DD-c of one or more embodiments may be employed in a personal computer, a laptop computer, a personal digital terminal, a game console, a portable electronic device, a television, a monitor, an outdoor billboard, and/or the like. In some embodiments, these are merely provided as embodiments, and thus may be employed in other electronic apparatuses unless departing from the present disclosure.

At least one of the first to fourth display apparatuses DD-1, DD-2, DD-3, and DD-4 may include the light emitting element ED of one or more embodiments described with reference to FIGS. 3 to 7. The light emitting element ED according to one or more embodiments may include an amine compound of one or more embodiments. At least one of the first to fourth display apparatuses DD-1, DD-2, DD-3, and DD-4 includes a light emitting element ED containing the amine compound of one or more embodiments, thereby increasing lifespan.

Referring to FIG. 12, the vehicle AM may include a steering wheel HA and a gear GR for driving the vehicle AM. In some embodiments, the vehicle AM may include a front window GL provided so as to face the driver.

The first display apparatus DD-1 may be provided in a first region overlapping the steering wheel HA. For example, the first display apparatus DD-1 may be a digital cluster which displays first information of the vehicle AM. The first information may include a first scale which indicates a driving speed of the vehicle AM, a second scale which indicates an engine speed (that is, revolutions per minute (RPM)), an image which indicates a fuel state, and/or the like. A first scale and a second scale may be indicated as a digital image.

The second display apparatus DD-2 may be provided in a second region facing the driver's seat and overlapping the front window GL. The driver's seat may be a seat in which the steering wheel HA is provided. For example, the second display apparatus DD-2 may be a head up display (HUD) which displays second information of the vehicle AM. The second display apparatus DD-2 may be optically transparent.

The second information may include digital numbers which indicate a driving speed, and may further include information such as the current time. Unlike the configuration illustrated, the second information of the second display apparatus DD-2 may be projected to the front window GL to be displayed.

The third display apparatus DD-3 may be provided in a third region adjacent to the gear GR. For example, the third display apparatus DD-3 may be provided between the driver's seat and the passenger seat and may be a center information display (CID) for a vehicle AM for displaying third information. The passenger seat may be a seat spaced apart from the driver's seat with the gear GR provided therebetween. The third information may include information about traffic (e.g., navigation information), playing music or radio or a video (or an image), temperatures inside the vehicle AM, and/or the like.

The fourth display apparatus DD-4 may be spaced apart from the steering wheel HA and the gear GR, and may be provided in a fourth region adjacent to the side of the vehicle AM. For example, the fourth display apparatus DD-4 may be a digital side-view mirror which displays fourth information. The fourth display apparatus DD-4 may display an image outside the vehicle AM taken by a camera module CM provided outside the vehicle AM. The fourth information may include an image outside the vehicle AM.

The herein-described first to fourth information may be examples, and the first to fourth display apparatuses DD-1, DD-2, DD-3, and DD-4 may further display information about the inside and outside of the vehicle AM. The first to fourth information may include different information. However, the embodiment of the present disclosure is not limited thereto, and a part of the first to fourth information may include the same information as one another.

Terms such as “substantially,” “about,” and “approximately” are used as relative terms and not as terms of degree, and are intended to account for the inherent deviations in measured or calculated values that would be recognized by those of ordinary skill in the art. They may be inclusive of the stated value and an acceptable range of deviation as determined by one of ordinary skill in the art, considering the limitations and error associated with measurement of that quantity. For example, “about” may refer to one or more standard deviations, or ±30%, 20%, 10%, 5% of the stated value.

Numerical ranges disclosed herein include and are intended to disclose all subsumed sub-ranges of the same numerical precision. For example, a range of “1.0 to 10.0” includes all subranges having a minimum value equal to or greater than 1.0 and a maximum value equal to or less than 10.0, such as, for example, 2.4 to 7.6. Applicant therefore reserves the right to amend this specification, including the claims, to expressly recite any sub-range subsumed within the ranges expressly recited herein.

The light emitting element and/or any other relevant devices or components according to embodiments of the present disclosure described herein may be implemented utilizing any suitable hardware, firmware (e.g., an application-specific integrated circuit), software, or a combination of software, firmware, and hardware. For example, the various components of the light emitting element may be formed on one integrated circuit (IC) chip or on separate IC chips. Further, the various components of the element may be implemented on a flexible printed circuit film, a tape carrier package (TCP), a printed circuit board (PCB), or formed on one substrate. Further, the various components of the element may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

In the present disclosure, when particles are spherical, “diameter” indicates an average particle diameter, and when the particles are non-spherical, the “diameter” indicates a major axis length. The diameter (or size) of the particles may be measured by particle size analysis, dynamic light scattering, scanning electron microscopy, and/or transmission electron microscope photography. When the size of the particles is measured utilizing a particle size analyzer, the average particle diameter (or size) may be referred to as D50. The term “D50” as utilized herein refers to the average diameter (or size) of particles whose cumulative volume corresponds to 50 vol % in the particle size distribution (e.g., cumulative distribution), and refers to the value of the particle size corresponding to 50% from the smallest particle when the total number of particles is 100% in the distribution curve accumulated in the order of the smallest particle size to the largest particle size. Particle size analysis may be performed with a HORIBA LA-950 laser particle size analyzer.

Hereinafter, with reference to Examples and Comparative Examples, an amine compound according to one or more embodiments of the present disclosure and a light emitting element of one or more embodiments will be specifically described. In some embodiments, Examples are shown only for the understanding of the present disclosure, and the scope of the present disclosure is not limited thereto.

### EXAMPLES

### 1. Synthesis of Amine Compounds

First, a process of synthesizing an amine compound according to one or more embodiments of the present disclosure will be described in more detail by presenting a process of synthesizing Compounds a01 to a07 as an example. In some embodiments, a process of synthesizing an amine compound, which will be described hereinafter, is provided as an example, and thus the process of synthesizing an amine compound according to one or more embodiments of the present disclosure is not limited to Examples.

### (1) Synthesis of Compound a01

Compound a01 according to one or more embodiments may be synthesized by, for example, Reaction Formula 1.

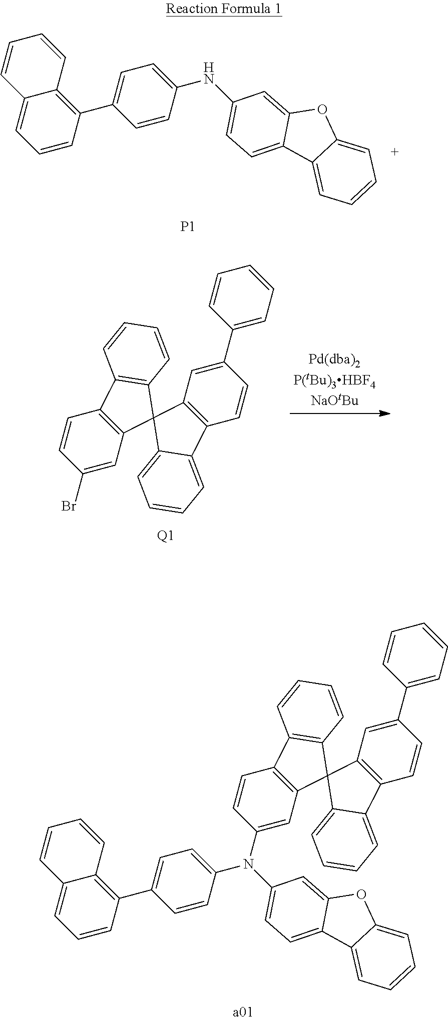

In an argon atmosphere, in a three-neck flask (200 mL), Intermediate Compound P1 (3.85 g, 10.0 mmol), Intermediate Compound Q1 (5.19 g, 11.0 mmol), Pd(dba)₂ (0.12 g, 0.20 mmol), P(^{t}Bu)₃·HBF4 (0.24 g, 0.8 mmol), and NaO^{t}Bu (1.16 g, 12.0 mmol) were added and stirred at 120° C. for 6 hours in a xylene solvent (50 mL). The stirred mixture was cooled, and then washed with water to separate an organic layer. The separated organic layer was purified through column chromatography (silica gel) to obtain Compound a01 (6.98 g, yield: 90%) as a white solid. Compound a01 has a molecular weight of 775 as measured by FAB-MS. Compound a01 had a chemical shift value δ of 7.92(2H), 7.88-7.78(6H), 7.69(1H), 7.59(1H), 7.55-7.33(13H), 7.31-7.06(11H), 6.89(1H), 6.80-6.74(2H) as determined through a measurement of ¹H-NMR (CDCl₃).

### (2) Synthesis of Compound a02

Compound a02 according to one or more embodiments may be synthesized by, for example, Reaction Formula 2.

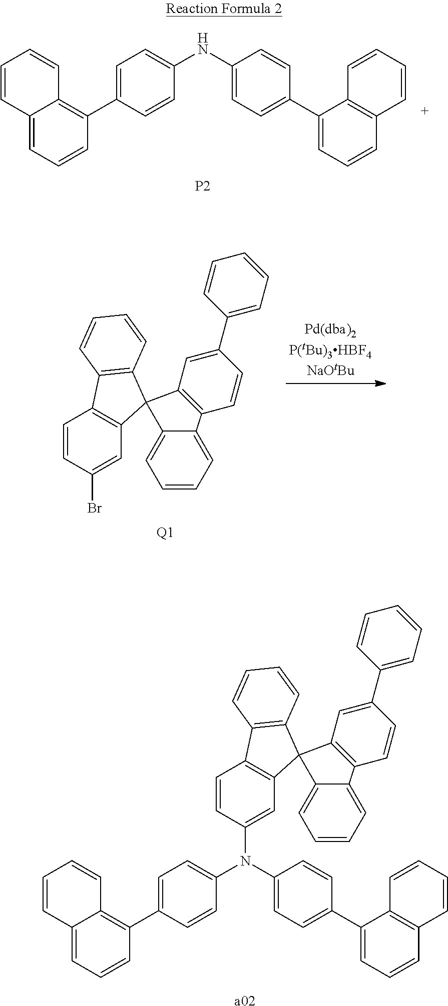

In an argon atmosphere, in a three-neck flask (200 mL), Intermediate Compound P2 (4.21 g, 10.0 mmol), Intermediate Compound Q1 (5.19 g, 11.0 mmol), Pd(dba)₂ (0.12 g, 0.20 mmol), P(^{t}Bu)₃·HBF₄ (0.24 g, 0.8 mmol), and NaO^{t}Bu (1.16 g, 12.0 mmol) were added and stirred at 120° C. for 6 hours in a xylene solvent (50 mL). The stirred mixture was cooled, and then washed with water to separate an organic layer. The separated organic layer was purified through column chromatography (silica gel) to obtain Compound a02 (7.47 g, yield: 92%) as a white solid. Compound a02 has a molecular weight of 812 as measured by FAB-MS. Compound a02 had a chemical shift value δ of 7.98-7.82(10H), 7.63(1H), 7.56-7.37(12H), 7.33-7.08(15H), 6.90(1H), 6.83-6.79(2H) as determined through a measurement of ¹H-NMR (CDCl₃).

### (3) Synthesis of Compound a03

Compound a03 according to one or more embodiments may be synthesized by, for example, Reaction Formula 3.

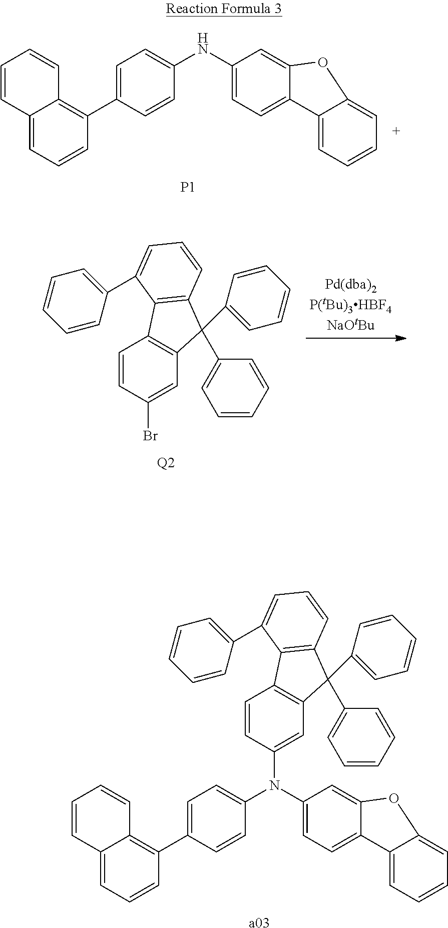

In an argon atmosphere, in a three-neck flask (200 mL), Intermediate Compound P1 (3.85 g, 10.0 mmol), Intermediate Compound Q2 (5.21 g, 11.0 mmol), Pd(dba)₂ (0.12 g, 0.20 mmol), P(^{t}Bu)₃·HBF₄ (0.24 g, 0.8 mmol), and NaO^{t}Bu (1.16 g, 12.0 mmol) were added and stirred at 120° C. for 6 hours in a xylene solvent (50 mL). The stirred mixture was cooled, and then washed with water to separate an organic layer. The separated organic layer was purified through column chromatography (silica gel) to obtain Compound a03 (6.92 g, yield: 89%) as a white solid. Compound a03 has a molecular weight of 777 as measured by FAB-MS. Compound a03 had a chemical shift value δ of 8.01(1H), 7.95-7.85(3H), 7.77(1H), 7.58-7.12(32H), 6.89-6.79(2H) as determined through a measurement of ¹H-NMR (CDCl₃).

### (4) Synthesis of Compound a04

Compound a04 according to one or more embodiments may be synthesized by, for example, Reaction Formula 4.

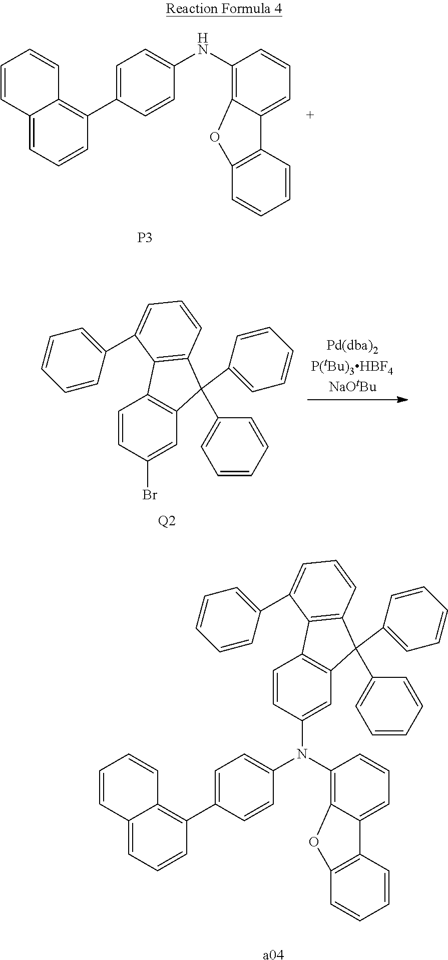

In an argon atmosphere, in a three-neck flask (200 mL), Intermediate Compound P3 (3.85 g, 10.0 mmol), Intermediate Compound Q₂ (5.21 g, 11.0 mmol), Pd(dba)₂ (0.12 g, 0.20 mmol), P(^{t}Bu)₃·HBF₄ (0.24 g, 0.8 mmol), and NaO^{t}Bu (1.16 g, 12.0 mmol) were added and stirred at 120° C. for 6 hours in a xylene solvent (50 mL). The stirred mixture was cooled, and then washed with water to separate an organic layer. The separated organic layer was purified through column chromatography (silica gel) to obtain Compound a04 (6.53 g, yield: 84%) as a white solid. Compound a04 has a molecular weight of 777 as measured by FAB-MS. Compound a04 had a chemical shift value δ of 7.95(1H), 7.80(1H), 7.72-7.66(3H), 7.58-7.45(5H), 7.37-7.08(28H), 6.87(1H) as determined through a measurement of ¹H-NMR (CDCl₃).

### (5) Synthesis of Compound a05

Compound a05 according to one or more embodiments may be synthesized by, for example, Reaction Formula 5.

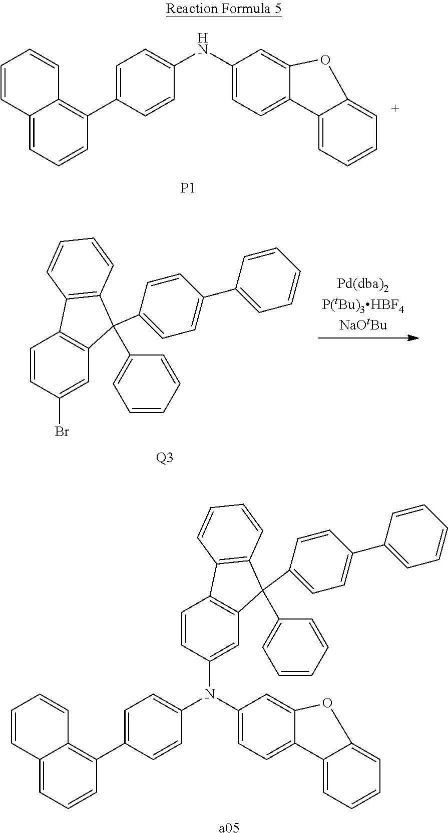

In an argon atmosphere, in a three-neck flask (200 mL), Intermediate Compound P1 (3.85 g, 10.0 mmol), Intermediate Compound Q₃ (5.21 g, 11.0 mmol), Pd(dba)₂ (0.12 g, 0.20 mmol), P(^{t}Bu)₃·HBF₄ (0.24 g, 0.8 mmol), and NaO^{t}Bu (1.16 g, 12.0 mmol) were added and stirred at 120° C. for 6 hours in a xylene solvent (50 mL). The stirred mixture was cooled, and then washed with water to separate an organic layer. The separated organic layer was purified through column chromatography (silica gel) to obtain Compound a05 (7.23 g, yield: 93%) as a white solid. Compound a05 has a molecular weight of 777 as measured by FAB-MS. Compound a05 had a chemical shift value δ of 7.95(1H), 7.80(1H), 7.72-7.66(4H), 7.62-7.43(6H), 7.37-7.08(26H), 6.81(1H) as determined through a measurement of ¹H-NMR (CDCl3).

### (6) Synthesis of Compound a06

Compound a06 according to one or more embodiments may be synthesized by, for example, Reaction Formula 6.

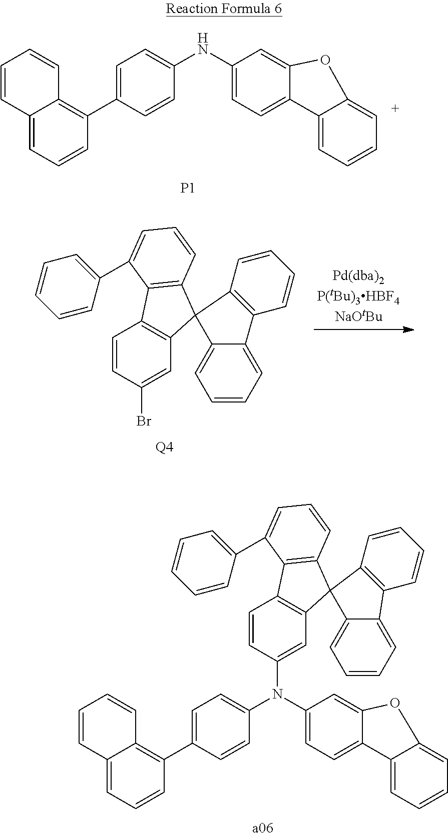

In an argon atmosphere, in a three-neck flask (200 mL), Intermediate Compound P1 (3.85 g, 10.0 mmol), Intermediate Compound Q4 (5.19 g, 11.0 mmol), Pd(dba)₂ (0.12 g, 0.20 mmol), P(^{t}Bu)₃·HBF₄ (0.24 g, 0.8 mmol), and NaO^{t}Bu (1.16 g, 12.0 mmol) were added and stirred at 120° C. for 6 hours in a xylene solvent (50 mL). The stirred mixture was cooled, and then washed with water to separate an organic layer. The separated organic layer was purified through column chromatography (silica gel) to obtain Compound a06 (6.36 g, yield: 82%) as a white solid. Compound a06 has a molecular weight of 775 as measured by FAB-MS. Compound a06 had a chemical shift value δ of 7.95(1H), 7.80(1H), 7.72-7.66(4H), 7.62-7.45(7H), 7.38(1H), 7.27-7.08(21H), 6.86(1H), 6.81(1H) as determined through a measurement of ¹H-NMR (CDCl₃).

### (7) Synthesis of Compound a07

Compound a07 according to one or more embodiments may be synthesized by, for example, Reaction Formula 7.

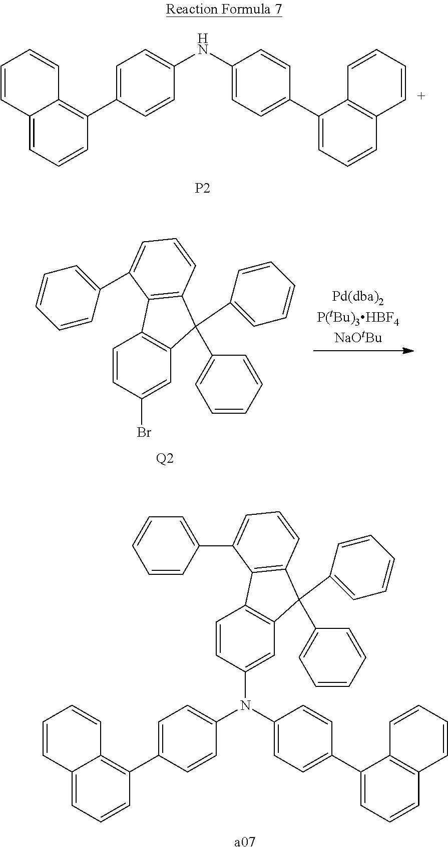

In an argon atmosphere, in a three-neck flask (200 mL), Intermediate Compound P2 (4.21 g, 10.0 mmol), Intermediate Compound Q2 (5.21 g, 11.0 mmol), Pd(dba)₂ (0.12 g, 0.20 mmol), P(^{t}Bu)₃·HBF₄ (0.24 g, 0.8 mmol), and NaO^{t}Bu (1.16 g, 12.0 mmol) were added and stirred at 120° C. for 6 hours in a xylene solvent (50 mL). The stirred mixture was cooled, and then washed with water to separate an organic layer. The separated organic layer was purified through column chromatography (silica gel) to obtain Compound a07 (7.65 g, yield: 94%) as a white solid. Compound a07 has a molecular weight of 814 as measured by FAB-MS. Compound a07 had a chemical shift value δ of 7.95(2H), 7.80(2H), 7.72-7.67(4H), 7.58-7.45(6H), 7.35(1H), 7.27-6.80(28H) as determined through a measurement of ¹H-NMR (CDCl₃).

In Table 1, structures and added mass of Intermediate Compounds P1 to P3, structures and added mass of Intermediate Compounds Q1 to Q4, an obtained amount and yield of the prepared target compounds, and FAB-MS are presented. ¹H-NMR of the target compounds are shown in Table 2. The molecular weight of Example Compounds was measured by FAB-MS utilizing JMS-700V from JEOL. The ¹H-NMR of the Example Compounds was measured utilizing AVAVCE300M from Bruker Biospin K.K.

TABLE 1

| | Intermediate | | Intermediate | | | | | |
|---|---|---|---|---|---|---|---|---|
| | Compounds | Mass | Compounds | Mass | Target | Mass | | FAB- |
| Item | P1 to P3 | (g) | Q1 to Q4 | (g) | compound | (g) | Yield | MS |
| a01 | 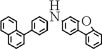 [SMILES: c1ccc2c(-c3ccc(Nc4ccc5c(c4)oc4ccccc45)cc3)cccc2c1] | 3.85 | 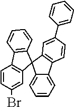 [SMILES: Brc1ccc2c(c1)C1(c3ccccc3-2)c2ccccc2-c2ccc(-c3ccccc3)cc21] | 5.19 | 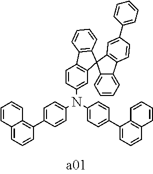 [SMILES: c1ccc(-c2ccc3c(c2)C2(c4ccccc4-3)c3ccccc3-c3ccc(N(c4ccc(-c5cccc6ccccc56)cc4)c4ccc(-c5cccc6ccccc56)cc4)cc32)cc1] | 6.98 | 90% | 775 |
| a02 | 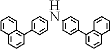 [SMILES: c1ccc2c(-c3ccc(Nc4ccc(-c5cccc6ccccc56)cc4)cc3)cccc2c1] | 4.21 | 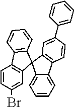 [SMILES: Brc1ccc2c(c1)C1(c3ccccc3-2)c2ccccc2-c2ccc(-c3ccccc3)cc21] | 5.19 | 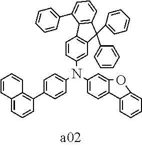 [SMILES: c1ccc(-c2cccc3c2-c2ccc(N(c4ccc(-c5cccc6ccccc56)cc4)c4ccc5c(c4)oc4ccccc45)cc2C3(c2ccccc2)c2ccccc2)cc1] | 7.47 | 92% | 812 |
| a03 | 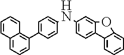 [SMILES: c1ccc2c(-c3ccc(Nc4ccc5c(c4)oc4ccccc45)cc3)cccc2c1] | 3.85 | 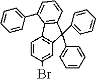 [SMILES: Brc1ccc2c(c1)C(c1ccccc1)(c1ccccc1)c1cccc(-c3ccccc3)c1-2] | 5.21 | 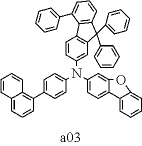 [SMILES: c1ccc(-c2cccc3c2-c2ccc(N(c4ccc(-c5cccc6ccccc56)cc4)c4ccc5c(c4)oc4ccccc45)cc2C3(c2ccccc2)c2ccccc2)cc1] | 6.92 | 89% | 777 |
| a04 | 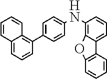 [SMILES: c1ccc2c(-c3ccc(Nc4cccc5c4oc4ccccc45)cc3)cccc2c1] | 3.85 | 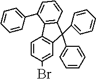 [SMILES: Brc1ccc2c(c1)C(c1ccccc1)(c1ccccc1)c1cccc(-c3ccccc3)c1-2] | 5.21 | 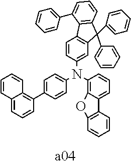 [SMILES: c1ccc(-c2cccc3c2-c2ccc(N(c4ccc(-c5cccc6ccccc56)cc4)c4cccc5c4oc4ccccc45)cc2C3(c2ccccc2)c2ccccc2)cc1] | 6.53 | 84% | 777 |
| a05 | 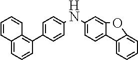 [SMILES: c1ccc2c(-c3ccc(Nc4ccc5c(c4)oc4ccccc45)cc3)cccc2c1] | 3.85 | 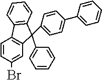 [SMILES: Brc1ccc2c(c1)C(c1ccccc1)(c1ccc(-c3ccccc3)cc1)c1ccccc1-2] | 5.21 | 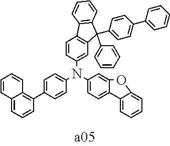 [SMILES: c1ccc(-c2ccc(C3(c4ccccc4)c4ccccc4-c4ccc(N(c5ccc(-c6cccc7ccccc67)cc5)c5ccc6c(c5)oc5ccccc56)cc43)cc2)cc1] | 7.23 | 93% | 777 |
| a06 | 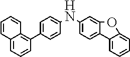 [SMILES: c1ccc2c(-c3ccc(Nc4ccc5c(c4)oc4ccccc45)cc3)cccc2c1] | 3.85 | 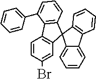 [SMILES: Brc1ccc2c(c1)C1(c3ccccc3-c3ccccc31)c1cccc(-c3ccccc3)c1-2] | 5.19 | 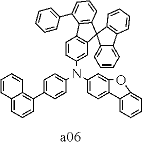 [SMILES: c1ccc(-c2cccc3c2-c2ccc(N(c4ccc(-c5cccc6ccccc56)cc4)c4ccc5c(c4)oc4ccccc45)cc2C32c3ccccc3-c3ccccc32)cc1] | 6.36 | 82% | 775 |
| a07 | 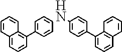 [SMILES: c1ccc2c(-c3ccc(Nc4ccc(-c5cccc6ccccc56)cc4)cc3)cccc2c1] | 4.21 | 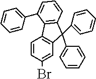 [SMILES: Brc1ccc2c(c1)C(c1ccccc1)(c1ccccc1)c1cccc(-c3ccccc3)c1-2] | 5.21 | 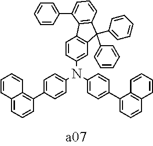 [SMILES: c1ccc(-c2cccc3c2-c2ccc(N(c4ccc(-c5cccc6ccccc56)cc4)c4ccc(-c5cccc6ccccc56)cc4)cc2C3(c2ccccc2)c2ccccc2)cc1] | 7.65 | 94% | 814 |

TABLE 2

| Item | ¹H-NMR (CDCl₃) |
|---|---|
| a01 | 7.92(2H), 7.88-7.78(6H), 7.69(1H), 7.59(1H), 7.55-7.33(13H), |
| | 7.31-7.06(11H), 6.89(1H), 6.80-6.74(2H) |
| a02 | 7.98-7.82(10H), 7.63(1H), 7.56-7.37(12H), 7.33-7.08(15H), |
| | 6.90(1H), 6.83-6.79(2H) |
| a03 | 8.01(1H), 7.95-7.85(3H), 7.77(1H), 7.58-7.12(32H), |
| | 6.89-6.79(2H) |
| a04 | 7.95(1H), 7.80(1H), 7.72-7.66(3H), 7.58-7.45(5H), |
| | 7.37-7.08(28H), 6.87(1H) |
| a05 | 7.95(1H), 7.80(1H), 7.72-7.66(4H), 7.62-7.43(6H), |
| | 7.37-7.08(26H), 6.81(1H) |
| a06 | 7.95(1H), 7.80(1H), 7.72-7.66(4H), 7.62-7.45(7H), 7.38(1H), |
| | 7.27-7.08(21H), 6.86(1H), 6.81(1H) |
| a07 | 7.95(2H), 7.80(2H), 7.72-7.67(4H), 7.58-7.45(6H), 7.35(1H), |
| | 7.27-6.80(28H) |

### 2. Preparation and Evaluation of Light Emitting Elements

Light emitting elements of one or more embodiments including an amine compound of one or more embodiments in an electron blocking layer were prepared utilizing a method. Light emitting elements of Examples 1 to 7 were prepared utilizing amine compounds of Compounds a01 to a07, which are Example Compounds described herein, as a hole transport layer material. Comparative Examples 1 to 20 correspond to light emitting elements prepared utilizing Comparative Example Compound b01 to b20 as an electronic blocking layer material.

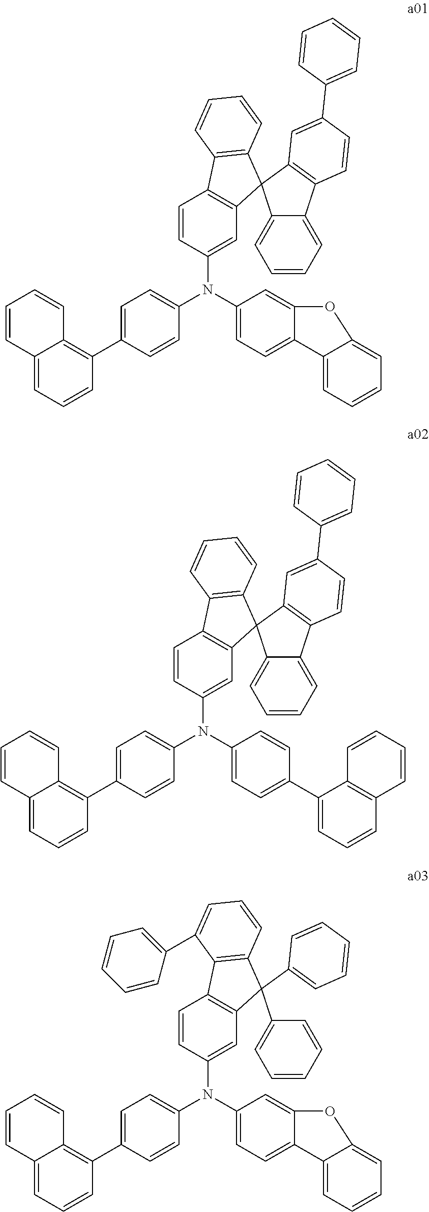 [SMILES: c1ccc(-c2ccc3c(c2)C2(c4ccccc4-3)c3ccccc3-c3ccc(N(c4ccc(-c5cccc6ccccc56)cc4)c4ccc5c(c4)oc4ccccc45)cc32)cc1 ; c1ccc(-c2ccc3c(c2)C2(c4ccccc4-3)c3ccccc3-c3ccc(N(c4ccc(-c5cccc6ccccc56)cc4)c4ccc(-c5cccc6ccccc56)cc4)cc32)cc1 ; c1ccc(-c2cccc3c2-c2ccc(N(c4ccc(-c5cccc6ccccc56)cc4)c4ccc5c(c4)oc4ccccc45)cc2C3(c2ccccc2)c2ccccc2)cc1]

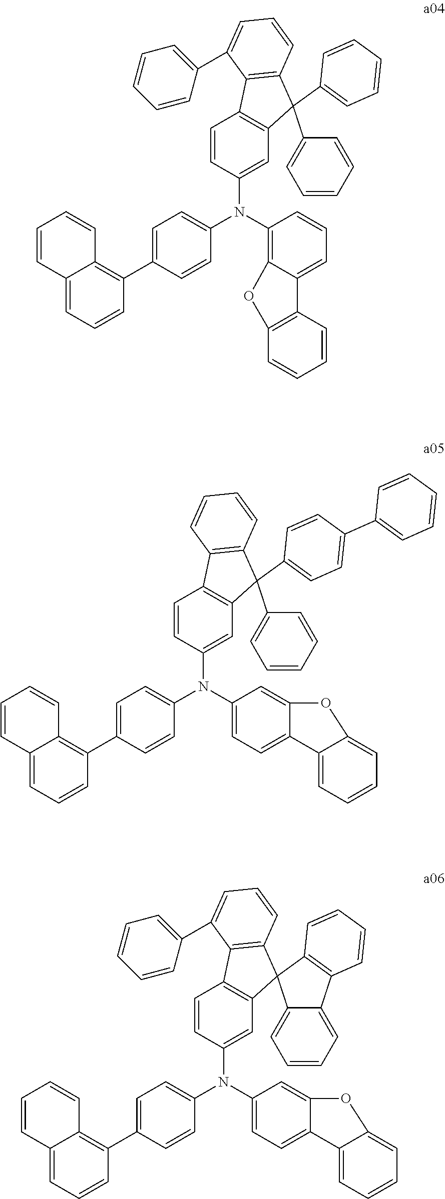 [SMILES: c1ccc(-c2cccc3c2-c2ccc(N(c4ccc(-c5cccc6ccccc56)cc4)c4cccc5c4oc4ccccc45)cc2C3(c2ccccc2)c2ccccc2)cc1 ; c1ccc(-c2ccc(C3(c4ccccc4)c4ccccc4-c4ccc(N(c5ccc(-c6cccc7ccccc67)cc5)c5ccc6c(c5)oc5ccccc56)cc43)cc2)cc1 ; c1ccc(-c2cccc3c2-c2ccc(N(c4ccc(-c5cccc6ccccc56)cc4)c4ccc5c(c4)oc4ccccc45)cc2C32c3ccccc3-c3ccccc32)cc1]

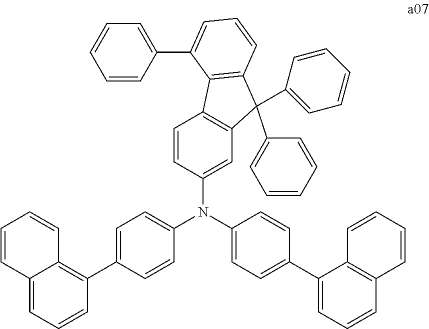 [SMILES: c1ccc(-c2cccc3c2-c2ccc(N(c4ccc(-c5cccc6ccccc56)cc4)c4ccc(-c5cccc6ccccc56)cc4)cc2C3(c2ccccc2)c2ccccc2)cc1]

### Comparative Example Compound

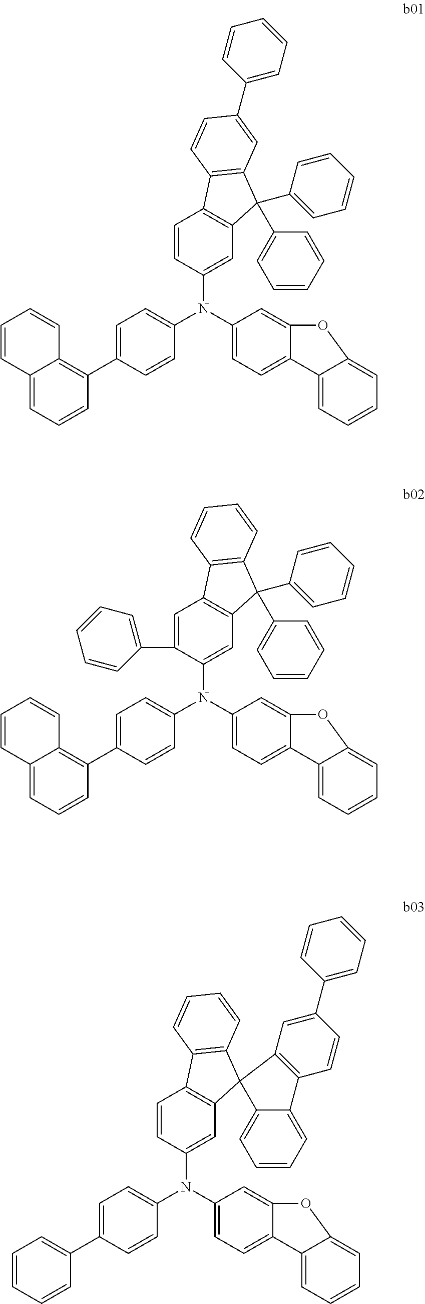 [SMILES: c1ccc(-c2ccc3c(c2)C(c2ccccc2)(c2ccccc2)c2cc(N(c4ccc(-c5cccc6ccccc56)cc4)c4ccc5c(c4)oc4ccccc45)ccc2-3)cc1 ; c1ccc(-c2cc3c(cc2N(c2ccc(-c4cccc5ccccc45)cc2)c2ccc4c(c2)oc2ccccc24)C(c2ccccc2)(c2ccccc2)c2ccccc2-3)cc1 ; c1ccc(-c2ccc(N(c3ccc4c(c3)C3(c5ccccc5-c5ccc(-c6ccccc6)cc53)c3ccccc3-4)c3ccc4c(c3)oc3ccccc34)cc2)cc1]

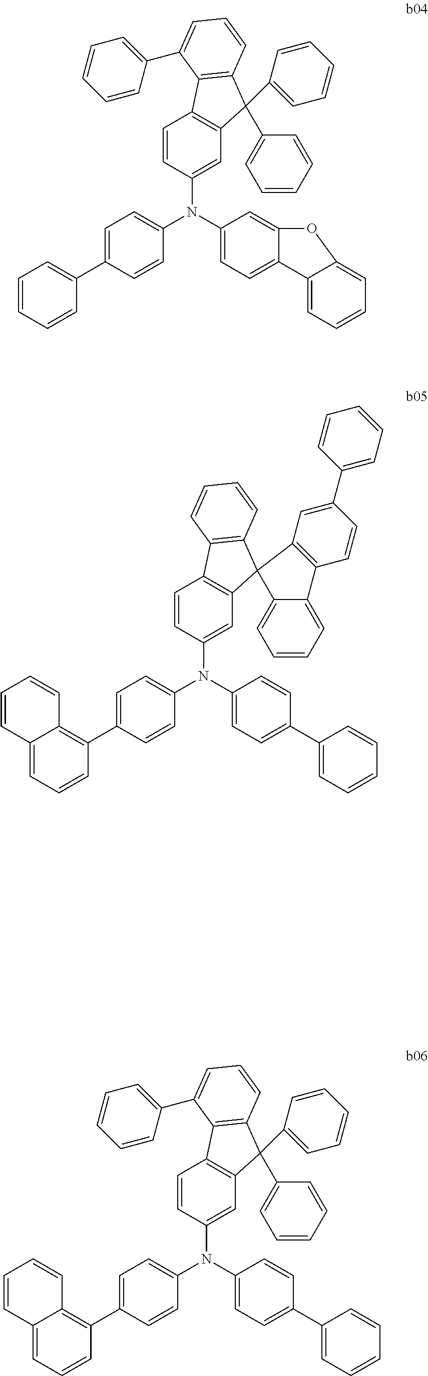 [SMILES: c1ccc(-c2ccc(N(c3ccc4c(c3)C(c3ccccc3)(c3ccccc3)c3cccc(-c5ccccc5)c3-4)c3ccc4c(c3)oc3ccccc34)cc2)cc1 ; c1ccc(-c2ccc(N(c3ccc(-c4cccc5ccccc45)cc3)c3ccc4c(c3)C3(c5ccccc5-c5ccc(-c6ccccc6)cc53)c3ccccc3-4)cc2)cc1 ; c1ccc(-c2ccc(N(c3ccc(-c4cccc5ccccc45)cc3)c3ccc4c(c3)C(c3ccccc3)(c3ccccc3)c3cccc(-c5ccccc5)c3-4)cc2)cc1]

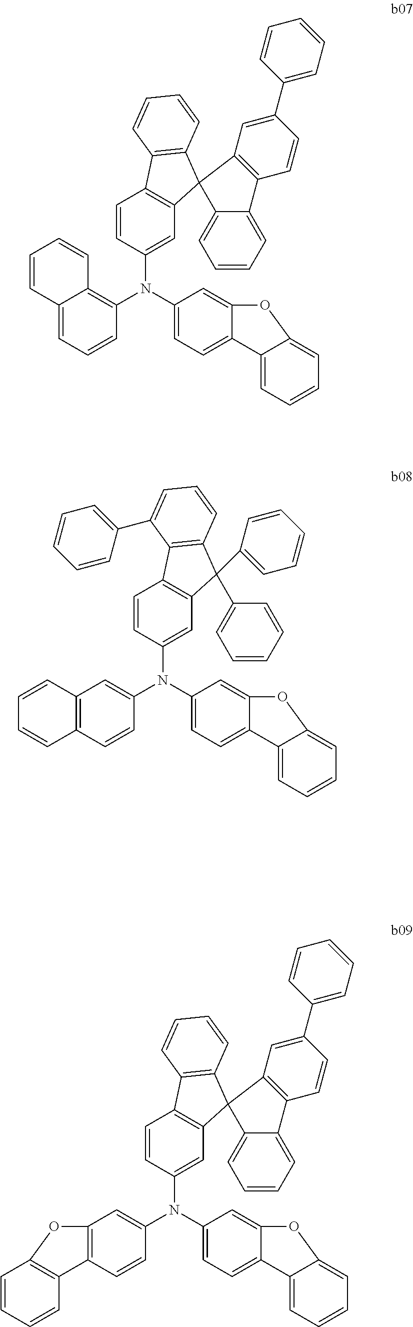 [SMILES: c1ccc(-c2ccc3c(c2)C2(c4ccccc4-3)c3ccccc3-c3ccc(N(c4ccc5c(c4)oc4ccccc45)c4cccc5ccccc45)cc32)cc1 ; c1ccc(-c2cccc3c2-c2ccc(N(c4ccc5ccccc5c4)c4ccc5c(c4)oc4ccccc45)cc2C3(c2ccccc2)c2ccccc2)cc1 ; c1ccc(-c2ccc3c(c2)C2(c4ccccc4-3)c3ccccc3-c3ccc(N(c4ccc5c(c4)oc4ccccc45)c4ccc5c(c4)oc4ccccc45)cc32)cc1]

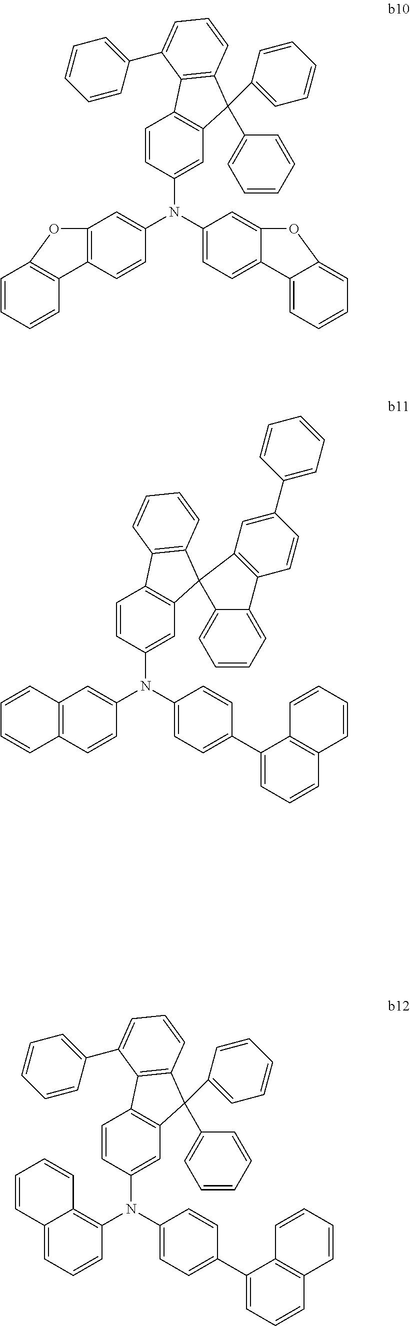 [SMILES: c1ccc(-c2cccc3c2-c2ccc(N(c4ccc5c(c4)oc4ccccc45)c4ccc5c(c4)oc4ccccc45)cc2C3(c2ccccc2)c2ccccc2)cc1 ; c1ccc(-c2ccc3c(c2)C2(c4ccccc4-3)c3ccccc3-c3ccc(N(c4ccc(-c5cccc6ccccc56)cc4)c4ccc5ccccc5c4)cc32)cc1 ; c1ccc(-c2cccc3c2-c2ccc(N(c4ccc(-c5cccc6ccccc56)cc4)c4cccc5ccccc45)cc2C3(c2ccccc2)c2ccccc2)cc1]

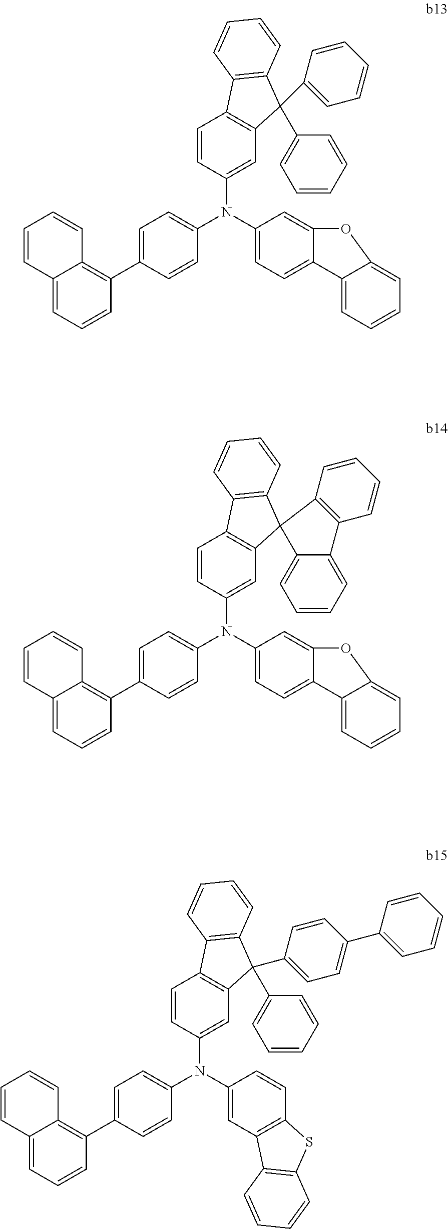 [SMILES: c1ccc(C2(c3ccccc3)c3ccccc3-c3ccc(N(c4ccc(-c5cccc6ccccc56)cc4)c4ccc5c(c4)oc4ccccc45)cc32)cc1 ; c1ccc2c(c1)-c1ccccc1C21c2ccccc2-c2ccc(N(c3ccc(-c4cccc5ccccc45)cc3)c3ccc4c(c3)oc3ccccc34)cc21 ; c1ccc(-c2ccc(C3(c4ccccc4)c4ccccc4-c4ccc(N(c5ccc(-c6cccc7ccccc67)cc5)c5ccc6sc7ccccc7c6c5)cc43)cc2)cc1]

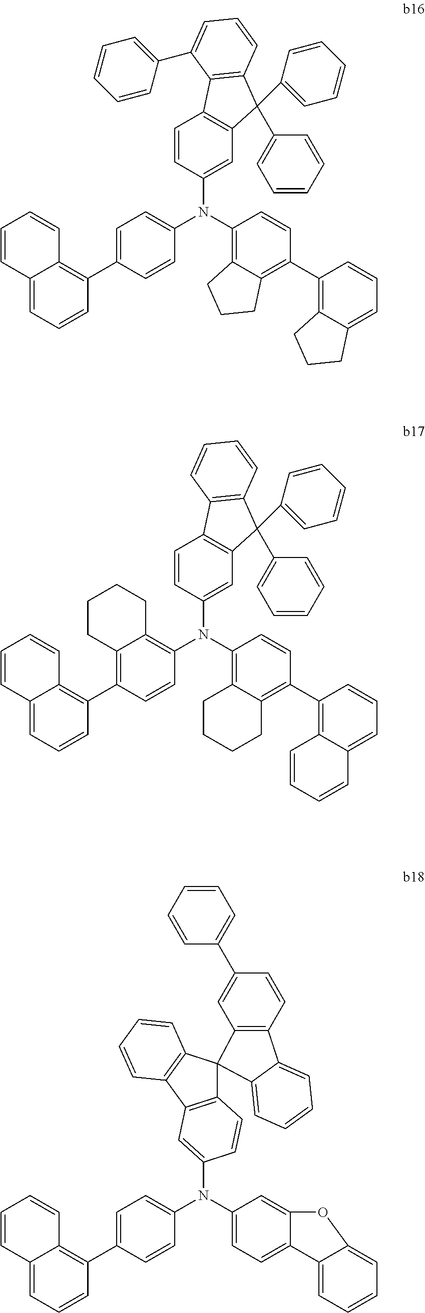 [SMILES: c1ccc(-c2cccc3c2-c2ccc(N(c4ccc(-c5cccc6ccccc56)cc4)c4ccc(-c5cccc6c5CCC6)c5c4CCC5)cc2C3(c2ccccc2)c2ccccc2)cc1 ; c1ccc(C2(c3ccccc3)c3ccccc3-c3ccc(N(c4ccc(-c5cccc6ccccc56)c5c4CCCC5)c4ccc(-c5cccc6ccccc56)c5c4CCCC5)cc32)cc1 ; c1ccc(-c2ccc3c(c2)C2(c4ccccc4-c4cc(N(c5ccc(-c6cccc7ccccc67)cc5)c5ccc6c(c5)oc5ccccc56)ccc42)c2ccccc2-3)cc1]

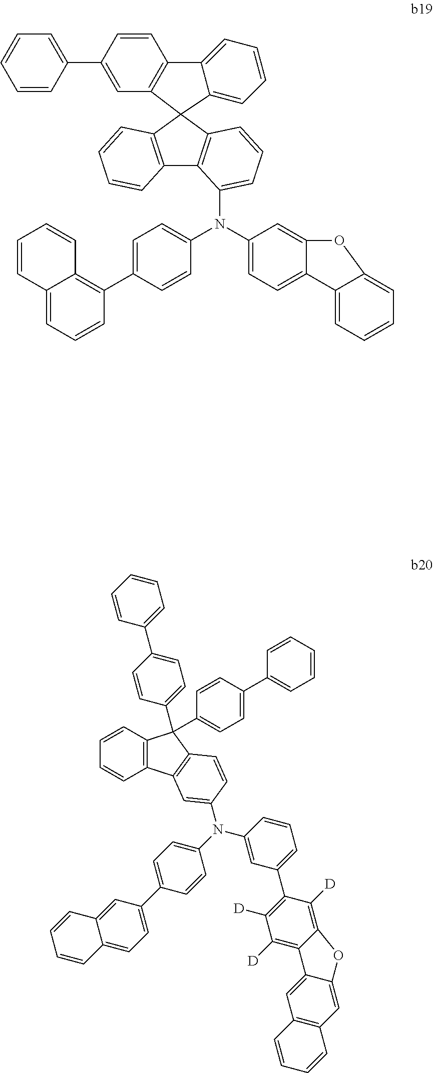 [SMILES: c1ccc(-c2ccc3c(c2)C2(c4ccccc4-3)c3ccccc3-c3c(N(c4ccc(-c5cccc6ccccc56)cc4)c4ccc5c(c4)oc4ccccc45)cccc32)cc1 ; [2H]c1c(-c2cccc(N(c3ccc(-c4ccc5ccccc5c4)cc3)c3ccc4c(c3)-c3ccccc3C4(c3ccc(-c4ccccc4)cc3)c3ccc(-c4ccccc4)cc3)c2)c([2H])c2oc3cc4ccccc4cc3c2c1[2H]]

### Preparation of Light Emitting Elements

An ITO glass substrate (Corning, 15 Ω/cm²(150 nm)) was cut to a size of 50 millimeter (mm)×50 mm×0.7 mm, washed with isopropyl alcohol and pure water, subjected to ultrasonic cleaning for 5 minutes, and then irradiated with UV for 30 minutes, and ozone-treated. Thereafter, 2-TNATA(4,4′,4″-tris{N-(2-naphthyl)-N-phenylamino}-triphenylamine was vacuum deposited to be 60 nm thick to form a hole injection layer, and then Example Compounds or Comparative Example Compounds were vacuum deposited to be 30 nanometer (nm) thick to form an electron blocking layer.

On the electron blocking layer, a blue fluorescent host, 9,10-di(naphthalen-2-yl)anthracene (ADN) and a fluorescent dopant, 2, 5, 8, 11-Tetra-t-butylperylene (TBP) were co-deposited at a ratio of 97:3 to form an emission layer having a thickness of 25 nm.

On the emission layer, tris(8-hydroxyquinolino)aluminum (Alq₃) was utilized to form an electron transport layer having a thickness of 25 nm, and then LiF (1 nm) was deposited to form an electron injection layer. On the electron injection layer, aluminum (AI) was utilized to form a second electrode having a thickness of 100 nm.

In some embodiments, the compounds of each functional layer utilized for the preparation of the light emitting elements are as follows.

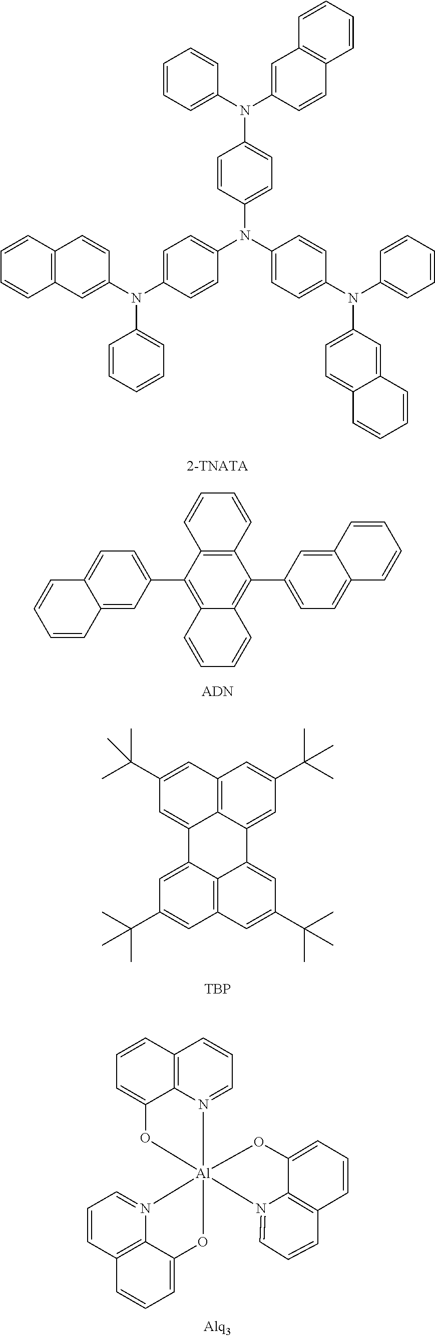

### Evaluation of Light Emitting Elements

Table 3 shows results of evaluation of the light emitting elements for Examples 1 to 7 and Comparative Examples 1 to 20. Table 3 shows results of evaluation on light emitting efficiency and lifespan of the prepared light emitting elements, and purity of the electron blocking layer.

In the property evaluation results for Examples and Comparative Examples shown in Table 3, “Light emitting efficiency” indicates an efficiency value at a current density of 10 milliampere per square centimeter (mA/cm²), “Lifespan LT50” indicates luminance half-life from an initial luminance of 1000 candela per square meter (cd/m²). “Purity of electron blocking layer” indicates a value measured by HPLC for the purity of the electron blocking layer material deposited on a substrate after depositing each electron blocking layer material at 0.2 nanometer per second (nm/s). The materials of all electron blocking layers were 99.9% pure before the deposition.

TABLE 3

| | Electron | Light emitting | Lifespan | Purity of |
|---|---|---|---|---|
| ID of prepared | blocking | efficiency | LT50 | electron |
| element | layer EBL | (cd/A) | (h) | blocking layer |
| Example 1 | a01 | 8.0 | 2000 | 99.9% |
| Example 2 | a02 | 8.0 | 2000 | 99.9% |
| Example 3 | a03 | 8.0 | 2000 | 99.9% |
| Example 4 | a04 | 8.0 | 2000 | 99.9% |
| Example 5 | a05 | 8.0 | 2000 | 99.9% |
| Example 6 | a06 | 8.0 | 2000 | 99.9% |
| Example 7 | a07 | 8.0 | 2000 | 99.9% |
| Comparative | b01 | 7.2 | 1600 | 99.9% |
| Example 1 | | | | |
| Comparative | b02 | 7.8 | 1500 | 99.6% |
| Example 2 | | | | |
| Comparative | b03 | 7.8 | 1700 | 99.9% |
| Example 3 | | | | |
| Comparative | b04 | 7.8 | 1700 | 99.9% |
| Example 4 | | | | |
| Comparative | b05 | 7.8 | 1700 | 99.9% |
| Example 5 | | | | |
| Comparative | b06 | 7.8 | 1700 | 99.9% |
| Example 6 | | | | |
| Comparative | b07 | 7.8 | 1400 | 99.5% |
| Example 7 | | | | |
| Comparative | b08 | 7.3 | 1200 | 99.9% |
| Example 8 | | | | |
| Comparative | b09 | 7.7 | 1600 | 99.9% |
| Example 9 | | | | |
| Comparative | b10 | 7.7 | 1500 | 99.9% |
| Example 10 | | | | |
| Comparative | b11 | 7.4 | 1100 | 99.9% |
| Example 11 | | | | |
| Comparative | b12 | 7.8 | 1500 | 99.4% |
| Example 12 | | | | |
| Comparative | b13 | 7.8 | 1800 | 99.9% |
| Example 13 | | | | |
| Comparative | b14 | 7.8 | 1800 | 99.9% |
| Example 14 | | | | |
| Comparative | b15 | 7.3 | 700 | 99.8% |
| Example 15 | | | | |
| Comparative | b16 | 7.0 | 300 | 99.6% |
| Example 16 | | | | |
| Comparative | b17 | 6.9 | 200 | 99.6% |
| Example 17 | | | | |
| Comparative | b18 | 7.7 | 500 | 99.8% |
| Example 18 | | | | |
| Comparative | b19 | 7.8 | 400 | 99.9% |
| Example 19 | | | | |
| Comparative | B20 | 7.6 | 300 | 99.8% |
| Example 20 | | | | |

Referring to the structures shown in Table 1, the Example Compounds utilized in Examples 1 to 7 include substituents having relatively high hole transport properties and high stability and high electron resistance. Accordingly, the light emitting elements of Examples 1 to 7 exhibited relatively high efficiency and long lifespan. For example, it is believed that, in the amine compounds according to one or more embodiments of the present disclosure, an aryl group is introduced at a specific position of a fluorene moiety such as a diphenylfluorene group to sterically suppress or reduce intermolecular proximity between fluorenes, thereby contributing to relatively high efficiency and long lifespan of a light emitting element. In some embodiments, it was determined that the amine compounds of one or more embodiments excluded (i.e., did not include) a substituent unit that was easily pyrolyzed, and thus were allowed to be utilized as a light emitting element material without being pyrolyzed upon deposition.

Comparative Example Compound b01 utilized in Comparative Example 1 is different in a substitution position of a phenyl group substituted on the fluorene moiety, from Example Compound a03 utilized in Example 3. In Comparative Example Compound b01, the phenyl group was substituted at the 7-position of fluorene, so that the light emitting element of Comparative Example 1 had low efficiency and short lifespan. The results may be because in Comparative Example Compound b01, excessive lengthening of wavelengths was caused in an absorption wavelength of molecules to lead to a partial absorption of the light emitted from an emission layer, and thus a transport layer itself was excited.

Comparative Example Compound b02 utilized in Comparative Example 2 is different in a substitution position of a phenyl group substituted on the fluorene moiety, from Example Compound a03 utilized in Example 3. In Comparative Example Compound b02, the phenyl group was substituted at 3-position of fluorene, so that the light emitting element of Comparative Example 2 had low efficiency and short lifespan. The results may be because in Comparative Example Compound b02, the phenyl group was bonded to be positioned ortho to the nitrogen atom of amine linked to the fluorene moiety to distort the fluorene moiety, resulting in reduced stability.

Comparative Example Compounds b03 and b04 utilized in Comparative Examples 3 and 4 are amine compounds in which one of the substituents linked to the nitrogen atom of amine is a biphenyl group, compared to Example Compounds a01 and a03 utilized in Examples 1 and 3. Accordingly, the light emitting elements of Comparative Example 3 and Comparative Example 4 showed slightly reduced lifespan, compared to the light emitting elements of Examples. The results may be because Comparative Example Compounds b03 and b04 included the biphenyl group as a substituent, and thus had slightly reduced electron resistance, compared to Example Compound in which 3 substituents all linked to the nitrogen atom of amine included a fluorene moiety having high electron resistance, a naphthyl group moiety, and a heteroaryl group (i.e., dibenzofuran group).

Comparative Example Compounds b05 and b06 utilized in Comparative Examples 5 and 6 are amine compounds in which one of the substituents linked to the nitrogen atom of amine is a biphenyl group, compared to Example Compounds a01, a02, a03, and a07 utilized in Examples 1, 2, 3, and 7. Accordingly, the light emitting elements of Comparative Example 5 and Comparative Example 6 showed slightly reduced lifespan, compared to the light emitting elements of Examples. The results may be because Comparative Example Compounds b05 and b06 included the biphenyl group as a substituent, and thus had slightly reduced electron resistance, compared to Example Compound in which 3 substituents all linked to the nitrogen atom of amine included a fluorene moiety having high electron resistance, a naphthyl group moiety, and a heteroaryl group (i.e., dibenzofuran group).

Comparative Example Compounds b07 and b12 utilized in Comparative Examples 7 and 12 are amine compounds in which an a-naphthyl group is directly bonded to the nitrogen atom of amine, compared to Example Compounds a01 and a03 utilized in Examples 1 and 3. Accordingly, the light emitting elements of Comparative Example 7 and Comparative Example 12 showed reduced lifespan. The results may be because Comparative Example Compounds b07 and b12, the a-naphthyl group was directly bonded to the nitrogen atom of amine to sterically distort a molecular structure, resulting in low stability.

Comparative Example Compounds b08 and b11 utilized in Comparative Examples 8 and 11 are amine compounds in which an P-naphthyl group is directly bonded to the nitrogen atom of amine, compared to Example Compounds a01 and a03 utilized in Examples 1 and 3. Accordingly, the light emitting elements of Comparative Example 8 and Comparative Example 11 showed low efficiency and short lifespan. The results may be because in Comparative Example Compounds b08 and b11, excessive lengthening of wavelengths was caused in an absorption wavelength of molecules to lead to a partial absorption of the light emitted from an emission layer, and thus a transport layer itself was excited.

Comparative Example Compounds b09 and b10 utilized in Comparative Examples 9 and 10 are amine compounds in which two of the substituents linked to the nitrogen atom of amine are heteroaryl groups, compared to Example Compounds a01 and a03 utilized in Examples 1 and 3. Accordingly, the light emitting elements of Comparative Example 9 and Comparative Example 10 showed slightly reduced lifespan. This may be because, unlike Example Compounds having at least one second substituent, Comparative Example Compounds b09 and b10 did not include the second substituent, and thus had slightly reduced hole transport properties and electron resistance.

Comparative Example Compounds b13 and b14 utilized in Comparative Examples 13 and 14 are amine compounds in which an aryl group and/or a heteroaryl group is not substituted at a specific position of the fluorene moiety, compared to Example Compounds a01, a03, a05, and a06 utilized in Examples 1, 3, 5, and 6. Accordingly, the light emitting elements of Comparative Example 13 and Comparative Example 14 showed slightly reduced lifespan. This may be because in Comparative Examples Compounds b13 and b14, the intermolecular proximity between fluorenes was not obscured sterically (e.g., sterically easy), and thus an interaction between an emission layer and molecules was more likely to occur (e.g., easily caused), resulting in reduced efficiency and lifespan of a light emitting element.

Comparative Example Compound b15 utilized in Comparative Example 15 is an amine compound in which a 2-dibenzothiophene group is linked to the nitrogen atom of amine, compared to Example Compound a05 utilized in Example 5.

Accordingly, the light emitting element of Comparative Example 15 showed low efficiency and short lifespan. The results may be because in Comparative Example Compound b15, when a sulfur atom (S) of a 2-dibenzothiophene group is positioned para to the nitrogen atom of amine, S is highly reactive (e.g., larger atomic size and more polarizable valence electrons), and thus an interaction between an emission layer and molecules was more likely to occur (e.g., easily caused), resulting in reduced efficiency and lifespan.

Comparative Example Compound b16 utilized in Comparative Example 16 is an amine compound in which one of the substituents linked to the nitrogen atom of amine is a cycloalkyl group, compared to Example Compound a07 utilized in Example 7. Accordingly, the light emitting element of Comparative Example 16 showed low efficiency and short lifespan. The results may be because the cycloalkyl group of Comparative Example Compound b16 was unstable to cause reduced efficiency and lifespan of a light emitting element.

Comparative Example Compound b17 utilized in Comparative Example 17 is an amine compound having no substituent at a specific position of the fluorene moiety (e.g., diphenylfluorene group), compared to Example Compounds a02 and a07 utilized in Examples 2 and 7. In some embodiments, in Comparative Example Compound b17, one of the substituents linked to the nitrogen atom of amine is a cycloalkyl group. Accordingly, the light emitting element of Comparative Example 17 showed low efficiency and short lifespan. The results may be because the intermolecular proximity between fluorenes was not obscured sterically (e.g., sterically easy) and the cycloalkyl group was unstable, causing reduced efficiency and lifespan.

Comparative Example Compounds b18, b19, and b20 utilized in Comparative Examples 18, 19, and 20 are amine compounds linked to the nitrogen atoms of amine at 3-position and 4-position of fluorene instead of 2-position, compared to Example Compound a01 utilized in Example 1. Accordingly, the light emitting elements of Comparative Examples 18, 19, and 20 showed short lifespan. The results may be because in Comparative Example Compounds b18, b19, and b20, connection of the nitrogen atom of the amine at the 3-position and 4-position of fluorene may greatly reduce the hole transport property of the amine compound.

A light emitting element of one or more embodiments may exhibit improved element characteristics of relatively high efficiency and long service life (i.e., lifespan).

An amine compound of one or more embodiments has excellent or suitable hole transport properties and electron resistance, and may thus contribute to relatively high efficiency and long service life (i.e., lifespan) of a light emitting element when applied to the light emitting element.

Although the present disclosure has been described with reference to a preferred embodiment of the present disclosure, it will be understood that the present disclosure should not be limited to these preferred embodiments but one or more suitable changes and modifications can be made by those skilled in the art without departing from the spirit and scope of the present disclosure. Accordingly, the technical scope of the present disclosure is not intended to be limited to the contents set forth in the detailed description of the specification, but is intended to be defined by the appended claims and equivalents thereof.

What is claimed is:

1. A light emitting element comprising:
a first electrode;
a second electrode on the first electrode; and
at least one functional layer between the first electrode and the second electrode,
wherein the at least one functional layer comprises an amine compound represented by Formula 1: 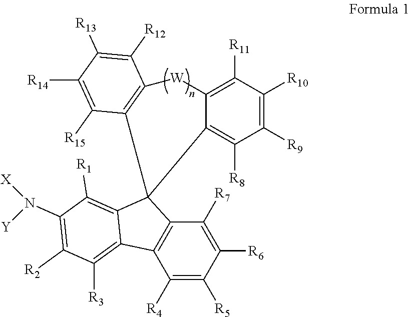
wherein in Formula 1,
R₁ to R₁₅ are each independently a hydrogen atom, a deuterium atom, a halogen atom, a substituted or unsubstituted amine group, a substituted or unsubstituted silyl group, a substituted or unsubstituted aryl group having 6 to 30 ring-forming carbon atoms, or a substituted or unsubstituted heteroaryl group having 2 to 30 ring-forming carbon atoms,
at least one selected from among R₃ to R₅, and R₇ to R₁₅ is a substituted or unsubstituted aryl group having 6 to 30 ring-forming carbon atoms, or a substituted or unsubstituted heteroaryl group having 2 to 30 ring-forming carbon atoms,
W is O, S, NRₐ₁, CRₐ₂Rₐ₃, or a direct linkage,
n is 0 or 1,
Rₐ₁ is a substituted or unsubstituted aryl group having 6 to 30 ring-forming carbon atoms, or a substituted or unsubstituted heteroaryl group having 2 to 30 ring-forming carbon atoms,
Rₐ₂ and Rₐ₃ are each independently a substituted or unsubstituted aryl group having 6 to 30 ring-forming carbon atoms, or a substituted or unsubstituted heteroaryl group having 2 to 30 ring-forming carbon atoms, and/or Rₐ₂ and Rₐ₃ are bonded to form a ring, and
X and Y are each independently represented by Formula 2 or Formula 3, and at least one of X or Y is represented by Formula 2 and the other is represented by Formula 3: 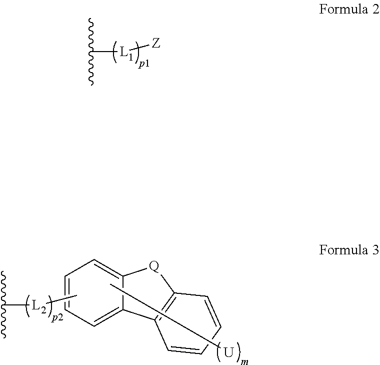
wherein in Formulas 2 and 3,
L₁ and L₂ are each independently a substituted or unsubstituted arylene group having 6 to 50 ring-forming carbon atoms, or a substituted or unsubstituted heteroarylene group having 2 to 50 ring-forming carbon atoms,
p1 is an integer of 1 to 3,
p2 is an integer of 0 to 3,
Z is a substituted or unsubstituted naphthyl group, or a substituted or unsubstituted phenanthrenyl group,
U is a hydrogen atom, a deuterium atom, a halogen atom, a substituted or unsubstituted amine group, a substituted or unsubstituted silyl group, a substituted or unsubstituted aryl group having 6 to 30 ring-forming carbon atoms, or a substituted or unsubstituted heteroaryl group having 2 to 30 ring-forming carbon atoms,
m is an integer of 0 to 7,
Q is O, S, NRₐ₄, or CRₐ₅Rₐ₆,
Rₐ₄ is a substituted or unsubstituted aryl group having 6 to 30 ring-forming carbon atoms, or a substituted or unsubstituted heteroaryl group having 2 to 30 ring-forming carbon atoms,
Rₐ₅ and Rₐ₆ are each independently a substituted or unsubstituted aryl group having 6 to 30 ring-forming carbon atoms, or a substituted or unsubstituted heteroaryl group having 2 to 30 ring-forming carbon atoms, or Rₐ₅ and Rₐ₆ are bonded to form a ring, and
a case in which Formula 3 is a substituted or unsubstituted 2-dibenzothiophene group is excluded.

2. The light emitting element of claim 1, wherein the at least one functional layer comprises an emission layer, a hole transport region between the first electrode and the emission layer, and an electron transport region between the emission layer and the second electrode,
the hole transport region comprising the amine compound.

3. The light emitting element of claim 2, wherein the hole transport region comprises at least one of a hole injection layer, a hole transport layer, or an electron blocking layer,
the at least one of the hole injection layer, the hole transport layer, or the electron blocking layer comprising the amine compound.

4. The light emitting element of claim 2, wherein the hole transport region comprises:
a hole injection layer or a hole transport layer on the first electrode; and
an electron blocking layer on the hole injection layer or the hole transport layer, the electron blocking layer comprising the amine compound.

5. The light emitting element of claim 1, wherein Formula 1 is represented by Formula 1-1 or Formula 1-2: 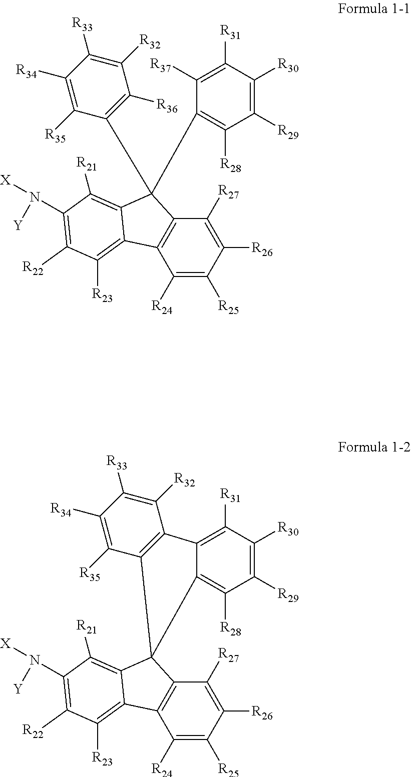
wherein in Formulas 1-1 and 1-2,
R₂₁ to R₃₅ are each independently a hydrogen atom, a deuterium atom, or a substituted or unsubstituted aryl group having 6 to 30 ring-forming carbon atoms,
at least one selected from among R₂₃ to R₂₅, and R₂₇ to R₃₅ is a substituted or unsubstituted aryl group having 6 to 30 ring-forming carbon atoms,
R₃₆ and R₃₇ are each independently a hydrogen atom or a deuterium atom, and
X and Y are as defined in Formula 1.

6. The light emitting element of claim 1, wherein Formula 1 is represented by Formula 4-1 or Formula 4-2: 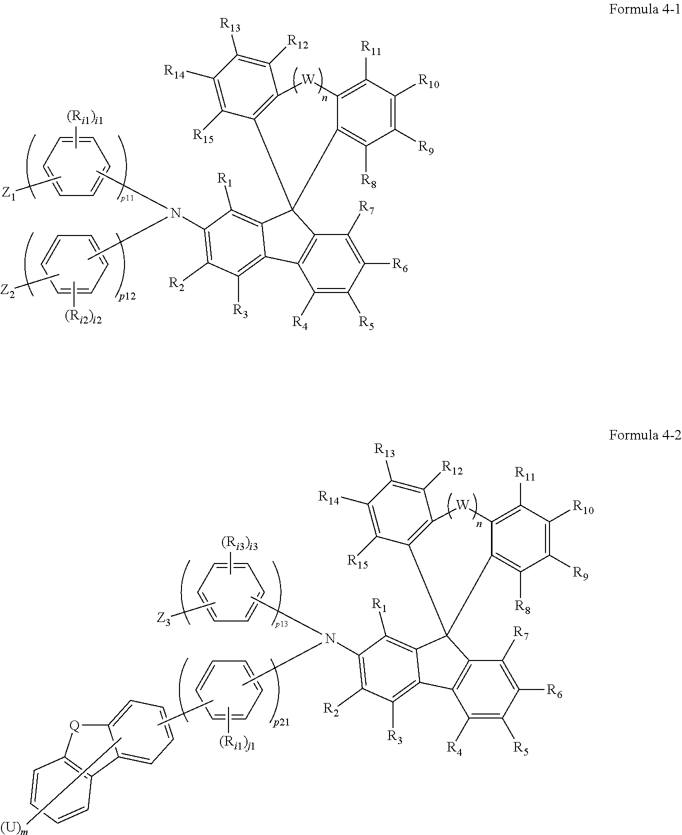
wherein in Formulas 4-1 and 4-2,
Z₁ to Z₃ are each independently a substituted or unsubstituted naphthyl group or a substituted or unsubstituted phenanthrenyl group,
Rᵢ₁ to Rᵢ₃, and Rⱼ₁ are each independently a hydrogen atom, a deuterium atom, a substituted or unsubstituted silyl group, a substituted or unsubstituted amine group, a substituted or unsubstituted aryl group having 6 to 15 ring-forming carbon atoms, or a substituted or unsubstituted heteroaryl group having 2 to 15 ring-forming carbon atoms,
i1 to i3, and j1 are each independently an integer of 0 to 4,
p11 to p13 are each independently an integer of 1 to 3,
p21 is an integer of 0 to 3, and
R₁ to R₁₅, W, Q, U, n, and m are as defined in Formula 1.

7. The light emitting element of claim 6, wherein Formula 4-1 is represented by Formula 4-1-1 or Formula 4-1-2: 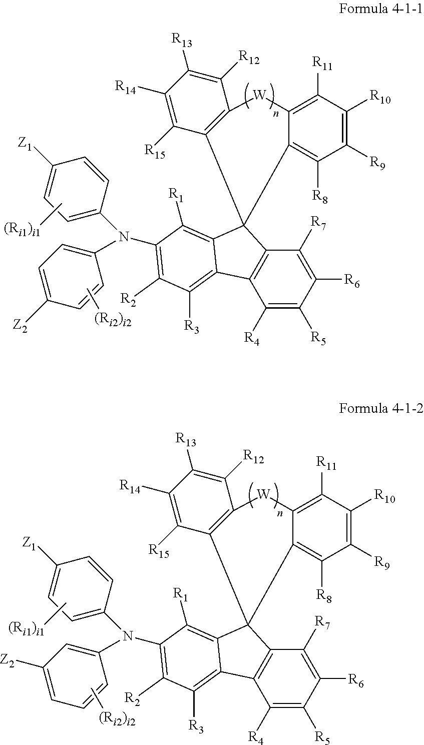
wherein in Formulas 4-1-1 and 4-1-2,
R₁ to R₁₅, Rᵢ₁, Rᵢ₂, W, Z₁, Z₂, i1, i2, and n are as defined in Formulas 1 and 4-1.

8. The light emitting element of claim 6, wherein Formula 4-2 is represented by Formula 4-2-1 or Formula 4-2-2: 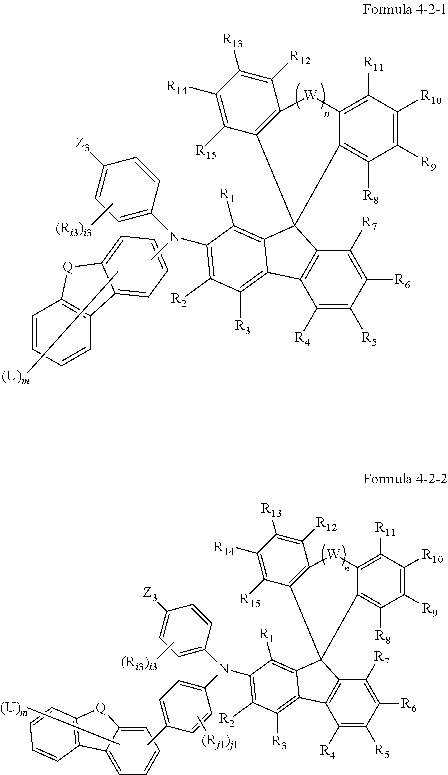
wherein in Formulas 4-2-1 and 4-2-2,
R₁ to R₁₅, Rᵢ₃, Rⱼ₁, W, Q, U, Z₃, i3, j1, n, and m are as defined in Formulas 1 and 4-2.

9. The light emitting element of claim 1, wherein Formula 1 is represented by Formula 5: 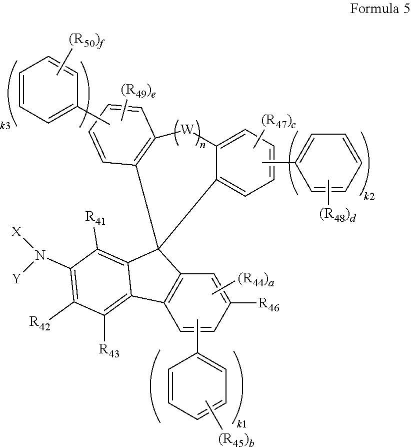
wherein in Formula 5,
R₄₁ to R₄₄, R₄₆, R₄₇, and R₄₉ are each independently a hydrogen atom, a deuterium atom, a halogen atom, a substituted or unsubstituted amine group, a substituted or unsubstituted silyl group, a substituted or unsubstituted aryl group having 6 to 30 ring-forming carbon atoms, or a substituted or unsubstituted heteroaryl group having 2 to 30 ring-forming carbon atoms,
R₄₅, R₄₈, and R₅₀ are each independently a hydrogen atom or a deuterium atom,
a is an integer of 0 to 3,
c and e are each independently an integer of 0 to 4,
b, d, and f are each independently an integer of 0 to 5,
k1 to k3 are each independently 0 or 1, and at least one of k1 to k3 is 1, and
X, Y, W, and n are as defined in Formula 1.

10. The light emitting element of claim 1, wherein the amine compound represented by Formula 1 is a monoamine compound.

11. The light emitting element of claim 1, wherein Formula 1 is represented by any one selected from among compounds of Compound Group 1: 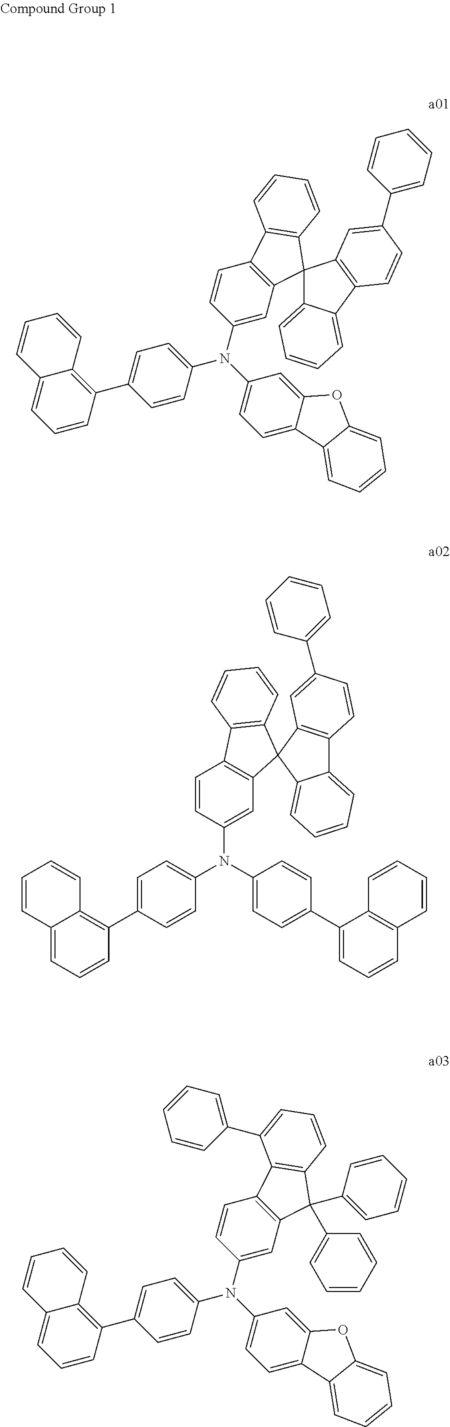 [SMILES: c1ccc(-c2ccc3c(c2)C2(c4ccccc4-3)c3ccccc3-c3ccc(N(c4ccc(-c5cccc6ccccc56)cc4)c4ccc5c(c4)oc4ccccc45)cc32)cc1 ; c1ccc(-c2ccc3c(c2)C2(c4ccccc4-3)c3ccccc3-c3ccc(N(c4ccc(-c5cccc6ccccc56)cc4)c4ccc(-c5cccc6ccccc56)cc4)cc32)cc1 ; c1ccc(-c2cccc3c2-c2ccc(N(c4ccc(-c5cccc6ccccc56)cc4)c4ccc5c(c4)oc4ccccc45)cc2C3(c2ccccc2)c2ccccc2)cc1] 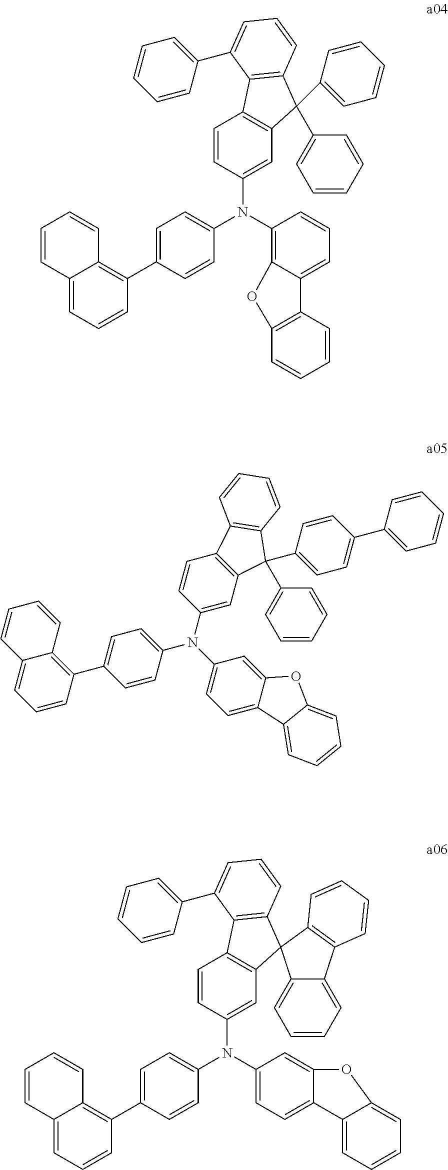 [SMILES: c1ccc(-c2cccc3c2-c2ccc(N(c4ccc(-c5cccc6ccccc56)cc4)c4cccc5c4oc4ccccc45)cc2C3(c2ccccc2)c2ccccc2)cc1 ; c1ccc(-c2ccc(C3(c4ccccc4)c4ccccc4-c4ccc(N(c5ccc(-c6cccc7ccccc67)cc5)c5ccc6c(c5)oc5ccccc56)cc43)cc2)cc1 ; c1ccc(-c2cccc3c2-c2ccc(N(c4ccc(-c5cccc6ccccc56)cc4)c4ccc5c(c4)oc4ccccc45)cc2C32c3ccccc3-c3ccccc32)cc1] 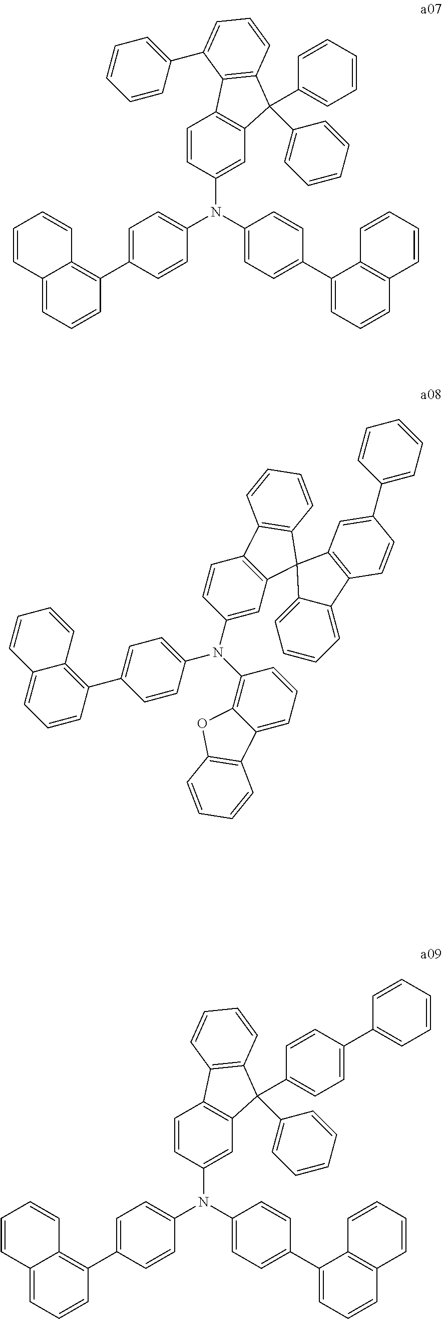 [SMILES: c1ccc(-c2cccc3c2-c2ccc(N(c4ccc(-c5cccc6ccccc56)cc4)c4ccc(-c5cccc6ccccc56)cc4)cc2C3(c2ccccc2)c2ccccc2)cc1 ; c1ccc(-c2ccc3c(c2)C2(c4ccccc4-3)c3ccccc3-c3ccc(N(c4ccc(-c5cccc6ccccc56)cc4)c4cccc5c4oc4ccccc45)cc32)cc1 ; c1ccc(-c2ccc(C3(c4ccccc4)c4ccccc4-c4ccc(N(c5ccc(-c6cccc7ccccc67)cc5)c5ccc(-c6cccc7ccccc67)cc5)cc43)cc2)cc1] 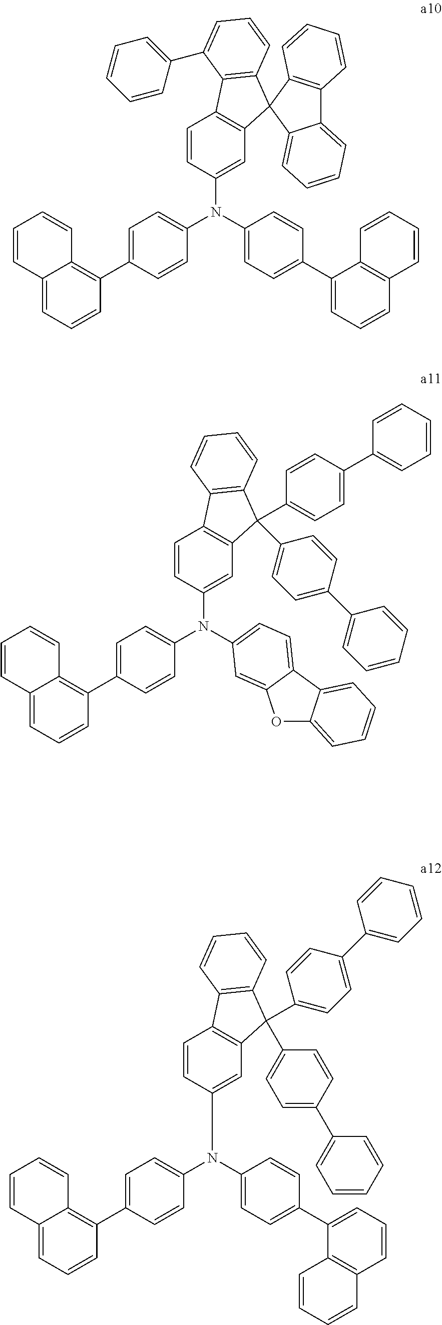 [SMILES: c1ccc(-c2cccc3c2-c2ccc(N(c4ccc(-c5cccc6ccccc56)cc4)c4ccc(-c5cccc6ccccc56)cc4)cc2C32c3ccccc3-c3ccccc32)cc1 ; c1ccc(-c2ccc(C3(c4ccc(-c5ccccc5)cc4)c4ccccc4-c4ccc(N(c5ccc(-c6cccc7ccccc67)cc5)c5ccc6c(c5)oc5ccccc56)cc43)cc2)cc1 ; c1ccc(-c2ccc(C3(c4ccc(-c5ccccc5)cc4)c4ccccc4-c4ccc(N(c5ccc(-c6cccc7ccccc67)cc5)c5ccc(-c6cccc7ccccc67)cc5)cc43)cc2)cc1] 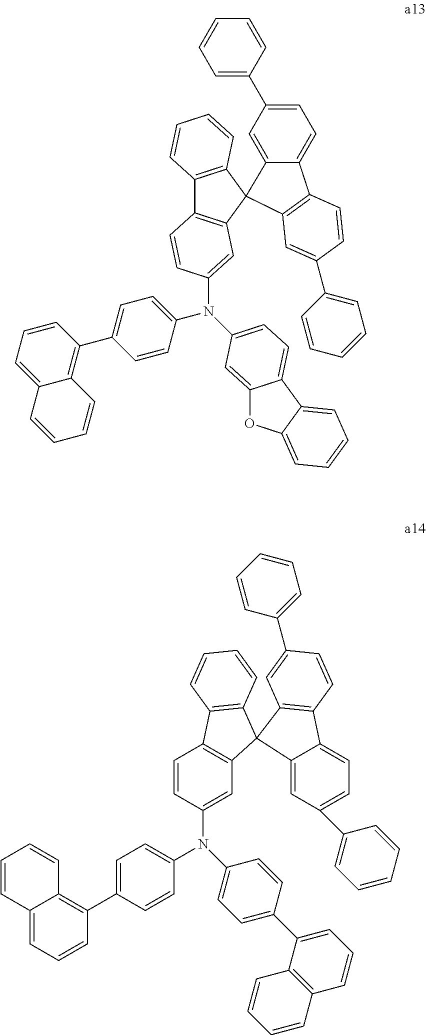 [SMILES: c1ccc(-c2ccc3c(c2)C2(c4ccccc4-c4ccc(N(c5ccc(-c6cccc7ccccc67)cc5)c5ccc6c(c5)oc5ccccc56)cc42)c2cc(-c4ccccc4)ccc2-3)cc1 ; c1ccc(-c2ccc3c(c2)C2(c4ccccc4-c4ccc(N(c5ccc(-c6cccc7ccccc67)cc5)c5ccc(-c6cccc7ccccc67)cc5)cc42)c2cc(-c4ccccc4)ccc2-3)cc1] 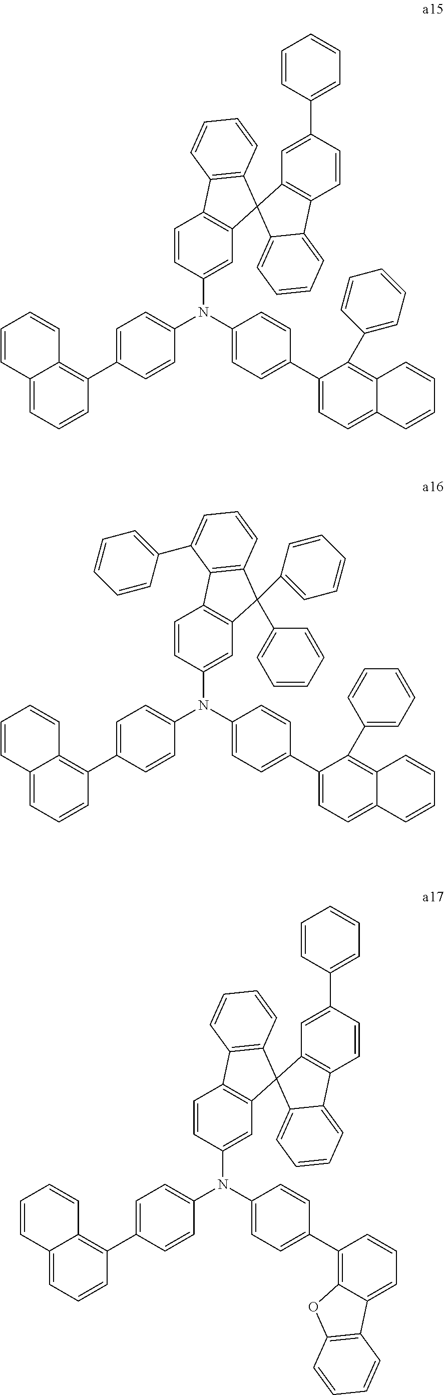 [SMILES: c1ccc(-c2ccc3c(c2)C2(c4ccccc4-3)c3ccccc3-c3ccc(N(c4ccc(-c5ccc6ccccc6c5-c5ccccc5)cc4)c4ccc(-c5cccc6ccccc56)cc4)cc32)cc1 ; c1ccc(-c2cccc3c2-c2ccc(N(c4ccc(-c5ccc6ccccc6c5-c5ccccc5)cc4)c4ccc(-c5cccc6ccccc56)cc4)cc2C3(c2ccccc2)c2ccccc2)cc1 ; c1ccc(-c2ccc3c(c2)C2(c4ccccc4-3)c3ccccc3-c3ccc(N(c4ccc(-c5cccc6ccccc56)cc4)c4ccc(-c5cccc6c5oc5ccccc56)cc4)cc32)cc1] 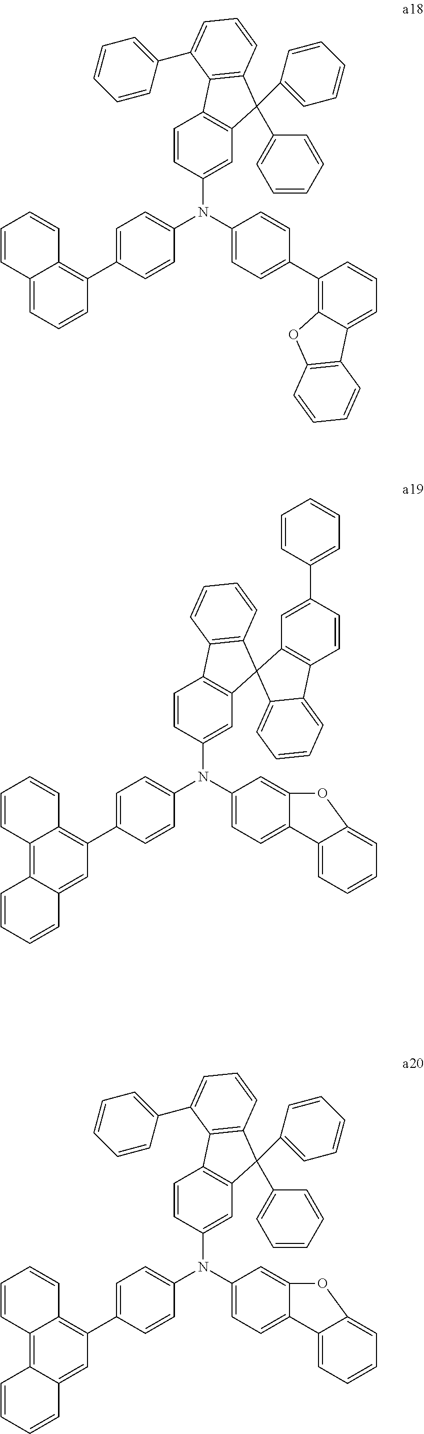 [SMILES: c1ccc(-c2cccc3c2-c2ccc(N(c4ccc(-c5cccc6ccccc56)cc4)c4ccc(-c5cccc6c5oc5ccccc56)cc4)cc2C3(c2ccccc2)c2ccccc2)cc1 ; c1ccc(-c2ccc3c(c2)C2(c4ccccc4-3)c3ccccc3-c3ccc(N(c4ccc(-c5cc6ccccc6c6ccccc56)cc4)c4ccc5c(c4)oc4ccccc45)cc32)cc1 ; c1ccc(-c2cccc3c2-c2ccc(N(c4ccc(-c5cc6ccccc6c6ccccc56)cc4)c4ccc5c(c4)oc4ccccc45)cc2C3(c2ccccc2)c2ccccc2)cc1] 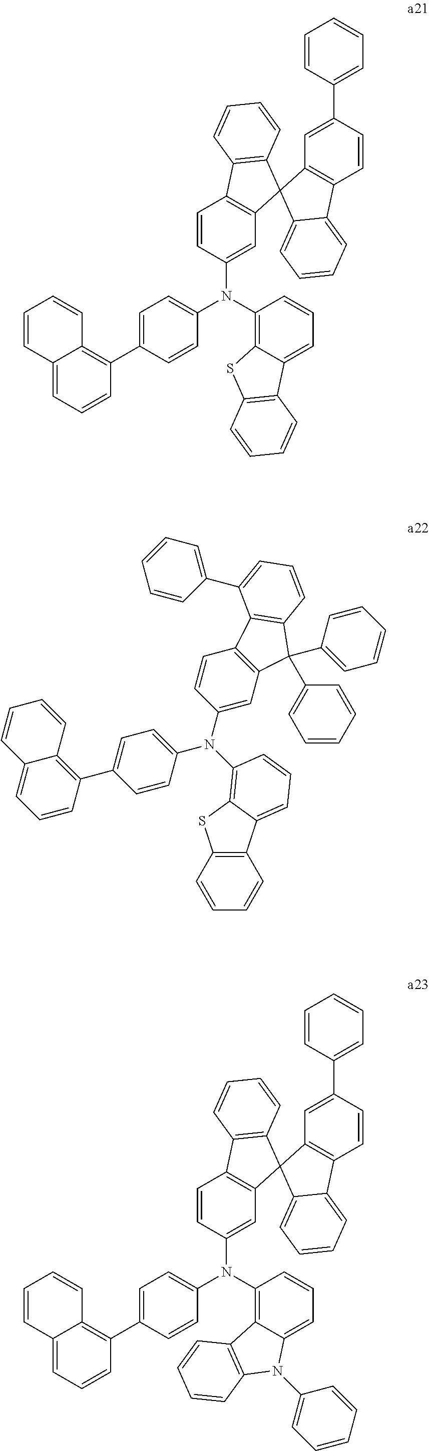 [SMILES: c1ccc(-c2ccc3c(c2)C2(c4ccccc4-3)c3ccccc3-c3ccc(N(c4ccc(-c5cccc6ccccc56)cc4)c4cccc5c4sc4ccccc45)cc32)cc1 ; c1ccc(-c2cccc3c2-c2ccc(N(c4ccc(-c5cccc6ccccc56)cc4)c4cccc5c4sc4ccccc45)cc2C3(c2ccccc2)c2ccccc2)cc1 ; c1ccc(-c2ccc3c(c2)C2(c4ccccc4-3)c3ccccc3-c3ccc(N(c4ccc(-c5cccc6ccccc56)cc4)c4cccc5c4c4ccccc4n5-c4ccccc4)cc32)cc1] 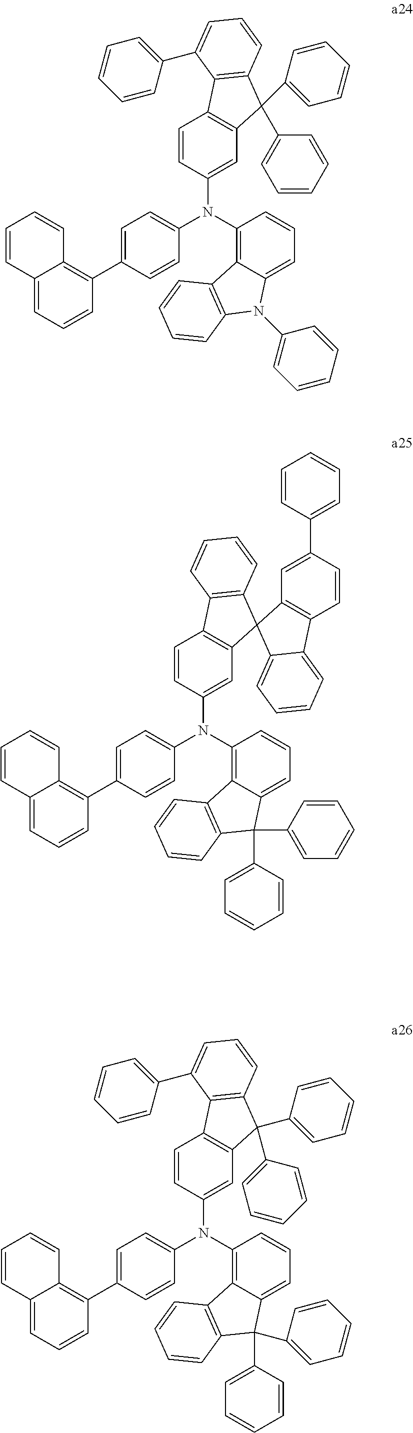 [SMILES: c1ccc(-c2cccc3c2-c2ccc(N(c4ccc(-c5cccc6ccccc56)cc4)c4cccc5c4c4ccccc4n5-c4ccccc4)cc2C3(c2ccccc2)c2ccccc2)cc1 ; c1ccc(-c2ccc3c(c2)C2(c4ccccc4-3)c3ccccc3-c3ccc(N(c4ccc(-c5cccc6ccccc56)cc4)c4cccc5c4-c4ccccc4C5(c4ccccc4)c4ccccc4)cc32)cc1 ; c1ccc(-c2cccc3c2-c2ccc(N(c4ccc(-c5cccc6ccccc56)cc4)c4cccc5c4-c4ccccc4C5(c4ccccc4)c4ccccc4)cc2C3(c2ccccc2)c2ccccc2)cc1] 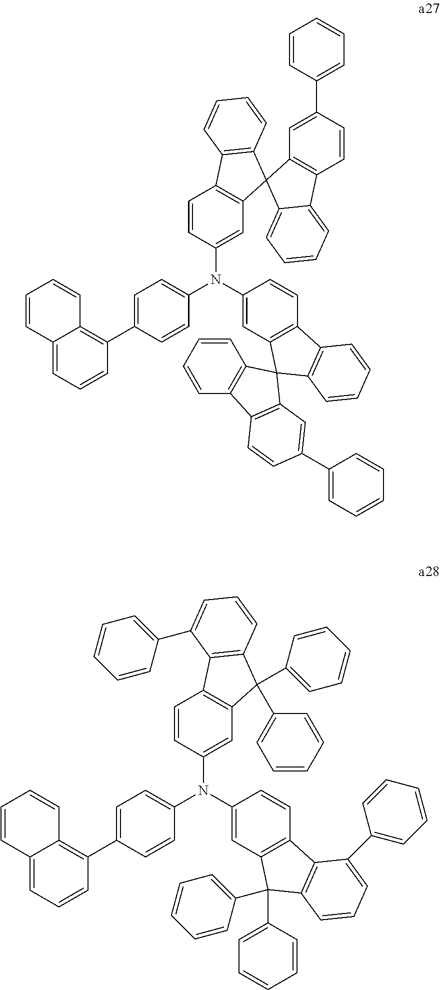 [SMILES: c1ccc(-c2ccc3c(c2)C2(c4ccccc4-3)c3ccccc3-c3ccc(N(c4ccc(-c5cccc6ccccc56)cc4)c4ccc5c(c4)C4(c6ccccc6-c6ccc(-c7ccccc7)cc64)c4ccccc4-5)cc32)cc1 ; c1ccc(-c2cccc3c2-c2ccc(N(c4ccc(-c5cccc6ccccc56)cc4)c4ccc5c(c4)C(c4ccccc4)(c4ccccc4)c4cccc(-c6ccccc6)c4-5)cc2C3(c2ccccc2)c2ccccc2)cc1] 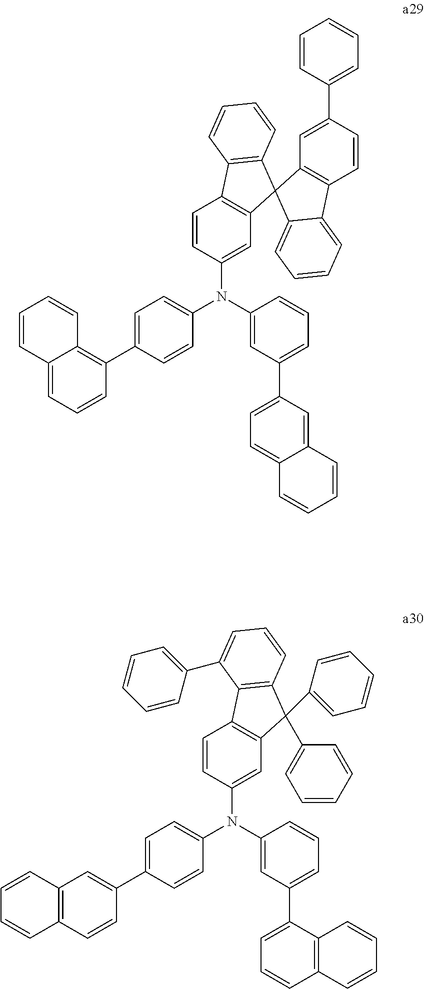 [SMILES: c1ccc(-c2ccc3c(c2)C2(c4ccccc4-3)c3ccccc3-c3ccc(N(c4ccc(-c5cccc6ccccc56)cc4)c4cccc(-c5ccc6ccccc6c5)c4)cc32)cc1 ; c1ccc(-c2cccc3c2-c2ccc(N(c4ccc(-c5ccc6ccccc6c5)cc4)c4cccc(-c5cccc6ccccc56)c4)cc2C3(c2ccccc2)c2ccccc2)cc1] 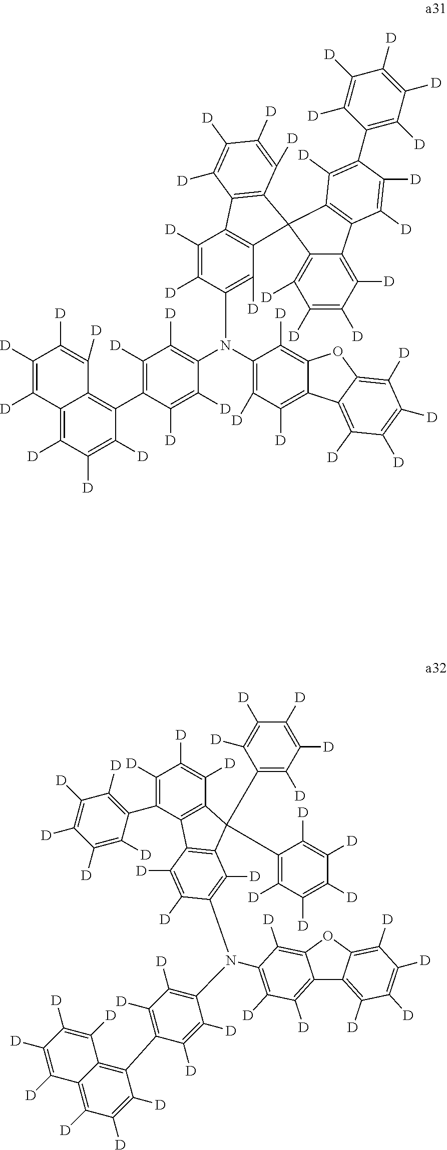 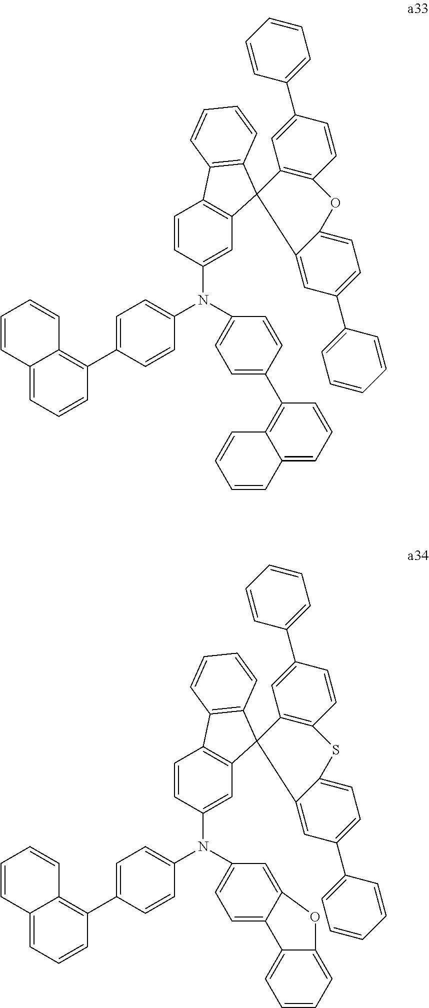 [SMILES: c1ccc(-c2ccc3c(c2)C2(c4cc(-c5ccccc5)ccc4O3)c3ccccc3-c3ccc(N(c4ccc(-c5cccc6ccccc56)cc4)c4ccc(-c5cccc6ccccc56)cc4)cc32)cc1 ; c1ccc(-c2ccc3c(c2)C2(c4cc(-c5ccccc5)ccc4S3)c3ccccc3-c3ccc(N(c4ccc(-c5cccc6ccccc56)cc4)c4ccc5c(c4)oc4ccccc45)cc32)cc1] 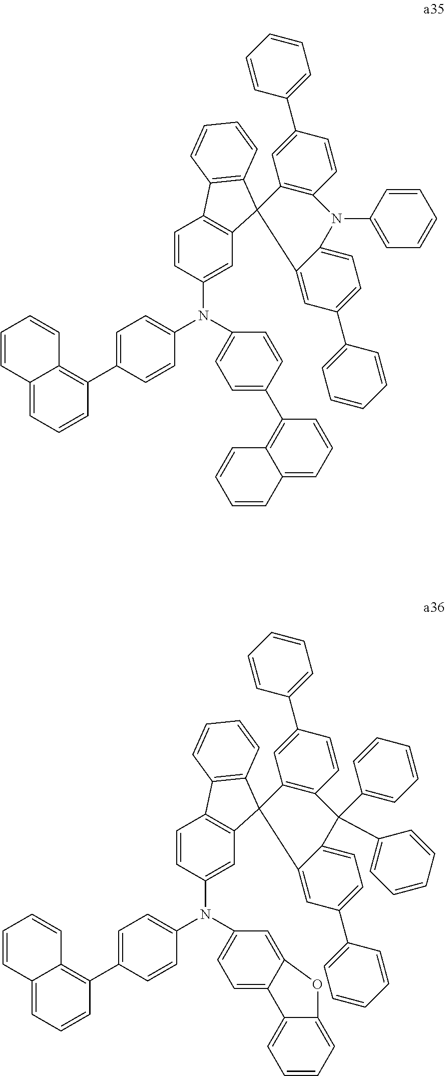 [SMILES: c1ccc(-c2ccc3c(c2)C2(c4ccccc4-c4ccc(N(c5ccc(-c6cccc7ccccc67)cc5)c5ccc(-c6cccc7ccccc67)cc5)cc42)c2cc(-c4ccccc4)ccc2N3c2ccccc2)cc1 ; c1ccc(-c2ccc3c(c2)C2(c4ccccc4-c4ccc(N(c5ccc(-c6cccc7ccccc67)cc5)c5ccc6c(c5)oc5ccccc56)cc42)c2cc(-c4ccccc4)ccc2C3(c2ccccc2)c2ccccc2)cc1] 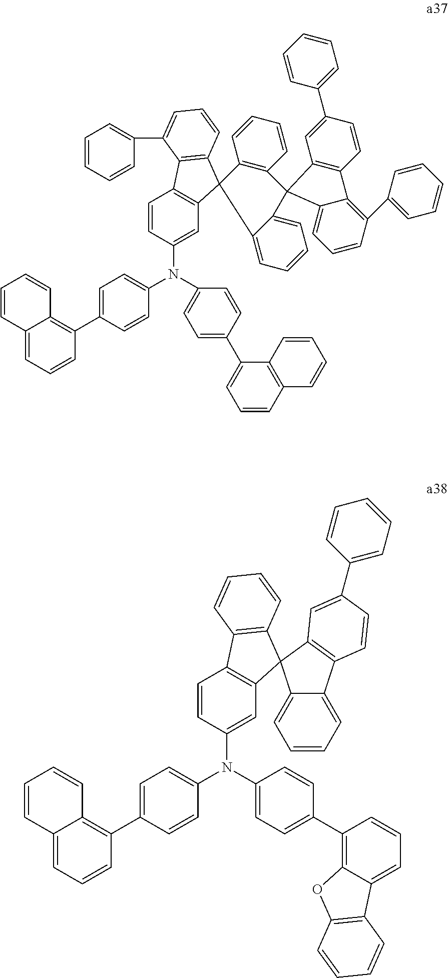 [SMILES: c1ccc(-c2ccc3c(c2)C2(c4ccccc4C4(c5cc(N(c6ccc(-c7cccc8ccccc78)cc6)c6ccc(-c7cccc8ccccc78)cc6)ccc5-c5c(-c6ccccc6)cccc54)c4ccccc42)c2cccc(-c4ccccc4)c2-3)cc1 ; c1ccc(-c2ccc3c(c2)C2(c4ccccc4-3)c3ccccc3-c3ccc(N(c4ccc(-c5cccc6ccccc56)cc4)c4ccc(-c5cccc6c5oc5ccccc56)cc4)cc32)cc1] 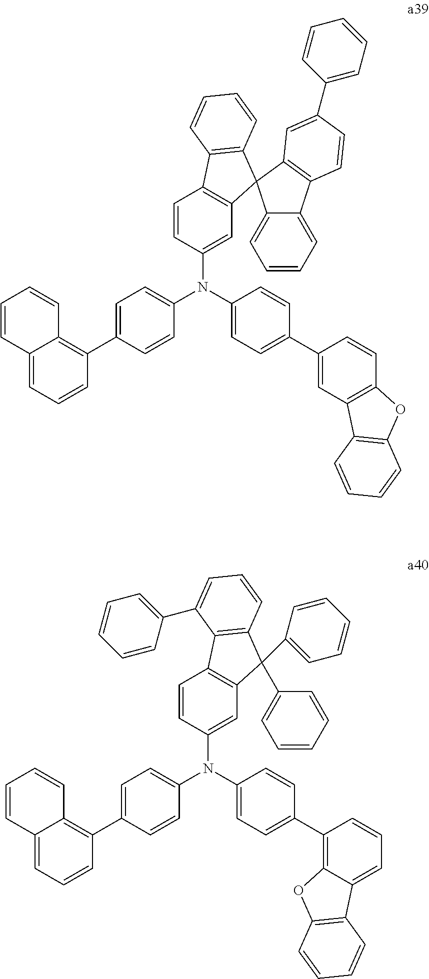 [SMILES: c1ccc(-c2ccc3c(c2)C2(c4ccccc4-3)c3ccccc3-c3ccc(N(c4ccc(-c5ccc6oc7ccccc7c6c5)cc4)c4ccc(-c5cccc6ccccc56)cc4)cc32)cc1 ; c1ccc(-c2cccc3c2-c2ccc(N(c4ccc(-c5cccc6ccccc56)cc4)c4ccc(-c5cccc6c5oc5ccccc56)cc4)cc2C3(c2ccccc2)c2ccccc2)cc1] 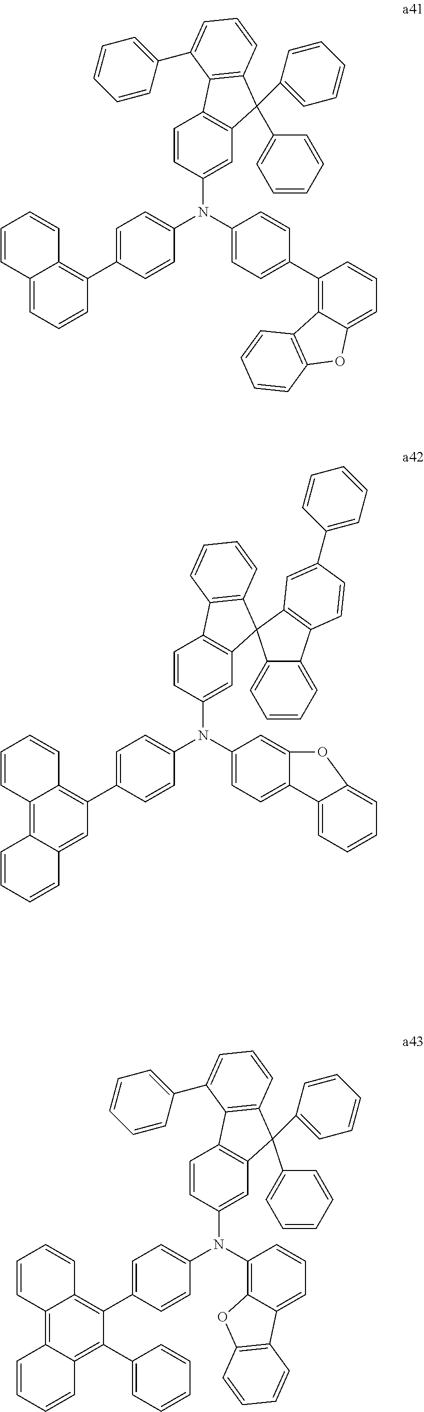 [SMILES: c1ccc(-c2cccc3c2-c2ccc(N(c4ccc(-c5cccc6ccccc56)cc4)c4ccc(-c5cccc6oc7ccccc7c56)cc4)cc2C3(c2ccccc2)c2ccccc2)cc1 ; c1ccc(-c2ccc3c(c2)C2(c4ccccc4-3)c3ccccc3-c3ccc(N(c4ccc(-c5cc6ccccc6c6ccccc56)cc4)c4ccc5c(c4)oc4ccccc45)cc32)cc1 ; c1ccc(-c2cccc3c2-c2ccc(N(c4ccc(-c5c(-c6ccccc6)c6ccccc6c6ccccc56)cc4)c4cccc5c4oc4ccccc45)cc2C3(c2ccccc2)c2ccccc2)cc1] 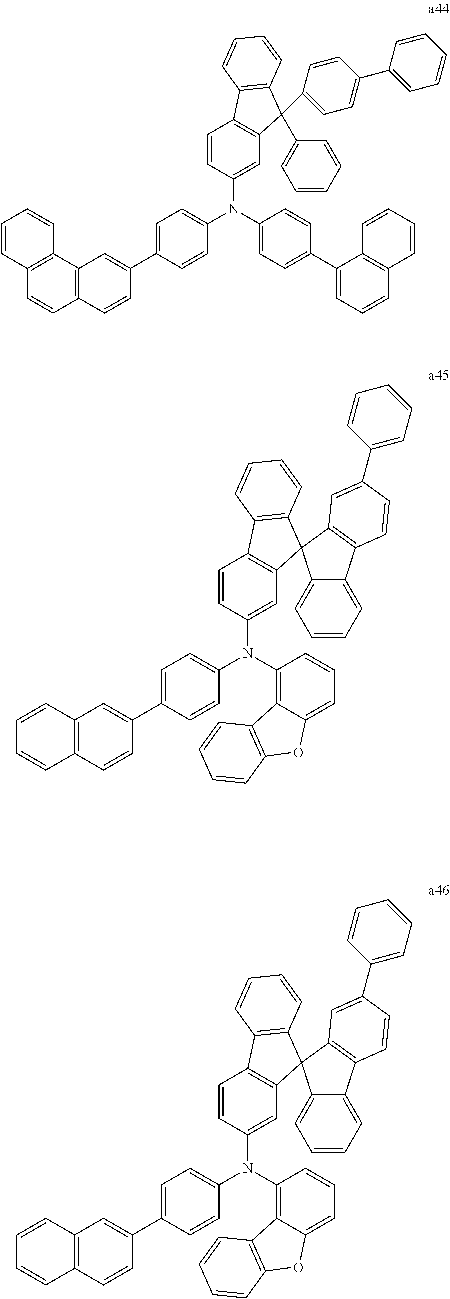 [SMILES: c1ccc(-c2ccc(C3(c4ccccc4)c4ccccc4-c4ccc(N(c5ccc(-c6ccc7ccc8ccccc8c7c6)cc5)c5ccc(-c6cccc7ccccc67)cc5)cc43)cc2)cc1 ; c1ccc(-c2ccc3c(c2)C2(c4ccccc4-3)c3ccccc3-c3ccc(N(c4ccc(-c5ccc6ccccc6c5)cc4)c4cccc5oc6ccccc6c45)cc32)cc1 ; c1ccc(-c2ccc3c(c2)C2(c4ccccc4-3)c3ccccc3-c3ccc(N(c4ccc(-c5ccc6ccccc6c5)cc4)c4cccc5oc6ccccc6c45)cc32)cc1] 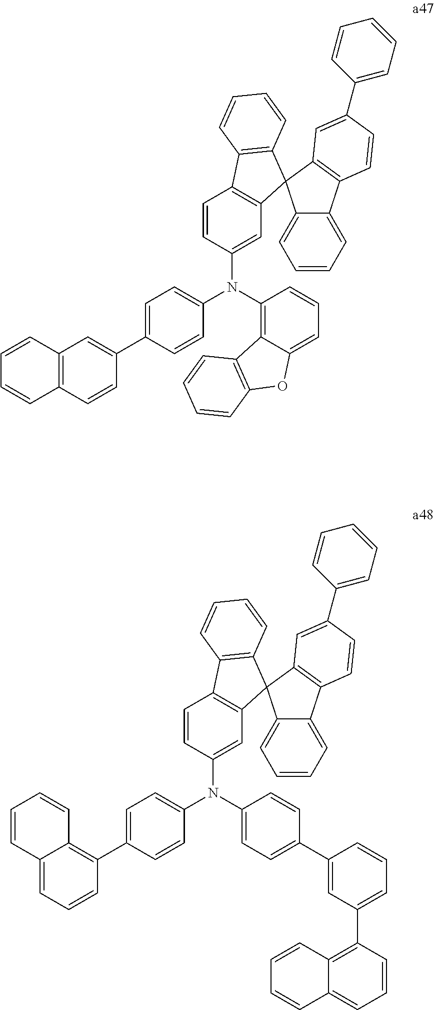 [SMILES: c1ccc(-c2ccc3c(c2)C2(c4ccccc4-3)c3ccccc3-c3ccc(N(c4ccc(-c5ccc6ccccc6c5)cc4)c4cccc5oc6ccccc6c45)cc32)cc1 ; c1ccc(-c2ccc3c(c2)C2(c4ccccc4-3)c3ccccc3-c3ccc(N(c4ccc(-c5cccc(-c6cccc7ccccc67)c5)cc4)c4ccc(-c5cccc6ccccc56)cc4)cc32)cc1] 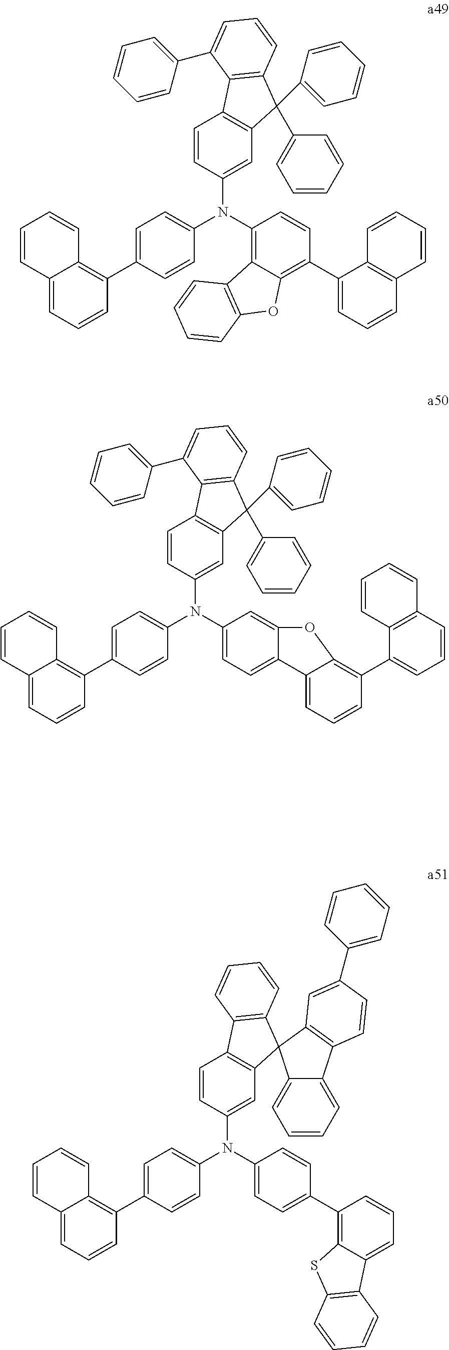 [SMILES: c1ccc(-c2cccc3c2-c2ccc(N(c4ccc(-c5cccc6ccccc56)cc4)c4ccc(-c5cccc6ccccc56)c5oc6ccccc6c45)cc2C3(c2ccccc2)c2ccccc2)cc1 ; c1ccc(-c2cccc3c2-c2ccc(N(c4ccc(-c5cccc6ccccc56)cc4)c4ccc5c(c4)oc4c(-c6cccc7ccccc67)cccc45)cc2C3(c2ccccc2)c2ccccc2)cc1 ; c1ccc(-c2ccc3c(c2)C2(c4ccccc4-3)c3ccccc3-c3ccc(N(c4ccc(-c5cccc6ccccc56)cc4)c4ccc(-c5cccc6c5sc5ccccc56)cc4)cc32)cc1] 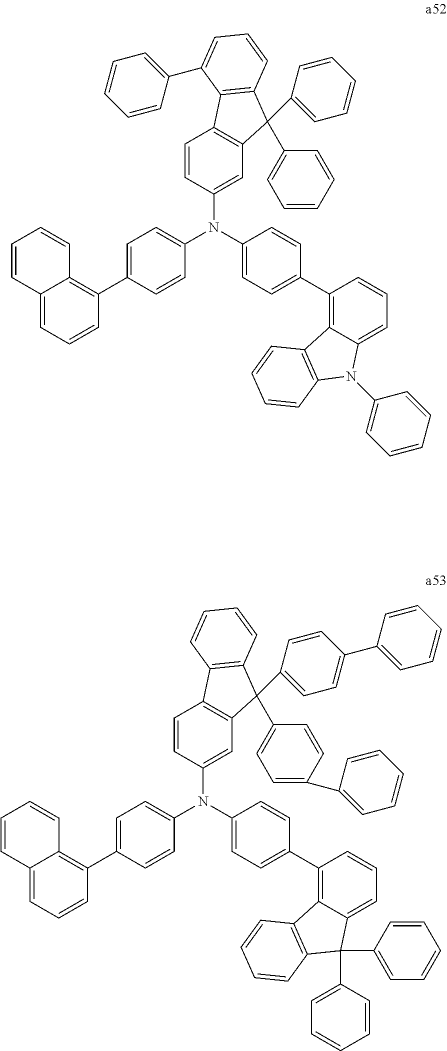 [SMILES: c1ccc(-c2cccc3c2-c2ccc(N(c4ccc(-c5cccc6ccccc56)cc4)c4ccc(-c5cccc6c5c5ccccc5n6-c5ccccc5)cc4)cc2C3(c2ccccc2)c2ccccc2)cc1 ; c1ccc(-c2ccc(C3(c4ccc(-c5ccccc5)cc4)c4ccccc4-c4ccc(N(c5ccc(-c6cccc7c6-c6ccccc6C7(c6ccccc6)c6ccccc6)cc5)c5ccc(-c6cccc7ccccc67)cc5)cc43)cc2)cc1]
wherein in Compound Group 1, D is a deuterium atom.

12. An amine compound represented by Formula 1: 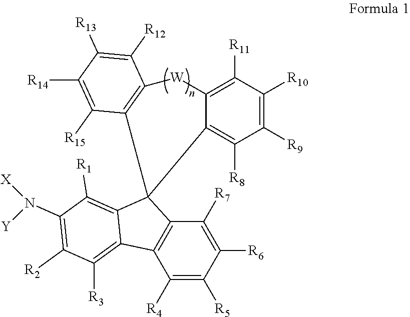
wherein in Formula 1,
R₁ to R₁₅ are each independently a hydrogen atom, a deuterium atom, a halogen atom, a substituted or unsubstituted amine group, a substituted or unsubstituted silyl group, a substituted or unsubstituted aryl group having 6 to 30 ring-forming carbon atoms, or a substituted or unsubstituted heteroaryl group having 2 to 30 ring-forming carbon atoms,
at least one selected from among R₃ to R₅, and R₇ to R₁₅ is a substituted or unsubstituted aryl group having 6 to 30 ring-forming carbon atoms, or a substituted or unsubstituted heteroaryl group having 2 to 30 ring-forming carbon atoms,
W is O, S, NRₐ₁, CRₐ₂Rₐ₃, or a direct linkage,
n is 0 or 1,
Rₐ₁ is a substituted or unsubstituted aryl group having 6 to 30 ring-forming carbon atoms, or a substituted or unsubstituted heteroaryl group having 2 to 30 ring-forming carbon atoms,
Rₐ₂ and Rₐ₃ are each independently a substituted or unsubstituted aryl group having 6 to 30 ring-forming carbon atoms, or a substituted or unsubstituted heteroaryl group having 2 to 30 ring-forming carbon atoms, and/or Rₐ₂ and Rₐ₃ are bonded to form a ring, and
X and Y are each independently represented by Formula 2 or Formula 3, and at least one of X or Y is represented by Formula 2 and the other is represented by Formula 3: 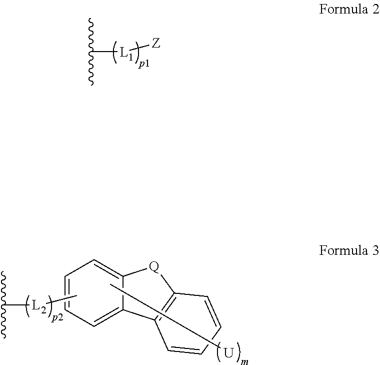
wherein in Formulas 2 and 3,
L₁ and L₂ are each independently a substituted or unsubstituted arylene group having 6 to 50 ring-forming carbon atoms, or a substituted or unsubstituted heteroarylene group having 2 to 50 ring-forming carbon atoms,
p1 is an integer of 1 to 3,
p2 is an integer of 0 to 3,
Z is a substituted or unsubstituted naphthyl group, or a substituted or unsubstituted phenanthrenyl group,
U is a hydrogen atom, a deuterium atom, a halogen atom, a substituted or unsubstituted amine group, a substituted or unsubstituted silyl group, a substituted or unsubstituted aryl group having 6 to 30 ring-forming carbon atoms, or a substituted or unsubstituted heteroaryl group having 2 to 30 ring-forming carbon atoms,
m is an integer of 0 to 7,
Q is O, S, NRₐ₄, or CRₐ₅Rₐ₆,
Rₐ₄ is a substituted or unsubstituted aryl group having 6 to 30 ring-forming carbon atoms, or a substituted or unsubstituted heteroaryl group having 2 to 30 ring-forming carbon atoms,
Rₐ₅ and Rₐ₆ are each independently a substituted or unsubstituted aryl group having 6 to 30 ring-forming carbon atoms, or a substituted or unsubstituted heteroaryl group having 2 to 30 ring-forming carbon atoms, and/or Rₐ₅ and Rₐ₆ are bonded to form a ring, and
a case in which Formula 3 is a substituted or unsubstituted 2-dibenzothiophene group is excluded.

13. The amine compound of claim 12, wherein Formula 1 is represented by Formula 1-1 or Formula 1-2: 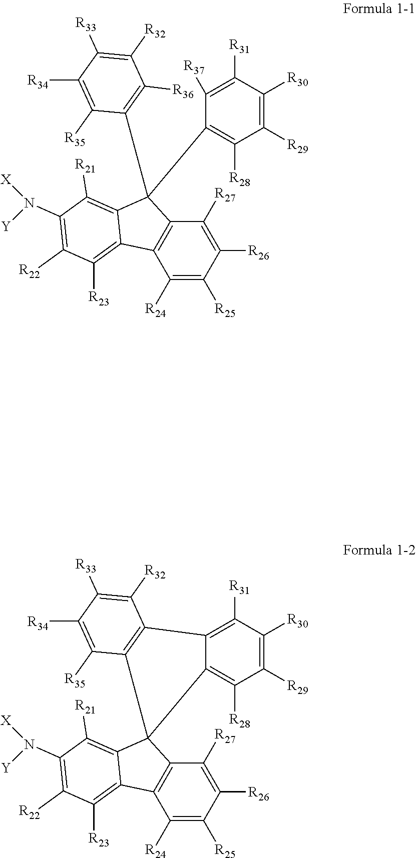
wherein in Formulas 1-1 and 1-2,
R₂₁ to R₃₅ are each independently a hydrogen atom, a deuterium atom, or a substituted or unsubstituted aryl group having 6 to 30 ring-forming carbon atoms,
at least one selected from among R₂₃ to R₂₅, and R₂₇ to R₃₅ is a substituted or unsubstituted aryl group having 6 to 30 ring-forming carbon atoms,
R₂₁ and R₃₇ are each independently a hydrogen atom or a deuterium atom, and
X and Y are as defined in Formula 1.

14. The amine compound of claim 12, wherein Formula 1 is represented by Formula 4-1 or Formula 4-2: 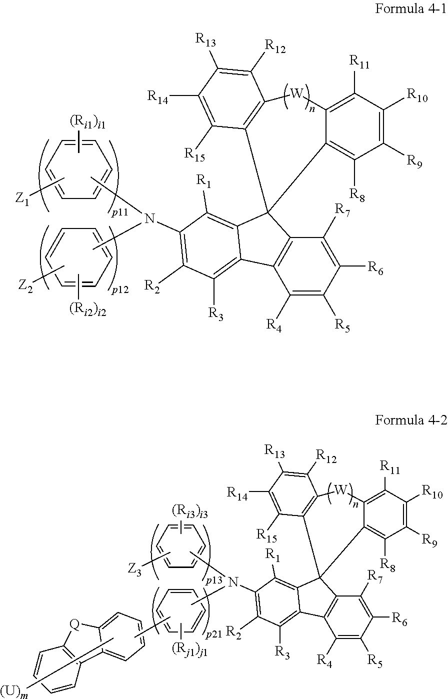
wherein in Formulas 4-1 and 4-2,
Z₁ to Z₃ are each independently a substituted or unsubstituted naphthyl group or a substituted or unsubstituted phenanthrenyl group,
Rᵢ₁ to Rᵢ₃, and Rⱼ₁ are each independently a hydrogen atom, a deuterium atom, a substituted or unsubstituted silyl group, a substituted or unsubstituted amine group, a substituted or unsubstituted aryl group having 6 to 15 ring-forming carbon atoms, or a substituted or unsubstituted heteroaryl group having 2 to 15 ring-forming carbon atoms,
i1 to i3, and j1 are each independently an integer of 0 to 4,
p11 to p13 are each independently an integer of 1 to 3,
p21 is an integer of 0 to 3, and
R₁ to R₁₅, W, Q, U, n, and m are as defined in Formula 1.

15. The amine compound of claim 14, wherein Formula 4-1 is represented by Formula 4-1-1 or Formula 4-1-2: 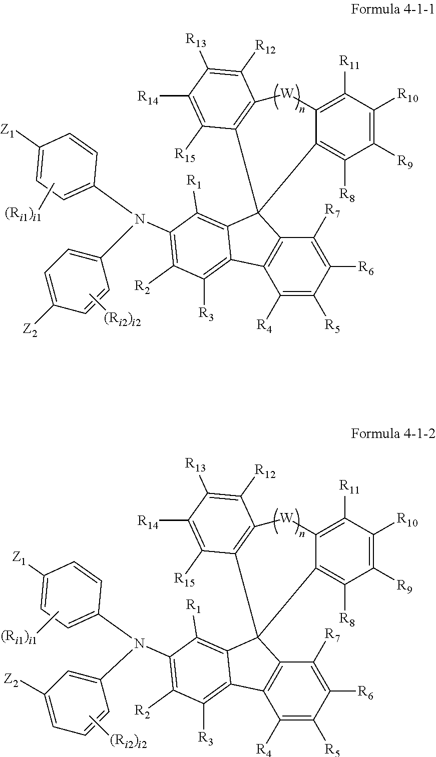
wherein in Formulas 4-1-1 and 4-1-2,
R₁ to R₁₅, Rᵢ₁, Rᵢ₂, W, Z₁, Z₂, i1, i2, and n are as defined in Formulas 1 and 4-1.

16. The amine compound of claim 14, wherein Formula 4-2 is represented by Formula 4-2-1 or Formula 4-2-2: 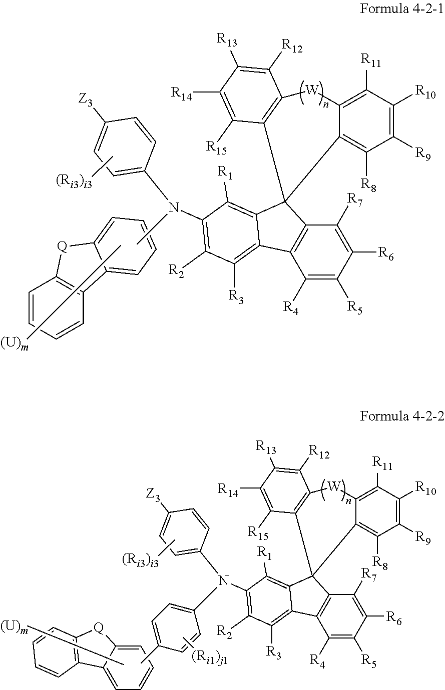
wherein in Formulas 4-2-1 and 4-2-2,
R₁ to R₁₅, Rᵢ₃, Rⱼ₁, W, Q, U, Z₃, i3, j1, n, and m are as defined in Formulas 1 and 4-2.

17. The amine compound of claim 12, wherein Formula 1 is represented by Formula 5: 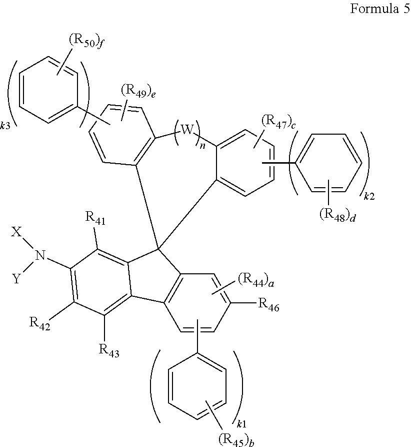
wherein in Formula 5,
R₄₁ to R₄₄, R₄₆, R₄₇, and R₄₉ are each independently a hydrogen atom, a deuterium atom, a halogen atom, a substituted or unsubstituted amine group, a substituted or unsubstituted silyl group, a substituted or unsubstituted aryl group having 6 to 30 ring-forming carbon atoms, or a substituted or unsubstituted heteroaryl group having 2 to 30 ring-forming carbon atoms,
R₄₅, R₄₈, and R₅₀ are each independently a hydrogen atom or a deuterium atom,
a is an integer of 0 to 3,
c and e are each independently an integer of 0 to 4,
b, d, and f are each independently an integer of 0 to 5,
k1 to k3 are each independently 0 or 1, and at least one of k1 to k3 is 1, and
X, Y, W, and n are as defined in Formula 1.

18. The amine compound of claim 17, wherein Formula 5 is represented by Formula 6-1 or Formula 6-2: 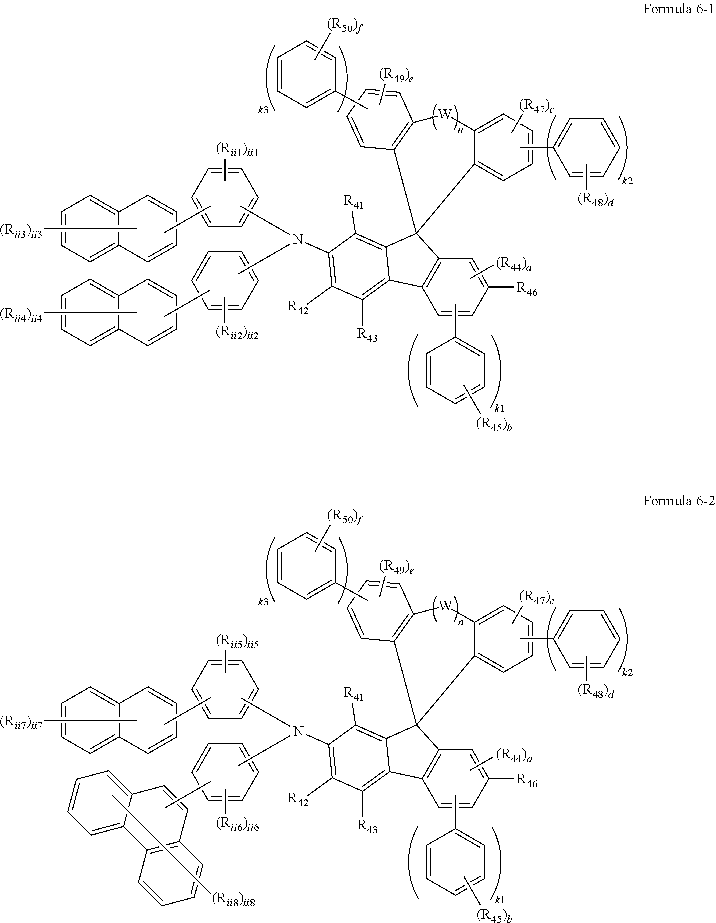
wherein in Formulas 6-1 and 6-2,
Rᵢᵢ₁ to Rᵢᵢ₈ are each independently a hydrogen atom, a deuterium atom, a substituted or unsubstituted silyl group, a substituted or unsubstituted amine group, a substituted or unsubstituted aryl group having 6 to 15 ring-forming carbon atoms, or a substituted or unsubstituted heteroaryl group having 2 to 15 ring-forming carbon atoms,
ii1, ii2, i, and ii6 are each independently an integer of 0 to 4,
ii3, ii4, and ii7 are each independently an integer of 0 to 7, and ii8 is an integer of 0 to 9, and
R₄₁ to R₅₀, W, a to f, k1 to k3, and n are as defined in Formula 1 and Formula 5.

19. The amine compound of claim 17, wherein Formula 5 is represented by any one among Formulas 7-1 to 7-3: 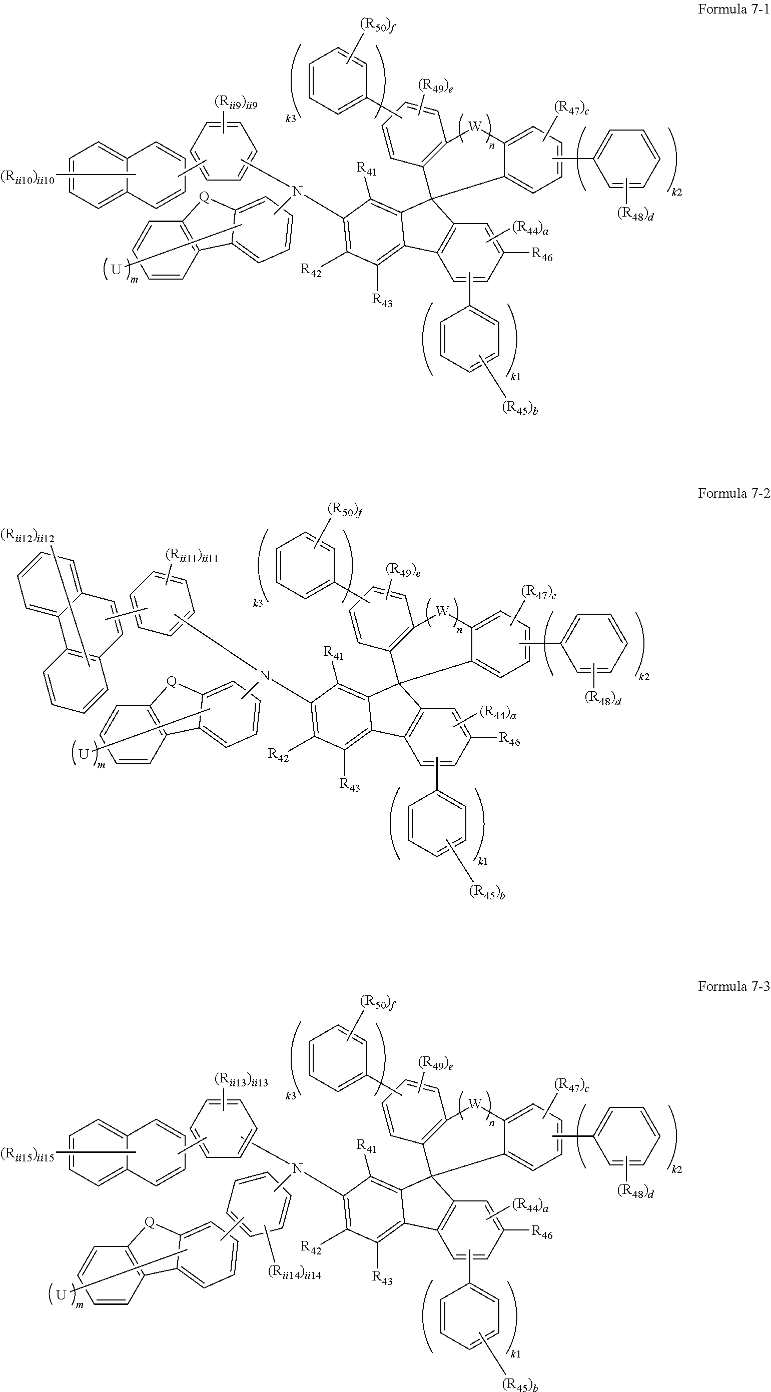
wherein in Formulas 7-1 to 7-3,
Rᵢᵢ₉ to Rᵢᵢ₁₅ are each independently a hydrogen atom, a deuterium atom, a substituted or unsubstituted silyl group, a substituted or unsubstituted amine group, a substituted or unsubstituted aryl group having 6 to 15 ring-forming carbon atoms, or a substituted or unsubstituted heteroaryl group having 2 to 15 ring-forming carbon atoms,
ii9, ii11, ii13, and ii14 are each independently an integer of 0 to 4,
ii10 and ii15 are each independently an integer of 0 to 7,
ii12 is an integer of 0 to 9, and
R₄₁ to R₅₀, Q, U, W, a to f, k1 to k3, m and n are as defined in Formula 1 and Formula 5.

20. The amine compound of claim 12, wherein Formula 1 is represented by any one selected from among compounds of Compound Group 1: 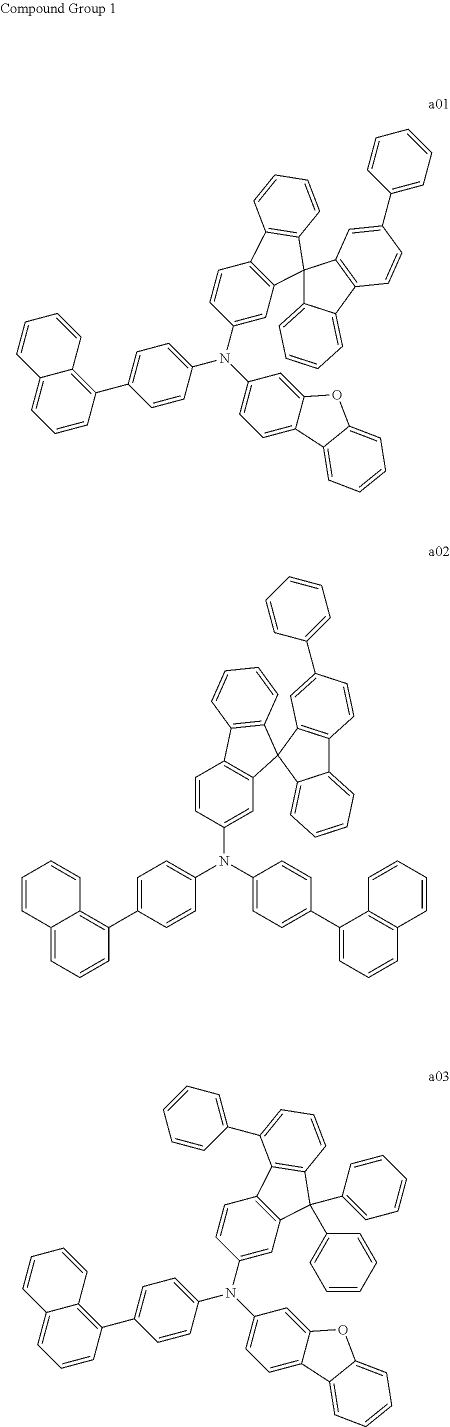 [SMILES: c1ccc(-c2ccc3c(c2)C2(c4ccccc4-3)c3ccccc3-c3ccc(N(c4ccc(-c5cccc6ccccc56)cc4)c4ccc5c(c4)oc4ccccc45)cc32)cc1 ; c1ccc(-c2ccc3c(c2)C2(c4ccccc4-3)c3ccccc3-c3ccc(N(c4ccc(-c5cccc6ccccc56)cc4)c4ccc(-c5cccc6ccccc56)cc4)cc32)cc1 ; c1ccc(-c2cccc3c2-c2ccc(N(c4ccc(-c5cccc6ccccc56)cc4)c4ccc5c(c4)oc4ccccc45)cc2C3(c2ccccc2)c2ccccc2)cc1] 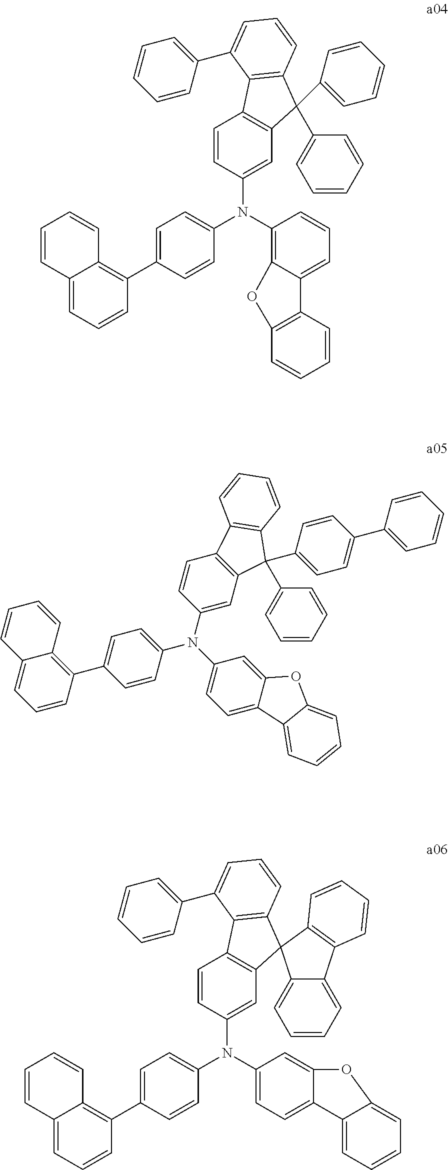 [SMILES: c1ccc(-c2cccc3c2-c2ccc(N(c4ccc(-c5cccc6ccccc56)cc4)c4cccc5c4oc4ccccc45)cc2C3(c2ccccc2)c2ccccc2)cc1 ; c1ccc(-c2ccc(C3(c4ccccc4)c4ccccc4-c4ccc(N(c5ccc(-c6cccc7ccccc67)cc5)c5ccc6c(c5)oc5ccccc56)cc43)cc2)cc1 ; c1ccc(-c2cccc3c2-c2ccc(N(c4ccc(-c5cccc6ccccc56)cc4)c4ccc5c(c4)oc4ccccc45)cc2C32c3ccccc3-c3ccccc32)cc1] 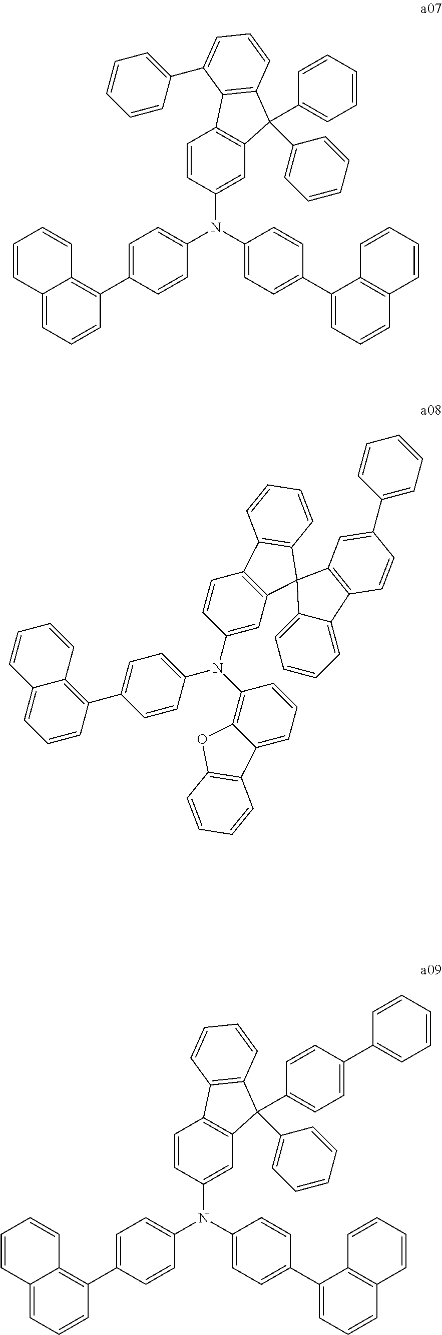 [SMILES: c1ccc(-c2cccc3c2-c2ccc(N(c4ccc(-c5cccc6ccccc56)cc4)c4ccc(-c5cccc6ccccc56)cc4)cc2C3(c2ccccc2)c2ccccc2)cc1 ; c1ccc(-c2ccc3c(c2)C2(c4ccccc4-3)c3ccccc3-c3ccc(N(c4ccc(-c5cccc6ccccc56)cc4)c4cccc5c4oc4ccccc45)cc32)cc1 ; c1ccc(-c2ccc(C3(c4ccccc4)c4ccccc4-c4ccc(N(c5ccc(-c6cccc7ccccc67)cc5)c5ccc(-c6cccc7ccccc67)cc5)cc43)cc2)cc1] 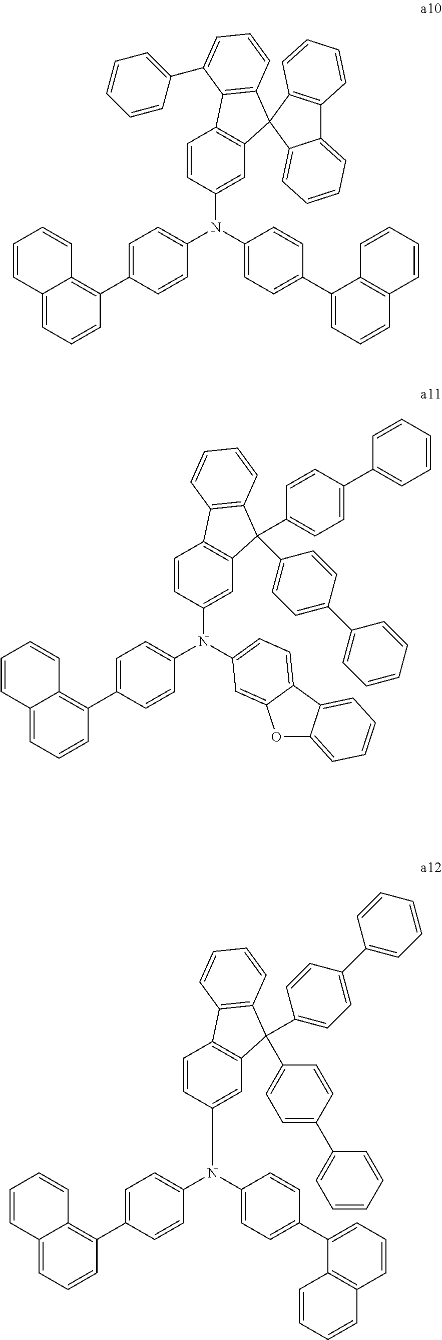 [SMILES: c1ccc(-c2cccc3c2-c2ccc(N(c4ccc(-c5cccc6ccccc56)cc4)c4ccc(-c5cccc6ccccc56)cc4)cc2C32c3ccccc3-c3ccccc32)cc1 ; c1ccc(-c2ccc(C3(c4ccc(-c5ccccc5)cc4)c4ccccc4-c4ccc(N(c5ccc(-c6cccc7ccccc67)cc5)c5ccc6c(c5)oc5ccccc56)cc43)cc2)cc1 ; c1ccc(-c2ccc(C3(c4ccc(-c5ccccc5)cc4)c4ccccc4-c4ccc(N(c5ccc(-c6cccc7ccccc67)cc5)c5ccc(-c6cccc7ccccc67)cc5)cc43)cc2)cc1] 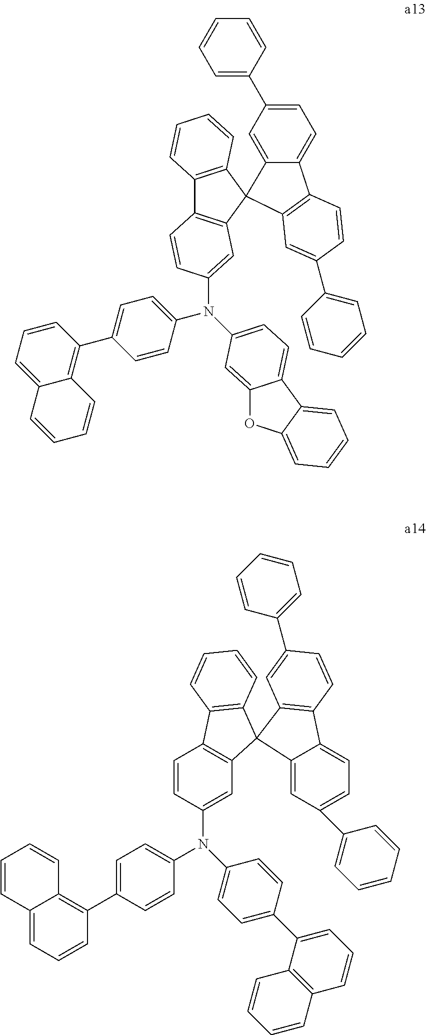 [SMILES: c1ccc(-c2ccc3c(c2)C2(c4ccccc4-c4ccc(N(c5ccc(-c6cccc7ccccc67)cc5)c5ccc6c(c5)oc5ccccc56)cc42)c2cc(-c4ccccc4)ccc2-3)cc1 ; c1ccc(-c2ccc3c(c2)C2(c4ccccc4-c4ccc(N(c5ccc(-c6cccc7ccccc67)cc5)c5ccc(-c6cccc7ccccc67)cc5)cc42)c2cc(-c4ccccc4)ccc2-3)cc1] 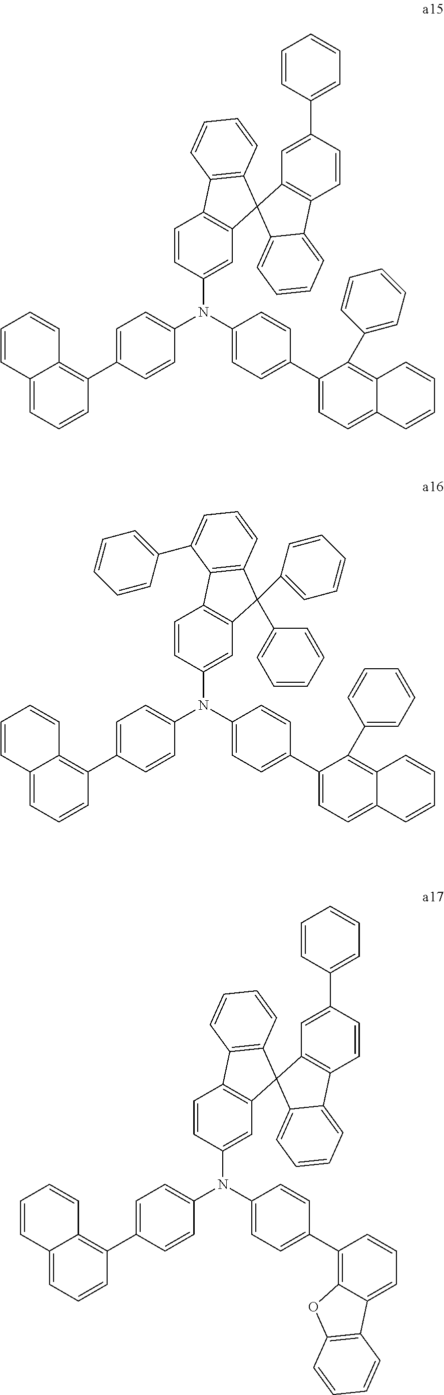 [SMILES: c1ccc(-c2ccc3c(c2)C2(c4ccccc4-3)c3ccccc3-c3ccc(N(c4ccc(-c5ccc6ccccc6c5-c5ccccc5)cc4)c4ccc(-c5cccc6ccccc56)cc4)cc32)cc1 ; c1ccc(-c2cccc3c2-c2ccc(N(c4ccc(-c5ccc6ccccc6c5-c5ccccc5)cc4)c4ccc(-c5cccc6ccccc56)cc4)cc2C3(c2ccccc2)c2ccccc2)cc1 ; c1ccc(-c2ccc3c(c2)C2(c4ccccc4-3)c3ccccc3-c3ccc(N(c4ccc(-c5cccc6ccccc56)cc4)c4ccc(-c5cccc6c5oc5ccccc56)cc4)cc32)cc1] 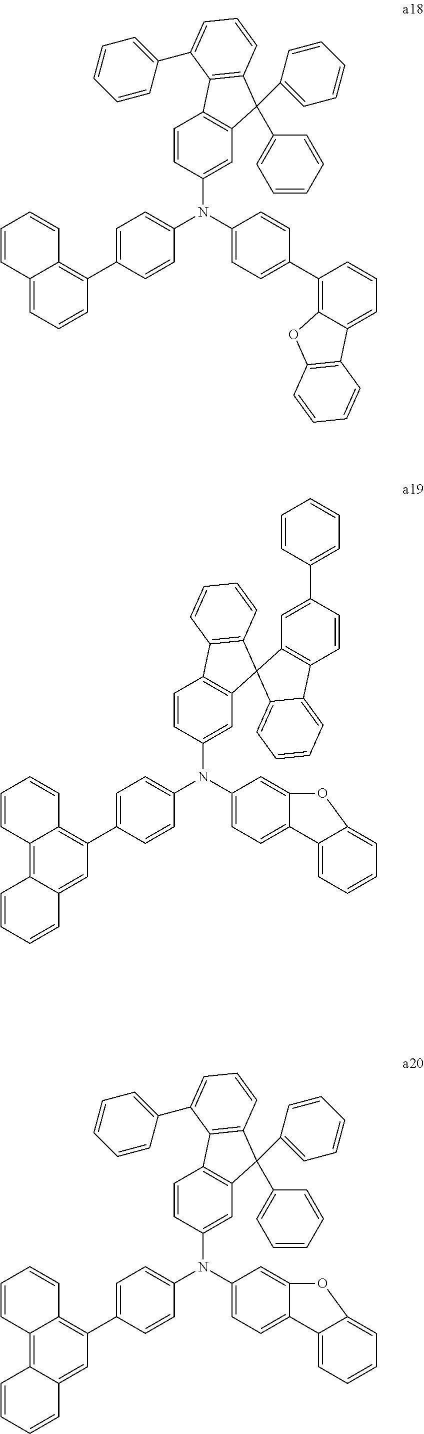 [SMILES: c1ccc(-c2cccc3c2-c2ccc(N(c4ccc(-c5cccc6ccccc56)cc4)c4ccc(-c5cccc6c5oc5ccccc56)cc4)cc2C3(c2ccccc2)c2ccccc2)cc1 ; c1ccc(-c2ccc3c(c2)C2(c4ccccc4-3)c3ccccc3-c3ccc(N(c4ccc(-c5cc6ccccc6c6ccccc56)cc4)c4ccc5c(c4)oc4ccccc45)cc32)cc1 ; c1ccc(-c2cccc3c2-c2ccc(N(c4ccc(-c5cc6ccccc6c6ccccc56)cc4)c4ccc5c(c4)oc4ccccc45)cc2C3(c2ccccc2)c2ccccc2)cc1] 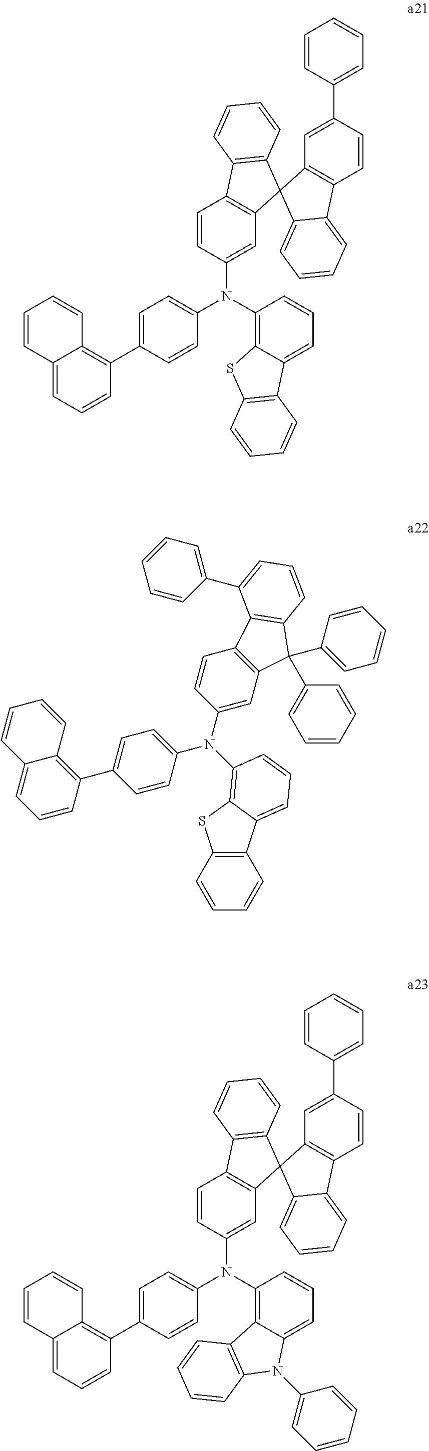 [SMILES: c1ccc(-c2ccc3c(c2)C2(c4ccccc4-3)c3ccccc3-c3ccc(N(c4ccc(-c5cccc6ccccc56)cc4)c4cccc5c4sc4ccccc45)cc32)cc1 ; c1ccc(-c2cccc3c2-c2ccc(N(c4ccc(-c5cccc6ccccc56)cc4)c4cccc5c4sc4ccccc45)cc2C3(c2ccccc2)c2ccccc2)cc1 ; c1ccc(-c2ccc3c(c2)C2(c4ccccc4-3)c3ccccc3-c3ccc(N(c4ccc(-c5cccc6ccccc56)cc4)c4cccc5c4c4ccccc4n5-c4ccccc4)cc32)cc1] 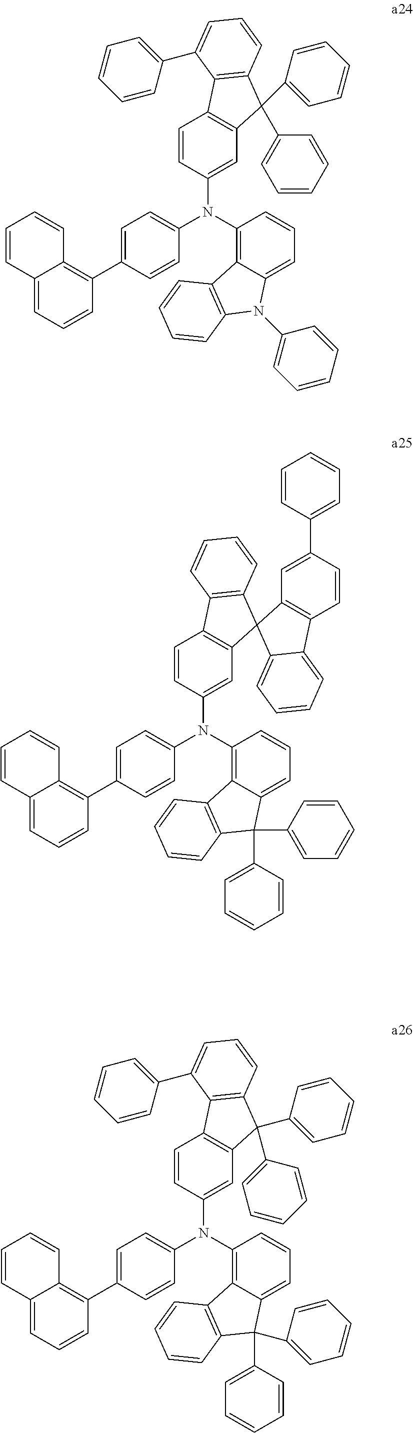 [SMILES: c1ccc(-c2cccc3c2-c2ccc(N(c4ccc(-c5cccc6ccccc56)cc4)c4cccc5c4c4ccccc4n5-c4ccccc4)cc2C3(c2ccccc2)c2ccccc2)cc1 ; c1ccc(-c2ccc3c(c2)C2(c4ccccc4-3)c3ccccc3-c3ccc(N(c4ccc(-c5cccc6ccccc56)cc4)c4cccc5c4-c4ccccc4C5(c4ccccc4)c4ccccc4)cc32)cc1 ; c1ccc(-c2cccc3c2-c2ccc(N(c4ccc(-c5cccc6ccccc56)cc4)c4cccc5c4-c4ccccc4C5(c4ccccc4)c4ccccc4)cc2C3(c2ccccc2)c2ccccc2)cc1] 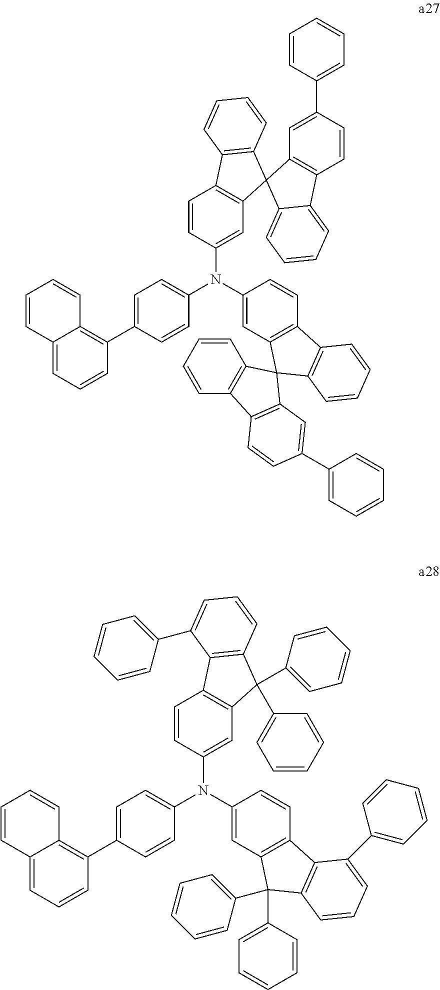 [SMILES: c1ccc(-c2ccc3c(c2)C2(c4ccccc4-3)c3ccccc3-c3ccc(N(c4ccc(-c5cccc6ccccc56)cc4)c4ccc5c(c4)C4(c6ccccc6-c6ccc(-c7ccccc7)cc64)c4ccccc4-5)cc32)cc1 ; c1ccc(-c2cccc3c2-c2ccc(N(c4ccc(-c5cccc6ccccc56)cc4)c4ccc5c(c4)C(c4ccccc4)(c4ccccc4)c4cccc(-c6ccccc6)c4-5)cc2C3(c2ccccc2)c2ccccc2)cc1] 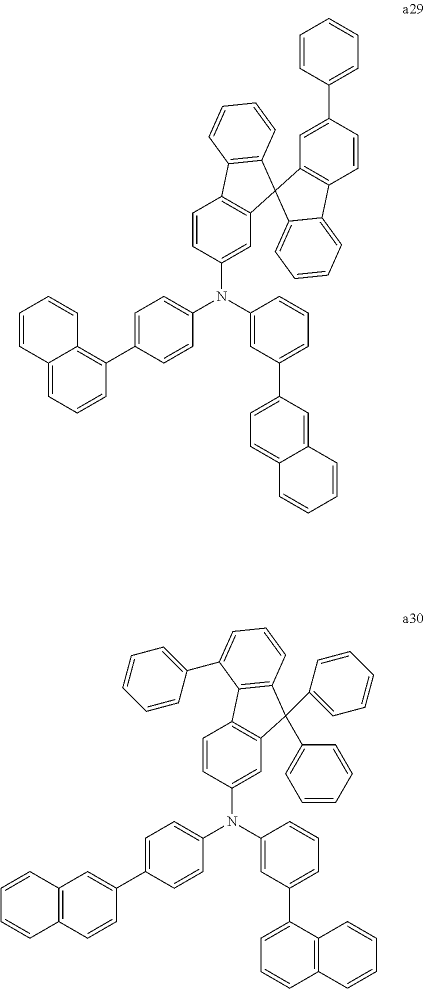 [SMILES: c1ccc(-c2ccc3c(c2)C2(c4ccccc4-3)c3ccccc3-c3ccc(N(c4ccc(-c5cccc6ccccc56)cc4)c4cccc(-c5ccc6ccccc6c5)c4)cc32)cc1 ; c1ccc(-c2cccc3c2-c2ccc(N(c4ccc(-c5ccc6ccccc6c5)cc4)c4cccc(-c5cccc6ccccc56)c4)cc2C3(c2ccccc2)c2ccccc2)cc1] 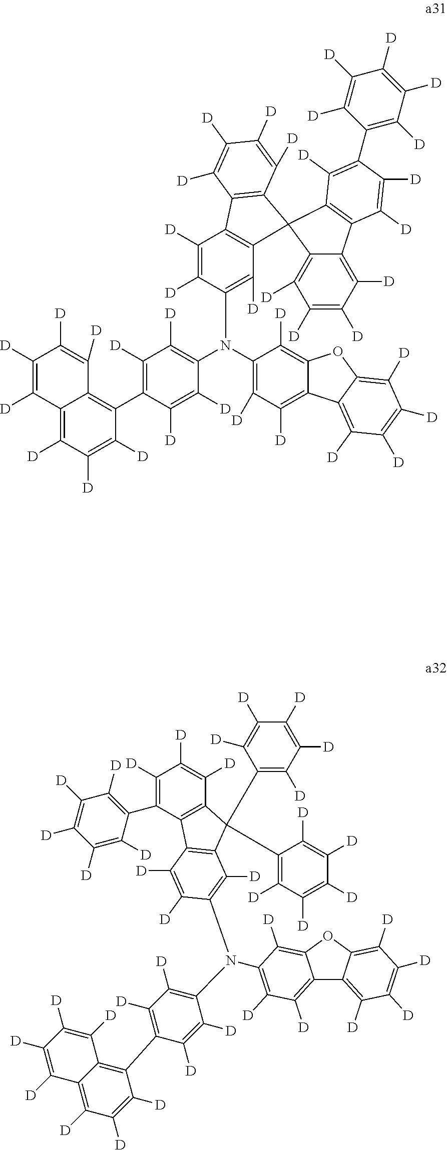 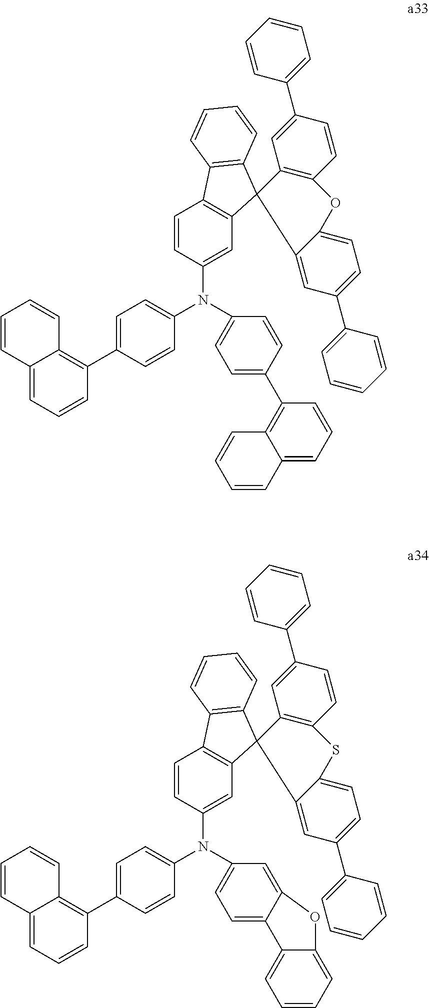 [SMILES: c1ccc(-c2ccc3c(c2)C2(c4cc(-c5ccccc5)ccc4O3)c3ccccc3-c3ccc(N(c4ccc(-c5cccc6ccccc56)cc4)c4ccc(-c5cccc6ccccc56)cc4)cc32)cc1 ; c1ccc(-c2ccc3c(c2)C2(c4cc(-c5ccccc5)ccc4S3)c3ccccc3-c3ccc(N(c4ccc(-c5cccc6ccccc56)cc4)c4ccc5c(c4)oc4ccccc45)cc32)cc1] 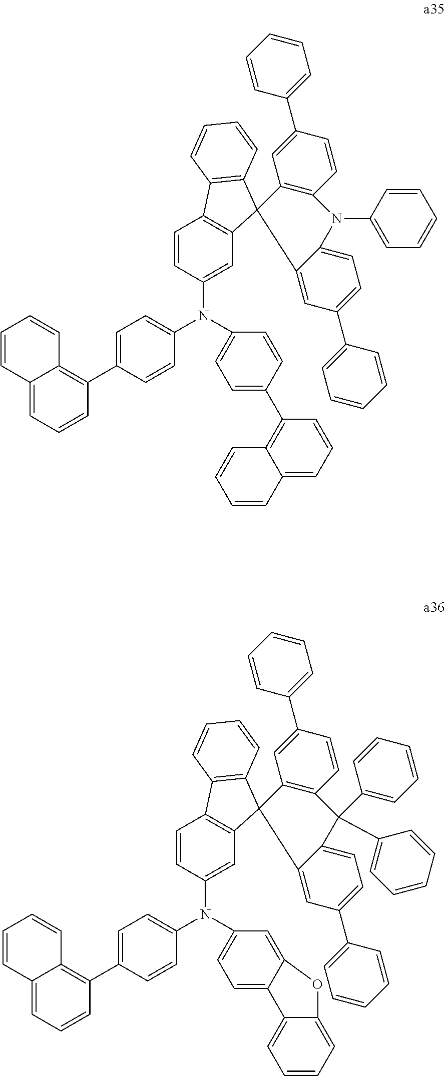 [SMILES: c1ccc(-c2ccc3c(c2)C2(c4ccccc4-c4ccc(N(c5ccc(-c6cccc7ccccc67)cc5)c5ccc(-c6cccc7ccccc67)cc5)cc42)c2cc(-c4ccccc4)ccc2N3c2ccccc2)cc1 ; c1ccc(-c2ccc3c(c2)C2(c4ccccc4-c4ccc(N(c5ccc(-c6cccc7ccccc67)cc5)c5ccc6c(c5)oc5ccccc56)cc42)c2cc(-c4ccccc4)ccc2C3(c2ccccc2)c2ccccc2)cc1] 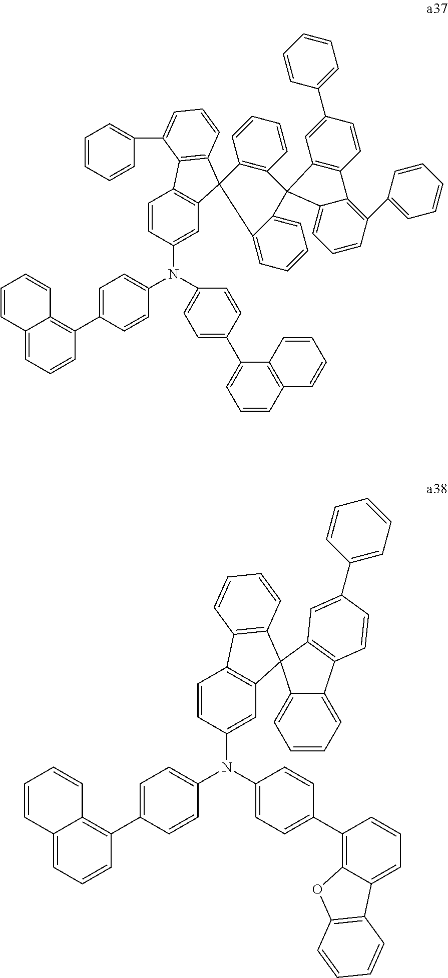 [SMILES: c1ccc(-c2ccc3c(c2)C2(c4ccccc4C4(c5cc(N(c6ccc(-c7cccc8ccccc78)cc6)c6ccc(-c7cccc8ccccc78)cc6)ccc5-c5c(-c6ccccc6)cccc54)c4ccccc42)c2cccc(-c4ccccc4)c2-3)cc1 ; c1ccc(-c2ccc3c(c2)C2(c4ccccc4-3)c3ccccc3-c3ccc(N(c4ccc(-c5cccc6ccccc56)cc4)c4ccc(-c5cccc6c5oc5ccccc56)cc4)cc32)cc1] 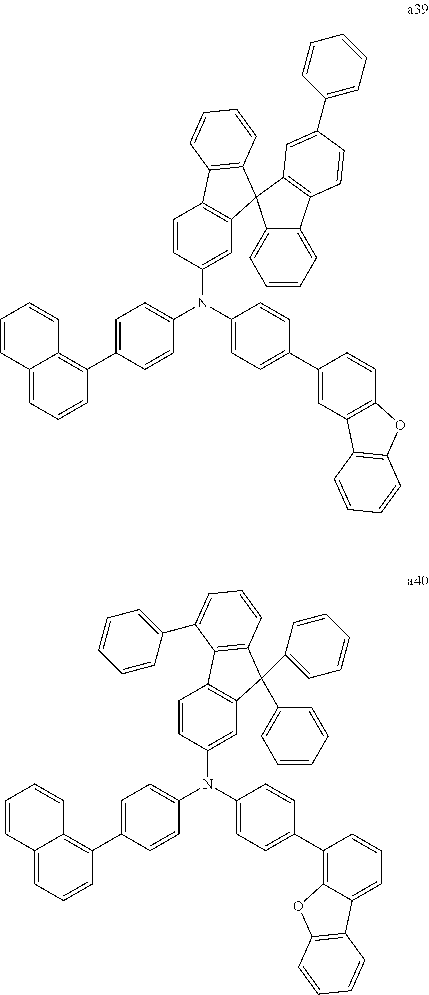 [SMILES: c1ccc(-c2ccc3c(c2)C2(c4ccccc4-3)c3ccccc3-c3ccc(N(c4ccc(-c5ccc6oc7ccccc7c6c5)cc4)c4ccc(-c5cccc6ccccc56)cc4)cc32)cc1 ; c1ccc(-c2cccc3c2-c2ccc(N(c4ccc(-c5cccc6ccccc56)cc4)c4ccc(-c5cccc6c5oc5ccccc56)cc4)cc2C3(c2ccccc2)c2ccccc2)cc1] 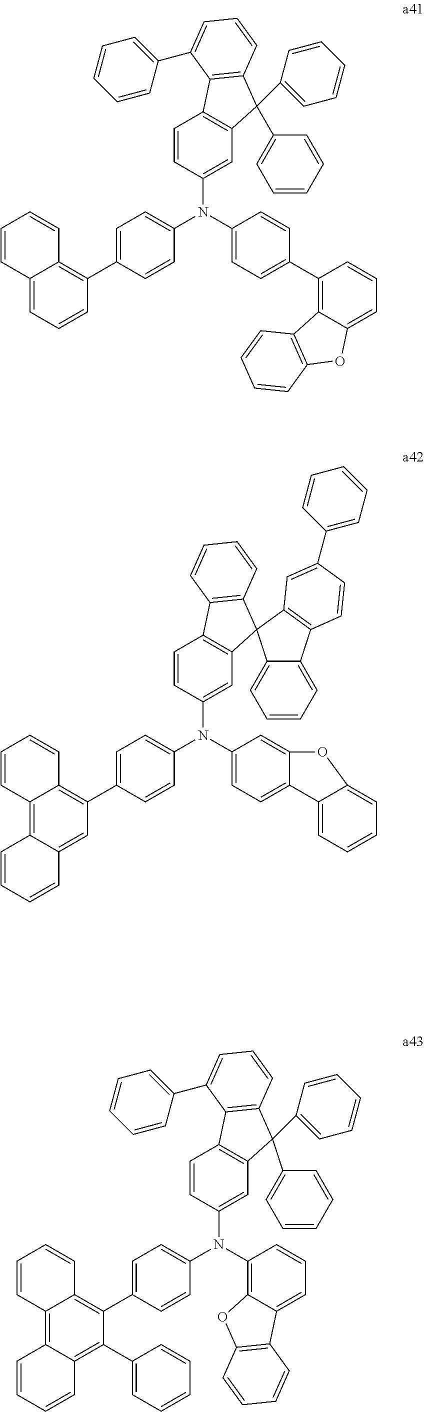 [SMILES: c1ccc(-c2cccc3c2-c2ccc(N(c4ccc(-c5cccc6ccccc56)cc4)c4ccc(-c5cccc6oc7ccccc7c56)cc4)cc2C3(c2ccccc2)c2ccccc2)cc1 ; c1ccc(-c2ccc3c(c2)C2(c4ccccc4-3)c3ccccc3-c3ccc(N(c4ccc(-c5cc6ccccc6c6ccccc56)cc4)c4ccc5c(c4)oc4ccccc45)cc32)cc1 ; c1ccc(-c2cccc3c2-c2ccc(N(c4ccc(-c5c(-c6ccccc6)c6ccccc6c6ccccc56)cc4)c4cccc5c4oc4ccccc45)cc2C3(c2ccccc2)c2ccccc2)cc1] 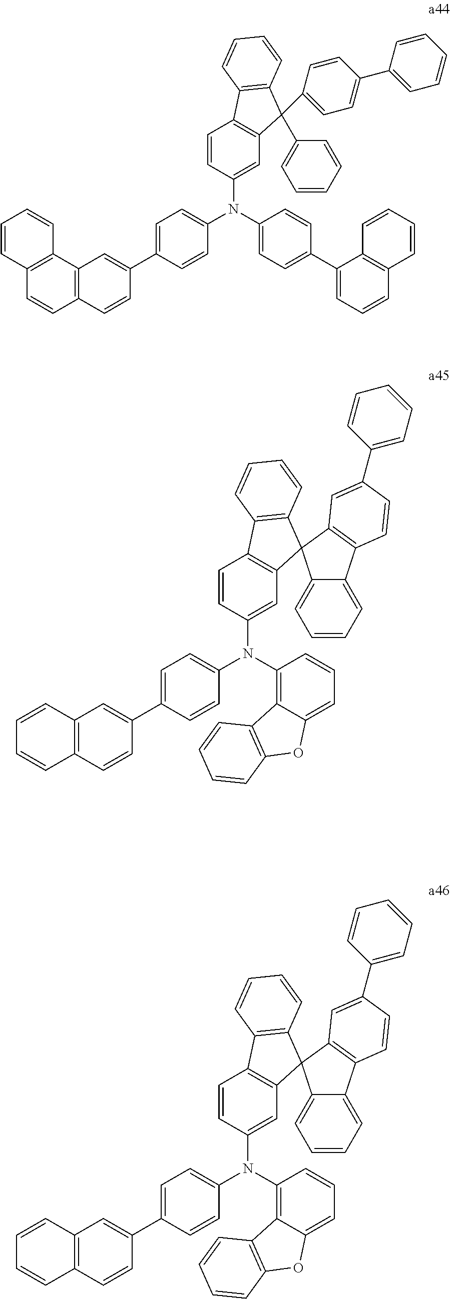 [SMILES: c1ccc(-c2ccc(C3(c4ccccc4)c4ccccc4-c4ccc(N(c5ccc(-c6ccc7ccc8ccccc8c7c6)cc5)c5ccc(-c6cccc7ccccc67)cc5)cc43)cc2)cc1 ; c1ccc(-c2ccc3c(c2)C2(c4ccccc4-3)c3ccccc3-c3ccc(N(c4ccc(-c5ccc6ccccc6c5)cc4)c4cccc5oc6ccccc6c45)cc32)cc1 ; c1ccc(-c2ccc3c(c2)C2(c4ccccc4-3)c3ccccc3-c3ccc(N(c4ccc(-c5ccc6ccccc6c5)cc4)c4cccc5oc6ccccc6c45)cc32)cc1] 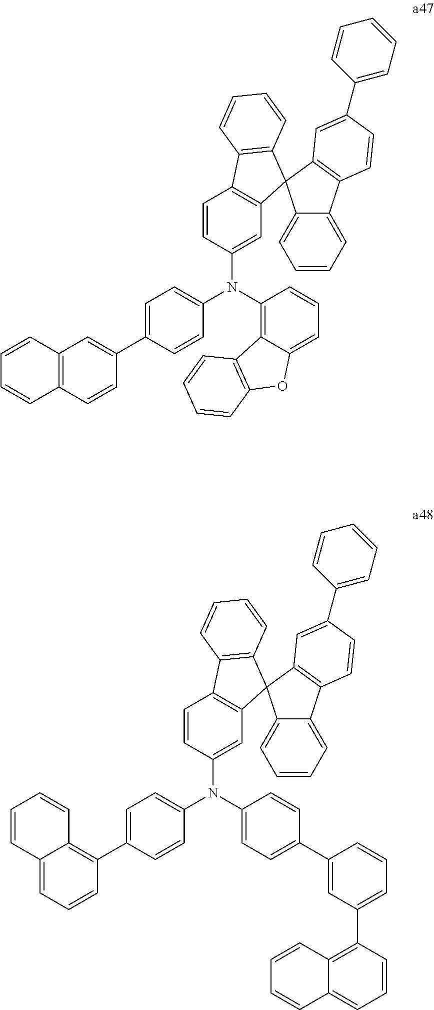 [SMILES: c1ccc(-c2ccc3c(c2)C2(c4ccccc4-3)c3ccccc3-c3ccc(N(c4ccc(-c5ccc6ccccc6c5)cc4)c4cccc5oc6ccccc6c45)cc32)cc1 ; c1ccc(-c2ccc3c(c2)C2(c4ccccc4-3)c3ccccc3-c3ccc(N(c4ccc(-c5cccc(-c6cccc7ccccc67)c5)cc4)c4ccc(-c5cccc6ccccc56)cc4)cc32)cc1] 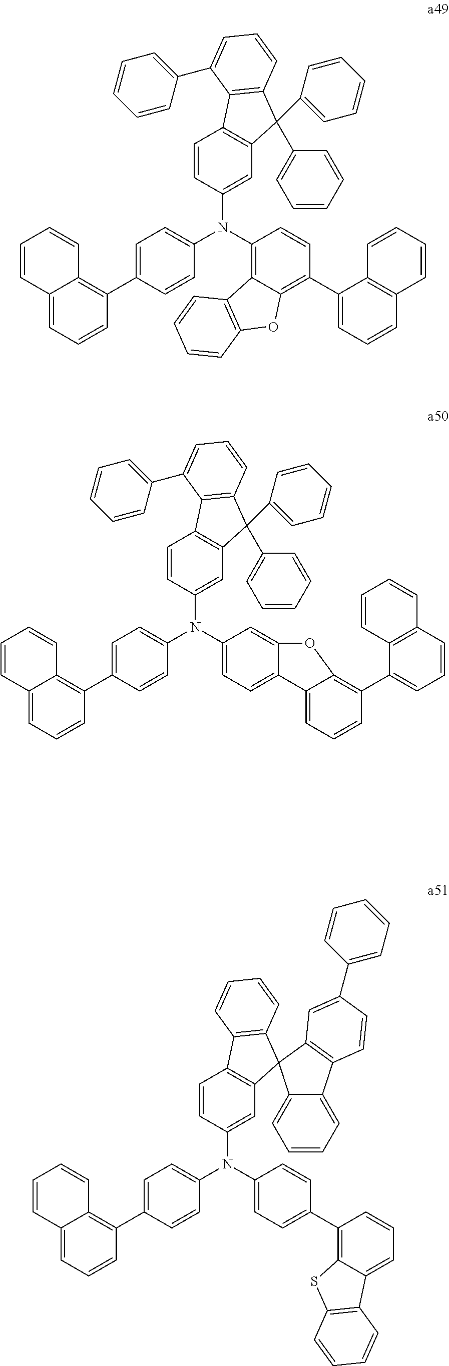 [SMILES: c1ccc(-c2cccc3c2-c2ccc(N(c4ccc(-c5cccc6ccccc56)cc4)c4ccc(-c5cccc6ccccc56)c5oc6ccccc6c45)cc2C3(c2ccccc2)c2ccccc2)cc1 ; c1ccc(-c2cccc3c2-c2ccc(N(c4ccc(-c5cccc6ccccc56)cc4)c4ccc5c(c4)oc4c(-c6cccc7ccccc67)cccc45)cc2C3(c2ccccc2)c2ccccc2)cc1 ; c1ccc(-c2ccc3c(c2)C2(c4ccccc4-3)c3ccccc3-c3ccc(N(c4ccc(-c5cccc6ccccc56)cc4)c4ccc(-c5cccc6c5sc5ccccc56)cc4)cc32)cc1] 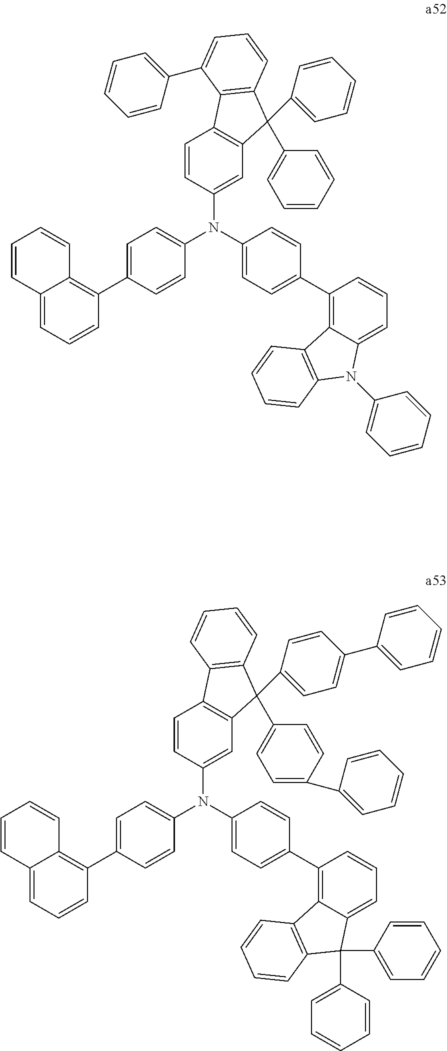 [SMILES: c1ccc(-c2cccc3c2-c2ccc(N(c4ccc(-c5cccc6ccccc56)cc4)c4ccc(-c5cccc6c5c5ccccc5n6-c5ccccc5)cc4)cc2C3(c2ccccc2)c2ccccc2)cc1 ; c1ccc(-c2ccc(C3(c4ccc(-c5ccccc5)cc4)c4ccccc4-c4ccc(N(c5ccc(-c6cccc7c6-c6ccccc6C7(c6ccccc6)c6ccccc6)cc5)c5ccc(-c6cccc7ccccc67)cc5)cc43)cc2)cc1]
wherein in Compound Group 1, D is a deuterium atom.
